(19) United States
(12) Patent Application Publication
(10) Pub. No.: US 2006/0025345 A1
(43) Pub. Date: Feb. 2, 2006

(54) Substituted ethane-1,2-diamines for the treatment of Alzheimer's disease

(75) Inventors: Stefan Peters, Biberach (DE); Christian Eickmeier, Mittelbiberach (DE); Klaus Fuchs, Schemmerhofen (DE); Werner Stransky, Gau-Algesheim (DE); Cornelia Dorner-Ciossek, Ravensburg (DE); Marcus Kostka, Biberach (DE); Sandra Handschuh, Warthausen (DE); Margit Bauer, Biberach (DE); Herbert Nar, Ochsenhausen (DE); Klaus Bornemann, Setzingen (DE); Klaus Klinder, Oggelshausen (DE); Joerg Rademann, Berlin (DE); Steffen Weik, Tuebingen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/133,948

(22) Filed: May 20, 2005

(30) Foreign Application Priority Data
May 22, 2004 (EP) 04012182

(51) Int. Cl. A61K 38/04; C07K 5/04

(52) U.S. Cl. 514/19; 564/152; 562/450

(57) ABSTRACT

The invention relates to a compound of the formula (I) 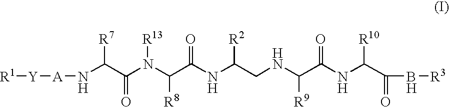
wherein R¹, R², R³, R⁴, R⁵, R⁶, R⁷, R⁸, R⁹, R¹⁰, R¹¹, R¹², R¹³, A, B, and Y are defined as in the specification and claims and to its use for treating or preventing Alzheimer's disease and other similar diseases.

This application claims the benefit under 35 U.S.C. 119(a) of German Patent Application No. 04012182, which was filed on May 22, 2004, and which is incorporated herein by reference in its entirety.

### BACKGROUND OF THE INVENTION

1. Technical Field

The invention relates to novel substituted ethylene diamines and to their use for treating or preventing Alzheimer's disease and other similar diseases.

2. Description of the Prior Art

Alzheimer's disease (AD) is a progressive degenerative disease of the brain primarily associated with aging. Clinical presentation of AD is characterized by loss of memory, cognition, reasoning, judgement, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD.

Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretases constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp2, am Memapsin2. See, for example, Sindha et. al., 1999, Nature 402: 537-554 and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6: 487-498. Release of A beta from neuronal cells grown in culture and the presence of A beta in cerebrospinal fluid (CSF) of both normal individuals and AD patients has been demonstrated. See, for example, Seubert et al., 1992, Nature 359: 325-327.

It has been proposed that A beta peptide accumulates as a result of APP processing by beta-secretase, thus inhibition of this enzyme's activity is desirable for the treatment of AD, see for example Vassar, R. 2002, Adv. Drug Deliv. Rev. 54, 1589-1602 In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1-19.

BACE1 knockout mice fail to produce A beta, and present a normal phenotype. When crossed with transgenic mice that overexpress APP, the progeny show reduced amounts of A beta in brain extracts as compared with control animals (Luo et. al., 2001 Nature Neuroscience 4: 231-232). This evidence further supports the proposal that inhibition of beta-secretase activity and reduction of A beta in the brain provides a therapeutic method for the treatment of AD and other beta amyloid disorders.

The International patent application WO0/47618 identifies the beta-secretase enzyme and methods of its use. This publication also discloses oligopeptide inhibitors that bind the enzyme's active site and are useful in affinity column purification of the enzyme. In addition, WO00/77030 discloses tetrapeptide inhibitors of beta-secretase activity that are based on a statine molecule.

Various pharmaceutical agents have been proposed for the treatment of Alzheimer's disease but without any real success. U.S. Pat. No. 5,175,281 discloses aminosteroids as being useful for treating Alzheimer's disease. U.S. Pat. No. 5,502,187 discloses bicyclic heterocyclic amines as being useful for treating Alzheimer's disease.

EP 652 009 AI discloses inhibitors of aspartyl protease which inhibit beta amyloid peptide production in cell culture and in vivo. The compounds which inhibit intracellular beta-amyloid peptide production are useful in treating Alzheimer's disease.

WO00/69262 discloses a new beta-secretase and its use in assays to screen for potential drug candidates against Alzheimer's disease. WO01/00663 discloses memapsin 2 (human beta-secretase) as well as catalytically active recombinant enzyme. In addition, a method of identifying inhibitors of memapsin 2, as well as two inhibitors are disclosed. Both inhibitors that are disclosed are peptides. WO01/00665 discloses inhibitors of memapsin 2 that are useful in treating Alzheimer's disease. Also, WO 03/057721 discloses substituted amino carboxamides for the treatment of Alzheimer's disease.

At present there are no effective treatments for halting, preventing, or reversing the progression of Alzheimer's disease. Therefore, there is an urgent need for pharmaceutical agents with sufficient plasma and/or brain stability capable of slowing the progression of Alzheimer's disease and/or preventing it in the first place.

Compounds that are effective inhibitors of beta-secretase, that inhibit beta secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

### BRIEF SUMMARY OF THE INVENTION

Surprisingly, it has been found that substituted ethane-1,2-diamines of formula (I) show superior inhibition of beta secretase-mediated cleavage of APP and sufficient plasma stability.

Thus the invention relates in a first embodiment to a compound of the formula (I) 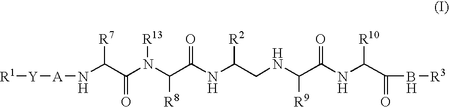
wherein
R¹ represents
   H, an alkyl-, alkenyl-, alkynyl-, haloalkyl-, alkoxy-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group, wherein
      each of said groups may be optionally substituted by one or more substituents independently selected from the group consisting of alkyl-, heteroaryl-, benzoyl-, haloalkyl-, alkoxy-, alkoxycarbonyl-, haloalkoxy-, halogen-, carboxy-, acetyl-, acylamino-, carboxyalkylaminoalkyl-, formyl-, hydroxy-, cyano-, nitro-, oxo-, —N(R⁴)₂, -alkyl-N(R⁴)₂, alkylsulfonylamino-, —SO₂-alkyl, —SO₂NH₂, —SO₃H, —SO₃N(R⁴)₂, and in case that the heteroaryl-, heteroaryl-alkyl-, or the heteroaryl-alkenyl-group contains a nitrogen atom in the ring, said nitrogen may be optionally oxidized to the corresponding N-oxide,
R² represents
   an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group,
      each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
R³ represents —OH or —N(R⁵)₂;
R⁴ each independently represent
   H, an alkyl-, haloalkyl-, alkoxy-, alkenyl-, cycloalkyl-, cycloalkyl-alkyl-, heterocyclyl-, heterocyclyl-alkyl-, aryl-, aryl-alkyl-, heteroaryl-, or a heteroaryl-alkyl-group,
      each of said groups may be optionally substituted by one or more substituents independently selected from the group consisting of alkyl-, alkoxy-, haloalkoxy-, halogen-, carboxy-, hydroxy-, cyano-, —N(R⁶)₂, —SO₂N(R⁶)₂,
R⁵ each independently represent
   H, an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
Y is absent if A is present or represents a carbonyl group if A is absent,
R⁷ represents
   an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group,
      each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —S(R⁴), —SO₃H, —SO₃N(R⁴)₂,
R⁸ represents
   an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, a heteroaryl-alkyl-, aryl-alkenyl-, or a heteroaryl-alkenyl-group,
      each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —CO—N(R⁴)₂, —S(R⁴), —SO—R⁴, —SO₃H, —SO₂-alkyl, —SO₃N(R⁴)₂,
R⁹ represents
   an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, heteroaryl-alkenyl-group,
      each of said groups may be optionally substituted by one or more substituents independently selected from the group consisting of alkyl-, alkoxy-, haloalkoxy-, halogen-, carboxy-, formyl-, hydroxy-, cyano, nitro, —N(R⁴)₂, -alkyl-N(R⁴)₂, —SO₃H, —SO₃N(R⁴)₂, —SO₂NH₂,
R¹⁰ represents
   an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group,
      each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
A is absent or represents a group of formula (II): 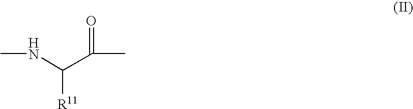
which is linked N-terminal via —NH— with Y, and C-terminal via —CO— with 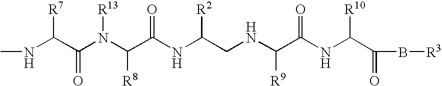
and wherein
   R¹¹ represents
      an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-group, aryl-group, heteroaryl-, aryl-alkyl-, heteroaryl-alkyl-, aryl-alkenyl-, or a heteroaryl-alkenyl-group,
         each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
B is absent or represents a group of formula (III): 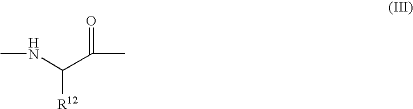
which is linked C-terminal via —CO— with R³ and N-terminal via —NH— with 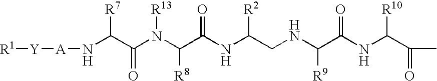
and wherein
   R¹² represents
      an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-group, or a heteroaryl-alkenyl-group,
      each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
R¹³ represents
   H, an alkyl-, alkenyl-, alkynyl-, cycloalkyl- or cycloalkyl-alkyl-group,
      each of said groups may be optionally substituted by one or more substituents independently selected from the group consisting of alkoxy-, fluoroalkoxy-, fluor- and cyano-,
         or pharmaceutically acceptable tautomers, enantiomers, diastereomers, salts or solvates thereof.

Furthermore, the invention relates to a pharmaceutical composition comprising a compound of formula (I) or a pharmaceutically acceptable salt or solvate thereof and a pharmaceutically acceptable carrier or diluent.

Another aspect of the present invention is the use of a compound of formula (I) or a pharmaceutically acceptable salt or solvate thereof in the manufacture of a medicamentation for use in treating a patient who has, or in preventing a patient from getting, a disease or condition selected from Alzheimer's disease, diffuse Lewy body type of Alzheimer's disease, Down's syndrome, MCI (“Mild Cognitive Impairment”), Heriditary Cerebral Hemorrhage with Amyloidosis of the Dutch-Type, Cerebral Amyloid Angiopathy, Traumatic Brain Injury, Stroke, Dementia, Parkinson's Disease and Parkinson's Syndrome, or central or peripheral amyloid diseases.

Furthermore the invention relates to a method for inhibiting β-secretase activity, comprising exposing said β-secretase to an effective inhibitory amount of a compound of formula I.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating patients with mild cognitive impairment (MCI), and preventing or delaying the onset of AD in those patients who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

The compounds of the invention possess beta-secretase inhibitory activity.

The inhibitory activities of the compounds of the invention are readily demonstrated, for example, using one or more of the assays described herein or known in the art.

### DETAILED DESCRIPTION OF THE INVENTION

The present invention are the substituted ethane-1,2-diamines of formula (I) that are useful in treating and preventing Alzheimer's disease.

Some expressions used hereinbefore and below to describe the compounds according to the invention will now be defined more fully.

The term alkyl in the present invention denotes, unless otherwise stated, a unbranched or branched hydrocarbon group having 1-8 carbon atoms, preferably 1 to 6 carbon atoms, most preferably 1 to 5 carbon atoms. Examples are methyl, ethyl, propyl, n-butyl, pentyl, hexyl etc. Unless otherwise stated the above terms propyl, isopropyl, butyl, 2-butyl, isobutyl, 2,2-dimethylpropyl, 3-methylbutyl, pentyl, or hexyl also include all the possible isomeric forms like isopropyl, sec. butyl, tert-butyl, 2-pentyl, isopentyl, neopentyl, 2-hexyl, 3-hexyl, and 3-methylpentyl. In some cases common abbreviations are also used to denote the above mentioned alkyl groups, such as Me for methyl, Et for ethyl etc.

The term haloalkyl (including those which are part of other groups, especially haloalkoxy) denotes, unless otherwise stated, branched or unbranched alkyl groups with 1 to 8 carbon atoms, preferably 1 to 6 carbon atoms, more preferably 1 to 5 carbon atoms, which are substituted by at least one halogen atom, particularly fluorine atom. Fluorinated groups of the formula
—(CH₂)ₚ—(CF₂)_{q}-Z
wherein
p denotes 0 or an integer from 1 to 3,
q denotes an integer from 1 to 3, and
Z denotes hydrogen or fluorine, are preferred.

Examples include: trifluoromethyl, trifluoromethoxy, difluoromethoxy, perfluoroethyl, perfluoropropyl, 2,2,2-trifluoroethyl, 2,2,2-trifluoroethoxy, 1,1,1-trifluoroprop-2-yl, etc.

The term halogen generally denotes fluorine, chlorine, bromine or iodine particularly F, Cl and Br.

The term alkoxy denotes an alkyl-O— group, wherein alkyl is defined as above. Examples are methoxy, ethoxy, propoxy, isopropoxy, n-butoxy, sec-butoxy, tert-butoxy, pentoxy, isopentoxy, neopentoxy, hexoxy, and 3-methylpentoxy.

The term alkenyl denotes, unless otherwise stated, branched or unbranched hydrocarbon groups having from 2 to 6 carbon atoms, preferably 1 to 4 carbon atoms, most preferably 1 to 3 carbon atoms and from one to three double bonds and includes, for example, ethenyl, propenyl, allyl, 1-butenyl, 1-pentenyl, 1-hexenyl and the like.

The term alkynyl denotes, unless otherwise stated, branched or unbranched hydrocarbon groups having from 2 to 6 carbon atoms, preferably 1 to 4 carbon atoms, most preferably 1 to 3 carbon atoms and one or two triple bonds and includes ethynyl, propynyl, propargyl, butynyl, pentynyl and the like.

The term cycloalkyl (including those which are part of other groups, especially cycloalkyl-alkyl- or cycloalkoxy-) denotes, unless otherwise stated, cyclic alkyl groups with 3 to 8 carbon atoms, preferably 3 to 6 carbon atoms, most preferably 3, 5 or 6 carbon atoms. The cycloalkyl group can be monocyclic, or a polycyclic fused system. Examples are: cyclopropyl, cyclobutyl, cyclopentyl, cyclohexyl, cycloheptyl etc. Preferably, the term C₃₋₇-cycloalkyl includes saturated monocyclic groups. Most preferred is cyclopropyl and cyclohexyl.

The term aryl group, unless otherwise stated, denotes an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl). Examples are: phenyl-, biphenyl-, 1-naphthyl, 2-naphthyl, indanyl, indenyl, dihydronaphthyl, tetralinyl, anthracenyl, phenanthrenyl, fluorenyl, pentalenyl, azulenyl, biphenylenyl. A particularly preferred meaning of “aryl” is phenyl.

The term heteroaryl group, unless otherwise stated, denotes one or more aromatic ring systems of 5-, 6-, or 7-membered rings which includes fused ring systems of 9-11 atoms containing at least one and up to four heteroatoms selected from nitrogen, oxygen, or sulfur. The term heteroaryl group embraces also heteroaryl groups containing an oxidized nitrogen atom in the ring (N-oxides). Examples are: pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyridin-2-yl-N-oxide, pyridin-3-yl-N-oxide, pyridin-4-yl-N-oxide, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, benzo[1,3]dioxol.

Within the present invention the term N-oxide designates compounds wherein the nitrogen atom in the ring of a heteroaryl-, heteroaryl-alkyl- or heteroaryl-alkeny Igroup is oxidized by oxygen. For example, the term pyridin-4-yl-N-oxide designates the following group:

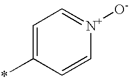

The term pyridin-3-yl-N-oxide designates the following group:

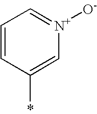

And the term pyridin-2-yl-N-oxide designates the following group:

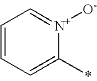

The term heterocyclyl group, unless otherwise stated, denotes one or more carbocyclic ring systems of 3-, 4-, 5-, 6-, or 7-membered rings which includes fused ring systems of 9-11 atoms containing at least one and up to four heteroatoms selected from nitrogen, oxygen, or sulfur. Preferred heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, morpholinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, azepanyl, diazepanyl, tetrahydrothienyl S-oxide, tetrahydrothienyl S,S-dioxide and homothiomorpholinyl S-oxide.

Terms such as cycloalkyl-alkyl-, heterocyclyl-alkyl-, aryl-alkyl-, heteroaryl-alkyl-, cycloalkyl-alkenyl-, heterocyclyl-alkenyl-, aryl-alkenyl-, heteroaryl-alkenyl-, refer to alkyl or alkenyl respectively, as defined above, which is substituted with a cycloylkyl, heterocyclyl, aryl or heteroaryl group. Examples of aryl-alkyl-groups are benzyl or phenylethyl. Examples for cycloalkyl-alkyl-groups are cyclopropylmethyl, cyclohexylmethyl or cyclopentylethyl.

Many of the terms given above may be used repeatedly in the definition of a formula or group and in each case have one of the meanings given above, independently of one another.

The term “optionally substituted” used in this application indicates that the group thus designated is either unsubstituted or mono- or polysubstituted by the substituents specified. If the group in question is polysubstituted, the substituents may be identical or different.

The compounds of the present invention contain asymmetric carbon atoms and may be present in the form of one of the possible isomers or as a mixture thereof, e.g. depending on the number, absolute and relative configurations of the asymmetric carbon atoms as pure isomers, such as antipodes and/or diastereoisomers, or as isomeric mixtures, such as enantiomeric mixtures, e.g. racemates, diastereoisomeric mixtures or racemic mixtures; the invention relates to both the pure isomers and all the possible isomeric mixtures, and is to be understood as such hereinbefore and hereinafter, even if stereochemical details are not specifically mentioned in each case.

The symbol “−” in general represents a bond between two atoms in a chain and the point of attachment of a group to the rest of the molecule as defined. For example, an aryl-alkyl-group (e.g. 2-phenylethyl-) indicates an group wherein the phenyl group is attached to the ethyl group and the ethyl group is attached to the rest of the molecule. The numeration of the atoms of a substituent starts with the atom which is closest to the rest of the moelcule to which the substituent is attached.

For example, the term “3-carboxypropyl-group” represents the following substituent: 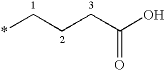
wherein the carboxy group is attached to the third carbon atom of the propyl group. The terms “2-butyl-”, “2,2-dimethylpropyl-” or “cyclopropylmethyl-” group represent the following groups:

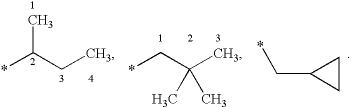

The asterisk is used in sub-formulas to indicate the bond which is connected to the rest of the molecule as defined.

Preferred are the compounds of the first embodiment of formula (I), wherein
A and B are absent, and
Y represents a carbonyl group,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, benzyl-, 4-hydroxybenzyl-, 2-phenylethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-butyl-, isobutyl-, propyl-, carbamoylmethyl-, 2-carbamoylethyl-, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-methanesulfinylethyl-, 2-cyanoethyl-, 2-phenylethyl-, benzyl-, methyl-, butyl-, 2,2-dimethylpropyl-, 3,3,3-trifluoropropyl-, methoxymethyl-, 3-methylbutyl-, cyanomethyl-, methanesulfonylmethyl-, 2-methanesulfonylethyl-, 2-methoxyethyl-, cyclopentyl-, propargyl-, benzyloxymethyl-, hydroxymethyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

Furthermore preferred are the compounds of the first embodiment of formula (I), wherein
A and B are absent, and
Y represents a carbonyl group,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, methyl-, 3-thienylmethyl-, 4-thiazolylmethyl-, 5-thiazolylmethyl-, 4-imidazolylmethyl-, 2-pyridinylmethyl-, 3-pyridinylmethyl-, or a 4-pyridinylmethylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, 2-butyl-, isobutyl-, propyl-, carbamoylmethyl, 2-carbamoylethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, methanesulfonylmethyl-, 2-methanesulfonyl-ethyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Furthermore preferred are the compounds of the first embodiment of formula (I), wherein
A and B are absent, and
Y represents a carbonyl group,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R⁷ represents
   a methyl-, 2-butyl-, 3-methylbutyl-, 2-phenylethyl-, methylthiomethyl-, allyl-ethyl-, propyl-, cyclopropylmethyl-, 2,2-dimethylpropyl-, 2-cyanoethyl-, benzyl-, cyclohexyl-, phenyl-, isopropyl- or a isobutylgroup,
R⁸ represents
   a cyclopropylmethyl-, methylthiomethyl-, 2-methylthioethyl-, allyl-, isobutyl, 2-butyl-, 3-methylbutyl-, 2-methoxyethyl-, methoxymethyl-, cyclopentyl-, 2-cyanoethyl-, 3,3,3-trifluoropropyl-, ethyl-, isopropyl-, methyl-, butyl-, 2-methanesulfinylethyl-, methanesulfonylmethyl-, 2-methanesulfonylethyl-, carbamoylmethyl-, 2-carbamoylethyl-, hydroxymethyl-, phenyl-, benzyl-, n-propyl or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup,
      or pharmaceutically acceptable tautomers, enantiomers, diastereomers, salts or solvates thereof.

Furthermore preferred are the compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
         or pharmaceutically acceptable tautomers, enantiomers, diastereomers, salts or solvates thereof.

Furthermore preferred are the compounds of the first embodiment of formula (I), wherein
A and B are absent, and
Y represents a carbonyl group,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
   or pharmaceutically acceptable tautomers, enantiomers, diastereomers, salts or solvates thereof.

Furthermore preferred are the compounds of the first embodiment of formula (I), wherein
A and B are absent, and
Y represents a carbonyl group,
R¹ represents
   a pyridin-2-yl-N-oxide, pyridin-3-yl-N-oxide, pyridin-4-yl-N-oxide, carboxyalkyl-, or a phenylgroup,
      wherein the phenylgroup may be optionally substituted by one or more substituents independently selected from the group consisting of carboxygroups or halogen atoms, preferably fluor atoms and hydroxy groups, and
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
         or pharmaceutically acceptable tautomers, enantiomers, diastereomers, salts or solvates thereof.

Also more preferred are the compounds of the first embodiment of formula (I), wherein
A and B are absent,
Y represents a carbonyl group,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
      or pharmaceutically acceptable tautomers, enantiomers, diastereomers, salts or solvates thereof.

Furthermore preferred are also such compounds of the first embodiment of formula (I), wherein
A and B are absent, and
Y represents a carbonyl group,
R¹ represents
   a pyridin-4-yl-N-oxide, 4-hydroxy-2,3,5,6-tetrafluorophenyl- or a 3-carboxypropyl-group,
      wherein the pyridin-4-yl-N-oxide group may be optionally substituted by one substituent selected from the group consisting of fluor, chlor, methyl and/or trifluoromethyl,
         or pharmaceutically acceptable tautomers, enantiomers, diastereomers, salts or solvates thereof.

More preferred are also compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

More preferred are also such compounds of the first embodiment of formula (I), wherein
A and B are absent, and
Y represents a carbonyl group,
R¹ represents
   a pyridin-2-yl-N-oxide, pyridin-3-yl-N-oxide, pyridin-4-yl-N-oxide, carboxyalkyl-, or a phenylgroup,
      wherein the phenylgroup may be optionally substituted by one or more substituents independently selected from the group consisting of hydroxy groups, carboxy groups and halogen atoms, preferably fluor atoms and hydroxy groups, and
      wherein the pyridin-2-yl-N-oxide, pyridin-3-yl-N-oxide and pyridin4-yl-N-oxide groups may be optionally substituted by one or more substituents independently selected from the group consisting of fluor, chlor, methyl and/or trifluoromethyl,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl-, or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

More preferred are also such compounds of the first embodiment of formula (I), wherein
A and B are absent, and
Y represents a carbonyl group,
R¹ represents
   a pyridin-4-yl-N-oxide, 4-hydroxy-2,3,5,6-tetrafluorophenyl- or a 3-carboxypropyl-group,
      wherein the pyridin-4-yl-N-oxide group may be optionally substituted one substituent selected from the group consisting of fluor, chlor, methyl and/or trifluoromethyl,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

More preferred are also such compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

More preferred are also such compounds of the first embodiment of formula (I), wherein
A and B are absent, and
Y represents a carbonyl group,
R¹ represents
   a pyridin-2-yl-N-oxide, pyridin-3-yl-N-oxide, pyridin-4-yl-N-oxide, carboxyalkyl-, or a phenylgroup,
      wherein the phenylgroup may be optionally substituted by one or more substituents independently selected from the group consisting of hydroxy groups, carboxy groups and halogen atoms, preferably fluor atoms and hydroxy groups, and
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

More preferred are also such compounds of the first embodiment of formula (I), wherein
A and B are absent, and
Y represents a carbonyl group,
R¹ represents
   a pyridin-4-yl-N-oxide, 4-hydroxy-2,3,5,6-tetrafluorophenyl- or a 3-carboxypropyl-group,
      wherein the pyridin-4-yl-N-oxide group may be optionally substituted by one substituent selected from the group consisting of fluor, chlor, methyl and/or trifluoromethyl,
R² represents
   a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
      wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
   a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl-, or an allylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
   an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
   a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
      wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
         or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl- or a methylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridin-2-yl-N-oxide, pyridin-3-yl-N-oxide, pyridin-4-yl-N-oxide, carboxyalkyl-, or a phenylgroup,
      wherein the phenylgroup may be optionally substituted by one or more substituents independently selected from the group consisting of hydroxy groups, carboxy groups and halogen atoms, preferably fluor atoms and hydroxy groups, and
      wherein the pyridin-2-yl-N-oxide, pyridin-3-yl-N-oxide and pyridin4-yl-N-oxide groups may be optionally substituted by one or more substituents independently selected from the group consisting of fluor, chlor, methyl and/or trifluoromethyl,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, or a methylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group, each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂;
R⁶ each independently represents H or an alkyl group
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridine-4-yl N-oxide, 4-hydroxy-2,3,5,6-tetrafluorophenyl- or a 3-carboxypropyl-group,
      wherein the pyridin-4-yl-N-oxide group may be optionally substituted by one substituent selected from the group consisting of fluor, chlor, methyl and/or trifluoromethyl,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl- or a methylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, or a methylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridin-2-yl N-oxide, pyridin-3-yl N-oxide, pyridin4-yl N-oxide, carboxyalkyl-, or a phenylgroup,
      wherein the phenylgroup may be optionally substituted by one or more substituents independently selected from the group consisting of hydroxy groups, carboxy groups and halogen atoms, preferably fluor atoms and hydroxy groups, and
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl- or a methylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin4-ylmethyl-, 4-acetylaminobenzyl- or a 2-methylsulfonylbenzyl-group,
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridin-4-yl N-oxide, 4-hydroxy-2,3,5,6-tetrafluorophenyl- or a 3-carboxypropyl-group,
      wherein the pyridin-4-yl-N-oxide group may be optionally substituted by one substituent selected from the group consisting of fluor, chlor, methyl and/or trifluoromethyl,
R² represents
   a 2-butyl-, isobutyl-, cyclohexylmethyl-, 2,2-dimethylpropyl-, benzyl-, 4-hydroxybenzyl-, propyl-, ethyl-, butyl-, cyclopropylmethyl, cyclopentylmethyl-, cyclobutylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl- or a methylgroup,
      wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R 5),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
      wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
   a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
   a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
   a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl N-oxide, pyridin-3-yl N-oxide, pyridin-4-yl N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group, each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
   a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
   a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridin-2-yl N-oxide, pyridin-3-yl N-oxide, pyridin-4-yl N-oxide, carboxyalkyl-, or a phenylgroup,
      wherein the phenylgroup may be optionally substituted by one or more substituents independently selected from the group consisting of hydroxy groups, carboxy groups and halogen atoms, preferably fluor atoms and hydroxy groups, and
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
   a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
   a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup,
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridin-4-yl N-oxide, 4-hydroxy-2,3,5,6-tetrafluorophenyl- or a 3-carboxypropyl-group,
      wherein the pyridin-4-yl-N-oxide group may be optionally substituted by one substituent selected from the group consisting of fluor, chlor, methyl and/or trifluoromethyl,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-,benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
   an alkyl-, aryl-, heteroaryl-, cycloalkyl-alkyl-, heterocyclyl-alkyl-, aryl-alkyl-, or a heteroaryl-alkyl-group,
      each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
   a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
   a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl N-oxide, pyridin-3-yl N-oxide, pyridin-4-yl N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
   a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup
      or pharmaceutically acceptable tautomers, enantiomers, diastereomers, salts or solvates thereof.

Even more preferred are also such compounds of the first embodiment of formula (I), wherein
A and B are absent, and
Y represents a carbonyl group,
R¹ represents
   a pyridin-2-yl N-oxide, pyridin-3-yl N-oxide, pyridin-4-yl N-oxide, carboxyalkyl-, or a phenylgroup,
      wherein the phenylgroup may be optionally substituted by one or more substituents independently selected from the group consisting of hydroxy groups, carboxy groups and halogen atoms, preferably fluor atoms and hydroxy groups, and
      wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
   a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup
      or pharmaceutically acceptable tautomers, enantiomers, diastereomers, salts or solvates thereof.

Further preferred are such compounds of the first embodiment of formula (I), wherein
A and B are absent, and
Y represents a carbonyl group,
R¹ represents
   a pyridin-4-yl N-oxide, 4-hydroxy-2,3,5,6-tetrafluorophenyl- or a 3-carboxypropyl-group,
      wherein the pyridin-4-yl-N-oxide group may be optionally substituted by one substituent selected from the group consisting of fluor, chlor, methyl and/or trifluoromethyl,
R² represents
   a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
   an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
   a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
   a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
   a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
   a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup
      or pharmaceutically acceptable tautomers, enantiomers, diastereomers, salts or solvates thereof.

Further preferred are those compounds according to formula (I) as described above wherein
R¹³ represents
   H, an alkyl-, alkenyl- or a cycloalkyl-group,
      each of said groups may be optionally substituted by one or more substituents independently selected from the group consisting of alkoxy-, fluoroalkoxy-, fluor-, and cyano-.

More preferred are those compounds according to formula (I) as described above wherein
R¹³ represents
   H or a methyl-group.

Even more preferred are those compounds according to formula (I) as described above wherein
R¹³ represents methyl.

Most preferred are the compounds of formulae (1) through (175):

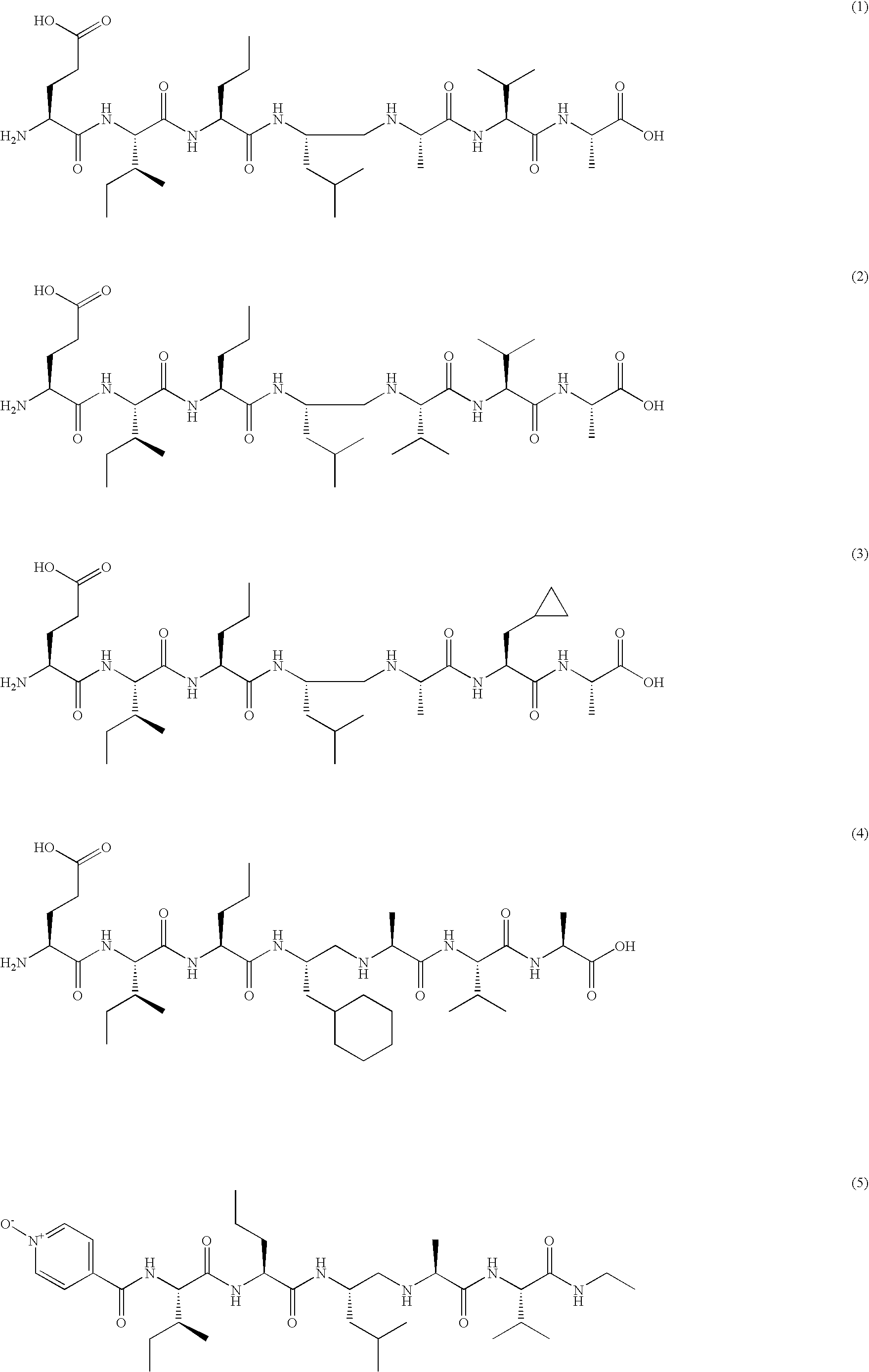

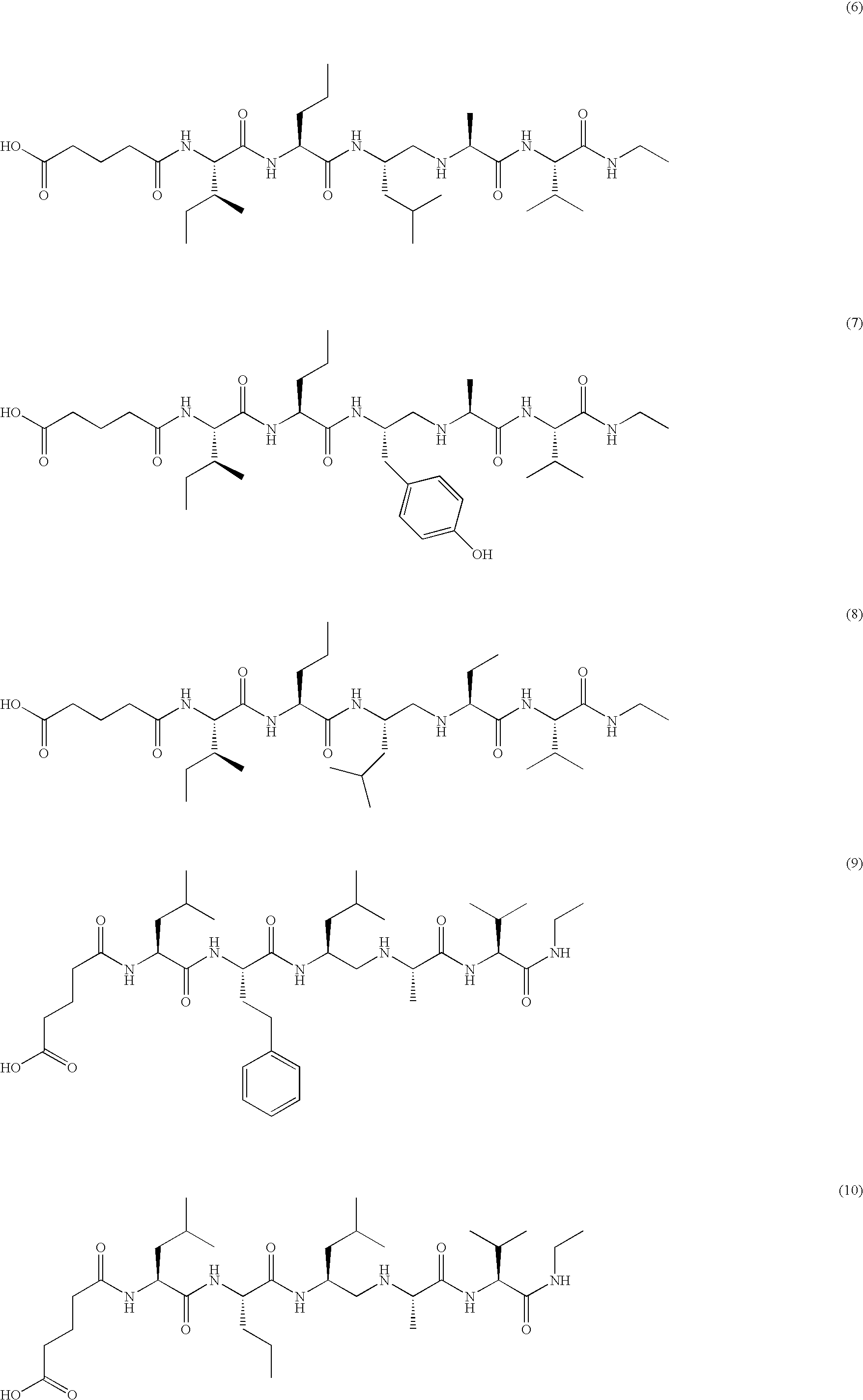

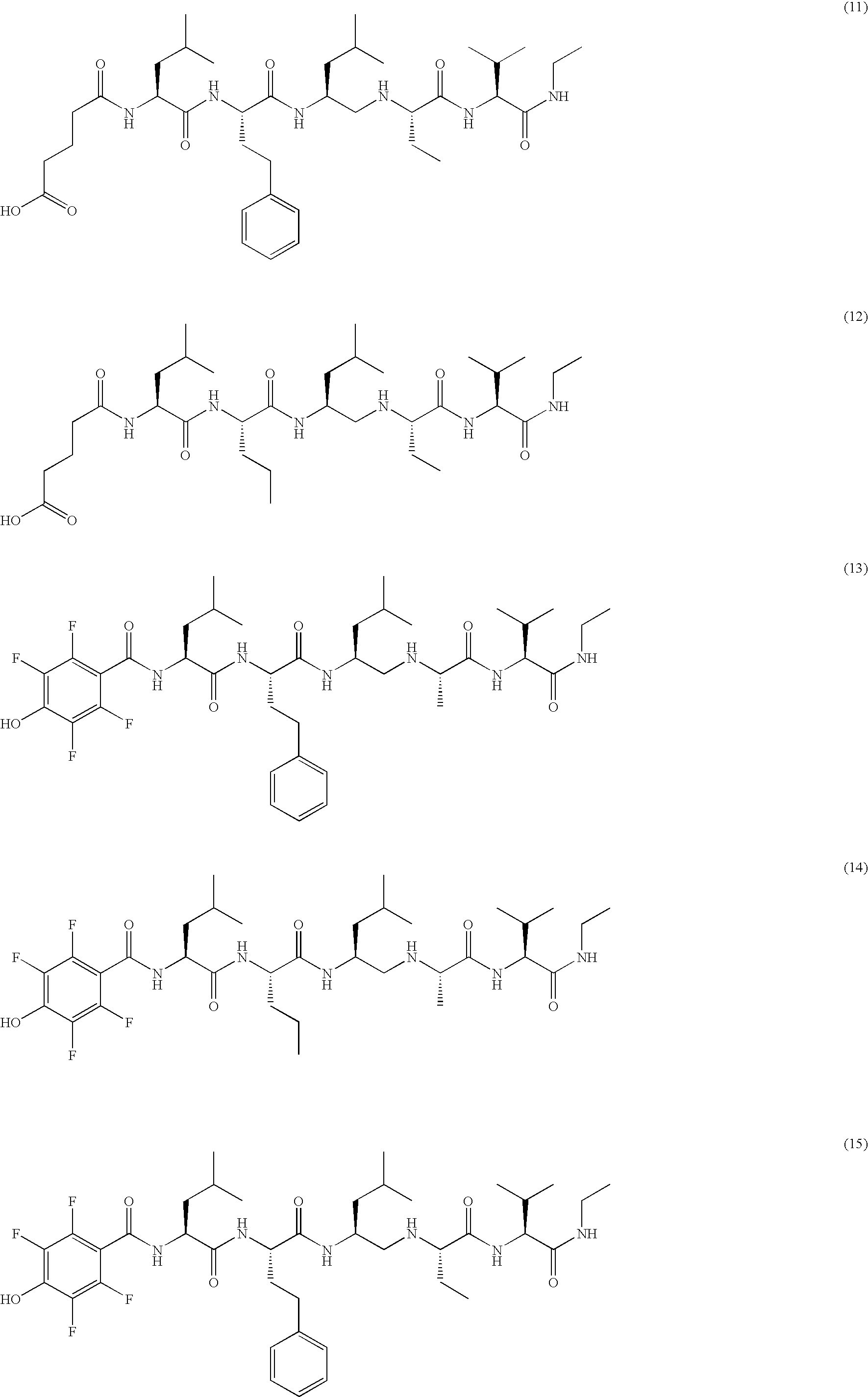

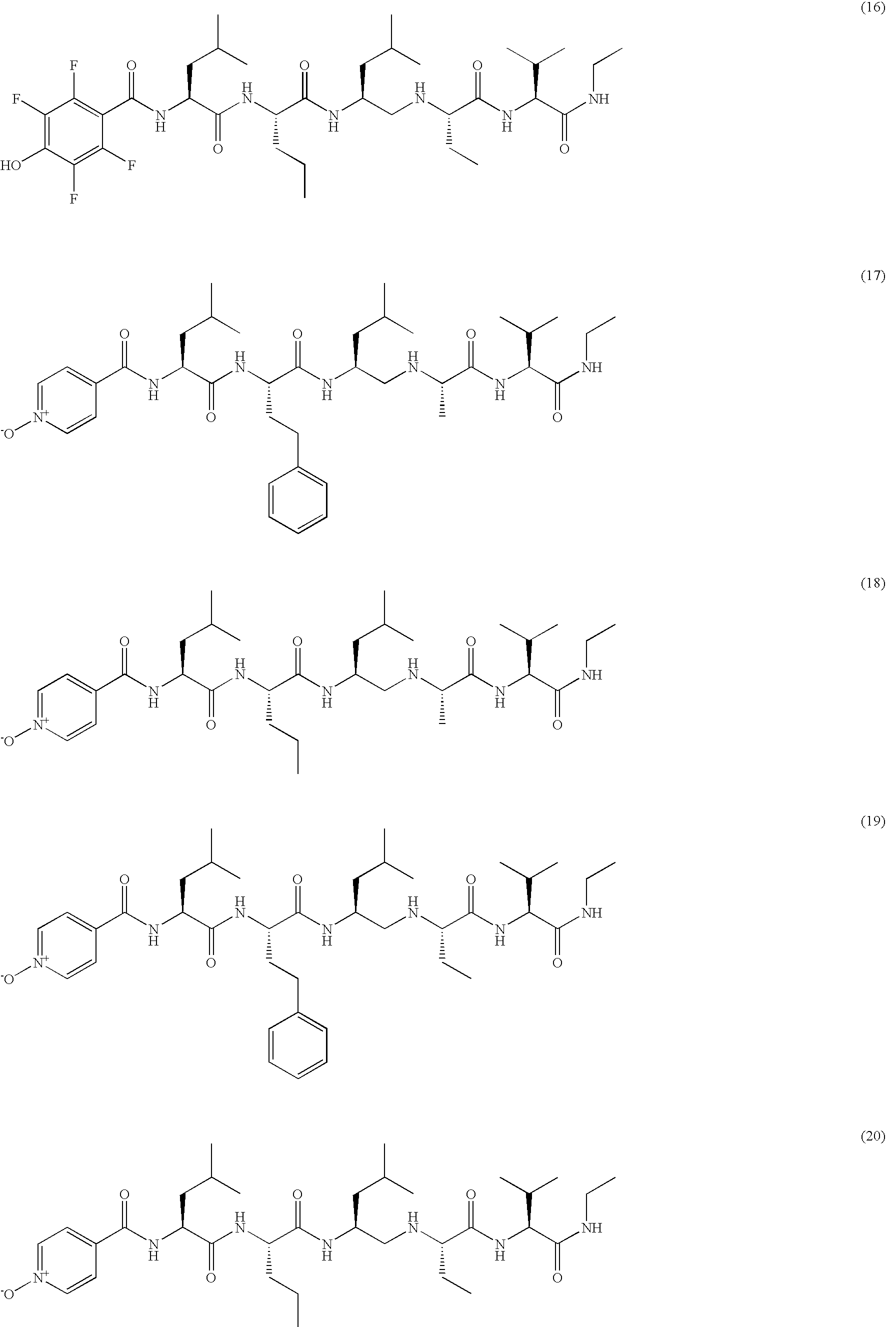

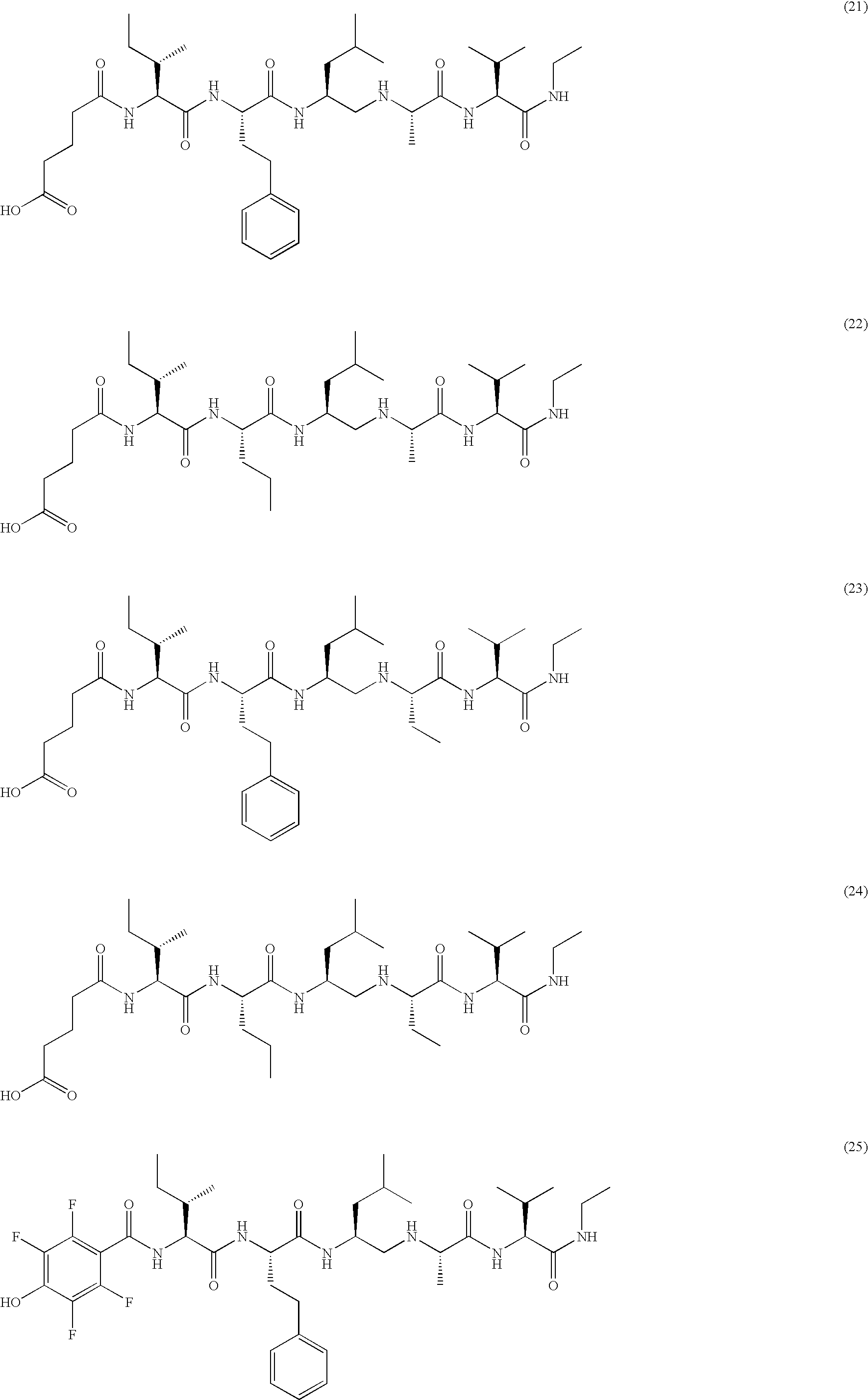

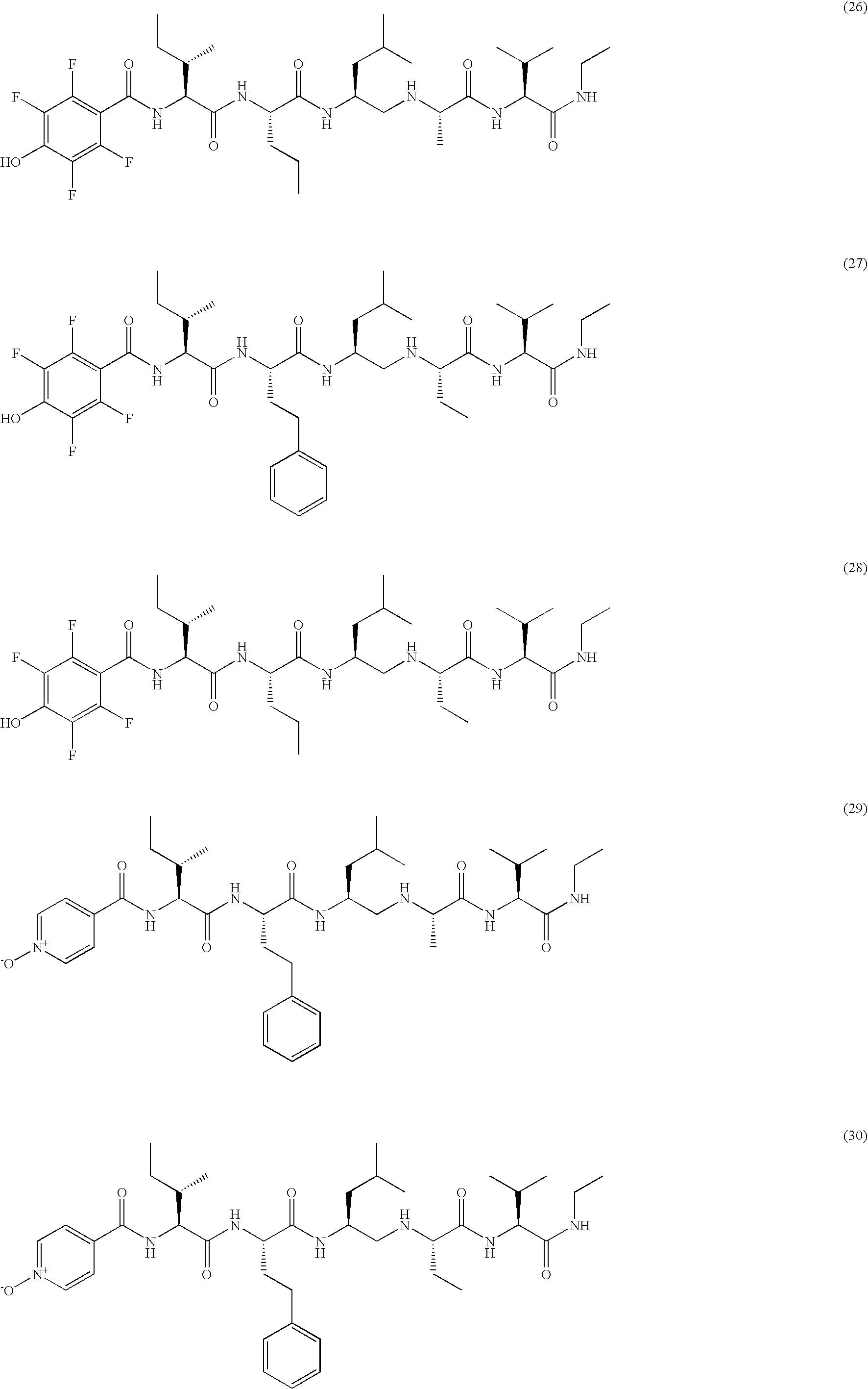

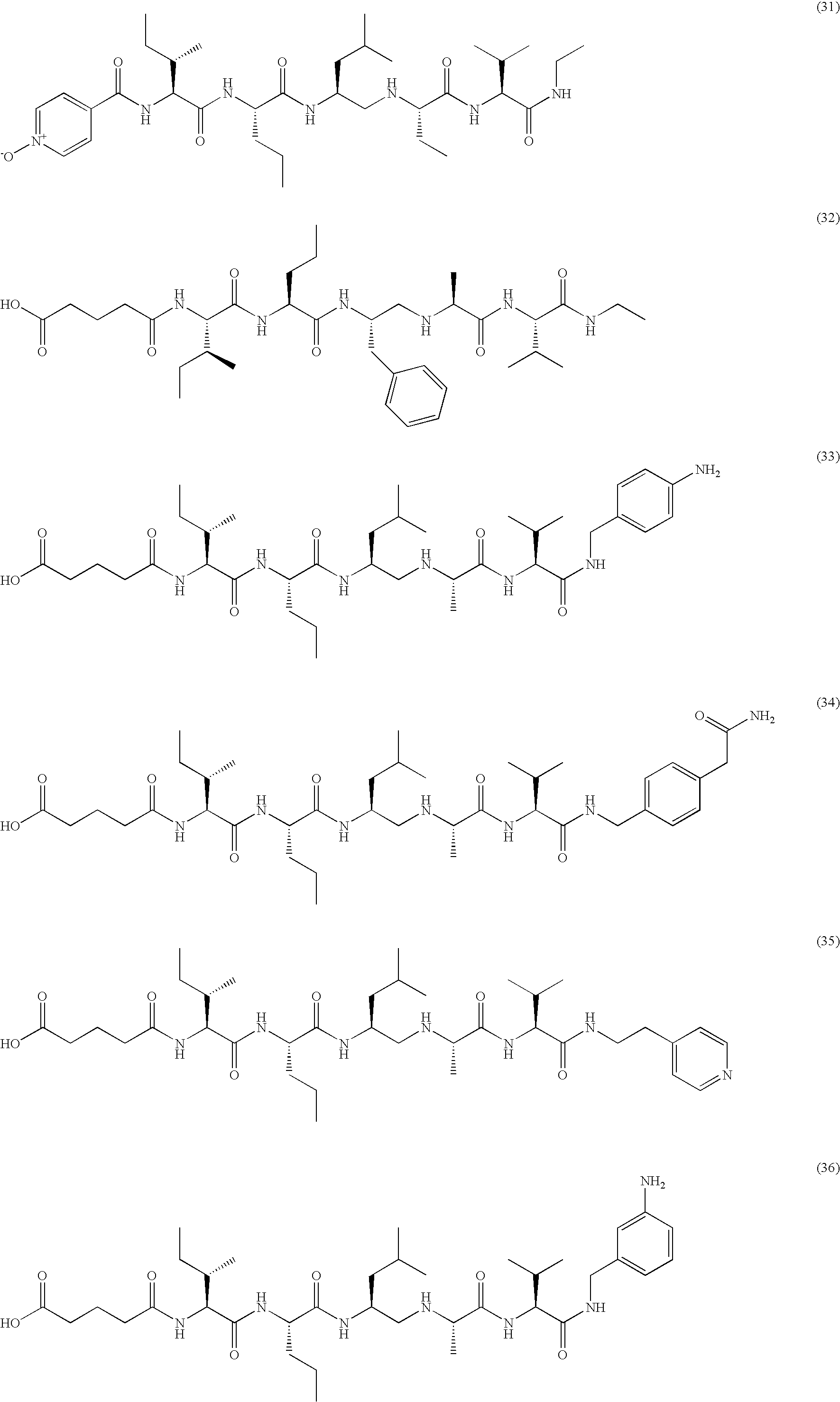

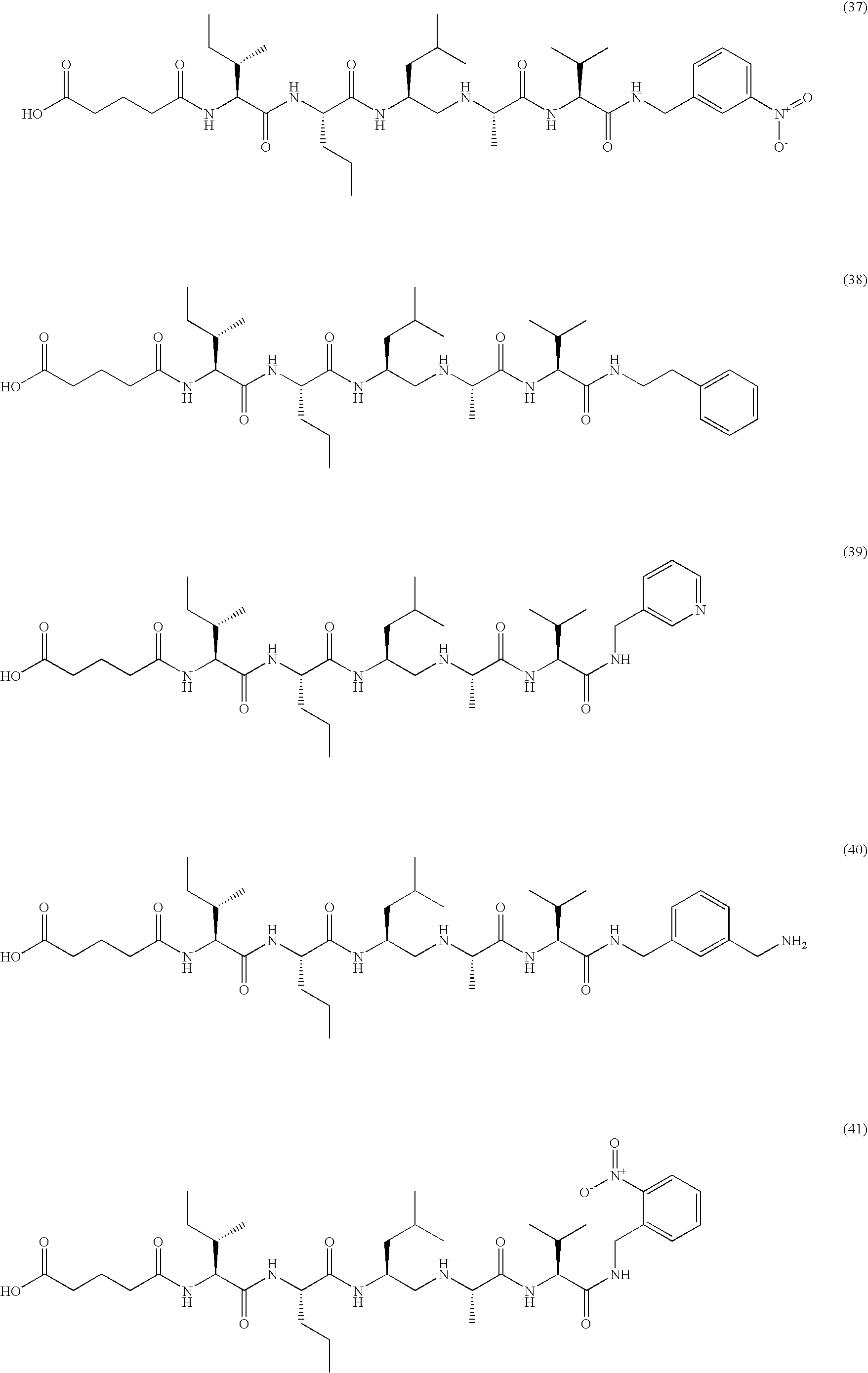

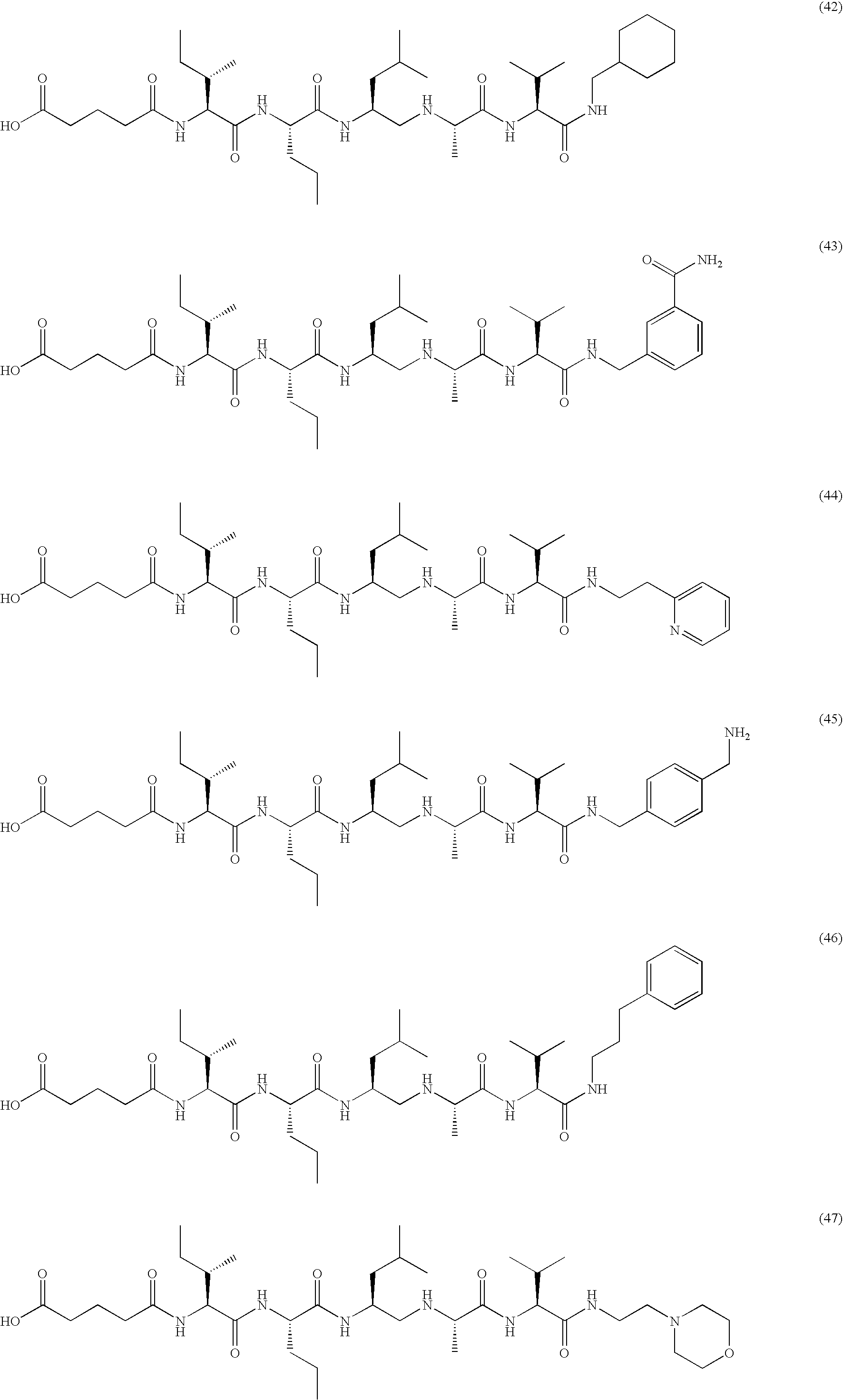

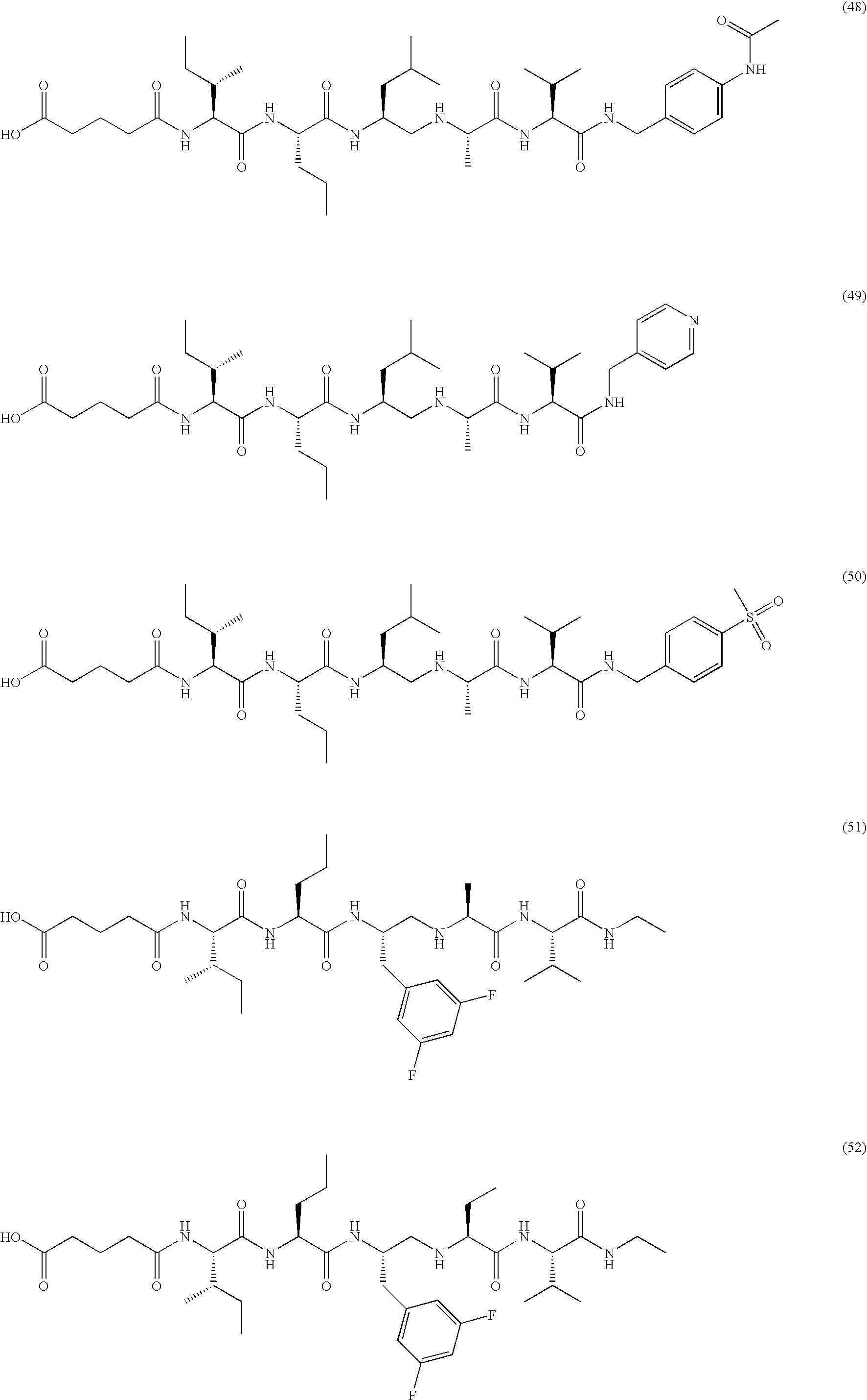

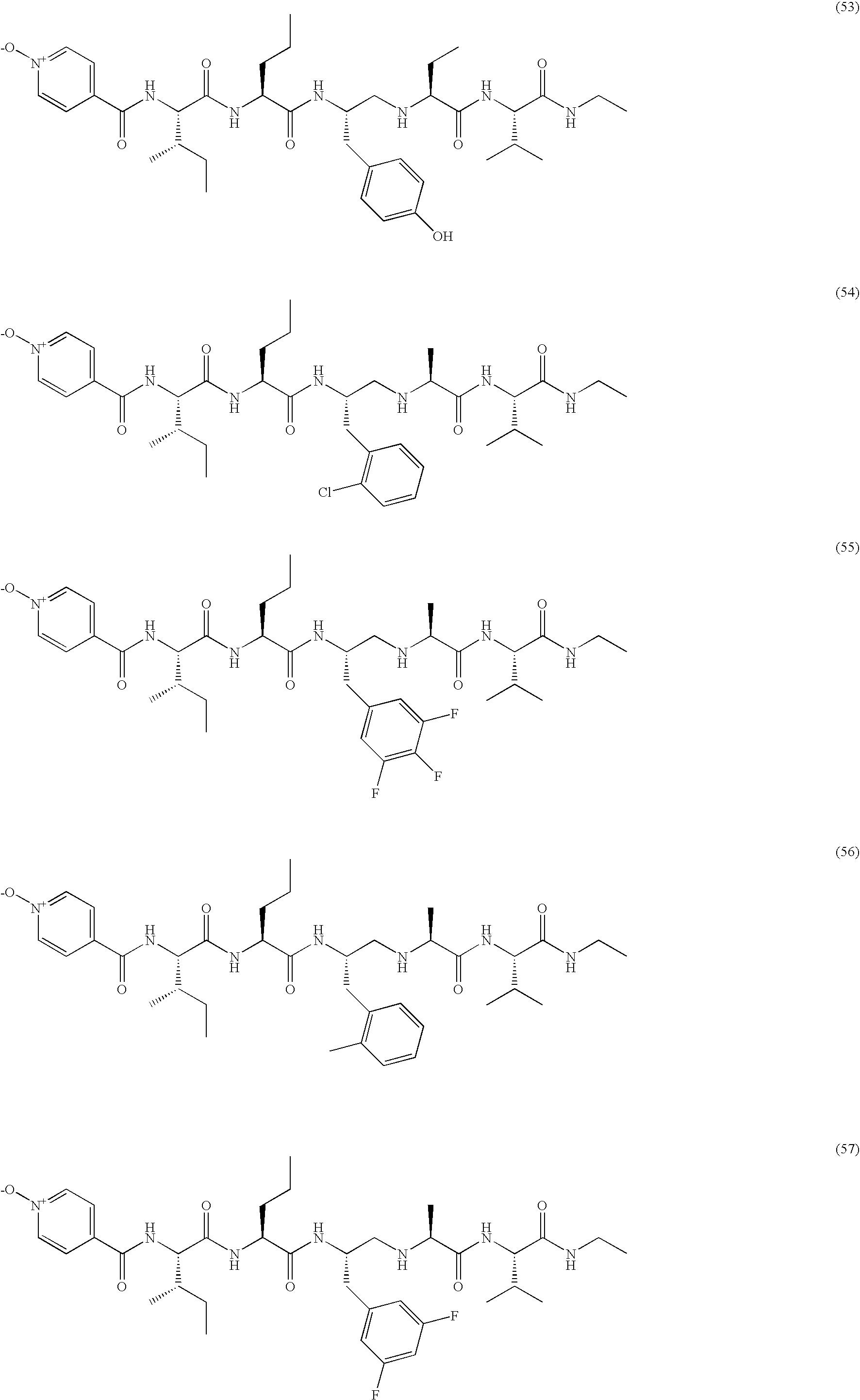

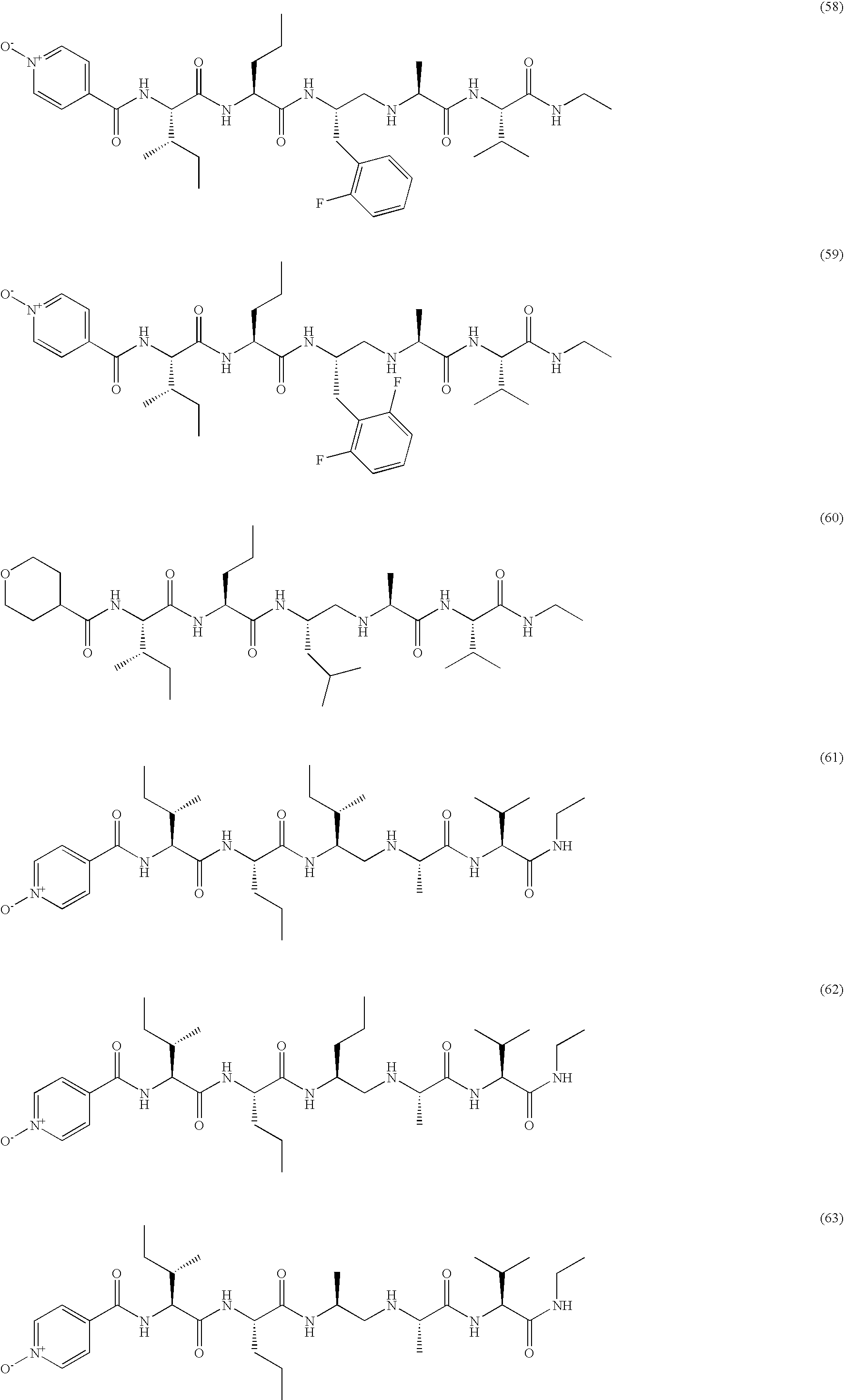

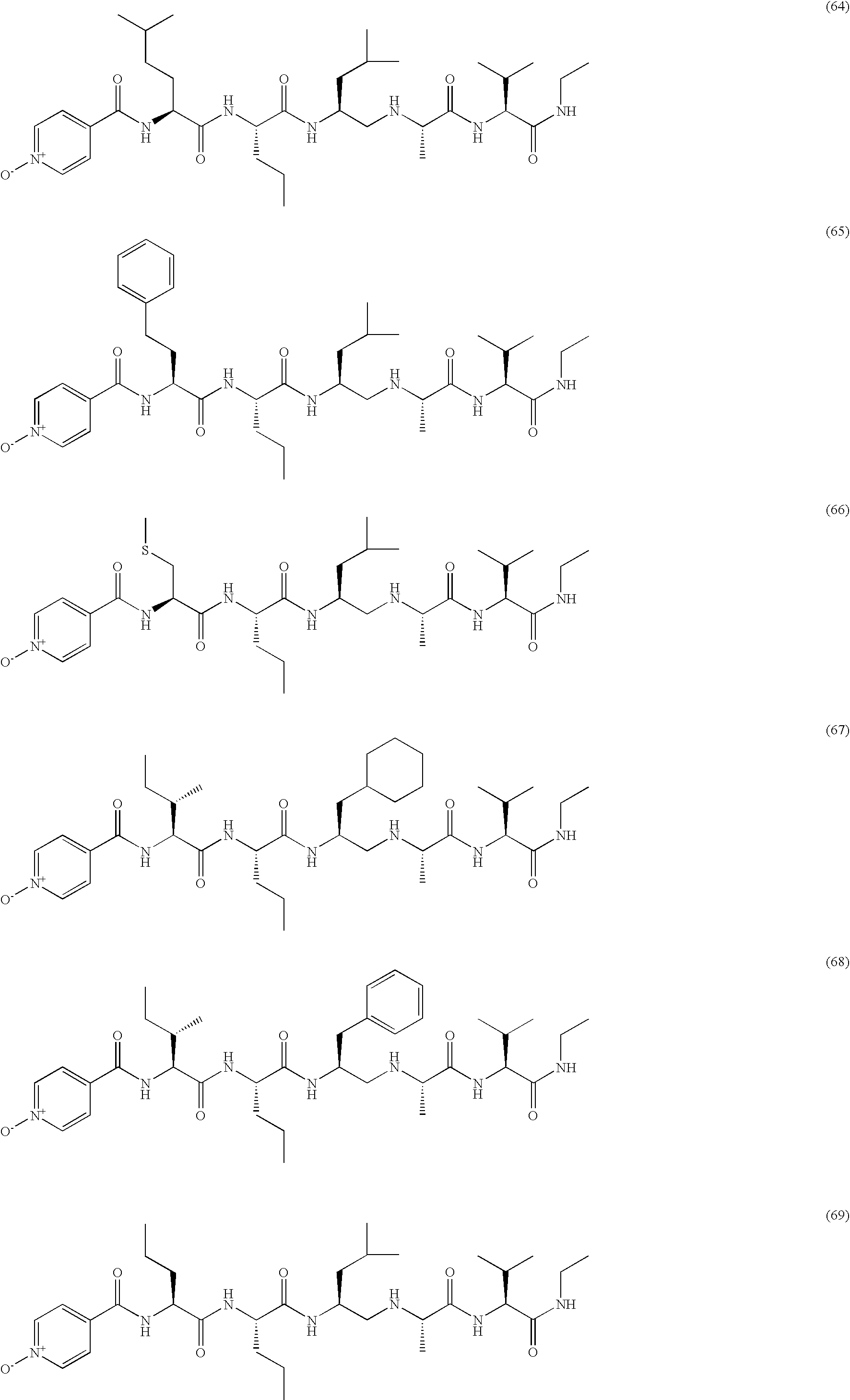

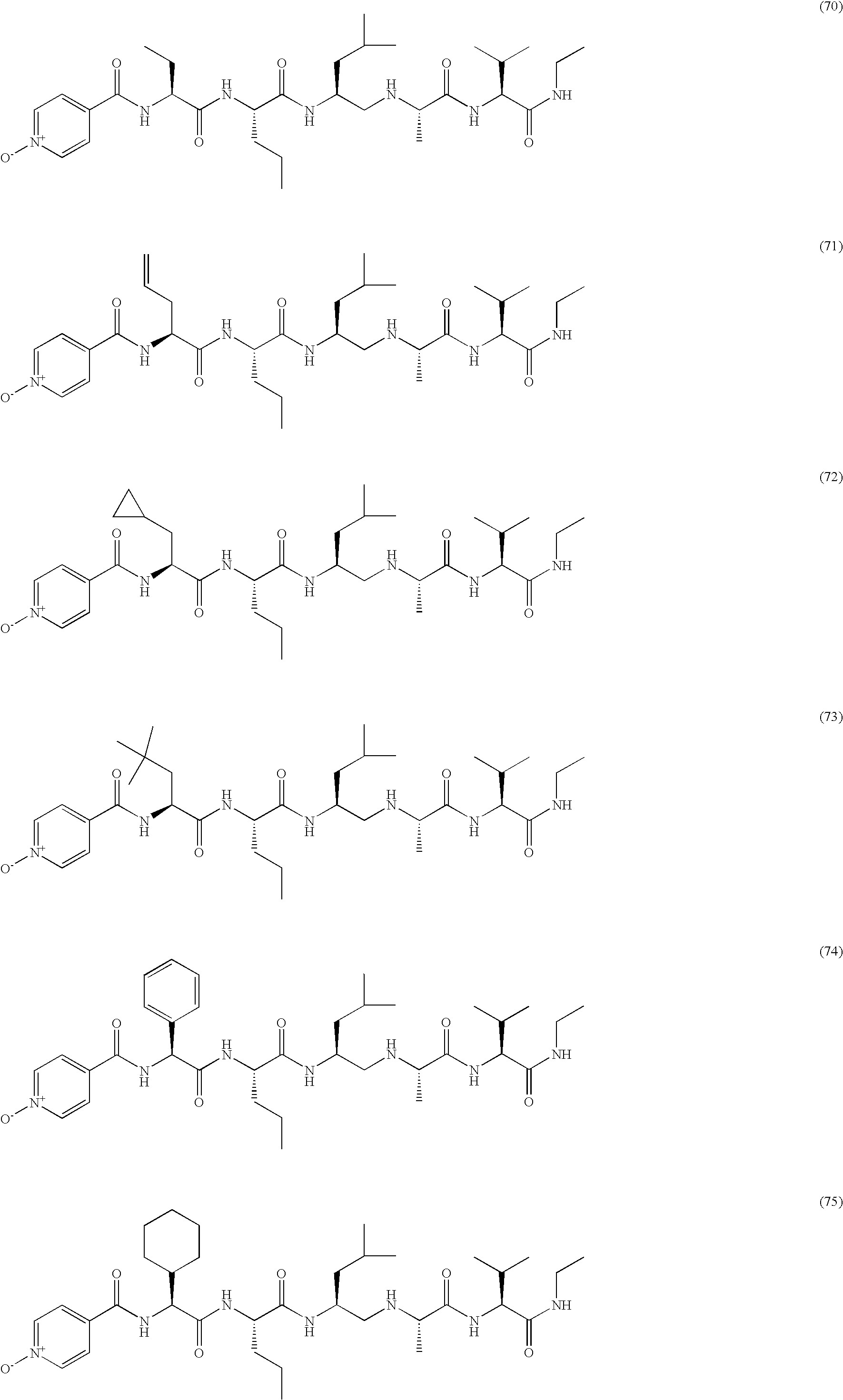

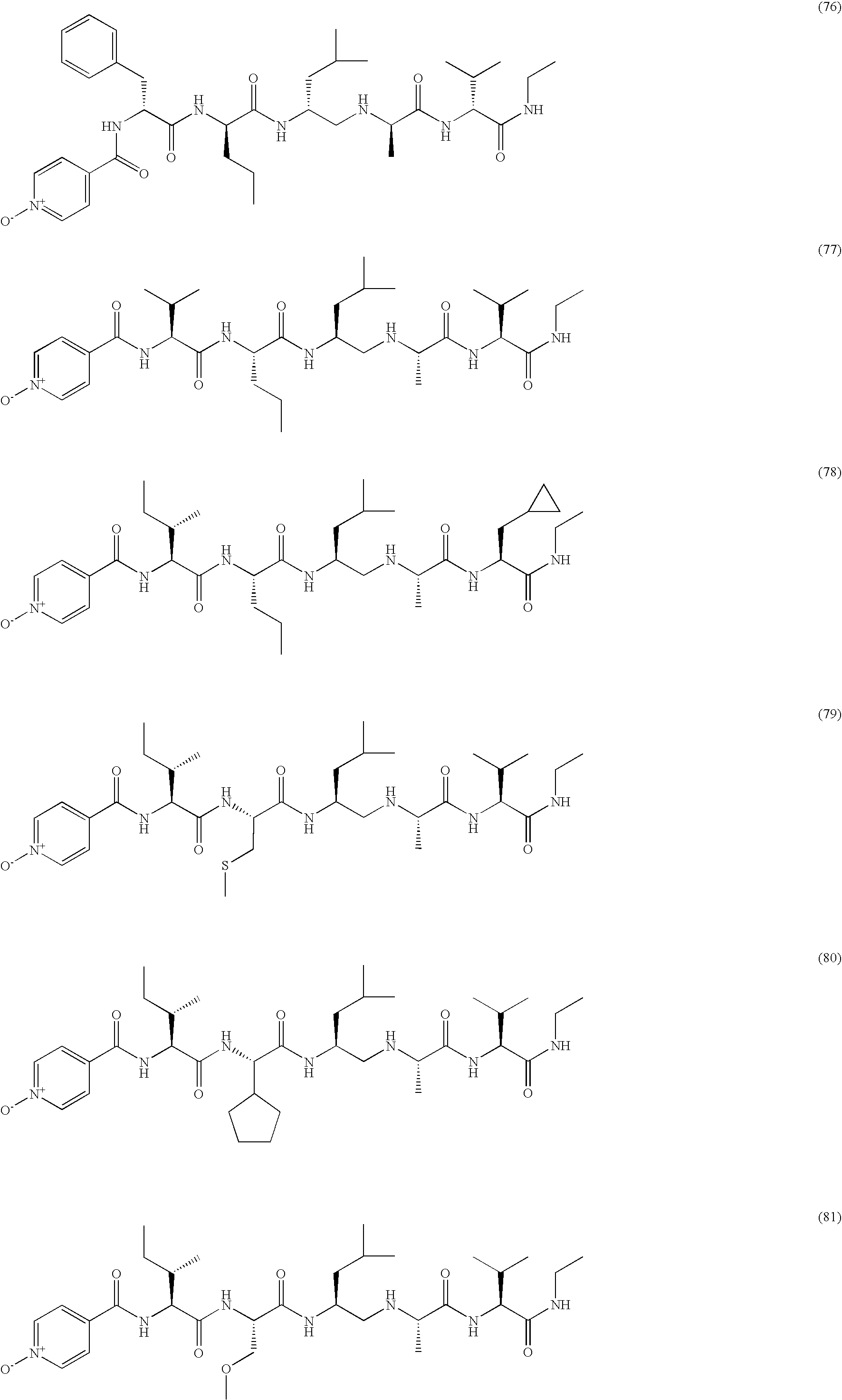

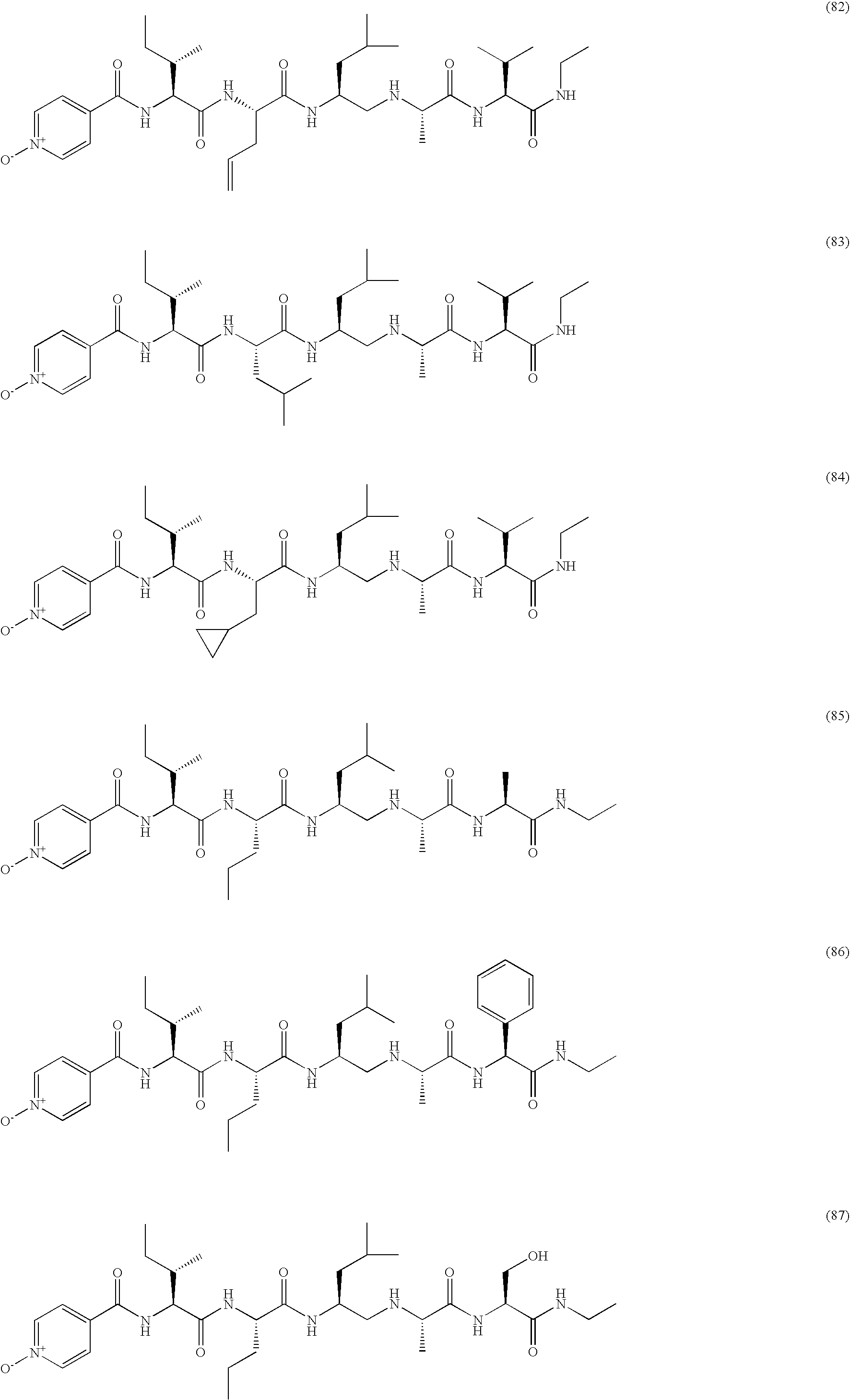

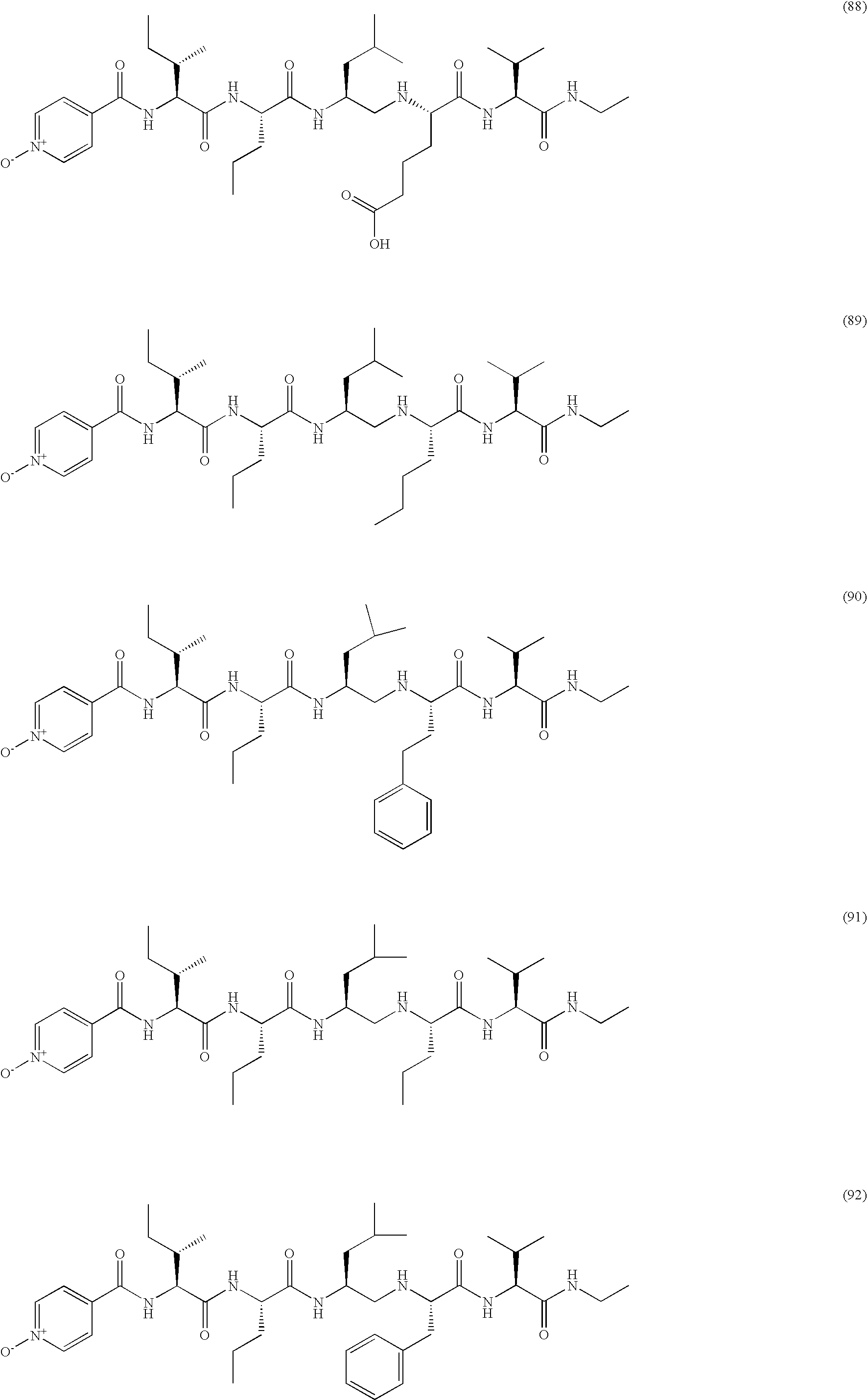

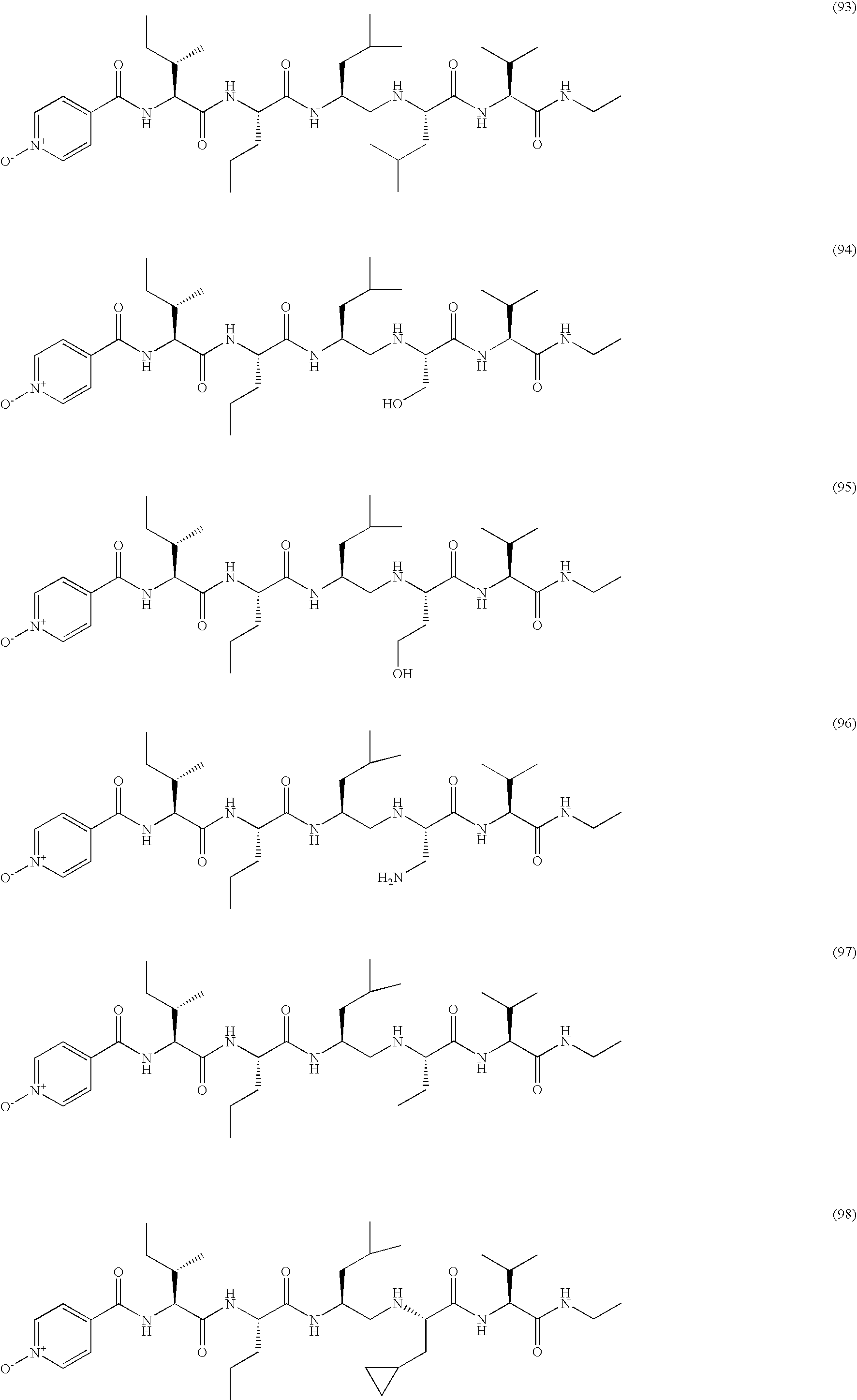

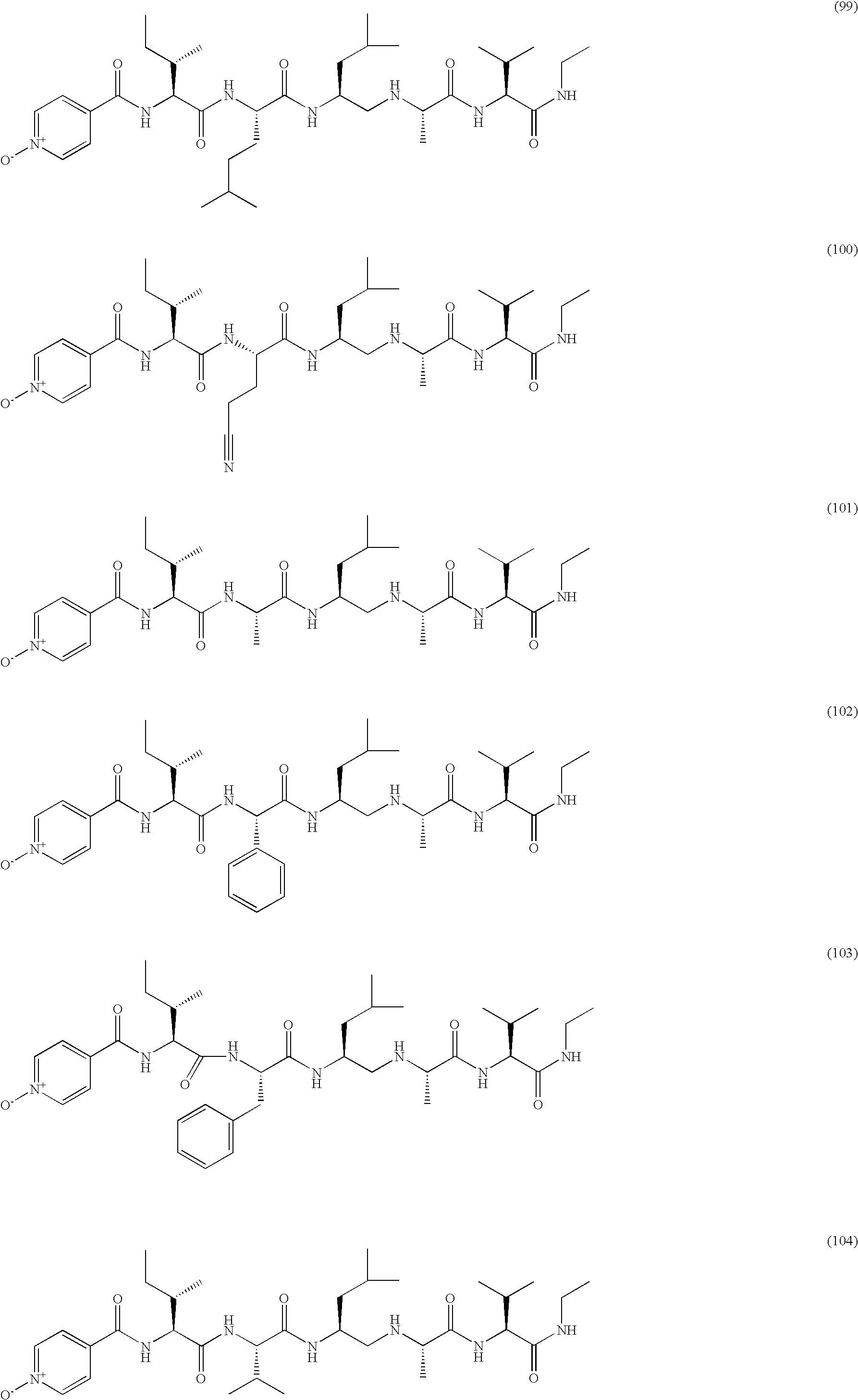

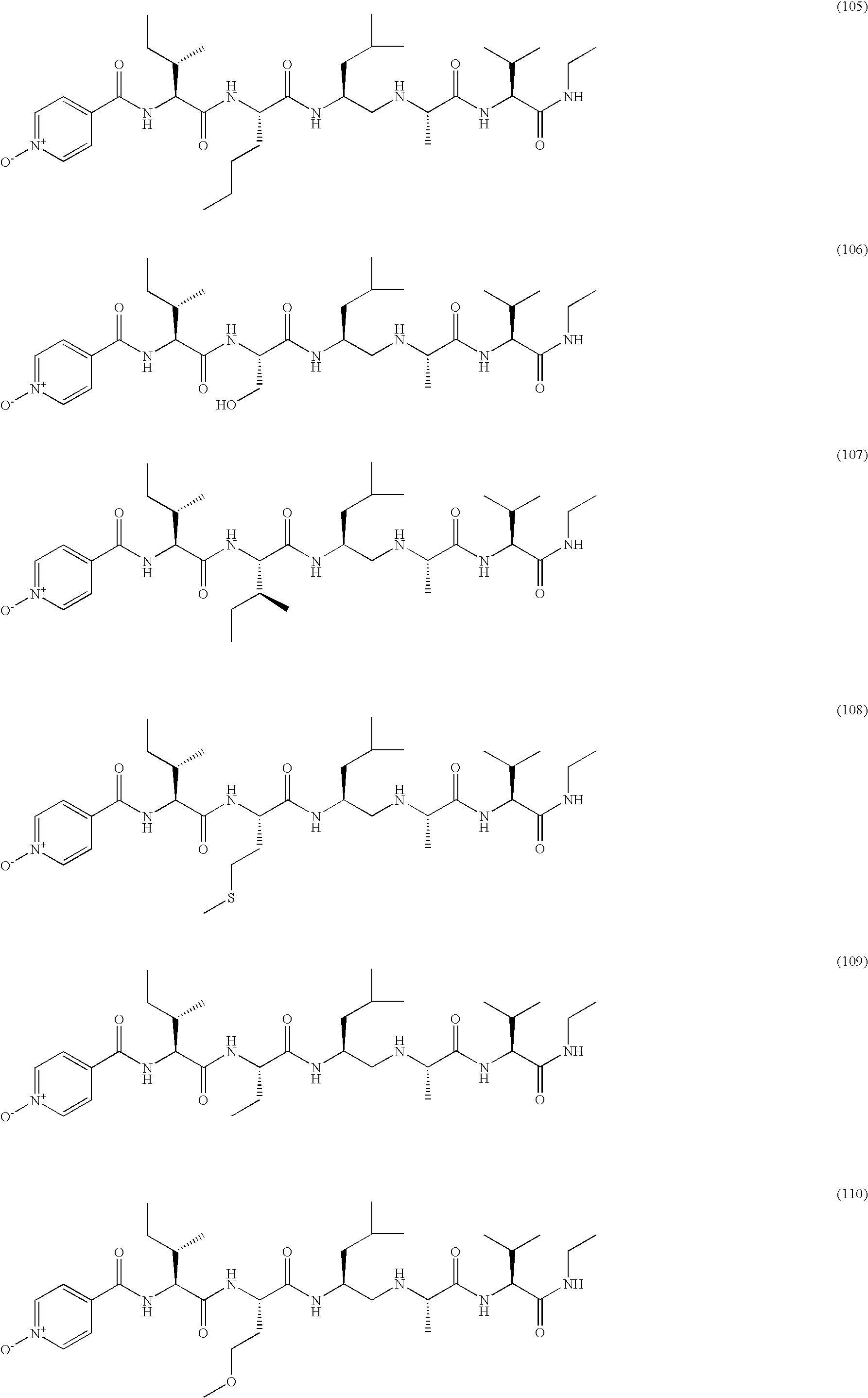

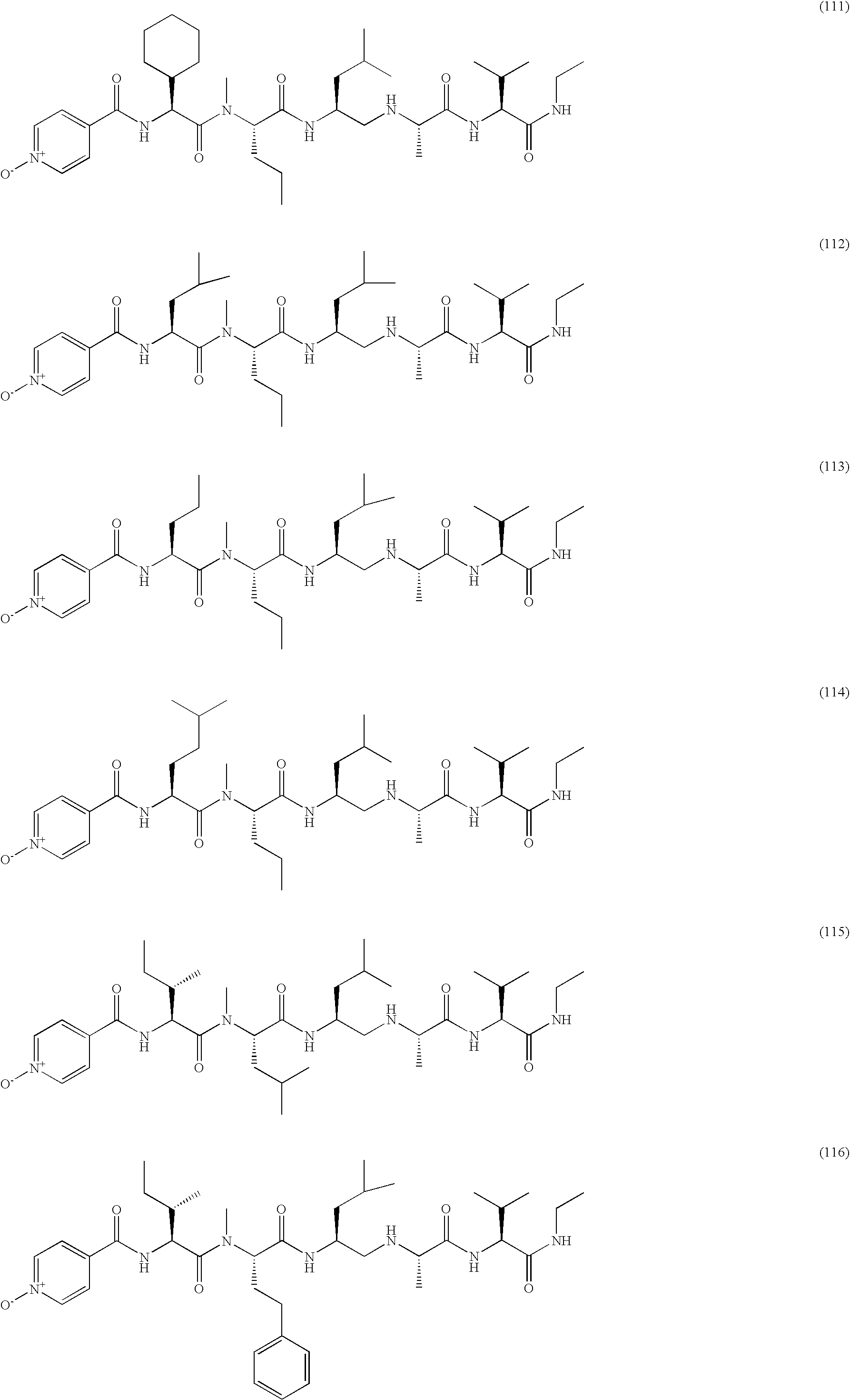

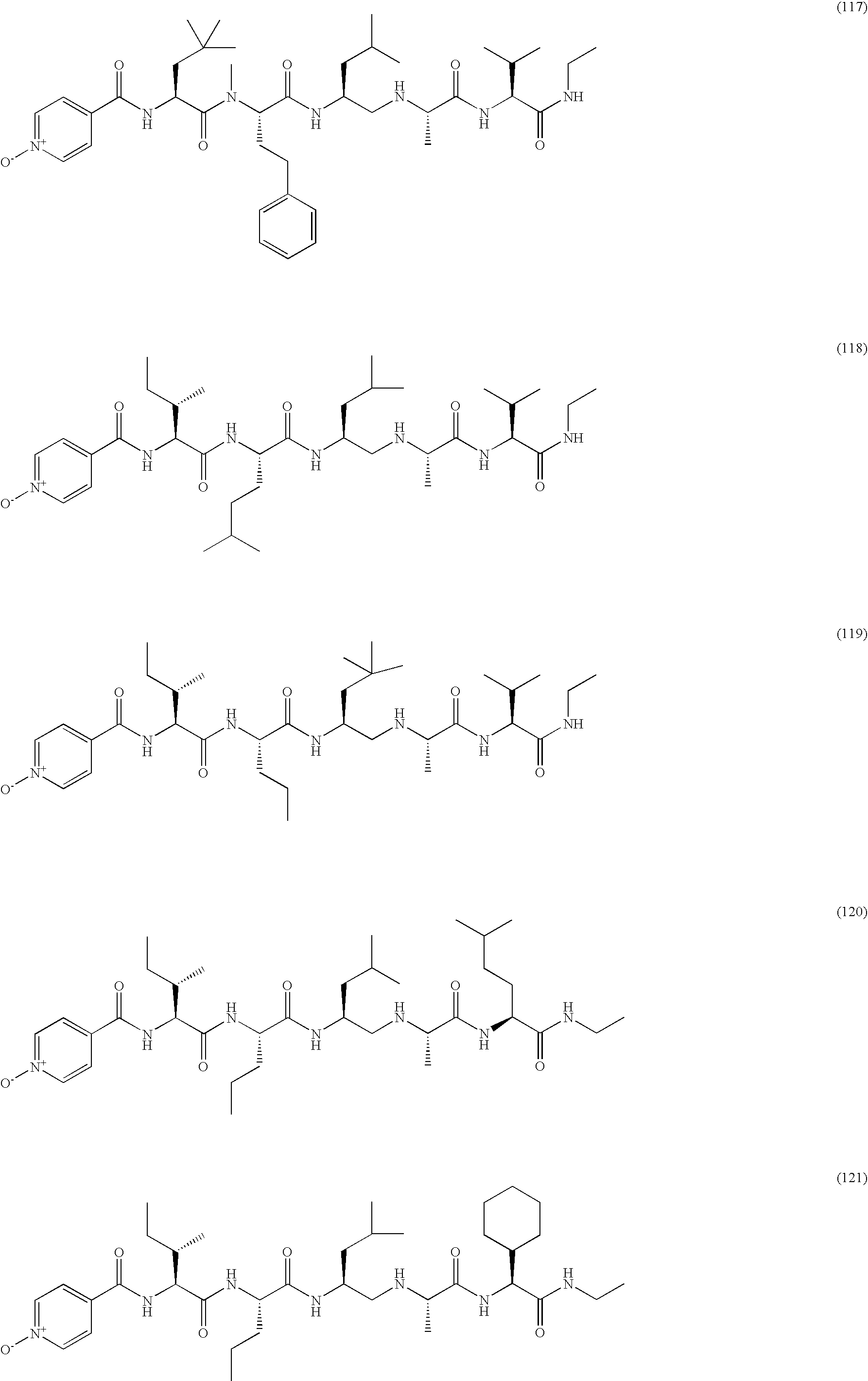

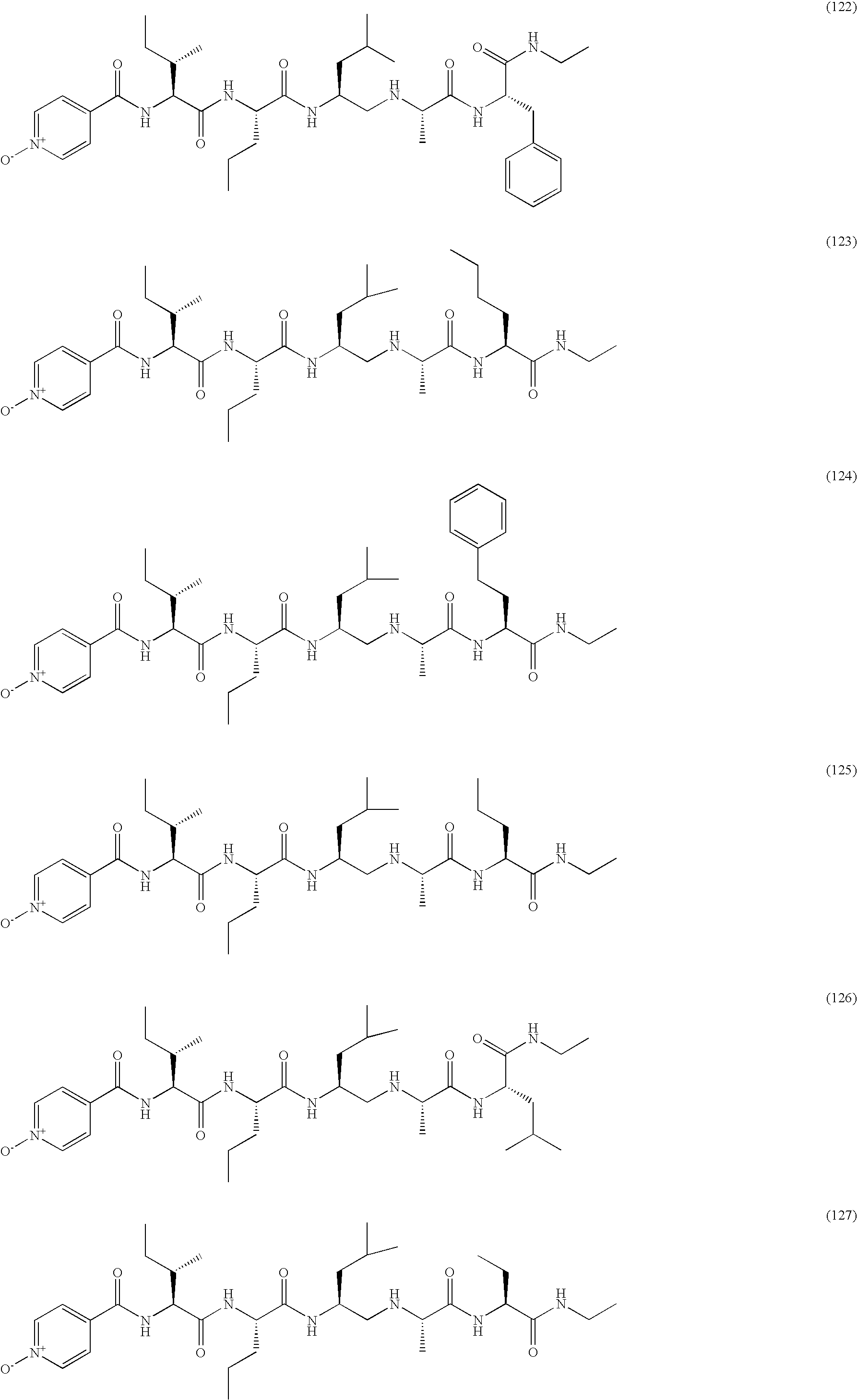

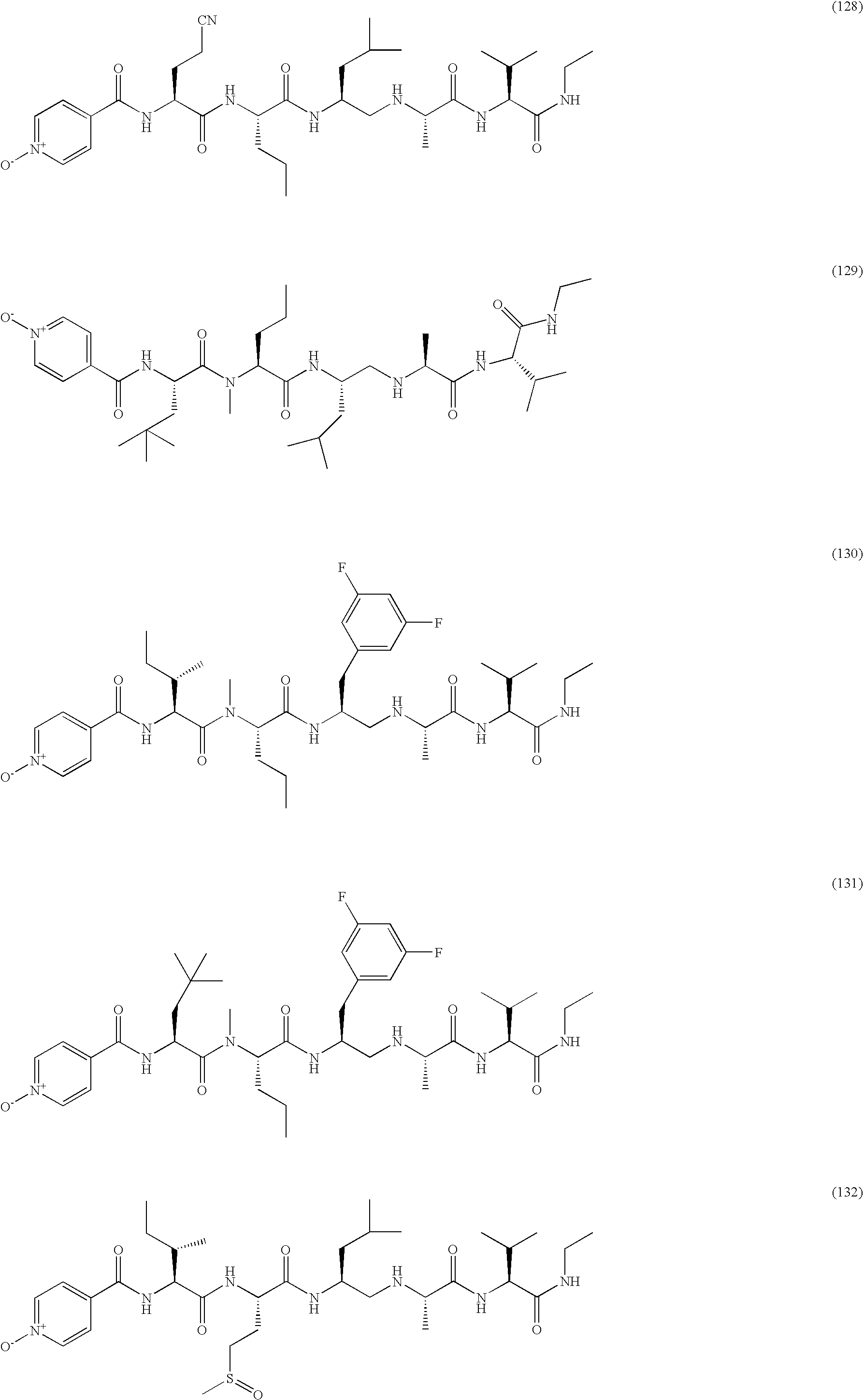

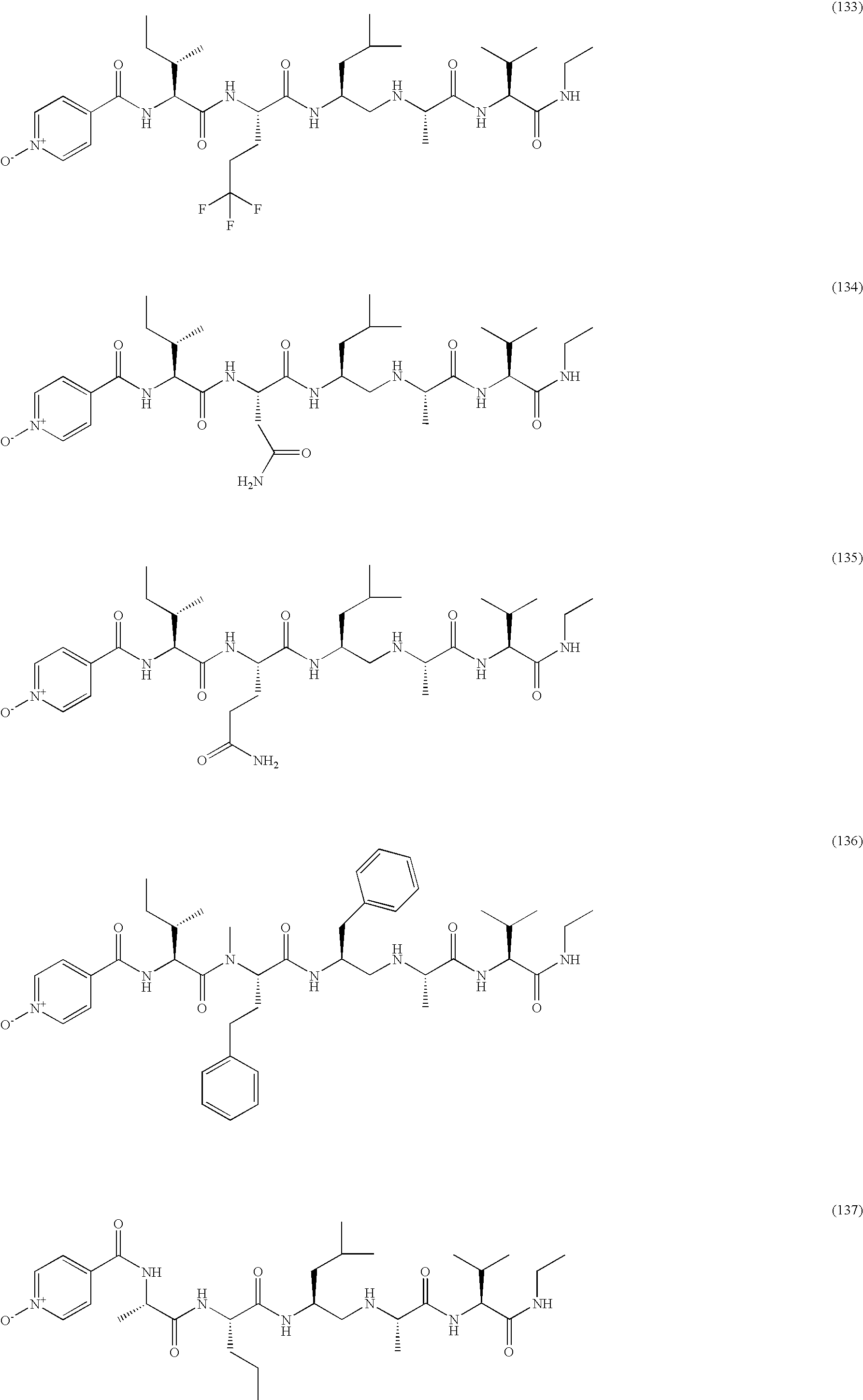

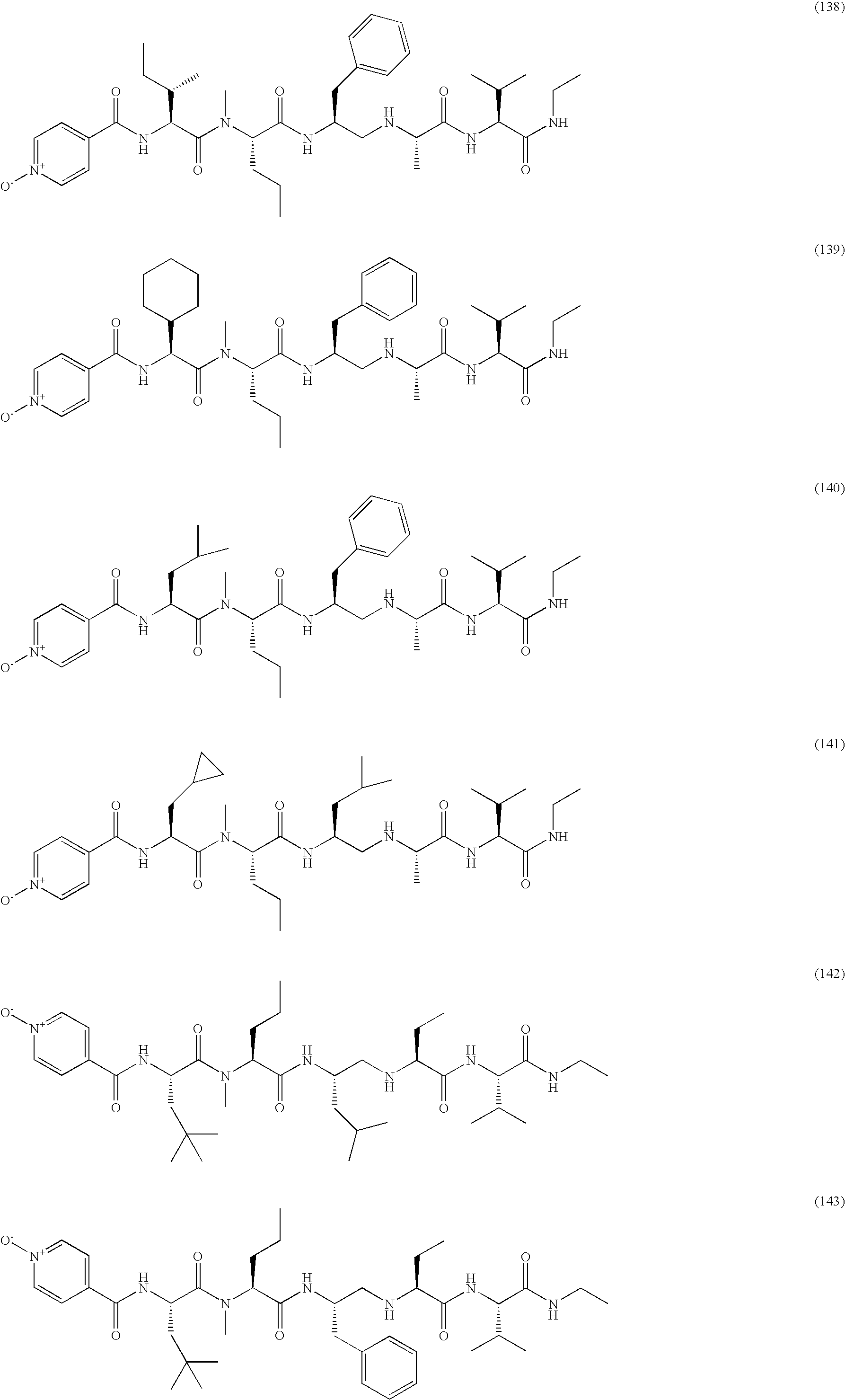

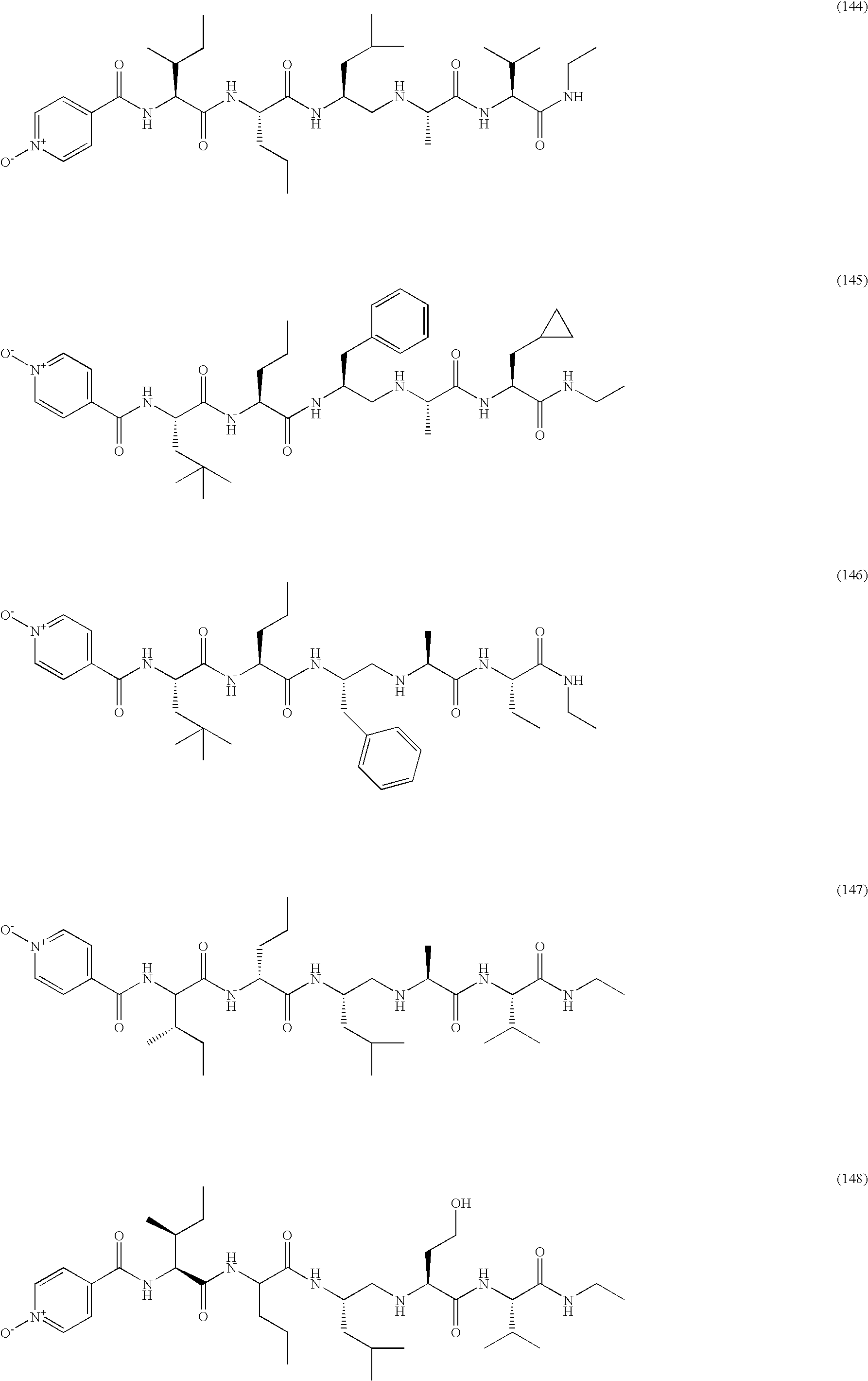

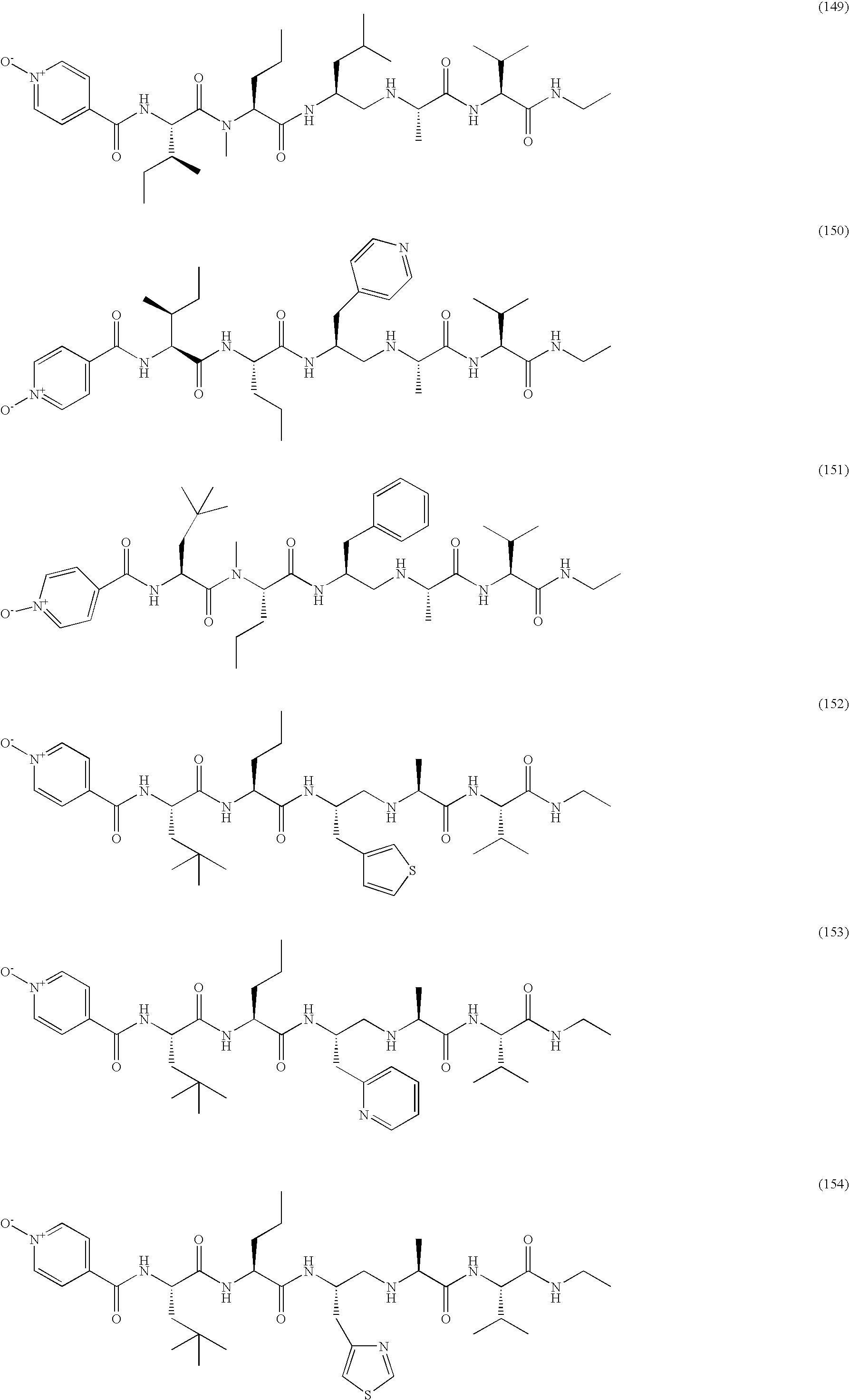

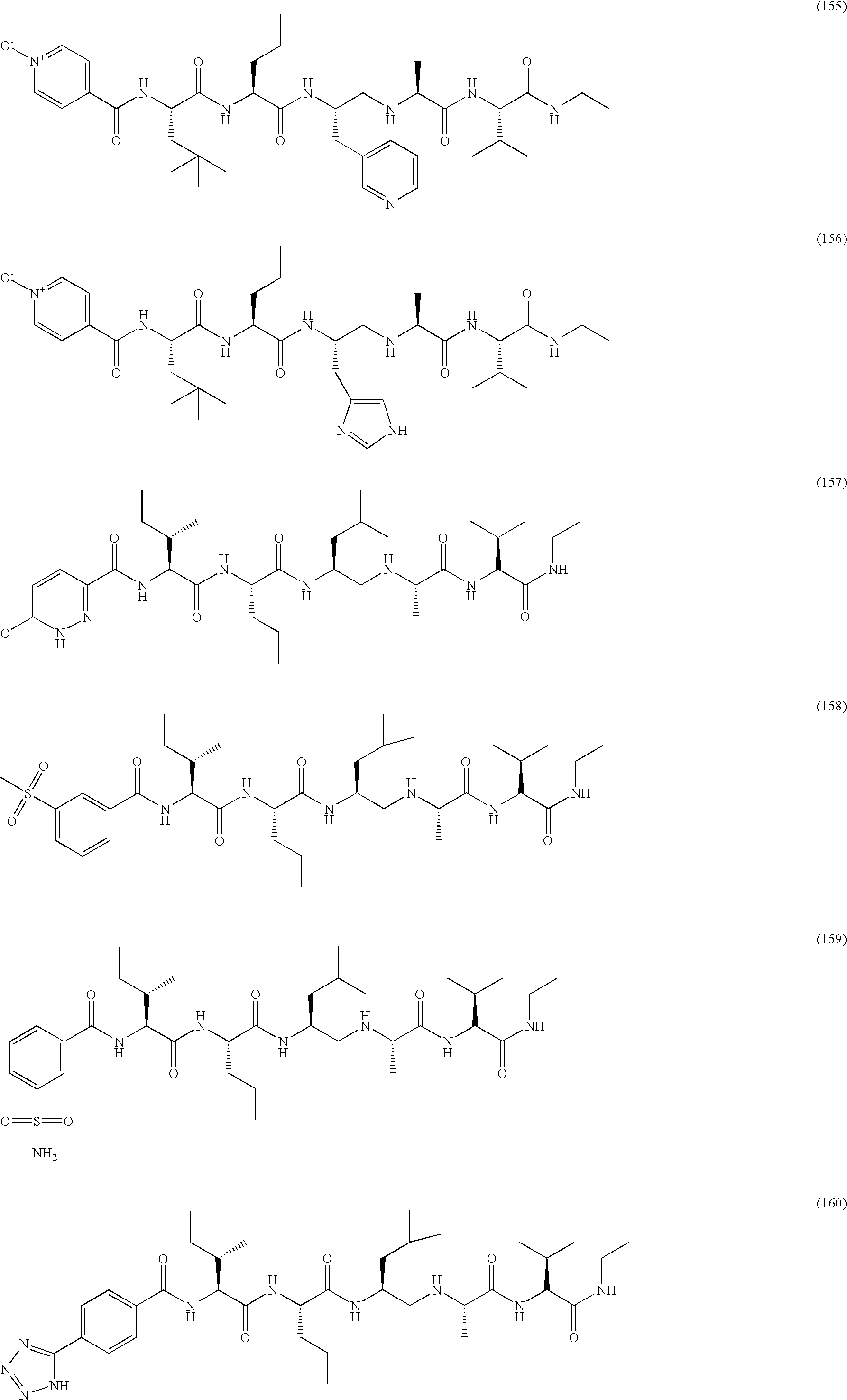

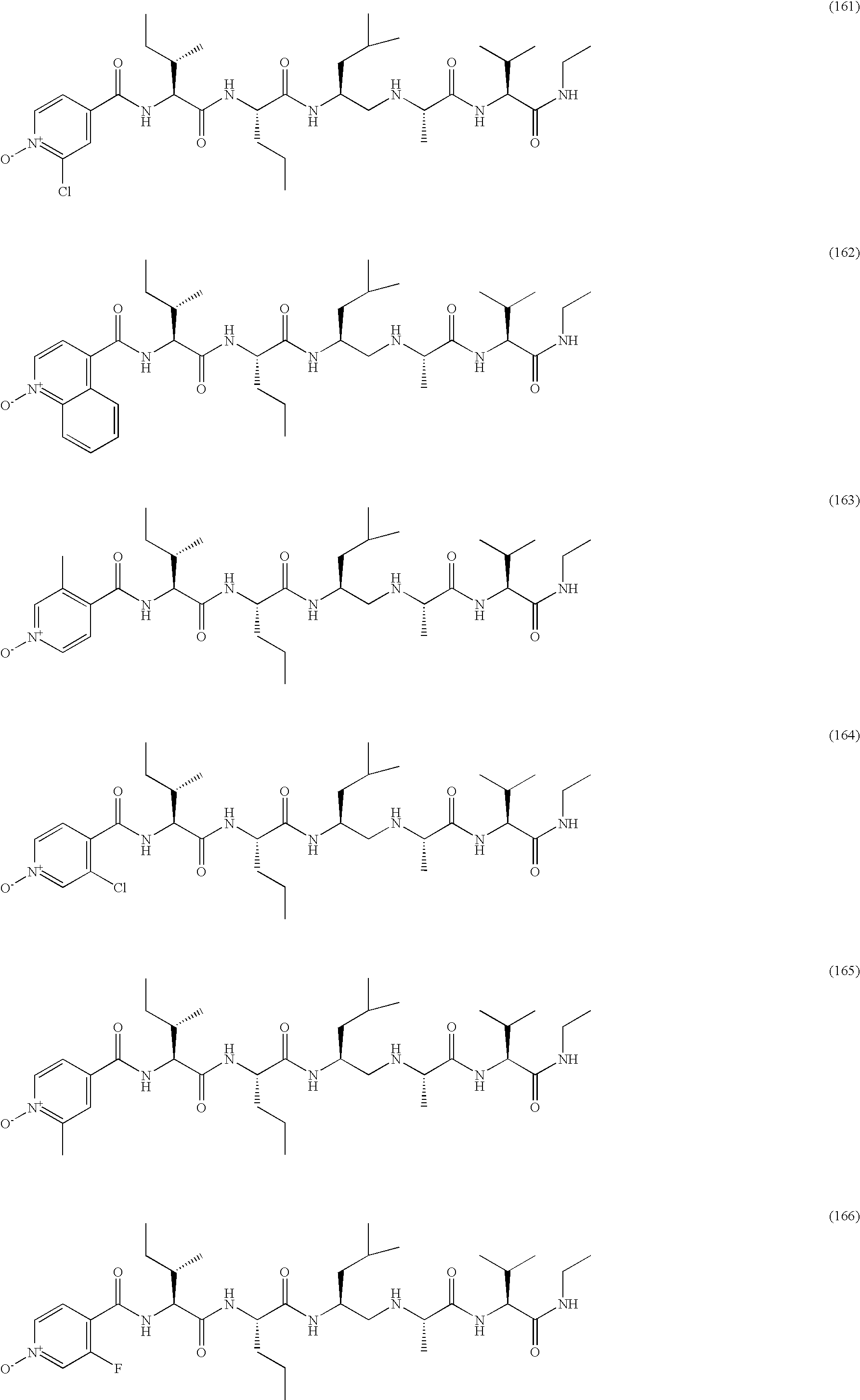

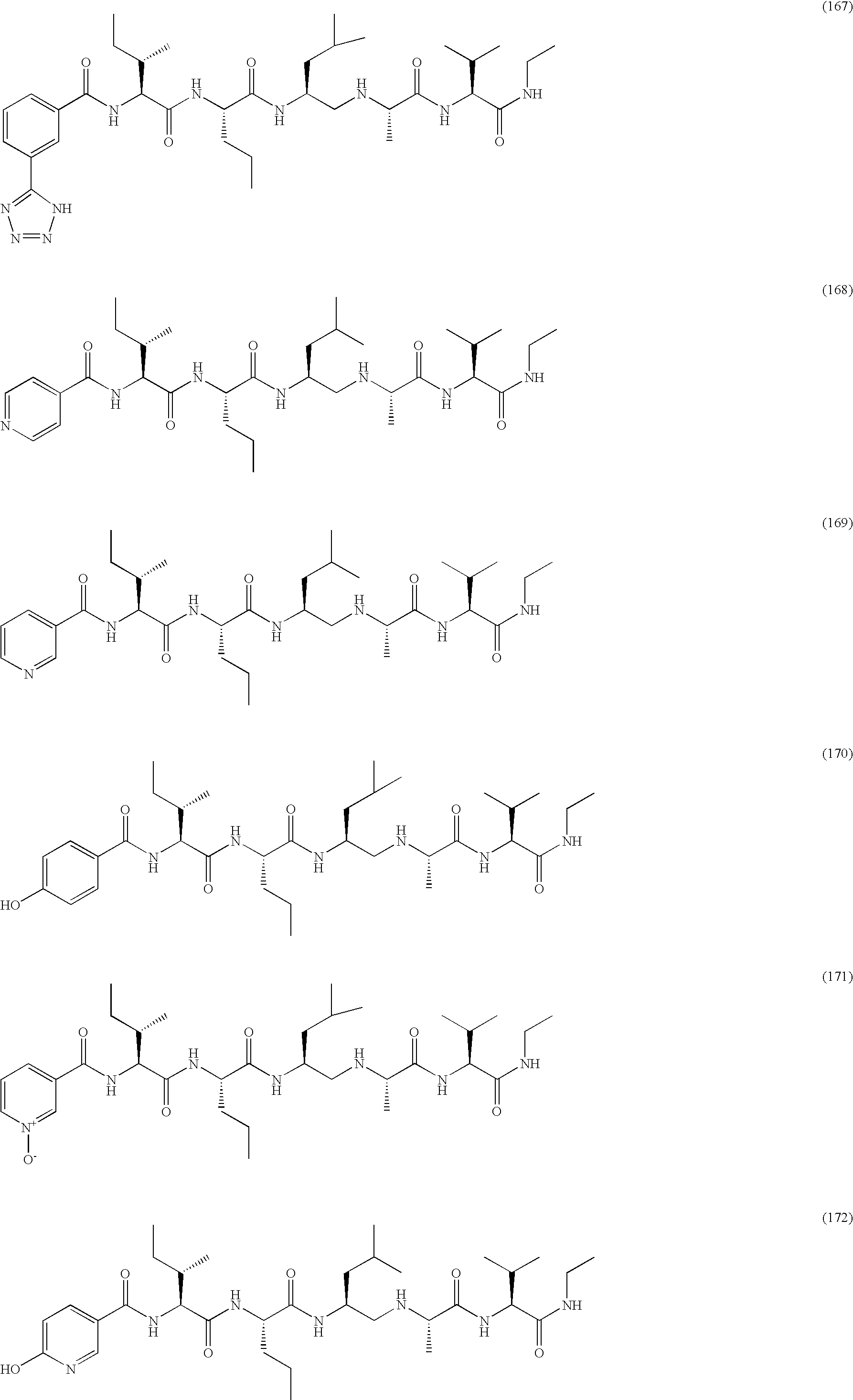

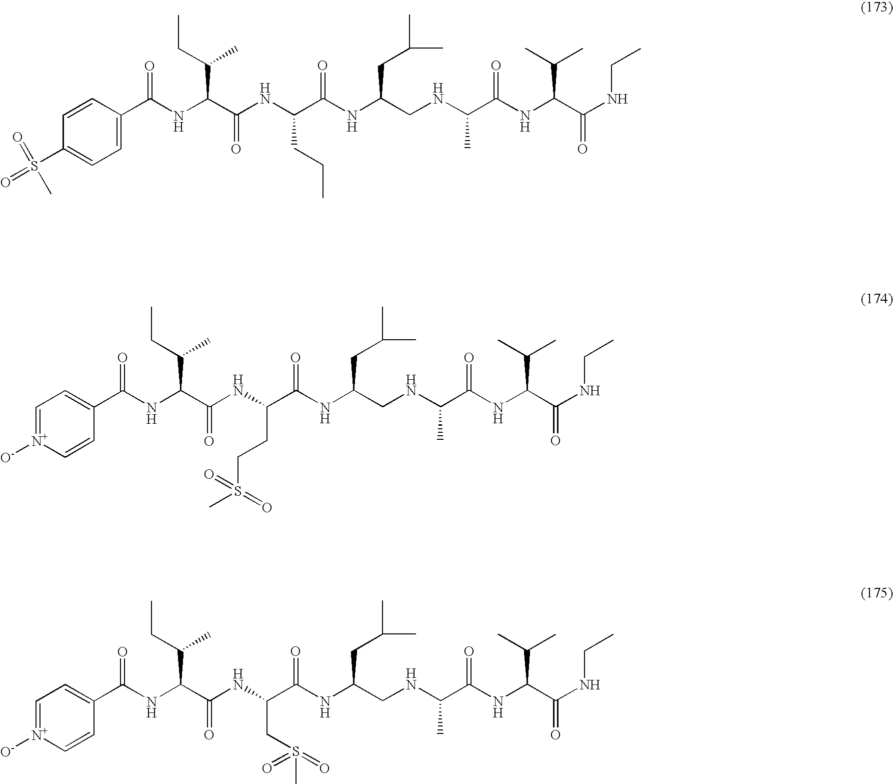

The anti-Alzheimer's amines of the present invention are made by methods well known to those skilled in the art from starting compounds known to those skilled in the art. The process chemistry is well known to those skilled in the art. The following reaction schemes illustrate the peptide synthesis of the compounds according to the present invention.

One skilled in the art will appreciate that these are all well known reactions in organic chemistry (Houben-Weyl—Methods of Organic Chemistry, Vol E22, Synthesis of Peptides and Peptidomimetics, M. Goodman, A. Felix, L. Moroder, C. Toniolo Eds., Georg Thieme Verlag Stuttgart, New York). A chemist skilled in the art, knowing the chemical structure of the biologically active compounds according to formula (I) of the invention would be able to prepare them by known methods from known starting materials without any additional information. The explanation below therefore is not necessary but is deemed helpful to those skilled in the art who desire to make the compounds of the present invention.

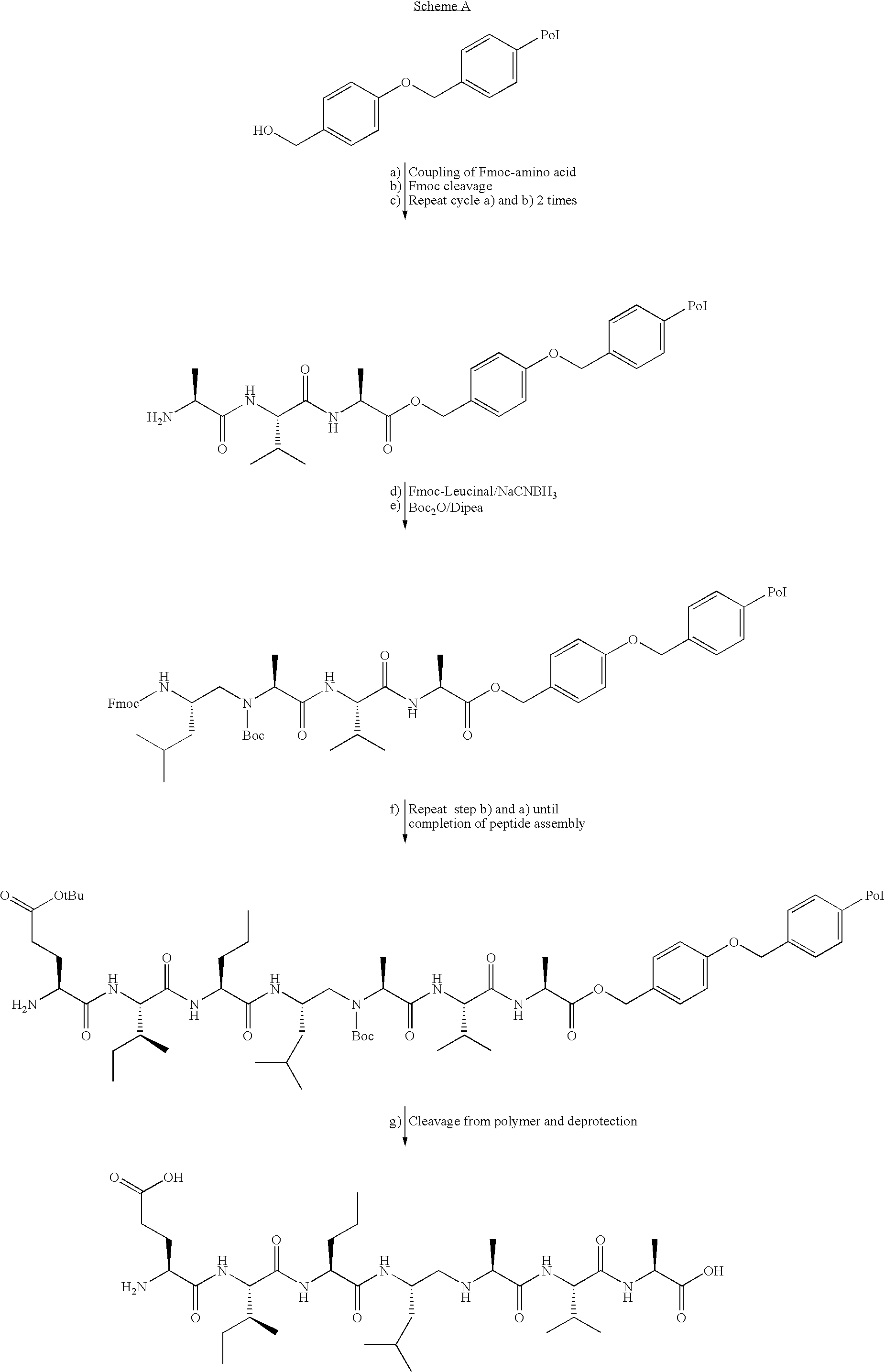

As illustrated in scheme A the synthesis of peptides bearing the free carboxy-terminus can be performed by standard peptide chemistry applying the Fmoc/tBu-protection. The first amino acid (Fmoc-alanine) has been esterified with the Wang-resin. The Fmoc-Ala-Wang resin is also commercially available. After deprotection of the Fmoc-group (step b) the next amino acid (Fmoc-valine) is coupled with a suitable peptide coupling reagent such as DIC/HOBt (step c). After introduction of the first three amino acids into the peptide a reductive alkylation with Fmoc-leucinal in presence of NaCNBH₃ as reducing agent is performed. The resulting secondary amine group is capped with (Boc)₂O. The peptide assembly has been completed applying step b), a) and b) and using the respective amino acids Fmoc-Nva, Fmoc-IIe and Fmoc-Glu(tBu). After removal of the terminal Fmoc-group the peptide is cleaved from the polymer with trifluoroacetic acid with concurrent removal of the tBu-side chain protecting group of the glutamic acid residue and the Boc-protecting group. The crude peptide can be purified by precipitation from diethyl ether and by reversed phase HPLC.

The synthesis protocol allows the incorporation of different residues in the position R², R⁷, R⁸, R⁹, R¹⁰, R¹¹ and R¹² of formula (I) and the variation of the peptide length in formula (I) as well.

A slightly modified solid-phase peptide synthesis is exemplified in scheme B.

As a polymer commercially available [3-{[ethyl-Fmoc-amino]-methyl}-indol-1-yl-acetyl AM resin (Indol resin, Novabiochem) is used. After cleavage of the Fmoc-group with piperidine in DMF (step a) the first amino acid is coupled with standard methods of peptide chemistry, e.g. HATU/HOBt (step b). The peptide assembly is performed in the same way as described in scheme A applying step a and b, the reductive alkylation and the removal of the terminal Fmoc-group. The introduction of the N-terminal capping group can be achieved by standard acylation methods (step g). The C-terminal peptide N-ethlylamide is cleaved from the polymer by reaction with acids e.g. trifluoroacetic acid.

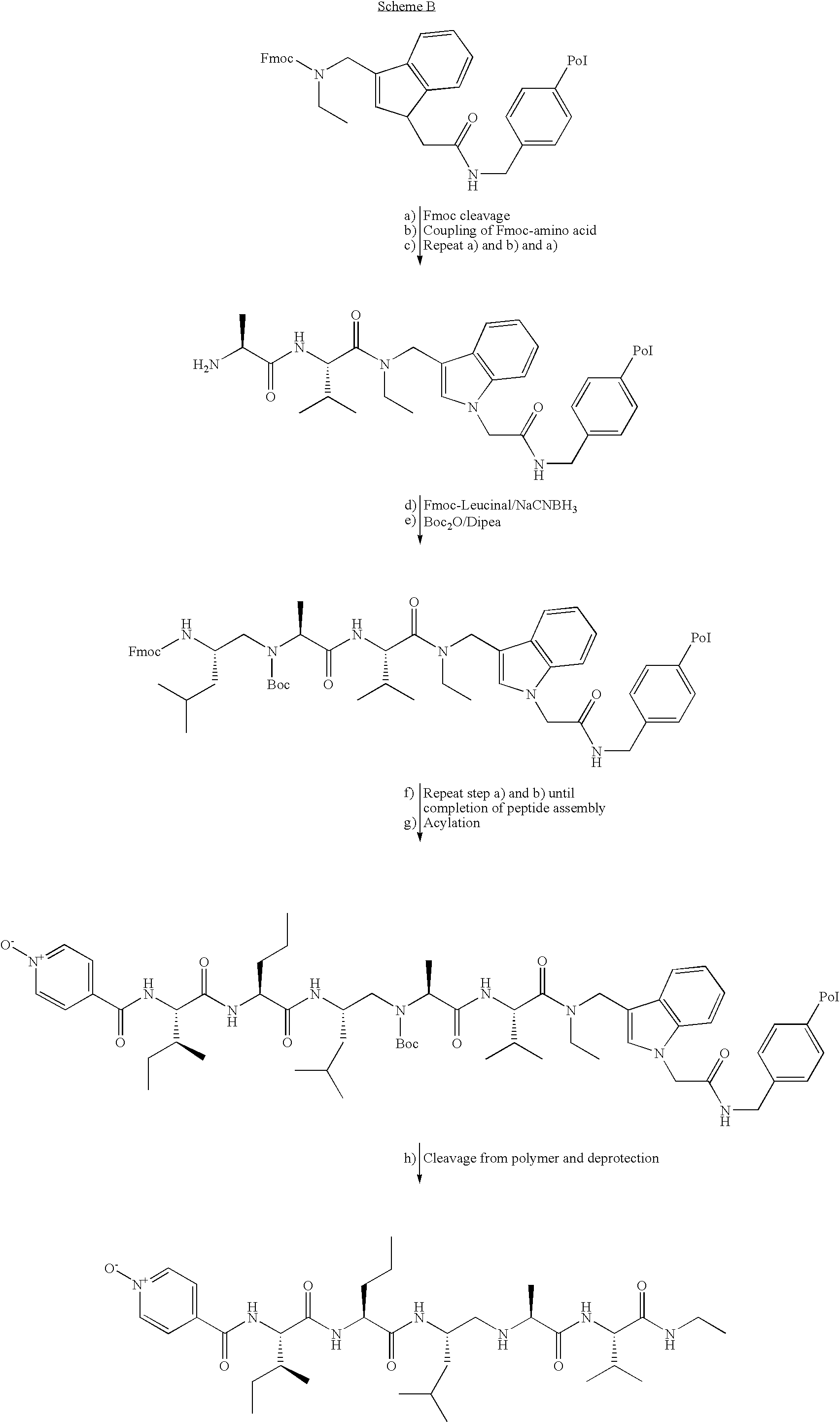

Scheme C illustrates the synthesis of peptides with variations of the C-terminal amide part. For this purpose a commercially available (formylindolyl)acetamidomethylpolystyrene resin is used. In the first reaction the aldehyde group has been reductively alkylated with a 4-(aminomethyl)-1-N-Boc-aniline in presence of NaCNBH₃. The further peptide assembly and the cleavage from the polymer has been done as described above.

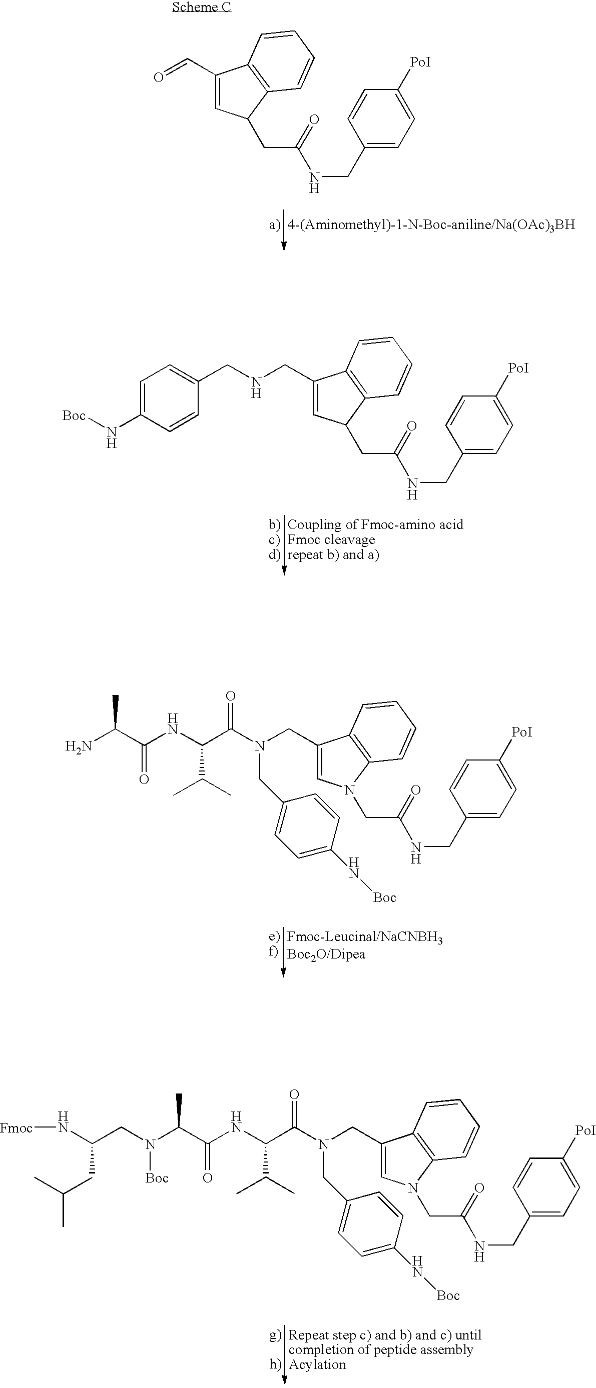

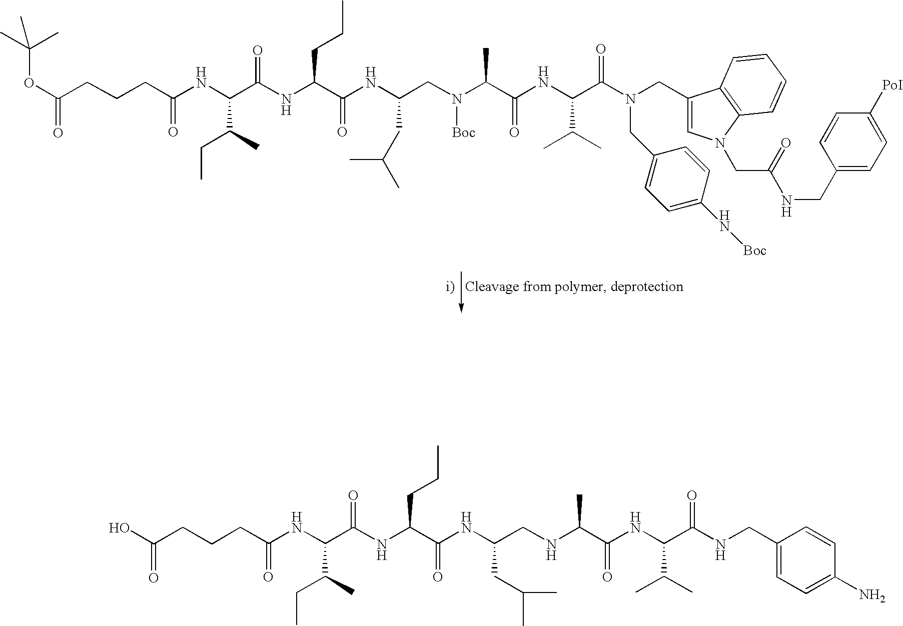

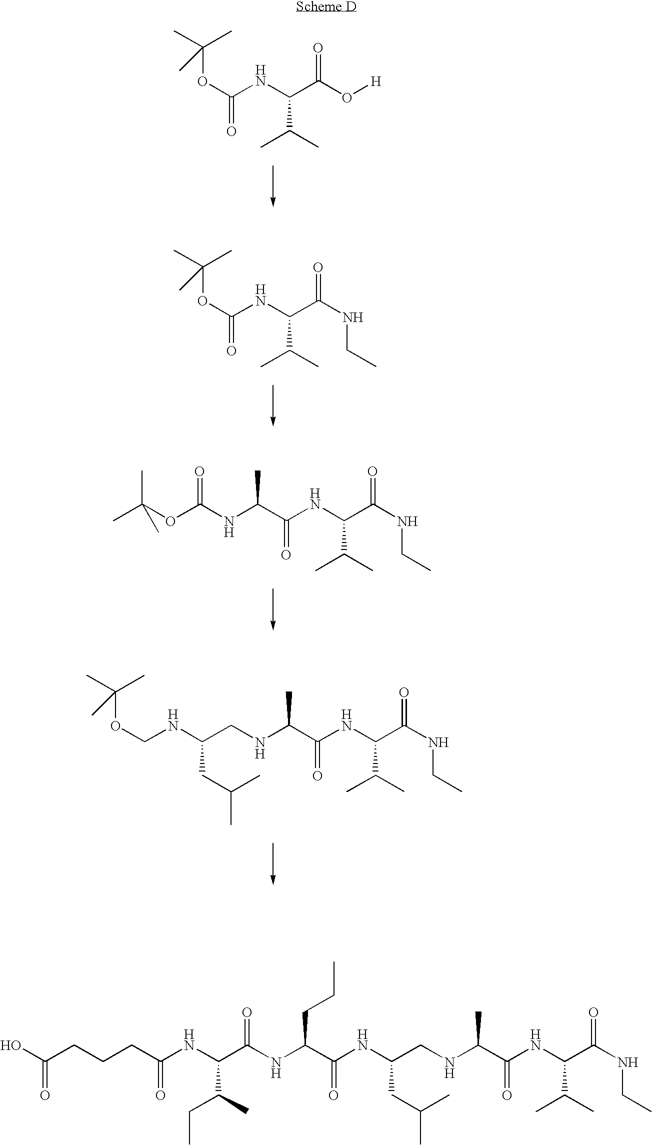

Scheme D illustrates a solution phase synthesis of the compounds of the invention. The starting Boc-protected amino acid is converted to an amide by standard coupling methods. After deprotection another coupling with a Boc-protected amino acid is performed. The resulting amine is reductively alkylated with a Boc-protected aminoaldehyde. Standard deprotection and coupling steps then lead to the final products.

The compounds of the invention, and pharmaceutically acceptable salts thereof, are useful for treating humans or animals suffering from a condition characterized by a pathological form of beta-amyloid peptide, such as beta-amyloid plaques or small A beta aggregates or simply A beta overproduction, and for helping to prevent or delay the onset of such a condition. For example, the compounds are useful for treating Alzheimer's disease, for helping prevent or delay the onset of Alzheimer's disease, for treating patients with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease. The compounds and compositions of the invention are particularly useful for treating or preventing Alzheimer's disease. When treating or preventing these diseases, the compounds of the invention can either be used individually or in combination, as is best for the patient.

As used herein, the term “treatment” means that the compounds of the invention can be used in humans with at least a tentative diagnosis of disease. The compounds of the invention will delay or slow the progression of the disease thereby giving the individual a more useful life span.

The term “prevention” means that the compounds of the present invention are useful when administered to a patient who has not been diagnosed as possibly having the disease at the time of administration, but who would normally be expected to develop the disease or be at increased risk for the disease. The compounds of the invention will slow the development of disease symptoms, delay the onset of the disease, or prevent the individual from developing the disease at all.

Prevention also includes administration of the compounds of the invention to those individuals thought to be predisposed to the disease due to age, familial history, genetic or chromosomal abnormalities, and/or due to the presence of one or more biological markers for the disease, such as a known genetic mutation of APP or APP cleavage products in brain tissues or fluids.

The compounds of the invention are administered in a therapeutically effective amount. The therapeutically effective amount will vary depending on the particular compound used and the route of administration, as is known to those skilled in the art.

The compounds of the invention can be administered orally, parenterally, (IV, IM, depo-IM, SQ, and depo SQ), sublingually, intranasally, inhalative, intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration or aerosols for inhalative administration. Typically the compounds described above are formulated into pharmaceutical compositions using techniques and procedures well known in the art.

About 0.1 to 1000 mg of a compound or mixture of compounds of the invention or a physiologically acceptable salt thereof is admixed with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 0.1 to about 1000 mg, preferably about 1 to about 300, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action.

The compounds may be formulated as the sole pharmaceutically active ingredient in the composition or may be combined with one or more different active ingredients.

The concentration of the compound is effective for delivery of an amount upon administration that lessens or ameliorates at least one symptom of the disorder for which the compound is administered. Typically, the compositions are formulated for single dosage administration.

The compounds and compositions of the invention can be enclosed in multiple or single dose containers. The compounds and compositions according to the invention can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampules, vials and the like for parenteral administration; and patches, medipads, creams, and the like for topical administration, and optionally pre-filled inhalators for inhalative administration.

The concentration of active compound in the drug composition will depend on absorption, inactivation, and excretion rates of the active compound, the dosage schedule, and amount administered as well as other factors known to those of skill in the art.

It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, lozenges or troches.

Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Methods for preparation of such formulations are known to those skilled in the art.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

The invention here is the new compounds of the invention and new methods of using the compounds of the invention. Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used in combination, with each other or with other therapeutic agents or approaches used to treat or prevent the conditions listed above. Such agents or approaches include: acetylcholine-esterase inhibitors such as tacrine (tetrahydroaminoacridine, marketed as COGNEXO), donepezil hydrochloride, (marketed as Aricept and rivastigmine; gamma-secretase inhibitors; anti-inflammatory agents such as cyclooxygenase 11 inhibitors; anti-oxidants such as Vitamin E and ginkolides; immunological approaches, such as, for example, immunization with A beta peptide or derivatives thereof or administration of anti-A beta peptide antibodies; neurotransmitter modulators like NS-2330; statins (HMG-CoA Reductase Inhibitors); and direct or indirect neurotropic agents such as Cerebrolysin (AIT-082) (Emilieu, 2000, Arch. Neurol. 57: 454), and other neurotropic agents of the future.

Most preferred are combinations with one or more additional active ingredient selected from the group consisting of atorvastatin, besipirdine, cevimeline, donepezil, eptastigmine, galantamine, glatiramer acetate, icopezil, ipidacrine, lazabemide, linopirdine, lubeluzole, memantine, metrifonate, milameline, nefiracetam, nimodipine, octreotide, rasagiline, rivastigmine, sabcomeline, sabeluzole, tacrine, valproate sodium, velnacrine, YM 796, Phenserine and zanapezil and/or with an antiinflammtory agents selected from the group consisting of rofecoxib, celecoxib, valdecoxib, nitroflurbiprofen, IQ-201, NCX-2216, CPI-1189, Colostrinin, ibuprofen, indomethacin, meloxicam and sulindac sulphide and/or one or more additional nerve growth factor and/or nerve growth modulator selected from the group consisting of: ABS-205, Inosine, KP-447, leteprinim, MCC-257, NS-521, NS-521, NS-2330, xaliproden.

It should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in this art.

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic and non-transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et. al., 1999, Mol. Cell. Neurosci. 14: 419-427; Vassar et. al., 1999, Science 286: 735-741; Yan et. al., 1999, Nature 402: 533-537; Sinha et. al., 1999, Nature 40: 537-540; and Lin et. al., 2000, PNAS USA 97: 1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Determination of BACE Activity In Vitro

Activity of BACE can be analyzed by different assay technologies, all incubating a catalytically active form of BACE with a potential substrate in a suitable buffer. The decrease in substrate concentration or the increase in product concentration can be monitored by applying different techniques depending on the nature of the substrate and include but are not limited to HPLC-MS analysis, fluorescence assays, fluorescence quenching assays. The substrate can be a peptide containing an amino acid sequence which is can be hydrolyzed by BACE which may be conjugated with dyes suitable for the detection system chosen or may extend to the protein substrate. As enzyme source, the full-length BACE enzyme can be used as well as the catalytically active ectodomain of the protein. An alternative assay format based on competition of the test compound with a BACE binding compound can be used. For IC₅₀ determination different concentrations of compound are incubated in the assay. The relative compound inhibition potency is determined by calculating the concentration of compound that showed a 50% reduction in detected signal compared to the enzyme reaction signal in the control wells with no added compound.

Useful inhibitory compounds are effective to inhibit 50% of beta-secretase enzymatic activity at a concentration of less than 50 micro molar, preferably at a concentration of 10 micro molar or less, more preferably 1 micro molar or less, and most preferably 10 nano molar or less.

In order to obtain the in vitro BACE inhibitory profile of the compounds of the invention they can be tested in the assays as outlined in the examples:

Example BACE Assay:

For each compound being tested, the BACE activity is monitored in a fluorescence quenching assay using the ectodomain of BACE (aa 1-454) fused to a myc-his tag and secreted from HEK293/APP/BACE_{ect}. cells into OptiMEM™ (Invitrogen) as enzyme source. The substrate peptide used has the amino acid sequence SEVNLDAEFK and possesses a Cy3-fluorophore at the N-terminus and a Cy5Q-quencher (Amersham) at the C-terminus. The substrate is dissolved at 1 mg/ml in DMSO.

The assay is performed in the presence of 10 μl OptiMEM containing the ectodomain of BACE, 100 μl water containing the desired concentration of compound with a max. conc. of 1% DMSO, 1 μM substrate peptide, and 20 mM NaOAc, pH 4.4 in a total assay volume of 200 μl in a 96 well plate. The reaction is incubated at 30° C. in a fluorimeter and the cleavage of the substrate is recorded as kinetic for 30 min. at ex: 530 nm, em: 590 nm.

The water used for preparation of the buffer or compound dilution is of highest purity. Blank wells containing either no inhibitor or no enzyme are included on each plate.

The compounds of formula (I) exemplified below as examples 1 to 175 show IC₅₀ values of less than 10 micro molar.

Aβ Secretion Assay

The secretion of Aβ can be monitored in cell lines of different origin. A representative set of such cells include but are not limited to human embryonic kidney 293 cells (HEK293), Chinese hamster ovary cells (CHO), human H4 neuroglimoa cells, human U373-MG astrocytoma glioblastoma cells, murine neuroblastoma N2a cells which are stably or transiently transfected with APP or mutated forms of APP which include but is not limited to the Swedish or London/Indiana mutations. Transfection of the cells can for example be achieved by introducing a pcDNA3 plasmid (Invitrogen) containing the human APP cDNA of interest using a transfection reagent like Lipofectamine (Invitrogen) according to the instructions of the manufacturer. Secretion of Aβ can also on a routine basis be analyzed from cells producing without genetic modification sufficient amounts of Aβ or by using highly sensitive Aβ detection assays. Cells suitable for an analysis of this kind include but are not limited to human IMR-32 neuroblastoma cells.

Secretion of Aβ from cells can also me analyzed from brain derived cells obtained from embryos or the new born offspring from APP transgenic mice as of example the mice described by Hsiao et al (Hsiao et al 1996 Science 274: 99-102). In addition brain derived cells from other organism such as rat or guinea pig may also be used.

Useful inhibitory compounds are effective to inhibit 50% of beta-secretase enzymatic activity in these cellular assays at a concentration of less than 50 micro molar, preferably at a concentration of 10 micro molar or less, more preferably 1 micro molar or less, and most preferably 10 nano molar or less.

Example Aβ Secretion Assay

In the following a protocol for the determination of Aβ from U373-MG cells which are stably expressing APP₇₅₁ under the control of a CMV promoter is given. The cells can be maintained in a culture medium like DMEM+glucose, sodium pyruvate, glutamine, pyridoxine-HCl, and 10% FCS. The cells are kept in an incubator at 37° C. in a water saturated atmosphere of 5% CO₂. For assaying compounds a confluent cell layer is incubated with compound concentrations in the range of 50 μM to 50 pM, originally dissolved in DMSO and for the assay diluted in 150 μl of the medium described, for 12-24 hours. The production of Aβ during this period of time in the presence or absence of compound is monitored by sandwich ELISA specific for Aβ40 and Aβ42. The antibodies 6E10 (Senetek) and SGY3160 (C. Eckman, Mayo Clinic, Jacksonville, Fla.) are used as capture antibodies and immobilized to the plate. Unspecific protein binding is blocked with Block Ace (Serotec) before adding the Aβ containing cell culture supernatant. The detection antibodies specific for Aβ40 and Aβ42 (Nanotools, Germany) are conjugated with alkaline phosphatase which activity is quantified using the substrate CSPD/Sapphire II (Applied Biosystems) according to the manufacturers instructions.

Potential effects of the compound in altering the Aβ level induced by an unspecific toxicity related mechanism are addressed by the reduction of AlamarBlue (Resazurin) after 60 min.

Potency of non-toxic compounds is determined by calculating the concentration of compound that showed a 50% reduction in the detected signal compared to the cells in the control wells with no added compound.

The compounds of formula (I) exemplified below as examples 1 to 175 show IC₅₀ values of less than 10 micro molar.

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015” and 5,811,633, and in Games et. al., 1995, Nature 373: 523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Unless defined otherwise, all scientific and technical terms used herein have the same meaning as commonly understood by one of skill in the art to which this invention belongs. All patents and publications referred to herein are hereby incorporated by reference for all purposes. The definitions and explanations below are for the terms as used throughout this entire document including both the specification and the claims.

All temperatures are in degrees Celsius,
(M+H)⁺ refers to the positive ion of a parent plus a hydrogen atom,
BOC refers to 1,1-dimethylethoxy carbonyl or t-butoxycarbonyl,
BOP refers to benzotriazol-1-yloxy-tris (dimethylamino) phosphonium hexafluoro-phosphate,
Bzl refers to benzyl,
CBZ refers to benzyloxycarbonyl,
CDI refers to 1,1′-carbonyldiimidazole,
Chromatography (column and flash chromatography) refers to purification/separation of compounds expressed as (support, eluent). It is understood that the appropriate fractions are pooled and concentrated to give the desired compound (s),
CMR refers to C-13 magnetic resonance spectroscopy, chemical shifts are reported in ppm (8) downfield from TMS,
DIC refers to dicyclohexyl carbodiimide,
DIPAMP refers to (R,R)-1,2-Ethanediylbis[(2-methoxyphenyl)phenylphosphine]
DCM refers to dichloromethane,
Dipea refers to diisopropylethylamine,
DIPEA refers to diisopropylethylamine,
DMF refers to dimethylformamide,
EDC refers to ethyl-1-(3-dimethylaminopropyl) carbodiimide or 1-(3-dimethylamino-propyl)-3-etliylcarbodiimide hydrochloride,
EI refers to electron impact. CI refers to chemical ionization. FAB refers to fast atom bombardment,
Ether refers to diethyl ether, unless specified otherwise,
FMOC refers to 9-fluorenylmethyl carbonate,
HATU refers to O-(7-azabenzotriazol-1-yl)-N,N,N′,N′-tetramethyluronium hexafluoro-phosphate,
HBTU refers to 2-(1H-Benzotriazole-1-yl)-1,1,3,3-tetramethyluronium hexafluoro-phosphate,
HOAC refers to acetic acid,
HOBt refers to 1-hydroxy benzotriazole hydrate,
HRMS refers to high resolution mass spectrometry,
IR refers to infrared spectroscopy,
MPLC refers to middle pressure liquid chromatography,
MS refers to mass spectrometry expressed as m/e, m/z or mass/charge unit,
NBS refers to N-bromosuccinimide,
NMM refers to N-methylmorpholine,
NMP refers to N-methylpyrrolidone,
NMR refers to nuclear (proton) magnetic resonance spectroscopy, chemical shifts are reported in ppm (d) downfield from TMS,
psi refers to pounds/in²,
RT refers to retention time,
Saline refers to an aqueous saturated sodium chloride solution,
Sta refers to (3S, 4S)-4-amino-3-hydroxy-6-methyl-heptanoic acid,
TBTU refers to 1-[Bis(dimethylamino)methylen]-1-H-benzotriazolim-tetrafluoroborate-3-oxide,
tBu refers to tert.-butyl,
TFA refers to trifluoracetic acid
THF refers to tetrahydrofurane
TMOF refers to trimethylorthoformate.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. When solvent pairs are used, the ratios of solvents used are volume/volume (v/v). When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent.

The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

The products were analyzed by analytical HPLC-MS and/or NMR.
HPLC-conditions 1: Column: Grom Nucleosil C18 250×2 mm, 5 μm; Flowrate: 0.3 ml/min; Buffer A: 0.1% TFA; Buffer B: 0.1% TFA in MeCN; Gradient: linear from 10% B to 100% B in 30 min;
HPLC-conditions 2: Column: Waters X-Terra 4.6×50 mm, 3.6 μm; Flowrate: 1 ml/min;
Buffer A: 0.1% TFA; Buffer B: 0.08% TFA in MeCN; Gradient: linear from 95% A to 2% A in 5 min;
HPLC-conditions 3: Column: XTerra MS C18 4.6×30 mm, 2.5 μm; Flowrate 1 ml/min.; Gradient: water:acetonitrile 95:5 to 2:98 in 4.5 minutes.
HPLC-conditions 4: Column: Waters Xterra MS. C18. 4.6×50 mm. 3.5 μm. Columntemp. 40° C.; Flowrate 1 ml/min. Solvent A: Water+0.1% TFA. Solvent B: MeCN+0.08% TFA. Gradient: from 95% A to 2% A in 5.10 min
HPLC-conditions 5: Column: Waters Xterra MS. C18. 2.1×50 mm. 3.5 μm. Columntemp. 40° C.; Flowrate 0.8 ml/min. Solvent A: Water+0.1% TFA. Solvent B: MeCN+0.08% TFA. Gradient: from 95% A to 2% A in 2.75 min

### Example 1

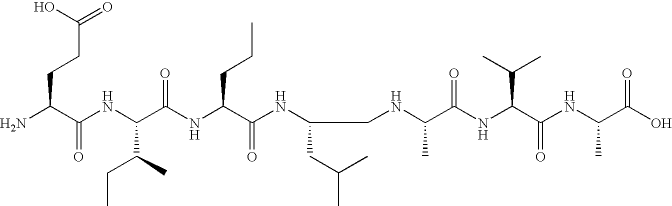

The compound was synthesized by standard solid phase peptide synthesis using a Wang resin (substitution 1.04 mmol/g) (Rapp-Polymere) (Scheme A).

Fmoc deprotections were performed by a 2 and 6 minute treatment with 20% piperidine in DMF. The resin was washed with DMF, MeOH, THF, DCM and DMF. The coupling of the first amino acid was performed with TBTU (3 equiv.), HOBt (3 equiv.), Dipea (8 equiv.) and Fmoc-protected amino acid (3 equiv.) in DMF as solvent for 2 hours. Non-reacted hydroxyl groups were capped by acetylation with acetic anhydride/Dipea/DMF (1:1:8) for 30 minutes. Coupling of the other amino acids were achieved with DIC as coupling reagent (5 equiv.), HOBt (5 equiv.) and the Fmoc-amino acid (5 equiv.) with DMF as solvent.

After coupling of the first three amino acids and Fmoc-deprotection the amino group was reductively alkylated with freshly prepared Fmoc-leucinal (3.5 equiv.) and NaCNBH₃ (10.5 equiv.) in DMF/HOAc (99:1, 4 ml) for 2.25 hours. After the alkylation the resin was carefully washed with DMF/HOAc (99:1), DMF, 5% Dipea in DMF and DMF. The resulting secondary amino group was protected by reaction with Boc₂O (10 equiv.) and Dipea (10 equiv.) in DMF for 16 hours.

The following Fmoc-amino acids and the N-terminal carboxylic acid were coupled until completion of the peptide chain as described above.

The cleavage from the resin and deprotection of tBu- and Boc-protecting groups was achieved by treatment with TFA/water (95:5) for 2 hour. The TFA solution was evaporated under reduced pressure and diethyl ether was added for precipitation of the peptide. The precipitate was dissolved in acetonitrile/water and purified by preparative reversed phase HPLC. The purified product was lyophilized .

The product was analyzed by analytical HPLC-MS and NMR. The analytical data were in agreement with the structure. Found [M+H]⁺700.5. RT=13.57 min (HPLC-conditions 1)

Examples 2, 3, and 4 were synthesized analogously. The analytical data were in agreement with the structures.

| Exam- | | Mass | HPLC- |
|---|---|---|---|
| ple | | Spectra | Retention |
| No. | Formula | [M + H]⁺ | Time |
| 2 | 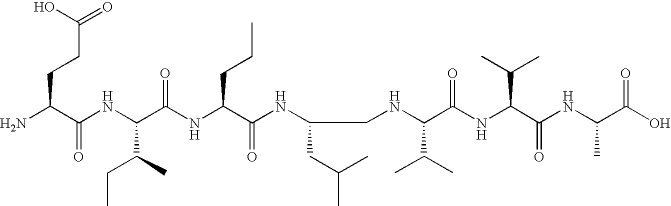 | 728.6 | RT = 14.62 min (HPLC- conditions 1) |
| 3 | 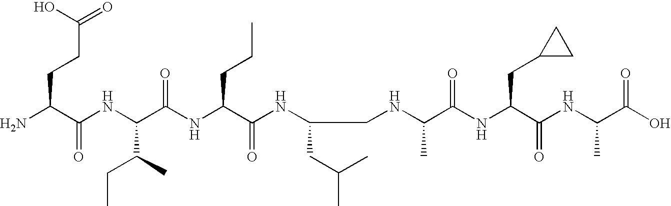 | 712.5 | RT = 14.19 min (HPLC- conditions 1) |
| 4 | 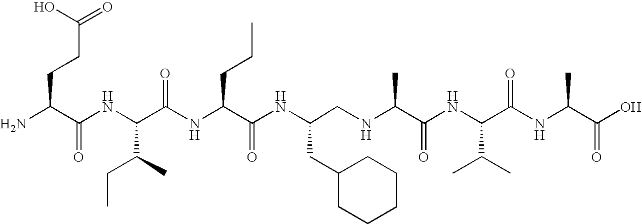 | 740.5 | RT = 18.92 min (HPLC- conditions 1) |

HPLC-conditions 1: Column: Grom Nucleosil C18 250×2 mm, 5 μm; Flowrate: 0.3 ml/min; Buffer A: 0.1% TFA; Buffer B: 0.1% TFA in MeCN; Gradient: linear from 10% B to 100% B in 30 min;

### Example 5

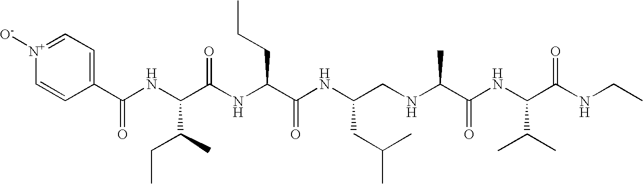

The compound was synthesized by standard solid phase peptide synthesis using a [3-((ethyl-Fmoc-amino)-methyl)-1-indol-yl]acetyl AM resin (277 mg, 0.2 mmol) (Novabiochem).

Fmoc-deprotections were performed by a 2 and 20 minute treatment with 30% piperidine in DMF. The coupling of the first amino acid was performed with HATU (5 equiv.), HOBt (5 equiv.), Dipea (5 equiv.) and Fmoc-protected amino acid (5 equiv.) in DMF as solvent for 16 hours. Coupling of the other amino acids were achieved with TBTU as coupling reagent (5 equiv.), HOBt (5 equiv.), Dipea (15 equiv.) and the amino acid (5 equiv.) with DMF as solvent.

After coupling of Fmoc-alanine and Fmoc-deprotection the amino group was reductively alkylated with freshly prepared Fmoc-leucinal (3.5 equiv.) and NaCNBH₃ (10.5 equiv.) in DMF/HOAc (99:1, 4 ml) for 2.25 hours. After the alkylation the resin was carefully washed with DMF/HOAc (99:1), DMF, 5% Dipea in DMF and DMF. The resulting secondary amino group was protected by reaction with Boc₂O (10 equiv.) and Dipea (10 equiv.) in DMF for 16 hours.

The following Fmoc-amino acids were coupled until completion of the peptide chain as described above. The terminal acetylation was performed with 4-nicotinic acid N-oxide (5 eq.), TBTU (5 eq.), HOBt (5 eq.), Dipea (15 eq.) in DMF as solvent.

The cleavage from the resin was achieved by treatment with TFA/water (95:5) for 1 hour. The TFA solution was evaporated under reduced pressure and diethyl ether was added for precipitation of the peptide. The precipitate was dissolved in acetonitrile/water and purified by preparative reversed phase HPLC. The purified product was lyophilized . Yield 90 mg (59%).

The product was analyzed by analytical HPLC-MS and NMR. The analytical data were in agreement with the structure. Found [M+H]⁺762.6; RT=3.55 min (HPLC-conditions 2).

The examples No. 6 to 32, 79 to 144 and 157 to 175 were synthesized analogously. N-oxides were obtained by oxidation of the corresponding heteroaryls with H₂O₂. Tetrazoles were synthesized according to S. J. Wittenberger et al., J. Org. Chem., (1993) 58, 4139-41; M. Alterman et al., J. Org. Chem. (2000), 65, 7984-89. The analytical data were in agreement with the structures.

| | | Mass | HPLC- |
|---|---|---|---|
| Example | | Spectra | Retention |
| No. | Formula | [M + H]⁺ | Time |
| 6 | 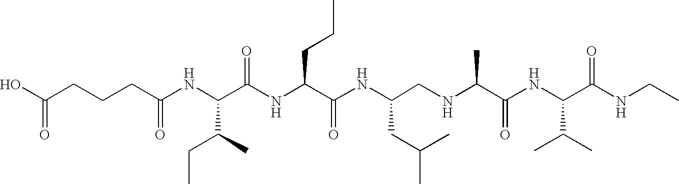 | 641.5 | RT = 20.53 min (HPLC- conditions 1) |
| 7 | 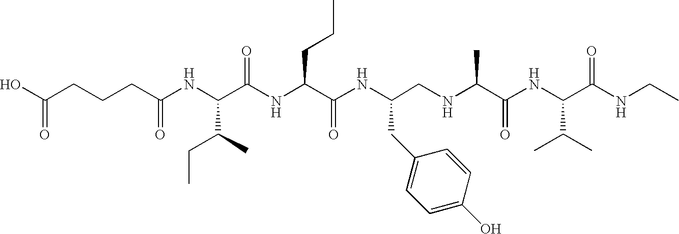 | 691.6; | RT = 19.43 min (HPLC- conditions 1) |
| 8 | 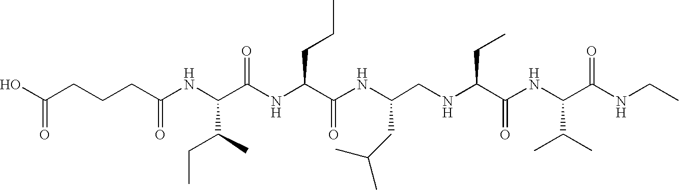 | 655.7 | RT = 21.22 min (HPLC- conditions 1) |
| 9 | 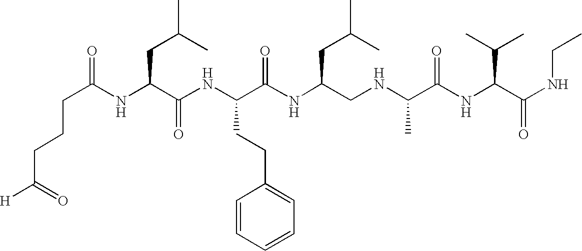 | 703.5 | RT = 4.62 min (HPLC- conditions 2) |
| 10 | 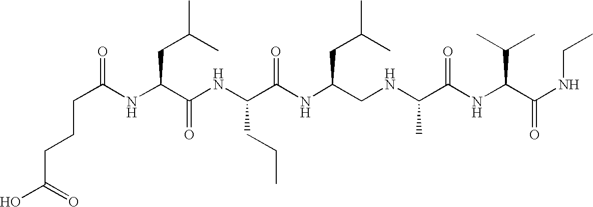 | 641.4 | RT = 4.41 min (HPLC- conditions 2) |
| 11 | 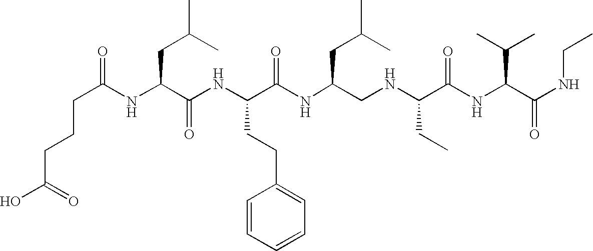 | 717.5 | RT = 4.69 min (HPLC- conditions 2) |
| 12 | 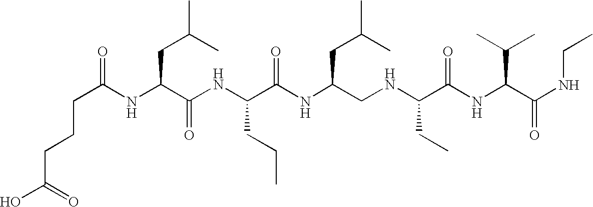 | 655.4 | RT = 4.48 min (HPLC- conditions 2) |
| 13 | 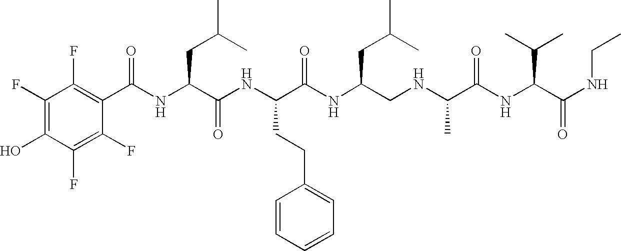 | 781.4 | RT = 4.97 min (HPLC- conditions 2) |
| 14 | 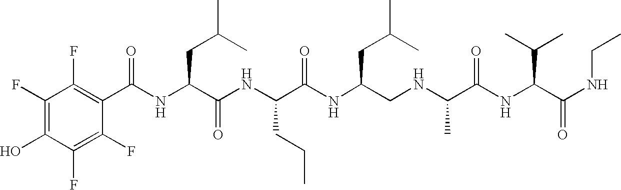 | 719.4 | RT = 4.75 min (HPLC- conditions 2) |
| 15 | 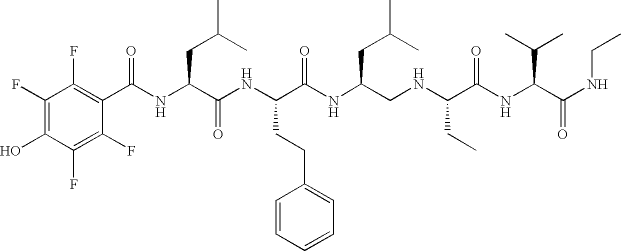 | 795.4 | RT = 5.02 min (HPLC- conditions 2) |
| 16 | 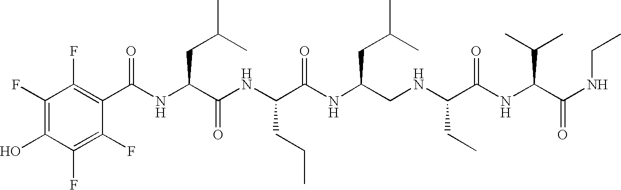 | 733.4 | RT = 4.82 min (HPLC- conditions 2) |
| 17 | 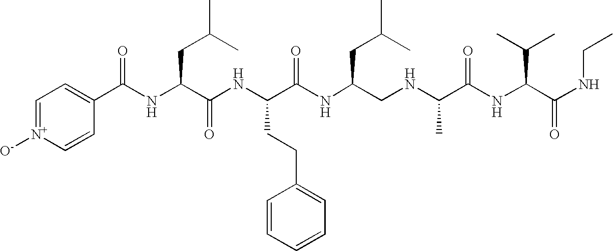 | 710.4 | RT = 4.55 min (HPLC- conditions 2) |
| 18 | 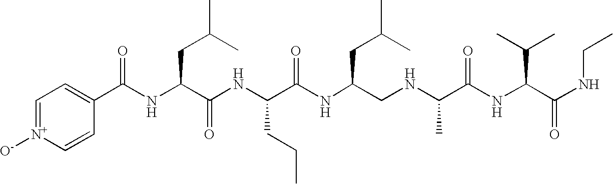 | 748.4 | RT = 4.29 min (HPLC- conditions 2) |
| 19 | 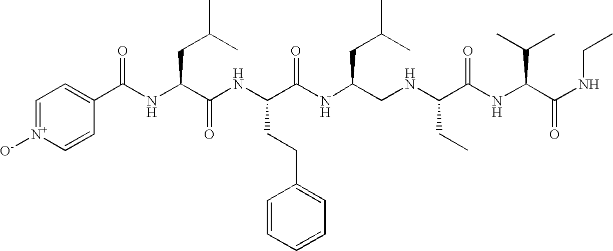 | 724.5 | RT = 4.61 min (HPLC- conditions 2) |
| 20 | 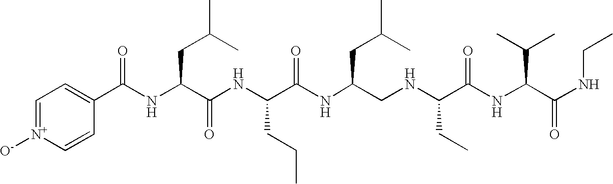 | 662.4 | RT = 4.37 min (HPLC- conditions 2) |
| 21 | 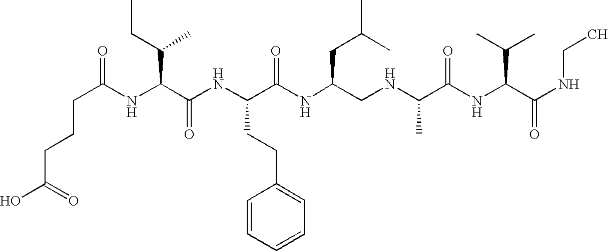 | 703.4 | RT = 4.65 min (HPLC- conditions 2) |
| 22 | 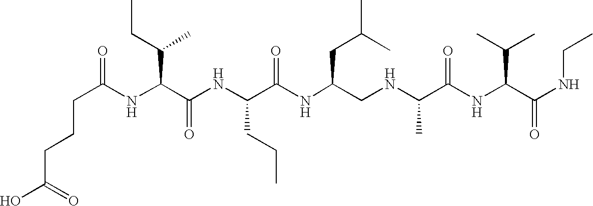 | 641.4 | RT = 4.41 min (HPLC- conditions 2) |
| 23 | 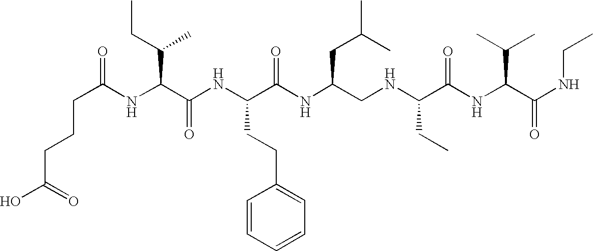 | 717.5 | RT = 4.70 min (HPLC- conditions 2) |
| 24 | 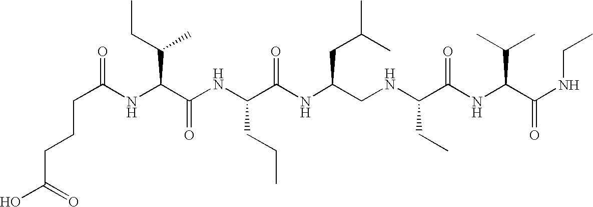 | 655.5 | RT = 4.47 min (HPLC- conditions 2) |
| 25 | 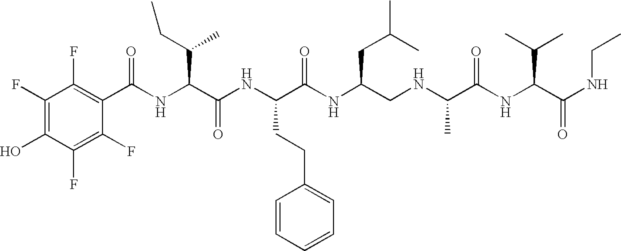 | 781.4 | RT = 4.94 min (HPLC- conditions 2) |
| 26 | 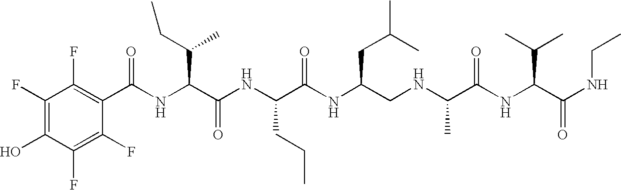 | 719.4 | RT = 4.73 min (HPLC- conditions 2) |
| 27 | 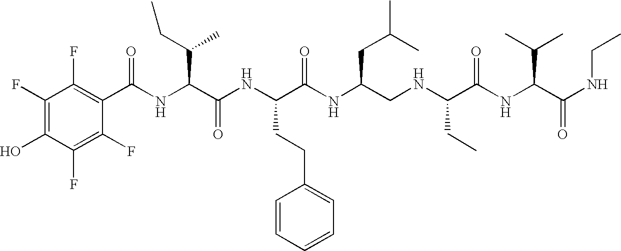 | 795.4 | RT = 4.99 min (HPLC- conditions 2) |
| 28 | 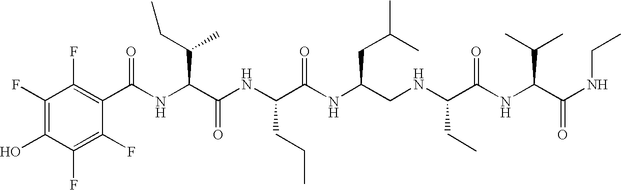 | 733.4 | RT = 4.80 min (HPLC- conditions 2) |
| 29 | 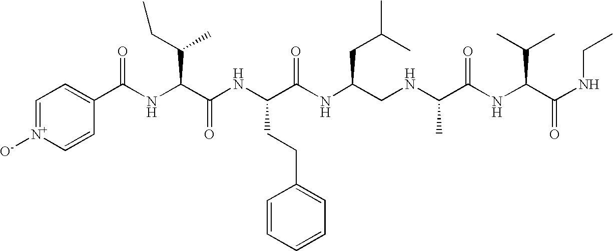 | 710.4 | RT = 4.52 min (HPLC- conditions 2) |
| 30 | 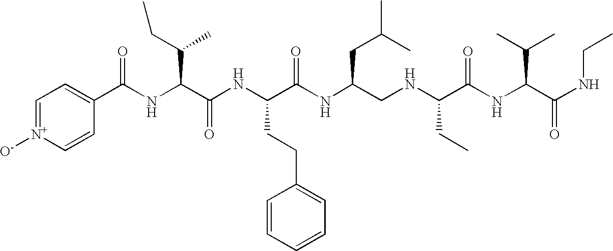 | 724.5 | RT = 4.59 min (HPLC- conditions 2) |
| 31 | 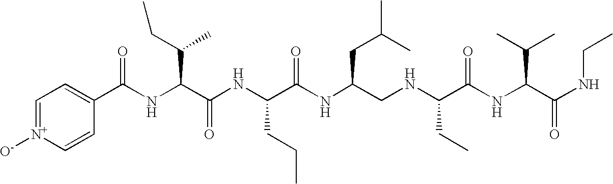 | 662.4 | RT = 4.32 min (HPLC- conditions 2) |
| 32 | 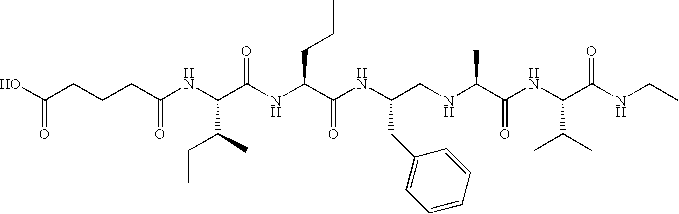 | 675.7 | RT = 21.53 min (HPLC- conditions 1) |
| 79 | 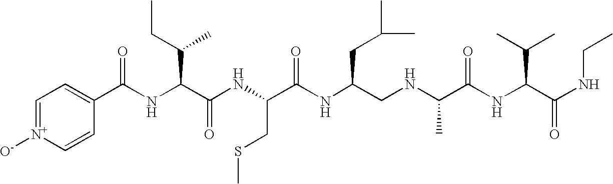 | 666.6 | RT = 1.68 min (HPLC- conditions 5) |
| 80 | 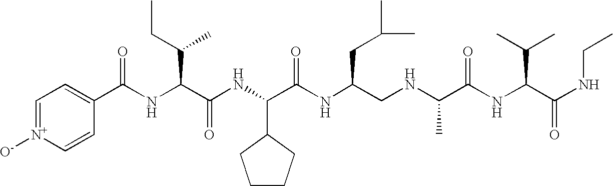 | 674.7 | RT = 1.74 min (HPLC- conditions 5) |
| 81 | 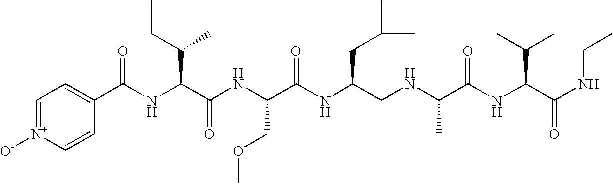 | 650.6 | RT = 1.59 min (HPLC- conditions 5) |
| 82 | 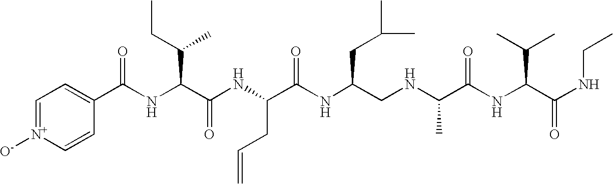 | 646.7 | RT = 1.65 min (HPLC- conditions 5) |
| 83 | 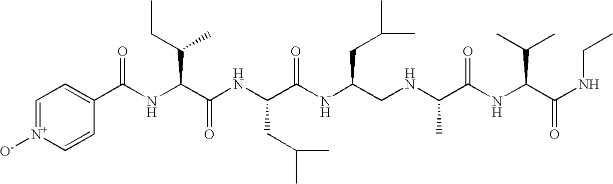 | 662.8 | RT = 1.74 min (HPLC- conditions 5) |
| 84 | 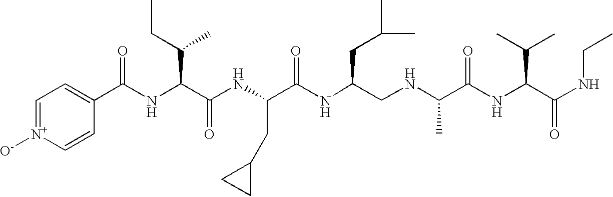 | 660.8 | RT = 1.69 min (HPLC- conditions 5) |
| 85 | 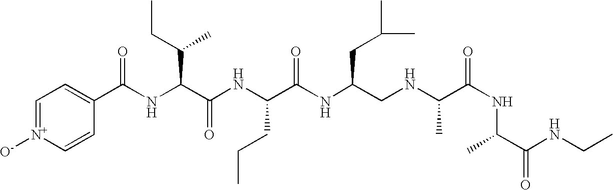 | 620.3 | RT = 2.93 min (HPLC- conditions 4) |
| 86 | 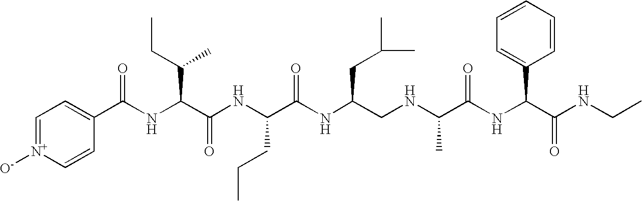 | 682.2 | RT = 3.29 min (HPLC- conditions 4) |
| 87 | 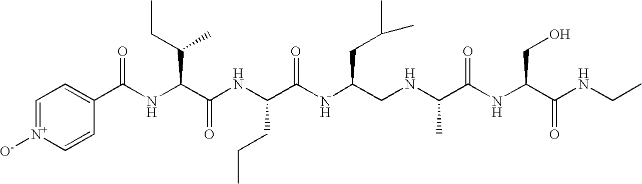 | 636.2 | RT = 2.86 min (HPLC- conditions 4) |
| 88 | 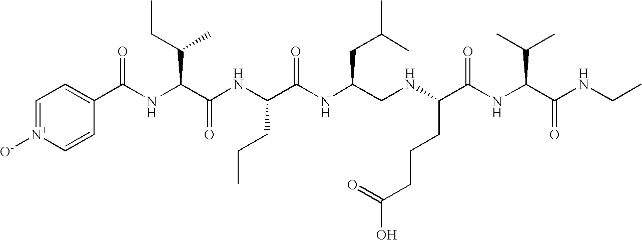 | 720.3 | RT = 3.15 min (HPLC- conditions 4) |
| 89 | 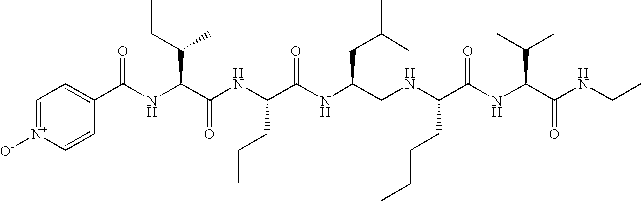 | 690.3 | RT = 3.46 min (HPLC- conditions 4) |
| 90 | 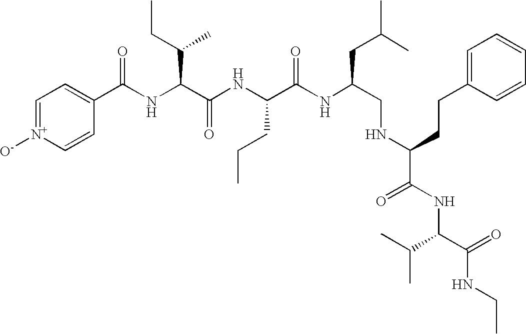 | 738.3 | RT = 3.66 min (HPLC- conditions 4) |
| 91 | 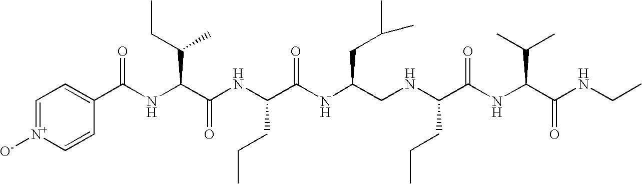 | 676.3 | RT = 3.34 min (HPLC- conditions 4) |
| 92 | 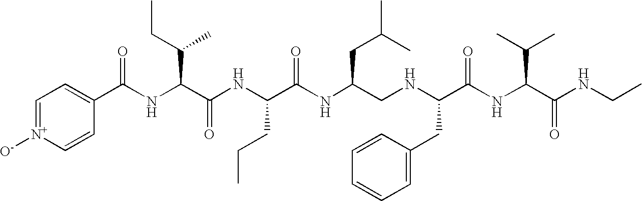 | 724.3 | RT = 3.5 min (HPLC- conditions 4) |
| 93 | 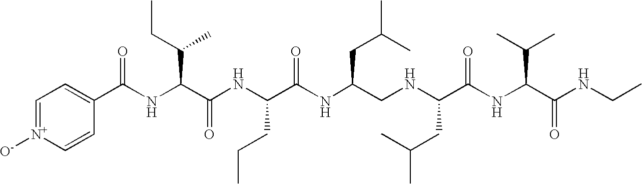 | 690.3 | RT = 3.44 min (HPLC- conditions 4) |
| 94 | 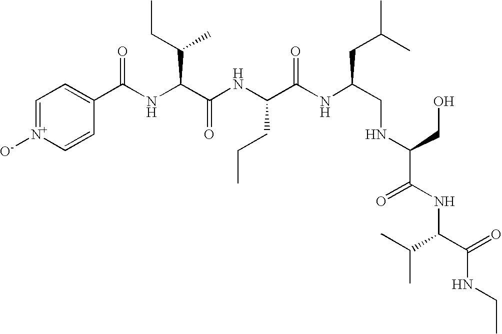 | 664.3 | RT = 3.11 min (HPLC- conditions 4) |
| 95 | 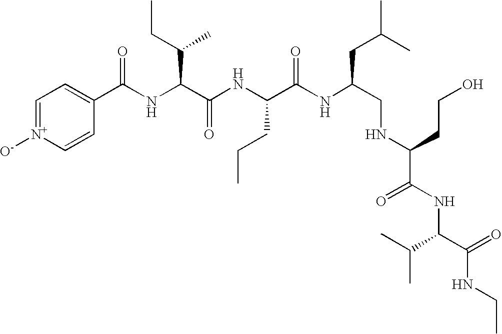 | 678.3 | RT = 3.09 min (HPLC- conditions 4) |
| 96 | 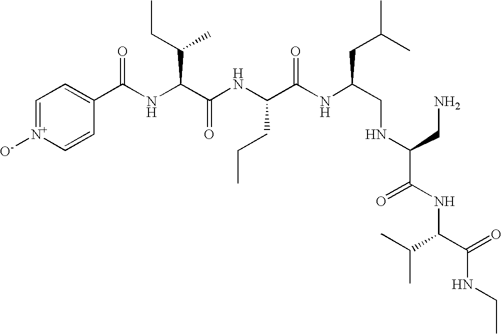 | 663.3 | RT = 2.95 min (HPLC- conditions 4) |
| 97 | 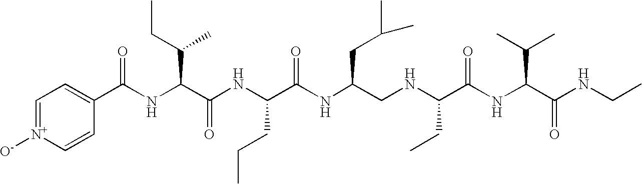 | 662.3 | RT = 3.2 min (HPLC- conditions 4) |
| 98 | 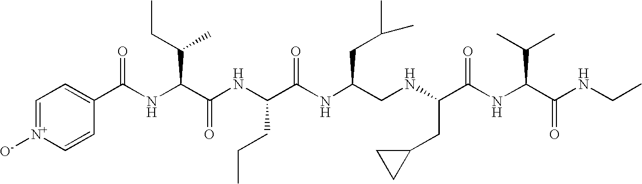 | 688.3 | RT = 3.35 min (HPLC- conditions 4) |
| 99 | 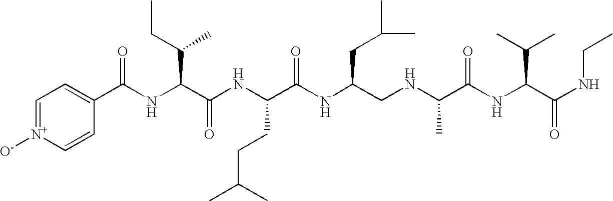 | 676.6 | RT = 3.32 min (HPLC- conditions 4) |
| 100 | 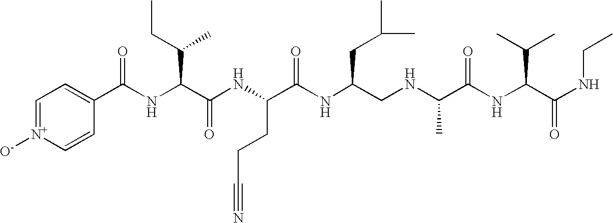 | 659.6 | RT = 2.98 min (HPLC- conditions 4) |
| 101 | 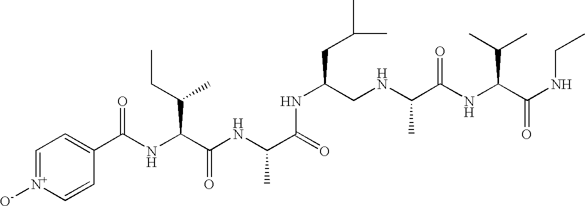 | 620.5 | RT = 2.93 min (HPLC- conditions 4) |
| 102 | 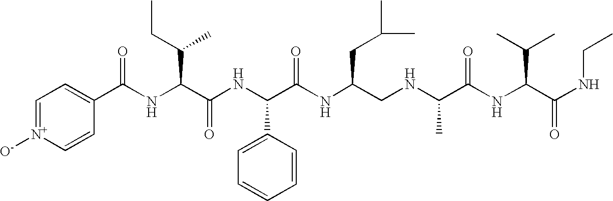 | 682.6 | RT = 3.16 min (HPLC- conditions 4) |
| 103 | 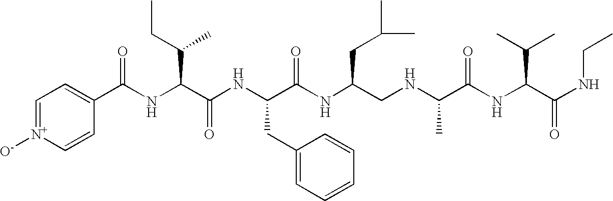 | 696.6 | RT = 3.23 min (HPLC- conditions 4) |
| 104 | 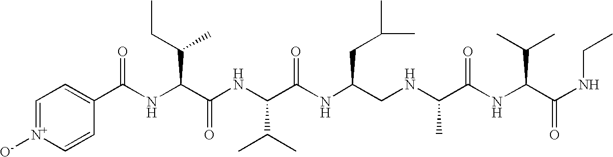 | 648.6 | RT = 3.05 min (HPLC- conditions 4) |
| 105 | 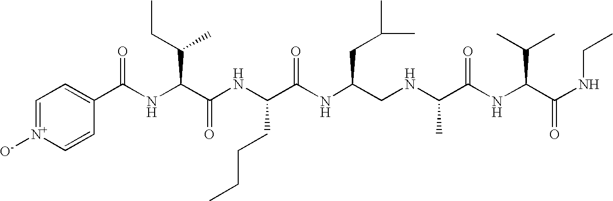 | 662.6 | RT = 3.24 min (HPLC- conditions 4) |
| 106 | 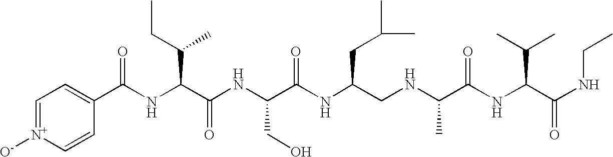 | 636.5 | RT = 2.91 min (HPLC- conditions 4) |
| 107 | 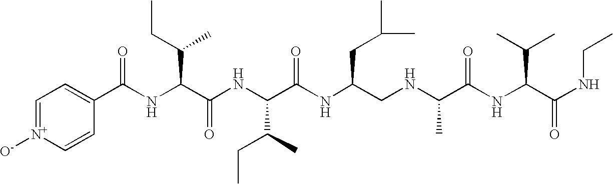 | 662.6 | RT = 3.18 min (HPLC- conditions 4) |
| 108 | 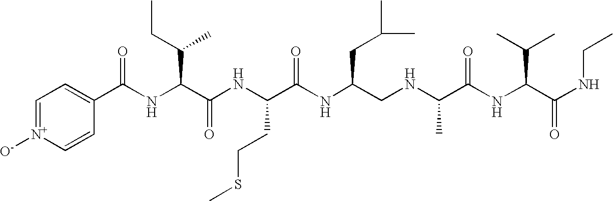 | 680.5 | RT = 3.08 min (HPLC- conditions 4) |
| 109 | 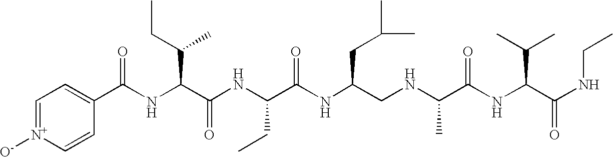 | 634.5 | RT = 3.03 min (HPLC- conditions 4) |
| 110 | 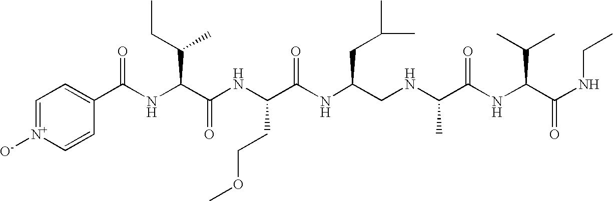 | 664.6 | RT = 2.98 min (HPLC- conditions 4) |
| 111 | 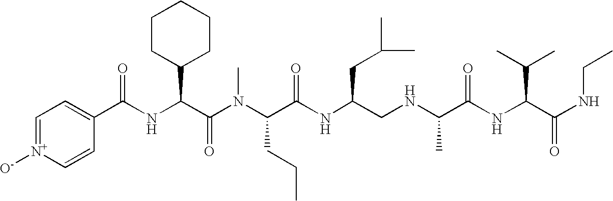 | 688.4 | RT = 3.51 min (HPLC- conditions 4) |
| 112 | 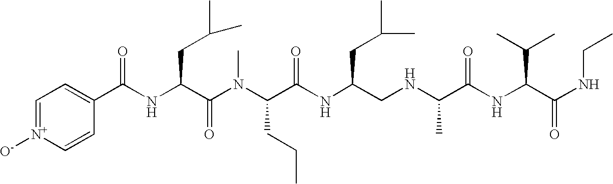 | 662.3 | RT = 3.36 min (HPLC- conditions 4) |
| 113 | 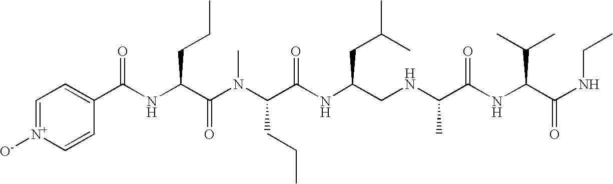 | 648.3 | RT = 3.25 min (HPLC- conditions 4) |
| 114 | 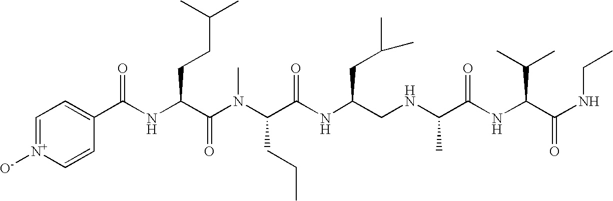 | 676.6 | RT = 3.77 min (HPLC- conditions 4) |
| 115 | 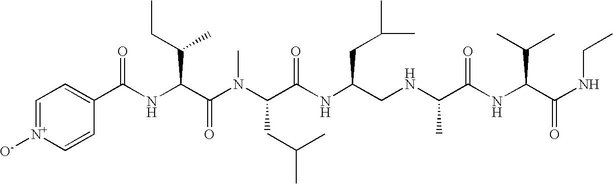 | 676.6 | RT = 3.67 min (HPLC- conditions 4) |
| 116 | 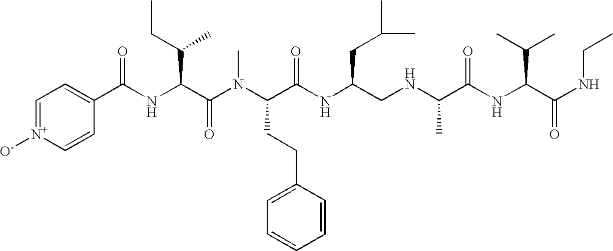 | 724.3 | RT = 3.61 min (HPLC- conditions 4) |
| 117 | 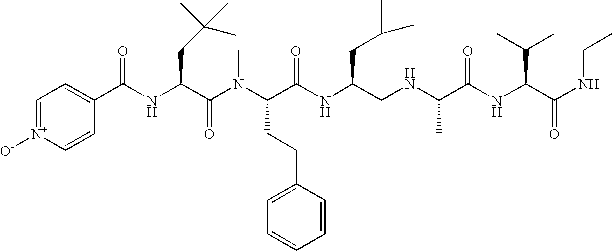 | 738.3 | RT = 3.69 min (HPLC- conditions 4) |
| 118 | 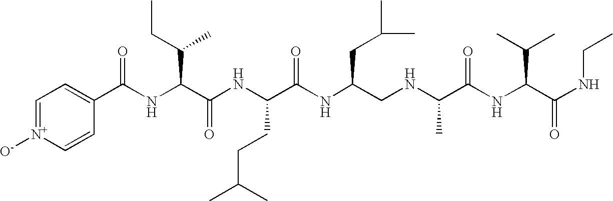 | 676.6 | RT = 3.86 min (HPLC- conditions 4) |
| 119 | 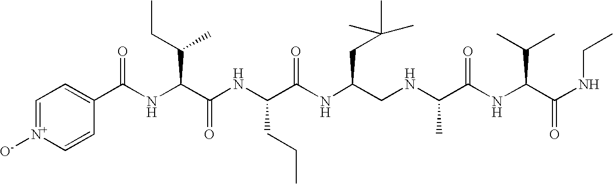 | 662.3 | RT = 3.21 min (HPLC- conditions 4) |
| 120 | 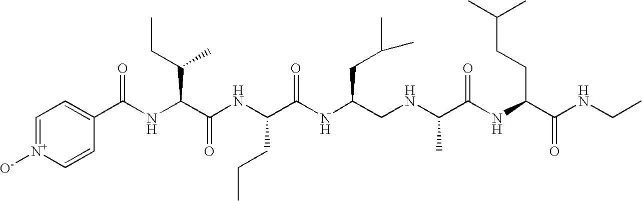 | 676.3 | RT = 3.42 min (HPLC- conditions 4) |
| 121 | 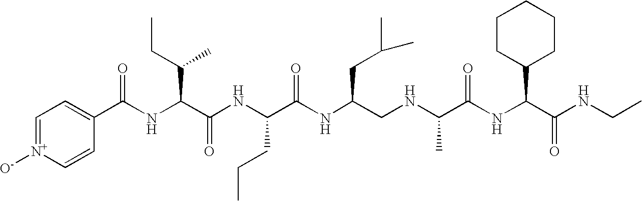 | 688.3 | RT = 3.42 min (HPLC- conditions 4) |
| 122 | 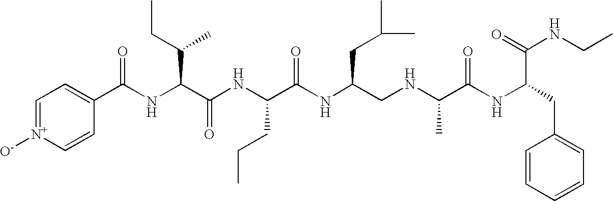 | 696.3 | RT = 3.33 min (HPLC- conditions 4) |
| 123 | 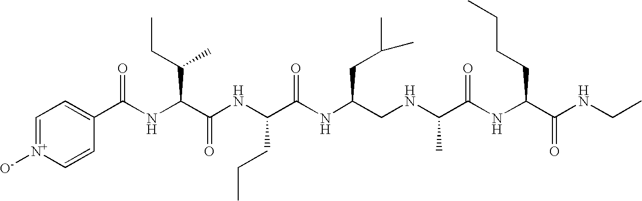 | 662.3 | RT = 3.3 min (HPLC- conditions 4) |
| 124 | 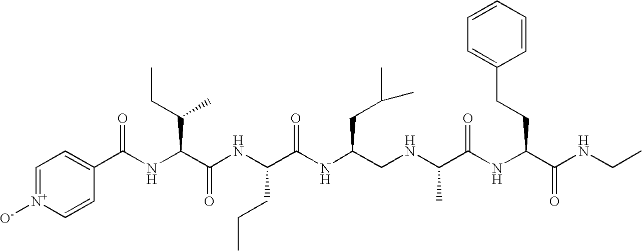 | 710.3 | RT = 3.46 min (HPLC- conditions 4) |
| 125 | 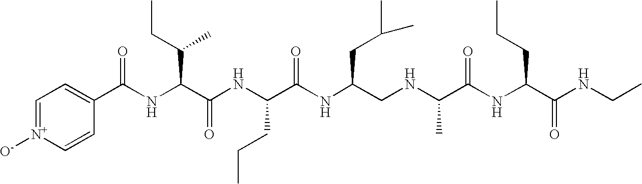 | 648.3 | RT = 3.17 min (HPLC- conditions 4) |
| 126 | 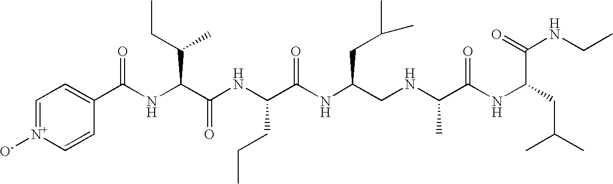 | 662.3 | RT = 3.28 min (HPLC- conditions 4) |
| 127 | 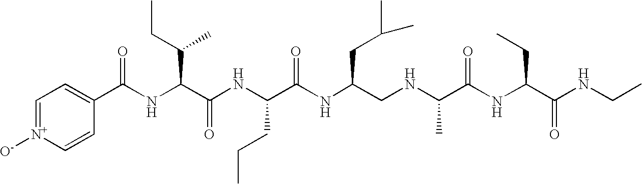 | 634.3 | RT = 3.03 min (HPLC- conditions 4) |
| 128 | 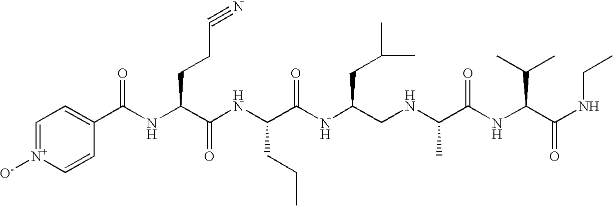 | 645.6 | RT = 3.25 min (HPLC- conditions 4) |
| 129 | 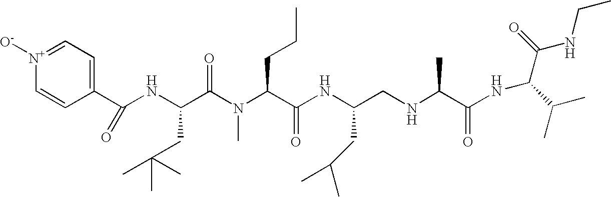 | 676.6 | RT = 3.69 min (HPLC- conditions 4) |
| 130 | 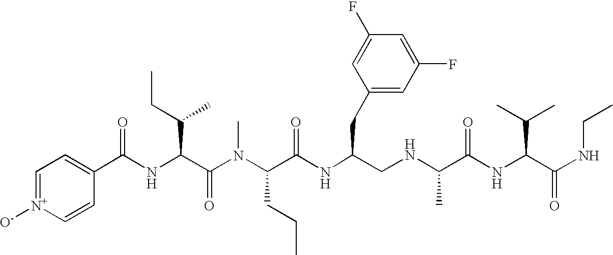 | 732.7 | RT = 3.88 min (HPLC- conditions 4) |
| 131 | 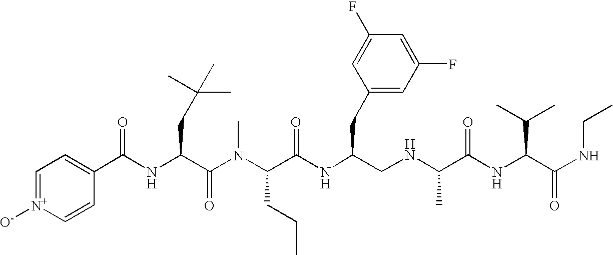 | 746.7 | RT = 3.93 min (HPLC- conditions 4) |
| 132 | 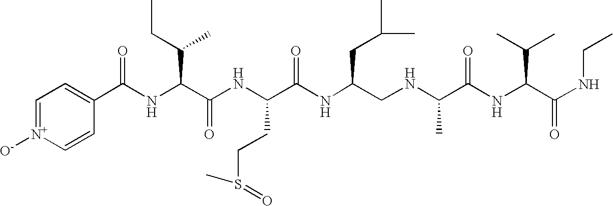 | 696.2 | RT = 2.87 min (HPLC- conditions 4) |
| 133 | 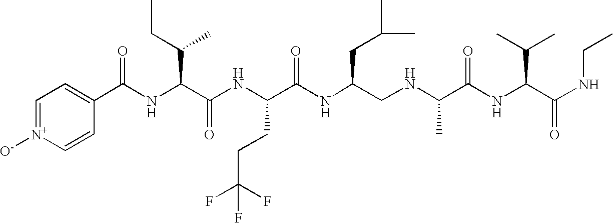 | 702.3 | RT = 3.27 min (HPLC- conditions 4) |
| 134 | 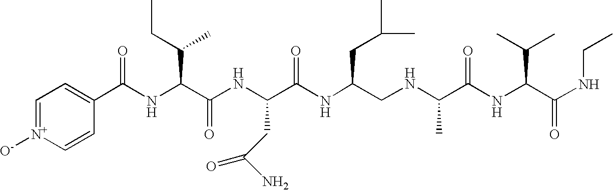 | 663.5 | RT = 3.19 min (HPLC- conditions 4) |
| 135 | 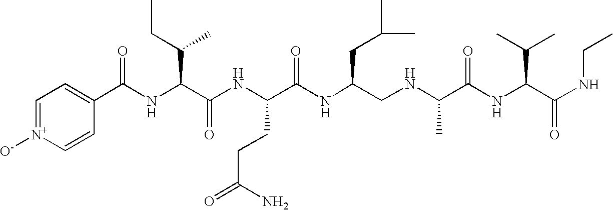 | 677.3 | RT = 2.86 min (HPLC- conditions 4) |
| 136 | 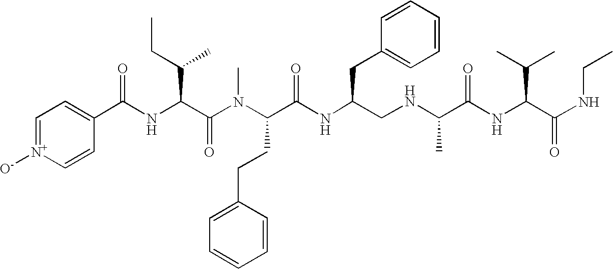 | 758.3 | RT = 3.66 min (HPLC- conditions 4) |
| 137 | 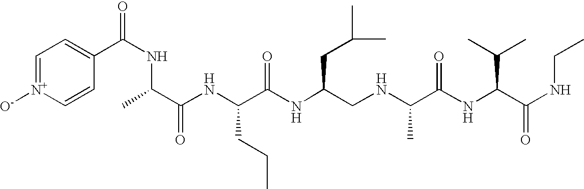 | 606.3 | RT = 1.53 min (HPLC- conditions 4) |
| 138 | 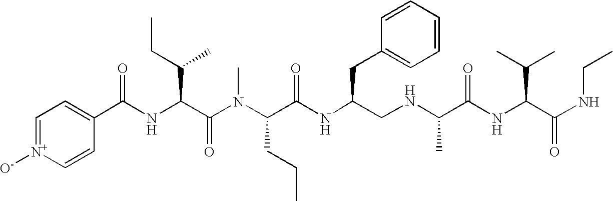 | 696.8 | RT = 3.37 min (HPLC- conditions 4) |
| 139 | 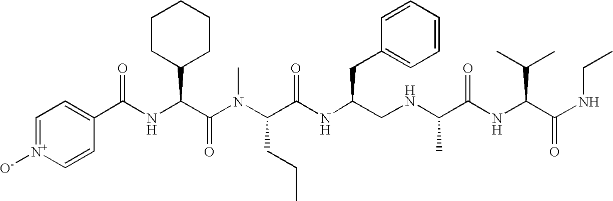 | 722.8 | RT = 3.52 min (HPLC- conditions 4) |
| 140 | 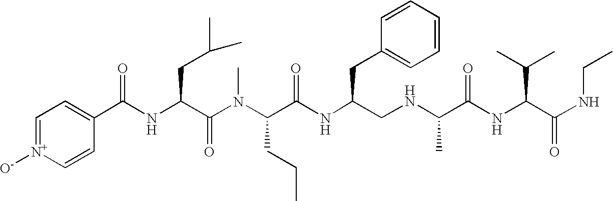 | 696.8 | RT = 3.37 min (HPLC- conditions 4) |
| 141 | 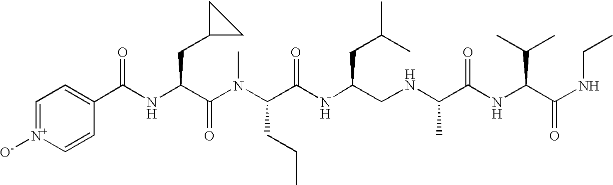 | 660.2 | RT = 3.22 min (HPLC- conditions 4) |
| 142 | 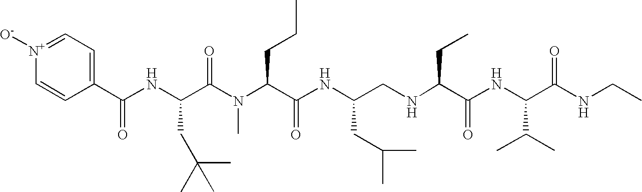 | 690.3 | RT = 3.49 min (HPLC- conditions 4) |
| 143 | 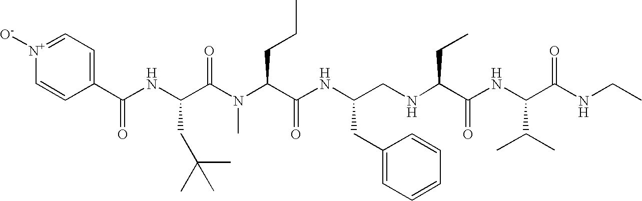 | 724.3 | RT = 3.57 min (HPLC- conditions 4) |
| 144 | 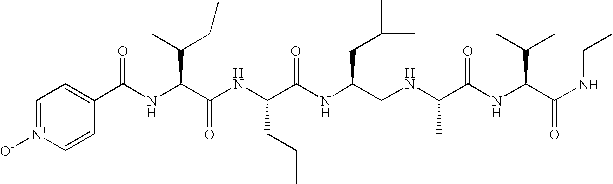 | 648.4 | RT = 4.3 min (HPLC- conditions 4) |
| 157 | 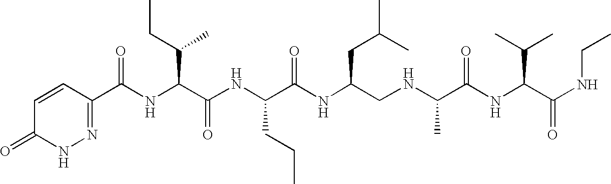 | 649.7 | RT = 3.19 min (HPLC- conditions 4) |
| 158 | 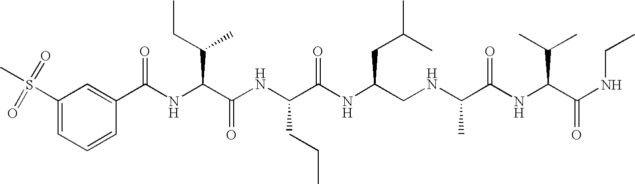 | 709.7 | RT = 3.59 min (HPLC- conditions 4) |
| 159 | 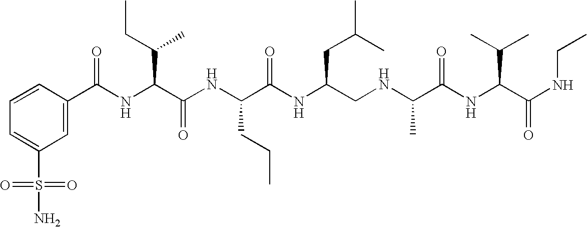 | 710.7 | RT = 3.46 min (HPLC- conditions 4) |
| 160 | 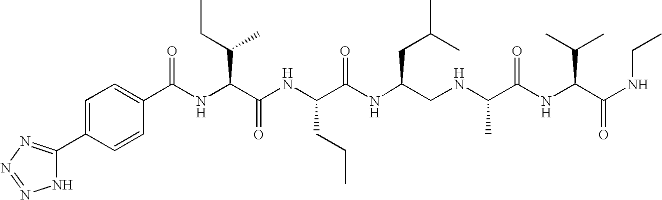 | 699.8 | RT = 3.47 min (HPLC- conditions 4) |
| 161 | 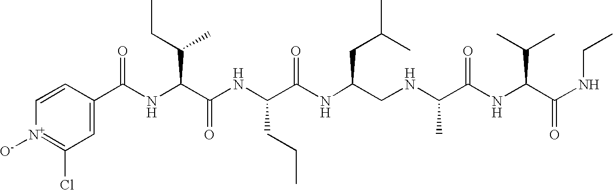 | 682.7 | RT = 3.27 min (HPLC- conditions 4) |
| 162 | 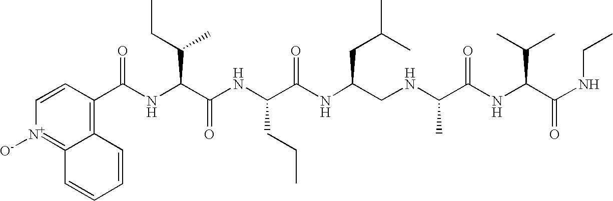 | 698.8 | RT = 3.29 min (HPLC- conditions 4) |
| 163 | 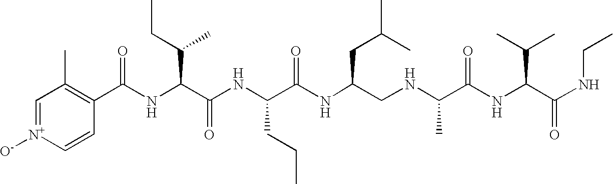 | 662.8 | RT = 3.06 min (HPLC- conditions 4) |
| 164 | 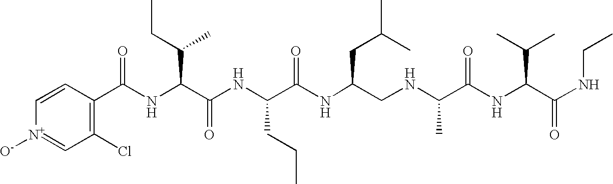 | 682.4 | RT = 3.51 min (HPLC- conditions 4) |
| 165 | 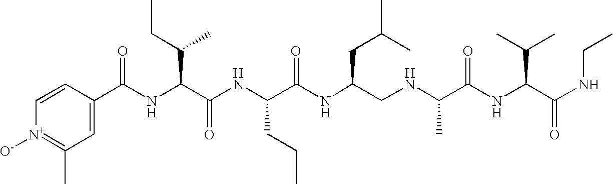 | 662.5 | RT = 3.48 min (HPLC- conditions 4) |
| 166 | 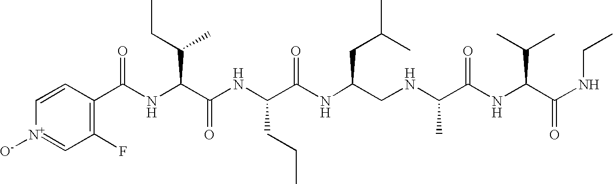 | 666.5 | RT = 3.46 min (HPLC- conditions 4) |
| 167 | 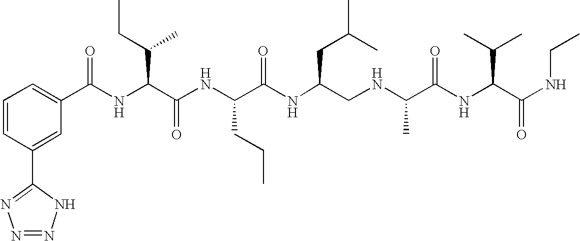 | 699.8 | RT = 3.52 min (HPLC- conditions 4) |
| 168 | 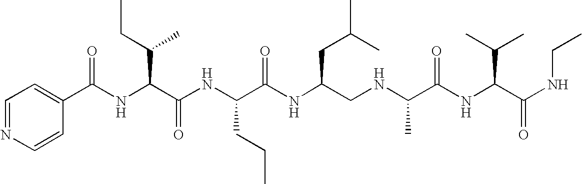 | 632.8 | RT = 2.99 min (HPLC- conditions 4) |
| 169 | 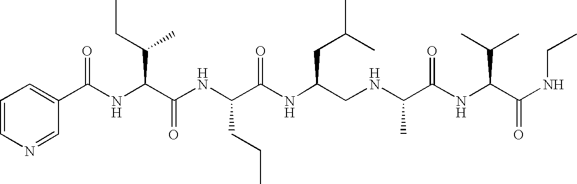 | 632.8 | RT = 3.07 min (HPLC- conditions 4) |
| 170 | 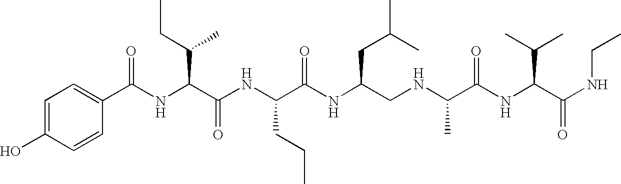 | 647.8 | RT = 3.52 min (HPLC- conditions 4) |
| 171 | 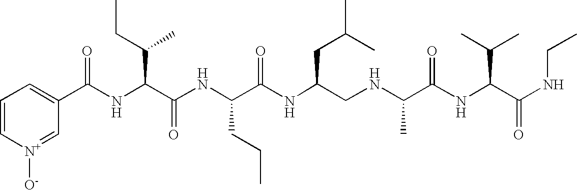 | 648.8 | RT = 3.06 min (HPLC- conditions 4) |
| 172 | 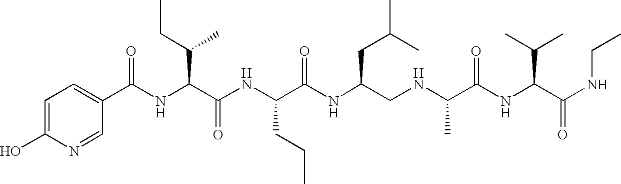 | 648.8 | RT = 3.16 min (HPLC- conditions 4) |
| 173 | 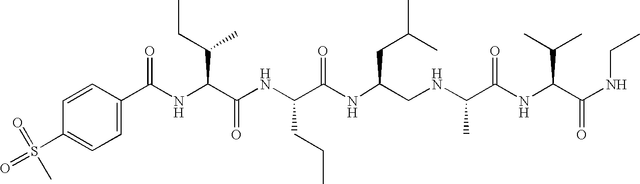 | 709.7 | RT = 3.56 min (HPLC- conditions 4) |
| 174 | 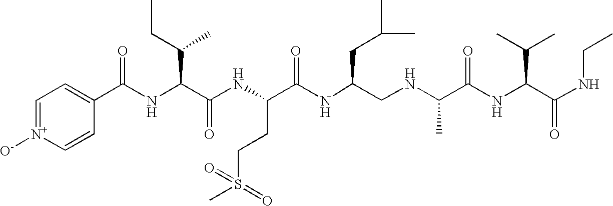 | 712.7 | RT = 2.86 min (HPLC- conditions 4) |
| 175 | 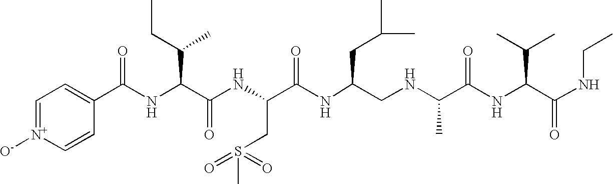 | 698.7 | RT = 2.86 min (HPLC- conditions 4) |

### Example 33

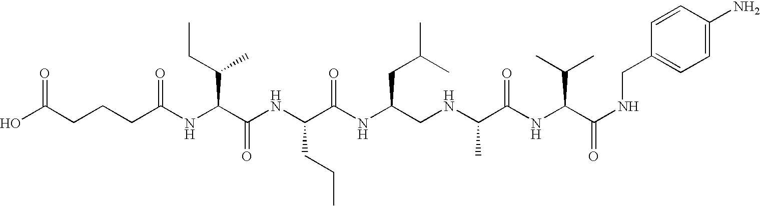

The compound was synthesized by standard solid phase peptide synthesis using a 3-(formylindolyl)acetamidomethylpolystyrene resin (200 mg, 0.196 mmol; substitution 0.98 mmol/g) (Merckbiosciences).

For the first reductive alkylation the resin which was suspended in 1,2-dichloroethane (5 ml) was reacted with a solution of 4-(aminomethyl)-1-N-Boc-aniline (10 eqiv.) in 1,2-dichloroethane/TMOF 2:1 (4 ml) and Na(OAc)₃BH (10 eqiv.) in 1,2-dichloroethane/TMOF 2:1 (2 ml) overnight at room temperature. Additional Na(OAc)₃BH (20 eqiv.) in MeOH were added and the suspension was shaken for 6 hours. The resin was carefully washed with DMF, MeOH, THF and DCM.

Fmoc-deprotections were performed by a 2 and 6 minute treatment with 20% piperidine in DMF. The resin was washed with DMF, MeOH, THF, DCM and DMF. The coupling of the first amino acid was performed with HATU (5 equiv.), Dipea (10 equiv.) and Fmoc-protected amino acid (5 equiv.) in DMF as solvent overnight. Coupling of the other amino acids were achieved with DIC as coupling reagent (5 equiv.), HOBt (5 equiv.) and the Fmoc-amino acid (5 equiv.) with DMF as solvent.

After coupling of the first two amino acids and Fmoc-deprotection the amino group was reductively alkylated with freshly prepared Fmoc-leucinal (3.5 equiv.) and NaCNBH₃ (10.5 equiv.) in DMF/HOAc (99:1,4 ml) for 2.25 hours. After the alkylation the resin was carefully washed with DMF/HOAc (99:1), DMF, 5% Dipea in DMF and DMF. The resulting secondary amino group was protected by reaction with Boc₂O (10 equiv.) and Dipea (10 equiv.) in DMF for 16 hours.

The following Fmoc-amino acids and the N-terminal carboxylic acid were coupled until completion of the peptide chain as described above.

The cleavage from the resin was achieved by treatment with TFA/DCM (5:95) for 2 hour. The solution was evaporated and treated with TFA/water (95:5) for 1 hour. The TFA solution was evaporated under reduced pressure and diethyl ether was added for precipitation of the peptide. The precipitate was dissolved in acetonitrile/water and purified by preparative reversed phase HPLC. The purified product was lyophilized.

The product was analyzed by analytical HPLC-MS and NMR. The analytical data were in agreement with the structure. Found [M+H]⁺718.7; RT=19.58 min (HPLC-conditions 1).

The examples 34 to 50 were synthesized analogously. The analytical data were in agreement with the structures.

| | | Mass | |
|---|---|---|---|
| Example | | Spectra | HPLC-Retention |
| No. | Formula | [M + H]⁺ | Time |
| 34 | 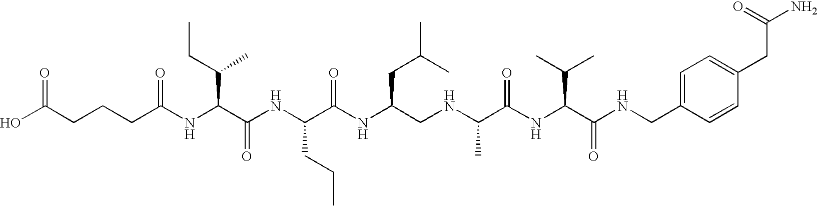 | 760.7 | RT = 3.71 min (HPLC-conditions 2) |
| 35 | 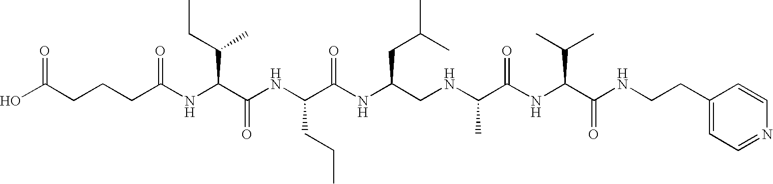 | 718.7 | RT = 3.43 min (HPLC-conditions 2) |
| 36 | 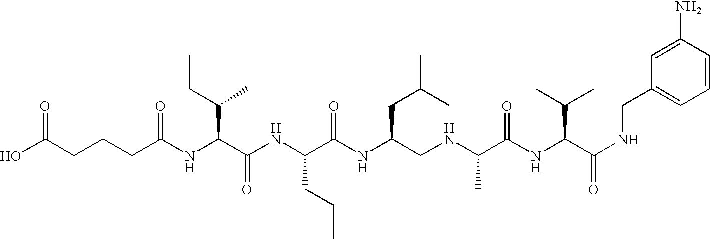 | 718.7 | RT = 3.50 min (HPLC-conditions 2) |
| 37 | 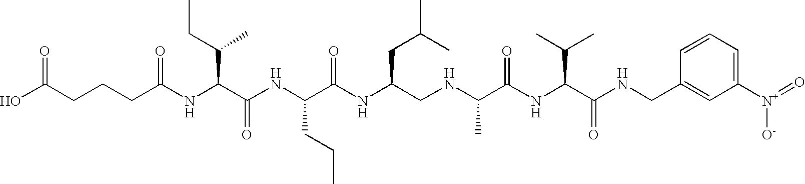 | 748.6 | RT = 4.20 min (HPLC-conditions 2) |
| 38 | 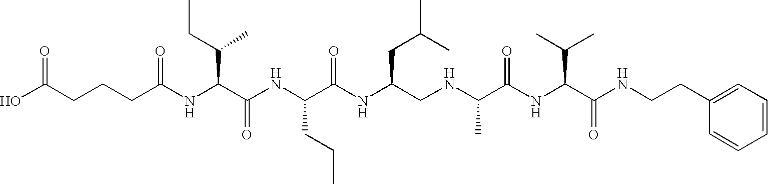 | 717.7 | RT = 4.26 min (HPLC-conditions 2) |
| 39 | 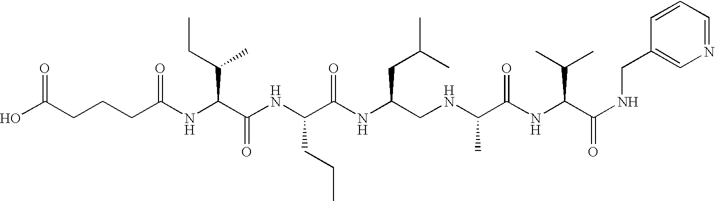 | 704.6 | RT = 3.45 min (HPLC-conditions 2) |
| 40 | 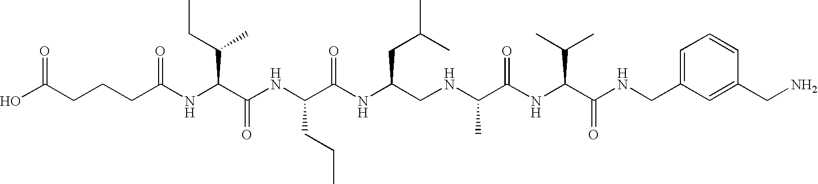 | 732.7 | RT = 3.51 min (HPLC-conditions 2) |
| 41 | 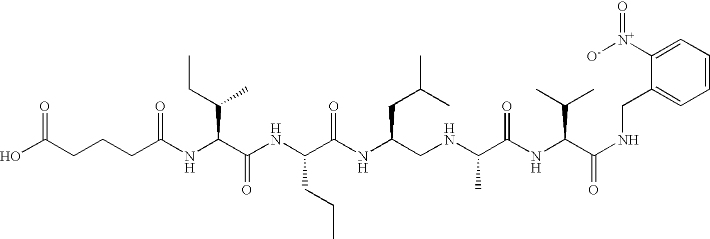 | 748.6 | RT = 4.18 min (HPLC-conditions 2) |
| 42 | 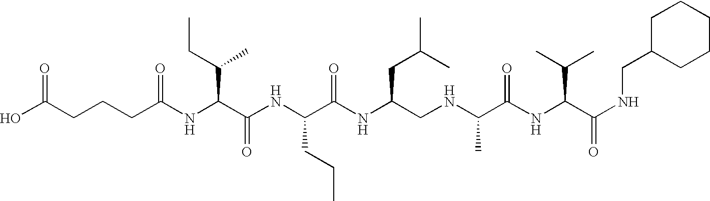 | 709.7 | RT = 4.37 min (HPLC-conditions 2) |
| 43 | 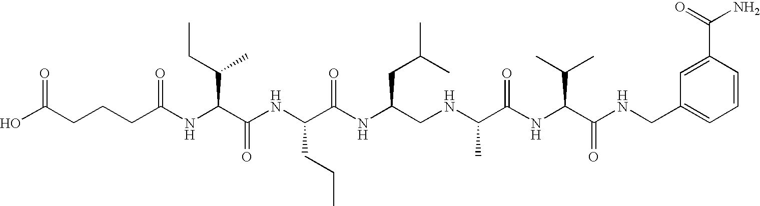 | 746.6 | RT = 3.72 min (HPLC-conditions 2) |
| 44 | 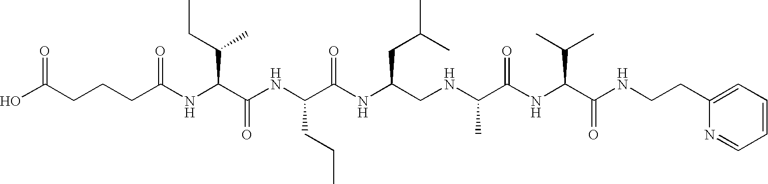 | 718.6 | RT = 3.65 min (HPLC-conditions 2) |
| 45 | 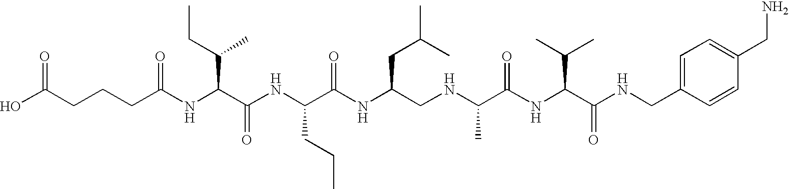 | 732.6 | RT = 3.52 min (HPLC-conditions 2) |
| 46 | 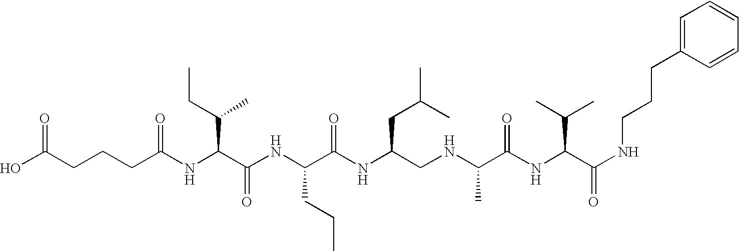 | 731.6 | RT = 4.38 min (HPLC-conditions 2) |
| 47 | 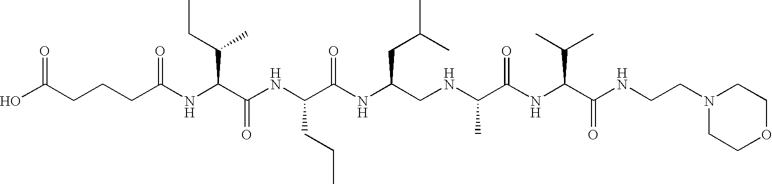 | 726.7 | RT = 3.45 min (HPLC-conditions 2) |
| 48 | 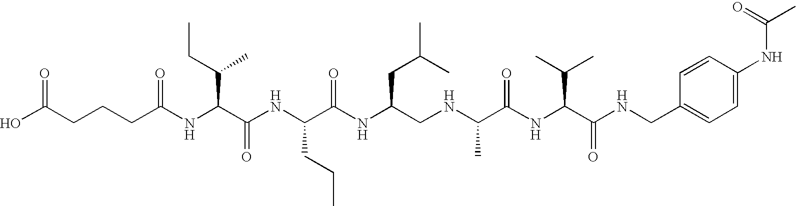 | 760.7 | RT = 3.81 min (HPLC-conditions 2) |
| 49 | 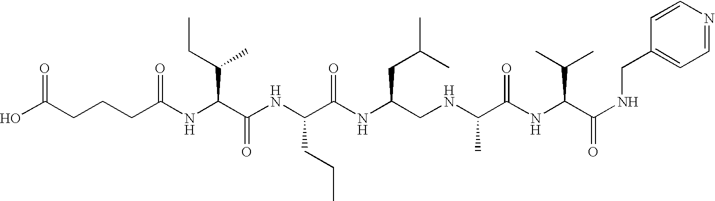 | 704.7 | RT = 3.40 min (HPLC-conditions 2) |
| 50 | 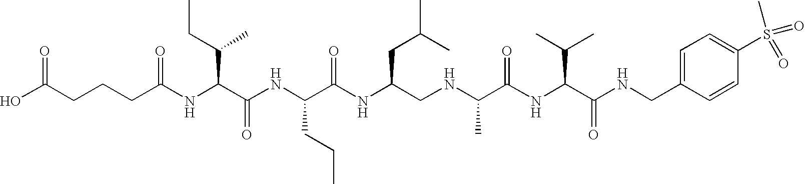 | 781.6 | RT = 3.90 min (HPLC-conditions 2) |

### Example 51

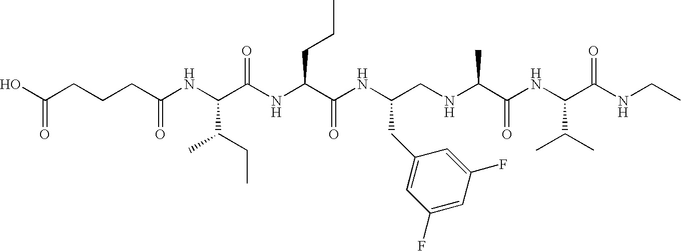 a) Preparation of 51-a:

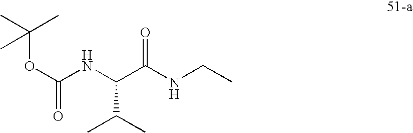

5 g Boc-L-valine (23,0 mmol), 7.4 g TBTU (23.0 mmol) and 12 ml DIPEA (70.1 mmol) were dissolved in 50 ml dichloromethane and 5 ml DMF. 1.9 g ethylamine hydrochloride (23.3 mmol) were added and the mixture was stirred at room temperature over night. The mixture was extracted with 20% KHSO₄ solution and water. The organic phase was separated with an isolute phase separator and evaporated. The residue was purified by MPLC (silica gel, ethylacetate/heptane=7:3) to yield 4.9 g 51-a (93%) as a white solid.

ES-MS (M+H-Boc)⁺145.2
b) Preparation of 51-b:

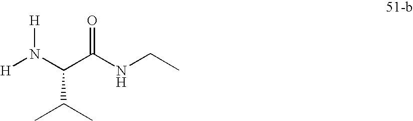

4.9 g (20.0 mmol) 51-a were dissolved in 25 ml methylene chloride. The mixture was cooled to 0° C. and 10 ml (128 mmol) TFA were added. After warming to room temperature the reaction was stirred over night. The mixture was concentrated, diluted with water and made basic with 4 N NaOH under ice cooling. The resulting solution was extracted with methylene chloride and phases were separated using an isolute phase separator. The organic phase was evaporated to yield a yellow oil (2.75 g, 95%).

ES-MS (M+H)⁺145.2
c) Preparation of 51-c:

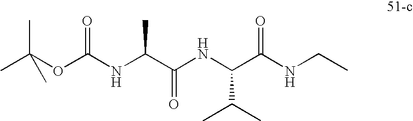

51-c was prepared from 1 g 51-b (6.93 mmol) and 1.3 g (6.87 mmol) Boc-alanine. Using a standard coupling procedure analogous to the preparation of 51-a yielded 1.4 g (65%) 51-c.

RT=2.47 min (HPLC-conditions 3)

ES-MS (M+H)⁺316.1
d) Preparation of 51-d:

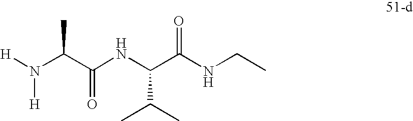

In analogy to the preparation of 51-b, 1.5 g 51-c (4.44 mmol) yielded 1.4 g (96%) of 51-d.

ES-MS (M+H)⁺216.1
e) Preparation of 51-e:

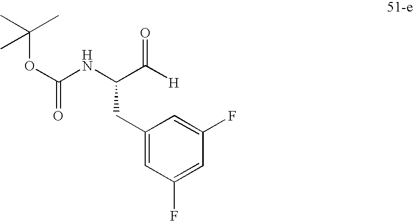

2 g (6.64 mmol) Boc-3,5-difluorophenylalanine in 10 ml THF were added slowly to a suspension of 380 mg (10.0 mmol) LiAlH₄ in 5 ml THF at −15° C. After warming to room temperature the mixture was stirred over night. 4 ml saturated diammoniumtartrate solution and MgSO₄ were added and the resulting suspension was stirred for 30 minutes, filtered over a short plug of silica gel and concentrated. The resulting oil was purified by MPLC (silica gel, ethylacetate:heptane=3:7) yielding 0.9 g white solid which was dissolved in 20 ml methylene chloride and added drop wise to a solution of 1.5 g (3.54 mmol) Dess-Martin-periodinane. The mixture was stirred at room temperature for 3 h. 30 ml saturated bicarbonate solution and 30 ml Na₂S₂O₃ (10% solution) were added and the resulting mixture stirred for 20 minutes and subjected to standard aqueous work up. The resulting organic phase was evaporated to yield 0.85 g (48%) aldehyde 51-e, which was used in the next step without further purification and storage.
f) Preparation of 51-f:

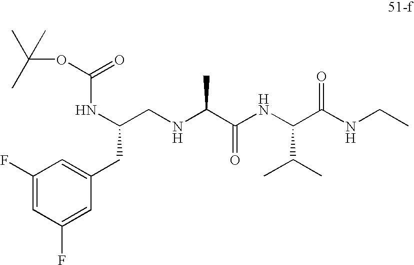

500 mg 51-e (1.75 mmol), 690 mg 51-d (2.1 mmol) and 742 mg sodiumtriacetoxyborohydride (95%, 3.33 mmol) 200 μl acetic acid and 10 ml THF were mixed and stirred at room temperature over night. After standard aqueous work up the residue was purified by flash chromatography (silica gel, dichloromethane:acetone=7:3) to yield 200 mg 51-f as a white solid (24%).

RT=2.8 min (HPLC-conditions 3)

ES-MS (M+H)⁺485.3
g) Preparation of 51-g:

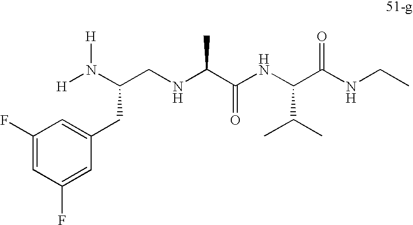

51-g was prepared from 51-f in analogy to the preparation of 51-b.

RT=2.1 min (HPLC-conditions 3)
h) Preparation of 51-h:

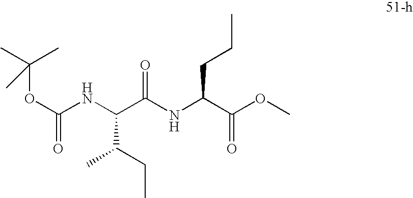

51-h was prepared in analogy to the preparation of 51-a from Boc-L-isoleucine and L-norvalinemethylester.

ES-MS (M+H)⁺345.1
i) Preparation of 51-i:

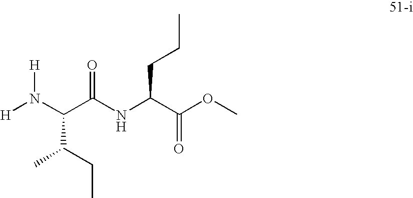

51-i was prepared from 51-h in anology to 51-b.

ES-MS (M+H)⁺245.2
j) Praparation of 51-j:

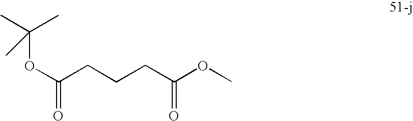

42 ml Glutaric acid monomethylester (95%, 318 mmol), 72.2 g DCC (350 mmol) and 9.8 g DMAP (80 mmol) were dissolved in 140 ml tert-butanol. The mixture was stirred for 24 h at room temperature. The resulting slurry was filtrated and diluted with methylene chloride. The organic phase was washed with 10% citric acid and 9% bicarbonate solution, dried over MgSO₄ and evaporated. The resulting oil was distilled to yield 47.1 g of a colourless oil (73%) boiling point: 60° C. (0.007 Torr)

ES-MS (M+H)⁺203.2
k) Preparation of 51-k:

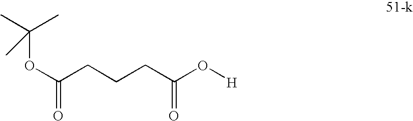

47.1 g (202 mmol) 51-j in 170 ml ethanol were treated with 130 ml 2N NaOH and stirred at room temperature for 3 h. Using 2N HCl the reaction mixture was neutralized to a pH of 7.0. Ethanol was distilled and the aqueous residue was diluted with water and ethylacetate. At 0° C. the pH was brought to 3.1 with 10% citric acid. Standard work up yielded 41.4 g 51-k (94%).

ES(−)-MS (M−H)⁺187.1
l) Preparation of 51-l:

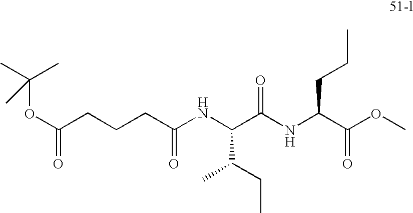

51-l was prepared from 51-i and 51-k in analogy to 51-a.

ES-MS (M+H)⁺415.5
m) Preparation of 51-m:

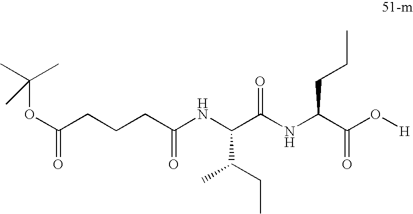

51-l was dissolved in 45 ml methanol and 10.8 ml 10% LiOH in water are added. The mixture was stirred at room temperature for 2 hours. Using 2N HCl the reaction mixture was neutralized to a pH of 6.4. Methanol was distilled and the aqueous residue was diluted with water and methylene chloride. At 0° C. the pH was brought to 3.2 with 10% citric acid. Standard work up and purification by MPLC (silica gel, methylene chloride:methanol=95:5) yielded 1.2 g (17%) of 51-m

ES-MS (M+H)⁺401.3
n) Preparation of 51-n

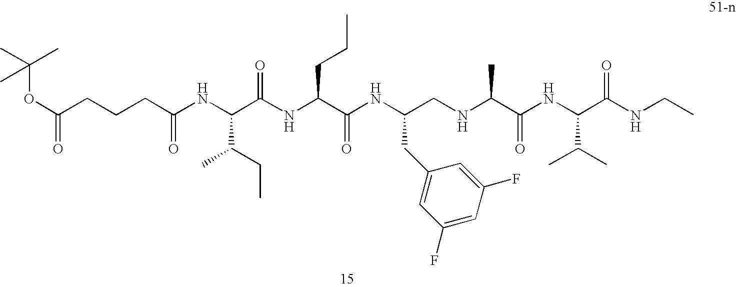

51-n was prepared from 51-m and 51-g in analogy to 51-a.

RT=3.0 min (HPLC-conditions 3)

ES-MS (M+H)⁺767.5
o) Preparation of Example 51:

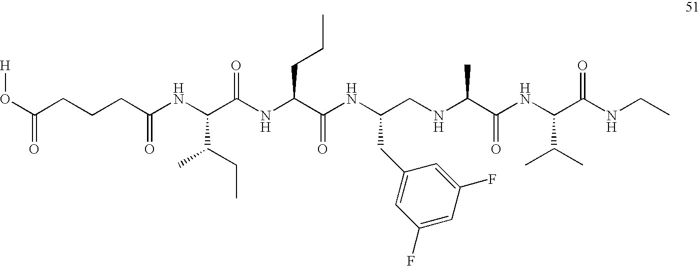

100 mg (0.13 mmol) 51-n were dissolved in 5 ml methylene chloride. 0.5 ml TFA were added and the mixture stirred at room temperature for 3 hours. The solution was concentrated. Addition of diethylether led to crystallization of 100 mg (93%) of 51.

RT=2.7 min (HPLC-conditions 3)

ES-MS (M+H-CF₃COOH)⁺711.4

Melting pointdecomp.>100° C.

### Example 52

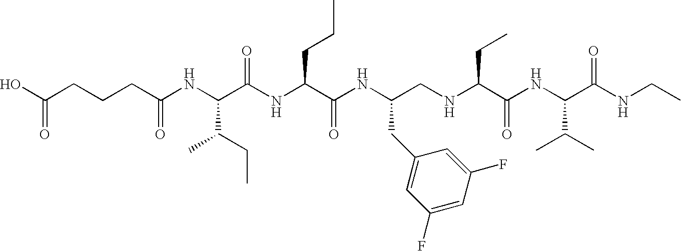

Example 52 was prepared like example 51, except that Boc-L-aminobutyric acid is used instead of Boc-L-alanine.

RT=2.7 min (HPLC-conditions 3)

ES-MS (M+H-CF₃COOH)⁺725.4

Melting point decomp.>200° C.

### Example 53

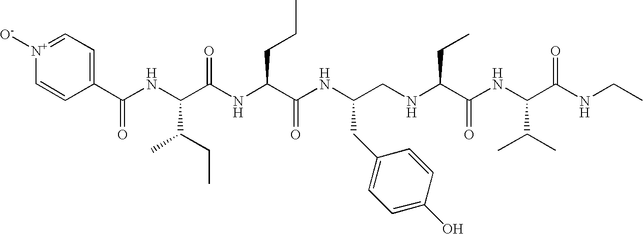

RT=2.4 min (HPLC-conditions 3)

ES-MS (M+H)⁺712.3

Melting point decomp.>205° C.

The intermediate 53-a 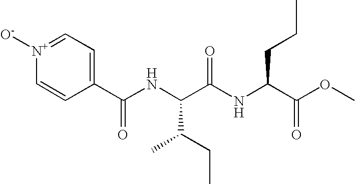
was prepared in analogy to example 51-l and the corresponding acid intermediate in analogy to example 51-m. Synthesis of example 53 is finished in analogy to 51-g.

The following examples 54 to 57 and 145 to 156 were synthesized in analogy to example 53.

### Example 54

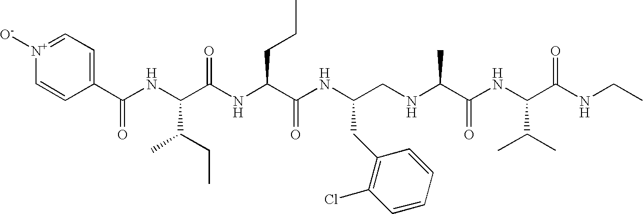

RT=2.5 min (HPLC-conditions 3)

ES-MS (M+H)⁺716.5

Melting point decomp.>230° C.

### Example 55

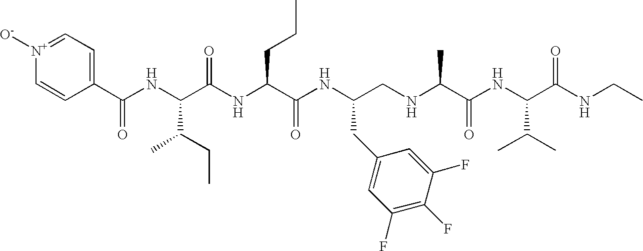

RT=2.6 min (HPLC-conditions 3)

ES-MS (M+H)⁺736.3

Melting point decomp.>240° C.

### Example 56

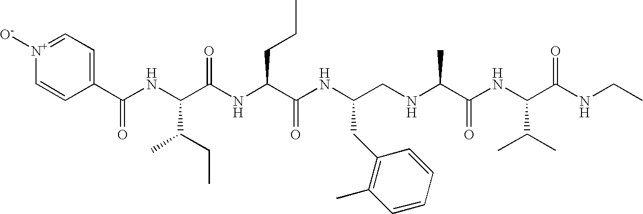

RT=2.6 min (HPLC-conditions 3)

ES-MS (M+H)⁺696.5

Melting point decomp.>237° C.

### Example 57

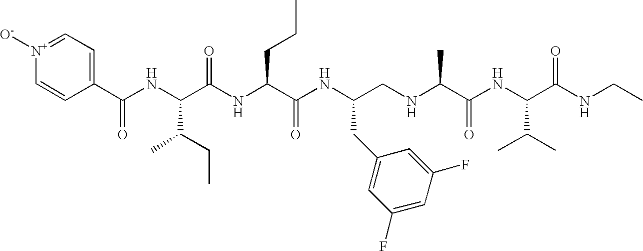

RT=2.6 min (HPLC-conditions 3)

ES-MS (M+H)⁺718.4

Melting point decomp.>220° C.

### Example 145

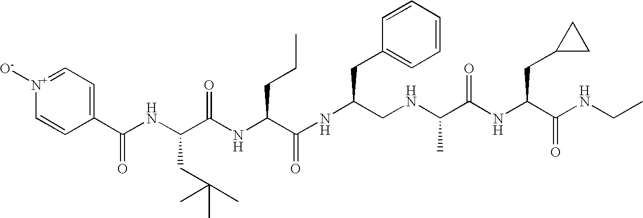

RT=2.5 min (HPLC-conditions 3)

ES-MS (M+H)⁺708.4

### Example 146

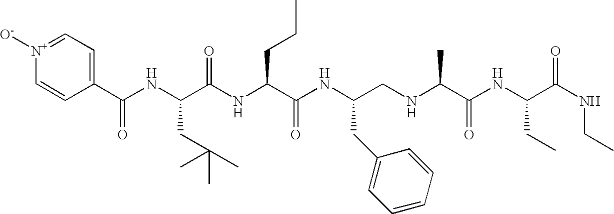

RT=2.46 min (HPLC-conditions 3)

ES-MS (M+H)⁺682

| | | Mass | |
|---|---|---|---|
| Example | | Spectra | HPLC-Retention |
| No. | Formula | [M + H]⁺ | Time |
| 147 | 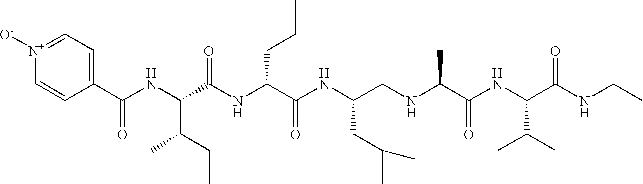 | 648 | RT = 2.53 min (HPLC- conditions 3) |
| 148 | 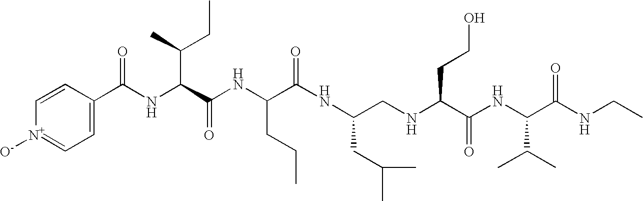 | 678 | |
| 149 | 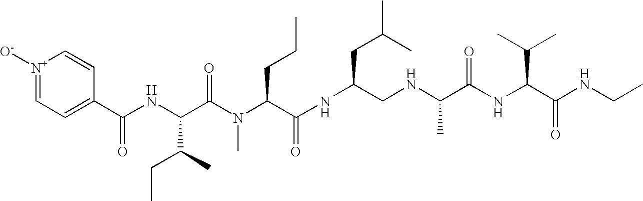 | 662 | |
| 150 | 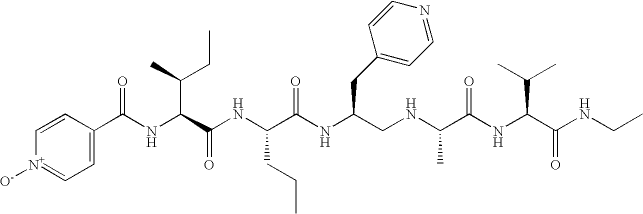 | 683 | |
| 151 | 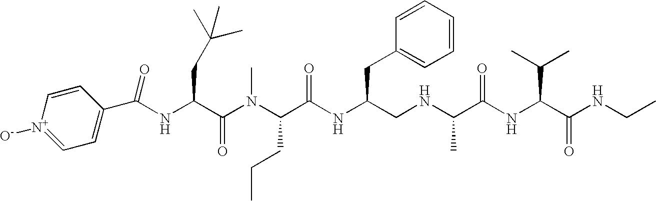 | 710 | RT = 2.66 min (HPLC- conditions 3) |
| 152 | 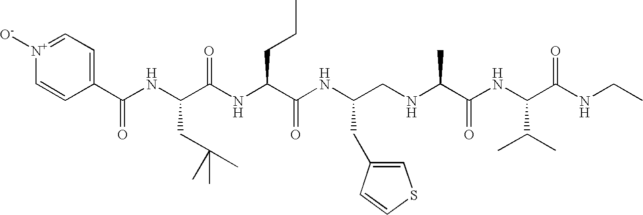 | 702 | |
| 153 | 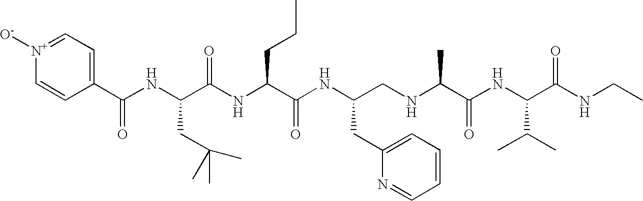 | 695 | |
| 154 | 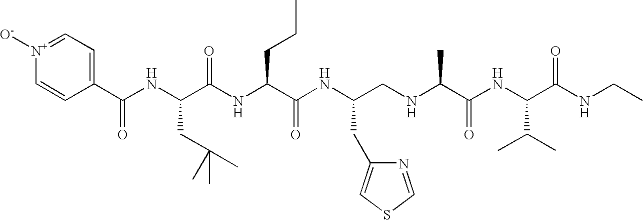 | 588 | |
| 155 | 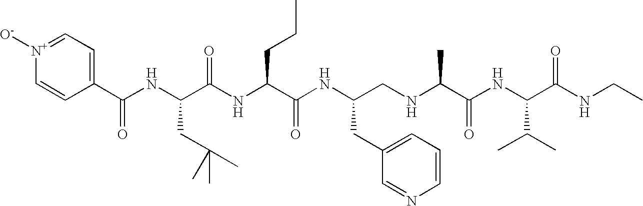 | 695 | |
| 156 | 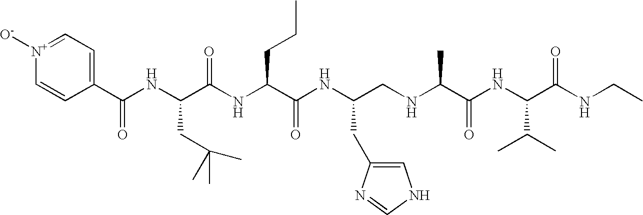 | 686 | |

### Example 58

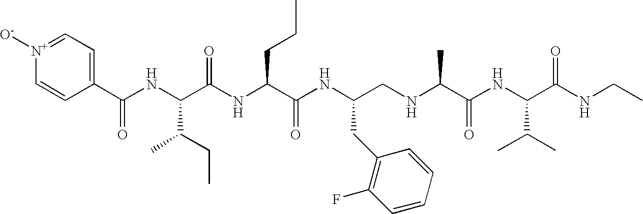

RT=2.5 min (HPLC-conditions 3)

ES-MS (M+H)⁺700,8
a) preparation of 58-a

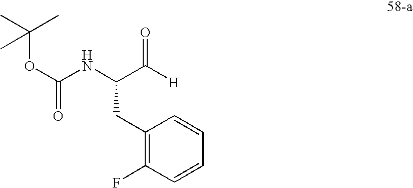

Compound 58-a was prepared starting with the BOC-protected amino acid by using the N,O-dimethylhydroxylamide (Weinreb amides) as intermediate which was reduced with LiAlH₄ to the corresponding aldehyde:

3 g (10,59 mmol) BOC-2-fluoro-L-phenylalanin, 4 ml Hünig base (23,38 mmol) and 3,6 g (11,21 mmol) TBTU were stirred in 70 ml THF at room temperature for 15 min and were than mixed with 1,2 g (12,3 mmol) N,O-dimethylhydroxylamine hydrochloride and stirred at room temperature over night. The solvent was removed, the residue extracted with ethylacetate and the extract washed with saturated bicarbonate solution and dried. The organic phase was evaporated to yield quantitative the Weinreb amide as yellow oil, (M+H)⁺=327.

Without any further purification 0.8 g (2,14 mmol) of the Weinreb amide was given to a suspension of 0.12 g (3,16 mmol) LiAlH₄ in 25 ml abs. THF at −35° C. and was stirred for additional 90 min. at −30° C. to −15° C. 0,3 ml saturated diammoniumtartrate solution and sodiumsulfat were added and the suspension filtered over Extrelut and concentrated. The resulting aldehyde 58-a (purity appr. 75%) was immediately used in the next step without any further purification.

Synthesis of Example 58 is Finished in Analogy to Example 53

### Example 59

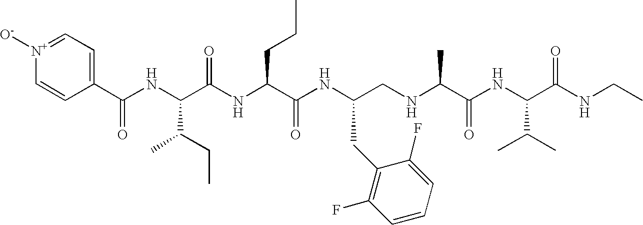

RT=2.46 min (HPLC-conditions 3)

ES-MS (M+H)⁺718.8
a) Preparation of 59-a

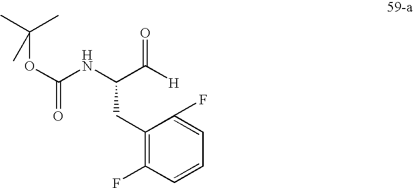

Compound 59-a was prepared in analogy to intermediate 58-a in 95% yield as a yellow oil, (M+H)⁺=345, starting from BOC-2.6-difluoro-L-alanine. BOC-2.6-difluoro-L-alanine can be optionally synthesized by starting with 2.6-difluorobenzaldehyde and BOC-alpha-phosphonoglycine trimethylester according to lit. U. Schmidt et al., Synthesis 1992, 487-490. Stereoselective hydrogenation of the intermediate with R.R-DIPAMP yielded in 98% BOC-2.6-difluoro-L-phenylalanine (ee=94%)

Reduction of the Weinreb amide resulted in the aldehyde 59-a (purity appr. 80%) which again was used immediately in the next step.

Synthesis of example 59 is finished in analogy to example 58.

### Example 60

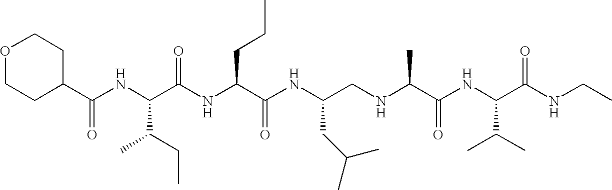

RT=2.51 min (HPLC-conditions 3)

ES-MS (M+H)+639.9
a) Preparation of 60-a

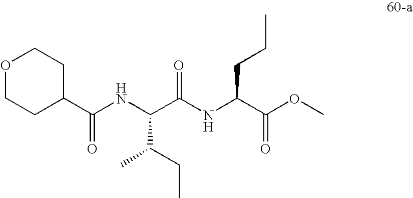

60 finished in analogy to example 53.

### Example A

Examples of Pharmaceutical Formulations

| a) Tablets | per tablet | |
|---|---|---|
| Active substance (Example 1) | 50 | mg |
| Lactose | 170 | mg |
| Corn starch | 260 | mg |
| Polyvinylpyrrolidone | 15 | mg |
| Magnesium stearate | 5 | mg |
| | 500 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| b) Tablets | per tablet | |
|---|---|---|
| Active substance (Example 1) | 40 | mg |
| Corn starch | 210 | mg |
| Lactose | 65 | mg |
| Microcrystalline cellulose | 40 | mg |
| Polyvinylpyrrolidone | 20 | mg |
| Sodium-carboxymethyl starch | 23 | mg |
| Magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| c) Coated tablets | per coated tablet | |
|---|---|---|
| Active substance (Example 1) | 5 | mg |
| Corn starch | 41.5 | mg |
| Lactose | 30 | mg |
| Polyvinylpyrrolidone | 3 | mg |
| Magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| d) Capsules | per capsule | |
|---|---|---|
| Active substance (Example 1) | 25 | mg |
| Corn starch | 283.5 | mg |
| Magnesium stearate | 1.5 | mg |
| | 310 | mg |

The substance and corn starch are mixed and moistened with water. The moist mass is screened and dried. The dry granules are screened and mixed with magnesium stearate. The finished mixture is packed into size 1 hard gelatine capsules.

| e) Ampoule solution | | |
|---|---|---|
| Active substance (Example 1) | 0.5 | mg |
| Sodium chloride | 50 | mg |
| Water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 0,5 mg, 2,5 mg and 5,0 mg of active substance.

| f) Suppositories | | |
|---|---|---|
| Active substance (Example 2) | 30 | mg |
| Solid fat | 1670 | mg |
| | 1700 | mg |

The solid fat is melted. The ground active substance is homogeneously dispersed at 40° C. It is cooled to 38° C. and poured into slightly chilled suppository moulds.

We claim:

1. A compound of formula (I) 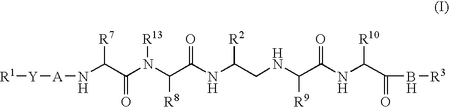
wherein
R¹ represents
H, an alkyl-, alkenyl-, alkynyl-, haloalkyl-, alkoxy-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group, wherein
each of said groups may be optionally substituted by one or more substituents independently selected from the group consisting of alkyl-, heteroaryl-, benzoyl-, haloalkyl-, alkoxy-, alkoxycarbonyl-, haloalkoxy-, halogen-, carboxy-, acetyl-, acylamino-, carboxyalkylaminoalkyl-, formyl-, hydroxy-, cyano-, nitro-, oxo-, —N(R⁴)₂, -alkyl-N(R⁴)₂, alkylsulfonylamino-, —SO₂-alkyl, —SO₂NH₂, —SO₃H, —SO₃N(R⁴)₂, and in case that the heteroaryl-, heteroaryl-alkyl-, or the heteroaryl-alkenyl-group contains a nitrogen atom in the ring, said nitrogen may be optionally oxidized to the corresponding N-oxide,
R² represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
R³ represents —OH or —N(R⁵)₂;
R⁴ each independently represent
H, an alkyl-, haloalkyl-, alkoxy-, alkenyl-, cycloalkyl-, cycloalkyl-alkyl-, heterocyclyl-, heterocyclyl-alkyl-, aryl-, aryl-alkyl-, heteroaryl-, or a heteroaryl-alkyl-group,
each of said groups may be optionally substituted by one or more substituents independently selected from the group consisting of alkyl-, alkoxy-, haloalkoxy-, halogen-, carboxy-, hydroxy-, cyano-, —N(R⁶)₂, —SO₂N(R⁶)₂,
R⁵ each independently represent
H, an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
Y is absent if A is present or represents a carbonyl group if A is absent,
R⁷ represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, S(R⁴), —SO₃H, —SO₃N(R⁴)₂,
R⁸ represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, a heteroaryl-alkyl-, aryl-alkenyl-, or a heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —CO—N(R⁴)₂, —S(R⁴), —SO—R⁴, —SO₃H, —SO₂-alkyl, —SO₃N(R⁴)₂,
R⁹ represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano, nitro, —N(R⁴)₂, -alkyl-N(R⁴)₂, —SO₃H, —SO₃N(R⁴)₂, —SO₂NH₂,
R¹⁰ represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-, or a heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
A is absent or represents a group of formula (II): 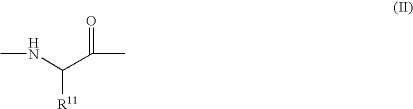
which is linked N-terminal via —NH— with Y, and C-terminal via —CO— with 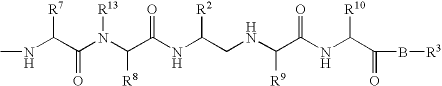
and wherein
R¹¹ represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-group, aryl-group, heteroaryl-, aryl-alkyl-, heteroaryl-alkyl-, aryl-alkenyl-, or a heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
B is absent or represents a group of formula (III): 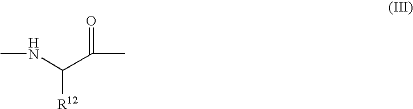
which is linked C-terminal via —CO— with R³ and N-terminal via —NH— with 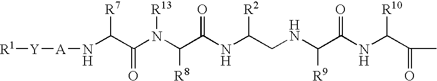
and wherein
R¹² represents
an alkyl-, alkenyl-, alkynyl-, haloalkyl-, cycloalkyl-, heterocyclyl-, cycloalkyl-alkyl-, heterocyclyl-alkyl-, cycloalkyl-alkenyl-, heterocyclyl-alkenyl-, aryl-, heteroaryl-, aryl-alkyl-, heteroaryl-alkyl-, aryl-alkenyl-group, or a heteroaryl-alkenyl-group,
each of said groups may be optionally substituted by one or more substituents independently selected from the group consisting of alkyl-, alkoxy-, haloalkoxy-, halogen-, carboxy-, formyl-, hydroxy-, cyano-, nitro-, —N(R⁴)₂, -alkyl-N(R⁴)₂, —SO₃H, —SO₃N(R⁴)₂,
R¹³ represents
H, an alkyl-, alkenyl-, alkynyl-, cycloalkyl- or cycloalkyl-alkyl-group,
each of said groups may be optionally substituted by one or more substituents independently selected from the group consisting of alkoxy-, fluoroalkoxy-, fluor- and cyano-,
or pharmaceutically acceptable tautomers, enantiomers, diastereomers, salts or solvates thereof.

2. A compound according to claim 1, wherein
A and B are absent, and
Y represents a carbonyl group,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

3. A compound according to claim 1, wherein
A and B are absent, and
Y represents a carbonyl group,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-Imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

4. A compound according to claim 1, wherein
A and B are absent, and
Y represents a carbonyl group,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-Imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, n-propyl or a 2-phenylethylgroup,
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

5. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2, 3, 5, 6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group.
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl.

6. A compound according to claim 1, wherein
A and B are absent, and
Y represents a carbonyl group,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group

7. A compound according to claim 1, wherein
A and B are absent, and
Y represents a carbonyl group,
R¹ represents
a pyridin-2-yl-N-oxide, pyridin-3-yl-N-oxide, pyridin-4-yl-N-oxide, carboxyalkyl-, or a phenylgroup,
wherein the phenylgroup may be optionally substituted by one or more substituents independently selected from the group consisting of carboxygroups or halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl.

8. A compound according to claim 1, wherein
A and B are absent,
Y represents a carbonyl group,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group.

9. A compound according to claim 1, wherein
A and B are absent, and
Y represents a carbonyl group,
R¹ represents
a pyridin-4-yl-N-oxide, 4-hydroxy-2,3,5,6-tetrafluorophenyl- or a 3-carboxypropyl-group;
wherein the pyridin-4-yl-N-oxide group may be optionally substituted by one substituent selected from the group consisting of fluor, chlor, methyl and/or trifluoromethyl,

10. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-Imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂;
R⁶ each independently represents H or an alkyl group,
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

11. A compound according to claim 1, wherein
A and B are absent, and
Y represents a carbonyl group,
R¹ represents
a pyridin-2-yl-N-oxide, pyridin-3-yl-N-oxide, pyridin-4-yl-N-oxide, carboxyalkyl-, or a phenylgroup,
wherein the phenylgroup may be optionally substituted by one or more substituents independently selected from the group consisting of hydroxy groups, carboxy groups and halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-Imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

12. A compound according to claim 1, wherein
A and B are absent, and
Y represents a carbonyl group,
R¹ represents
a pyridin-4-yl-N-oxide, 4-hydroxy-2,3,5,6-tetrafluorophenyl- or a 3-carboxypropyl-group,
wherein the pyridin-4-yl-N-oxide group may be optionally substituted by one substituent selected from the group consisting of fluor, chlor, methyl and/or trifluoromethyl,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyrid inylmethyl-, 4-pyrid inylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

13. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-d ichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

14. A compound according to claim 1, wherein
A and B are absent, and
Y represents a carbonyl group,
R¹ represents
a pyridin-2-yl-N-oxide, pyridin-3-yl-N-oxide, pyridin-4-yl-N-oxide, carboxyalkyl-, or a phenylgroup,
wherein the phenylgroup may be optionally substituted by one or more substituents independently selected from the group consisting of hydroxy groups, carboxy groups and halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin4-ylmethyl-, 4-acetylaminobenzyl- or a 2-methylsulfonylbenzyl-group,
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

15. A compound according to claim 1, wherein
A and B are absent, and
Y represents a carbonyl group,
R¹ represents
a pyridin-4-yl-N-oxide, 4-hydroxy-2,3,5,6-tetrafluorophenyl- or a 3-carboxypropyl-group,
wherein the pyridin-4-yl-N-oxide group may be optionally substituted by one substituent selected from the group consisting of fluor, chlor, methyl and/or trifluoromethyl,
R² represents
a 2-butyl-, isobutyl-, cyclopropylmethyl-, cyclobutylmethyl-, cyclopentylmethyl-, cyclohexylmethyl-, cycloheptylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, 2-phenylethyl-, butyl-, propyl-, ethyl-, or a methylgroup,
wherein the substituents comprising a phenyl group may be optionally substituted at the phenyl group by one or more substituents independently selected from the group consisting of halogen atoms, alkyl-, hydroxy-, alkoxy- and haloalkyl groups, preferably by F, Cl, methyl and/or CF₃,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a 2-butyl-, isobutyl-, propyl-, butyl-, ethyl-, phenyl-, isopropyl-, cyclopropylmethyl-, cyclobutylmethyl-, cyclopentylmethyl-, cyclohexylmethyl-, cyclohexyl-, methylthiomethyl-, 2-methylthioethyl-, 2-cyanoethyl-, 2-phenylethyl-, 4-hydroxybenzyl-, benzyl-, methyl-, 2,2-dimethylpropyl-, 3,3-dimethylbutyl-, 3-methylbutyl-, cyanomethyl-, 1-hydroxyethyl-, propargyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁸ represents
a 2-methylthioethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, cylobutylmethyl-, cyclopentylmethyl-, cyclohexylmethyl-, 2-methylthioethyl-, cyclohexyl-, methylthiomethyl-, 2-cyanoethyl-, 2-phenylethyl-, benzyl-, methyl-, butyl-, 2,2-dimethylpropyl-, methoxymethyl-, 3-methylbutyl-, cyanomethyl-, 2-methoxyethyl-, cyclopentyl-, propargyl-, benzyloxymethyl-, hydroxymethyl- or an allylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R⁹ represents
an aminomethyl-, 2-hydroxyethyl-, 3-carboxypropyl-, 2-phenylethyl-, 2-butyl-, isobutyl-, propyl-, ethyl-, phenyl-, isopropyl-, cyclopropylmethyl-, cyclohexylmethyl-, 2-phenylethyl-, benzyl-, methyl-, hydroxyethyl-, hydroxymethyl-, propargyl-, allyl- or a butylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl,
R¹⁰ represents
a isobutyl-, propyl-, ethyl-, phenyl-, isopropyl-, cyclopropylmethyl-, cylobutylmethyl-, cyclopentylmethyl-, cyclohexylmethyl-, cyclopentyl-, cyclohexyl-, 2-phenylethyl-, benzyl-, methyl-, methylthiomethyl-, 2-methylthioethyl-, 3-methylbutyl-, propargyl-, butyl-, hydroxyethyl-, allyl- or a hydroxymethylgroup,
wherein the substituents comprising a phenyl group may be optionally substituted by one or more substituents independently selected from the group consisting of halogen atoms, alkyl-, hydroxy-, alkoxy- and haloalkyl groups preferably by fluor, chlor, methyl and/or trifluoromethyl.

16. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

17. A compound according to claim 1, wherein
A and B are absent, and
Y represents a carbonyl group,
R¹ represents
a pyridin-2-yl-N-oxide, pyridin-3-yl-N-oxide, pyridin-4-yl-N-oxide, carboxyalkyl-, or a phenylgroup,
wherein the phenylgroup may be optionally substituted by one or more substituents independently selected from the group consisting of hydroxy groups, carboxy groups and halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂;
R⁶ each independently represents H or an alkyl group
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

18. A compound according to claim 1, wherein
A and B are absent, and
Y represents a carbonyl group,
R¹ represents
a pyridine-4-yl N-oxide, 4-hydroxy-2,3,5,6-tetrafluorophenyl- or a 3-carboxypropyl-group,
wherein the pyridin-4-yl-N-oxide group may be optionally substituted by one substituent selected from the group consisting of fluor, chlor, methyl and/or trifluoromethyl,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

19. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl-N-oxide, pyridin-3-yl-N-oxide, pyridin-4-yl-N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-d ichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

20. A compound according to claim 1, wherein
A and B are absent, and
Y represents a carbonyl group,
R¹ represents
a pyridin-2-yl N-oxide, pyridin-3-yl N-oxide, pyridin-4-yl N-oxide, carboxyalkyl-, or a phenylgroup,
wherein the phenylgroup may be optionally substituted by one or more substituents independently selected from the group consisting of hydroxy groups, carboxy groups and halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

21. A compound according to claim 1, wherein
A and B are absent, and
Y represents a carbonyl group,
R¹ represents
a pyridin-4-yl N-oxide, 4-hydroxy-2,3,5,6-tetrafluorophenyl- or a 3-carboxypropyl-group,
wherein the pyridin-4-yl-N-oxide group may be optionally substituted by one substituent selected from the group consisting of fluor, chlor, methyl and/or trifluoromethyl,
R² represents
a 2-butyl-, isobutyl-, cyclohexylmethyl-, 2,2-dimethylpropyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 4-hydroxybenzyl-, propyl-, ethyl-, butyl-, cyclopropylmethyl, cyclopentylmethyl-, cyclobutylmethyl- or a methylgroup,
wherein the substituents comprising a phenyl group may be optionally substituted at the phenyl group by one or more substituents independently selected from the group consisting of halogen atoms, alkyl groups and haloalkyl groups, preferably by F, Cl, methyl and/or CF₃,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a 2-butyl-, isobutyl-, propyl-, ethyl-, phenyl-, isopropyl-, cyclopropylmethyl-, cyclohexyl-, methylthiomethyl-, 2-methylthioethyl-, 2-phenylethyl-, 4-hydroxybenzyl-, benzyl-, methyl-, 2,2-dimethylpropyl-, 3-methylbutyl-, cyanomethyl-, 2-cyanoethyl-, 1-hydroxyethyl- or an allylgroup,
wherein the substituents comprising a phenyl group may be optionally substituted by one or more substituents independently selected from the group consisting of halogen atoms, alkyl groups and haloalkyl groups, preferably by fluor, chlor, methyl and/or trifluoromethyl,
R⁸ represents
a cyclopentyl-, 2-cyanoethyl-, 3,3,3-trifluoropropyl-, 2-methanesulfinylethyl-, methanesulfonylmethyl-, 2-methanesulfonylethyl-, 2-carbamoylethyl-, 2-butyl-, isobutyl-, propyl-, carbamoylmethyl, ethyl-, phenyl-, isopropyl-, cyclopropylmethyl-, 2-methylthioethyl-, methylthiomethyl-, 2-phenylethyl-, benzyl-, methyl-, butyl-, hydroxymethyl-, 2,2-dimethylpropyl-, methoxymethyl-, 3-methylbutyl-, 2-methoxyethyl-, cyclohexylmethyl- or an allylgroup,
R⁹ represents
a 2-hydroxyethyl-, 3-carboxypropyl-, 2-phenylethyl-, benzyl-, aminomethyl-, isobutyl-, propyl-, ethyl-, phenyl-, isopropyl-, cyclopropylmethyl-, methyl-, hydroxymethyl-, hydroxyethyl- or a butylgroup,
R¹⁰ represents
a isobutyl-, propyl-, ethyl-, phenyl-, isopropyl-, cyclopropylmethyl-, cyclohexyl-, 2-phenylethyl-, benzyl-, methyl-, butyl-, 3-methylbutyl-, 2-methylthioethyl-, methylthiomethyl-, hydroxyethyl- or a hydroxymethylgroup.

22. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl N-oxide, pyridin-3-yl N-oxide, pyridin-4-yl N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

23. A compound according to claim 1, wherein
A and B are absent, and
Y represents a carbonyl group,
R¹ represents
a pyridin-2-yl N-oxide, pyridin-3-yl N-oxide, pyridin-4-yl N-oxide, carboxyalkyl-, or a phenylgroup,
wherein the phenylgroup may be optionally substituted by one or more substituents independently selected from the group consisting of hydroxy groups, carboxy groups and halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group,
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

24. A compound according to claim 1, wherein
A and B are absent, and
Y represents a carbonyl group,
R¹ represents
a pyridin-4-yl N-oxide, 4-hydroxy-2,3,5,6-tetrafluorophenyl- or a 3-carboxypropyl-group,
wherein the pyridin-4-yl-N-oxide group may be optionally substituted by one substituent selected from the group consisting of fluor, chlor, methyl and/or trifluoromethyl,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
an alkyl-, aryl-, heteroaryl-, cycloalkyl-alkyl-, heterocyclyl-alkyl-, aryl-alkyl-, or a heteroaryl-alkyl-group,
each of said groups may be optionally substituted by one or more substituents independently selected from the group consisting of alkyl-, aminocarbonylalkyl-, carbamoyl-, alkoxy-, haloalkoxy-, halogen-, carboxy-, acylamino-, formyl-, hydroxy-, cyano-, nitro-, methylsulfonyl-, —N(R⁶)₂, -alkyl-N(R⁶)₂ and —SO₂N(R⁶)₂,
R⁶ each independently represents H or an alkyl group
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesu Ifonylethyl-, carbamoylmethyl-, 2-carbamoylethyl-, phenyl-, benzyl-, propyl- or a 2-phenylethylgroup,
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

25. A compound according to claim 1, wherein
A and B are absent, and
Y represents a carbonyl group,
R¹ represents
a 3-methanesulfonyl-phenyl-, 4-methanesulfonylphenyl-, 3-(1H-tetrazol-5-yl)-phenyl-, 4-(1H-tetrazol-5-yl)-phenyl-, quinolin-4-yl N-oxide, 1-acetylpiperidin-3-yl-, 1-benzoylpiperidin-3-yl-, 1-carboxy-2-naphthyl-, pyridin-2-yl N-oxide, pyridin-3-yl N-oxide, pyridin-4-yl N-oxide, 2-carboxy-1-naphthyl-, 2-carboxycyclobutyl-, 2-carboxycycloheptyl-, 2-carboxycyclohexyl-, 2-carboxycyclopentyl-, 2-carboxycyclopropyl-, 2-carboxycyclopropylmethyl-, 2-carboxyethyl-, 2-carboxymethylcyclopropyl-, 2-carboxyphenyl-, 2-fluoro-3-hydroxyphenyl-, 3,4-dihydroxyphenyl-, 3,5-dicarboxycyclohexyl-, 3-acetylaminophenyl-, 3-acetylphenyl-, 3-carboxy-1-naphthyl-, 3-carboxy-2-naphthyl-, 3-carboxycyclobutyl-, 3-carboxycycloheptyl-, 3-carboxycyclohexyl-, 3-carboxycyclopentyl-, 3-carboxyphenyl-, 3-chloro-4-hydroxyphenyl-, 3-fluoro-4-hydroxyphenyl-, 3-hydroxybenzyl-, 3-hydroxyphenyl-, 3-methoxy-4-hydroxyphenyl-, 3-methoxycarbonylphenyl-, 3-methoxycarbonylpropyl-, 3-methoxyphenyl-, 3-nitro-4-hydroxyphenyl-, 3-nitrophenyl-, 3-propionylaminophenyl-, 3-trifluoromethyl-4-hydroxyphenyl-, 4-acetylaminophenyl-, 4-acetylaminopropyl-, 4-aminophenyl-, 4-carboxy-1-naphthyl-, 4-carboxy-2-naphthyl-, 4-carboxycycloheptyl-, 4-carboxycyclohexyl-, 4-carboxyphenyl-, 4-hydroxy-2,3,5,6-tetrafluorophenyl-, 4-hydroxy-3,5-dichlorophenyl-, 4-hydroxybenzyl-, 4-hydroxyphenyl-, 4-methanesulfonylaminophenyl-, 4-methoxyphenyl-, 4-nitrophenyl-, 4-propionylaminophenyl-, 4-sulfamoylphenyl-, 5-carboxy-1-naphthyl-, 5-carboxy-2-naphthyl-, 5-hydroxypyrazin-2-yl-, 6-hydroxypyridin-3-yl-, 6-hydroxypyrimidin-4-yl-, 6-oxo-1,6-dihydropyridazin-3-yl-, 4-carboxybutyl-, carboxymethoxymethyl-, carboxymethyl-, carboxymethylaminomethyl-, 5-carboxypentyl-, 3-carboxypropyl-, pyridin-2-yl-, pyridin-3-yl-, pyridin-4-yl-, tetrahydropyran-4-yl-, or a tetrahydropyran-4-ylmethyl-group,
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

26. A compound according to claim 1, wherein
A and B are absent, and
Y represents a carbonyl group,
R¹ represents
a pyridin-2-yl N-oxide, pyridin-3-yl N-oxide, pyridin-4-yl N-oxide, carboxyalkyl-, or a phenylgroup,
wherein the phenylgroup may be optionally substituted by one or more substituents independently selected from the group consisting of hydroxy groups, carboxy groups and halogen atoms, preferably fluor atoms and hydroxy groups, and
wherein the pyridin-2-yl-N-oxide, pyridin-3-yl-N-oxide and pyridin-4-yl-N-oxide groups may be optionally substituted by one or more substituents independently selected from the group consisting of fluor, chlor, methyl and/or trifluoromethyl,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin-4-ylmethyl-, 4-acetylaminobenzyl- or a 2-methylsulfonylbenzyl-group,
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup,
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

27. A compound according to claim 1, wherein
A and B are absent, and
Y represents a carbonyl group,
R¹ represents
a pyridin-4-yl N-oxide, 4-hydroxy-2,3,5,6-tetrafluorophenyl- or a 3-carboxypropyl-group,
wherein the pyridin-4-yl-N-oxide group may be optionally substituted by one substituent selected from the group consisting of fluor, chlor, methyl and/or trifluoromethyl,
R² represents
a 2-butyl-, propyl-, isobutyl-, 2,2-dimethylpropyl-, methyl-, cyclohexylmethyl-, 3-thienylmethyl-, 4-thiazolylmethyl-, 5-thiazolylmethyl-, 4-imidazolylmethyl-, 2-pyridinylmethyl-, 3-pyridinylmethyl-, 4-pyridinylmethyl-, benzyl-, 2-methylbenzyl-, 4-hydroxybenzyl-, 2-fluorobenzyl-, 2,6-difluorobenzyl-, 3,5-difluorobenzyl-, 3,4,5-trifluorobenzyl-, or a 2-chlorobenzylgroup,
R³ represents —NH(R⁵),
R⁵ represents
an ethyl-, cyclohexylmethyl-, 3-aminomethylbenzyl-, 4-aminomethylbenzyl-, 2-nitrobenzyl-, 3-nitrobenzyl-, 3-aminobenzyl-, 4-aminobenzyl-, 3-carbamoylbenzyl-, 2-pyridin-2-ylethyl-, 2-phenylethyl-, 3-phenylpropyl-, 2-morpholin-4-ylethyl-, 4-aminocarbonylmethylbenzyl-, 2-pyridin-4-ylethyl-, pyridin-3-ylmethyl-, pyridin4-ylmethyl-, 4-acetylaminobenzyl- or a 2-methylsulfonylbenzyl-group,
R⁷ represents
a methyl-, 2-butyl-, 3-methylbutyl-, 2-phenylethyl-, methylthiomethyl-, allyl-ethyl-, propyl-, 2-cyanoethyl-, cyclopropylmethyl-, 2,2-dimethylpropyl-, benzyl-, cyclohexyl-, phenyl-, isopropyl- or a isobutylgroup,
R⁸ represents
a cyclopropylmethyl-, methylthiomethyl-, 2-methylthioethyl-, methyl-, hydroxymethyl-, ethyl-, isopropyl-, butyl-, allyl-, isobutyl-, 2-butyl-, 3
-methylbutyl-, methoxymethyl-, 2-methoxyethyl-, cyclopentyl-, 2-cyanoethyl-, 3,3,3-trifluoropropyl-, 2-methanesulfinylethyl-, methanesulfonylmethyl-, 2-methanesulfonylethyl-, carbamoylmethyl-, 2-carbamoylethyl-, phenyl-, benzyl-, propyl- or a 2-phenylethylgroup
R⁹ represents
a methyl-, ethyl-, propyl-, butyl-, isobutyl-, cyclopropylmethyl-, aminomethyl-, 3-carboxypropyl-, benzyl-, 2-phenylethyl-, 2-hydroxyethyl-, hydroxymethyl- or a isopropylgroup,
R¹⁰ represents
a methyl-, ethyl-, propyl-, butyl-, isobutyl-, 3-methylbutyl-, hydroxymethyl-, phenyl-, benzyl-, 2-phenylethyl-, isopropyl-, cyclohexyl- or a cyclopropylmethylgroup.

28. The compound according to claim 1, selected from the group consisting of:

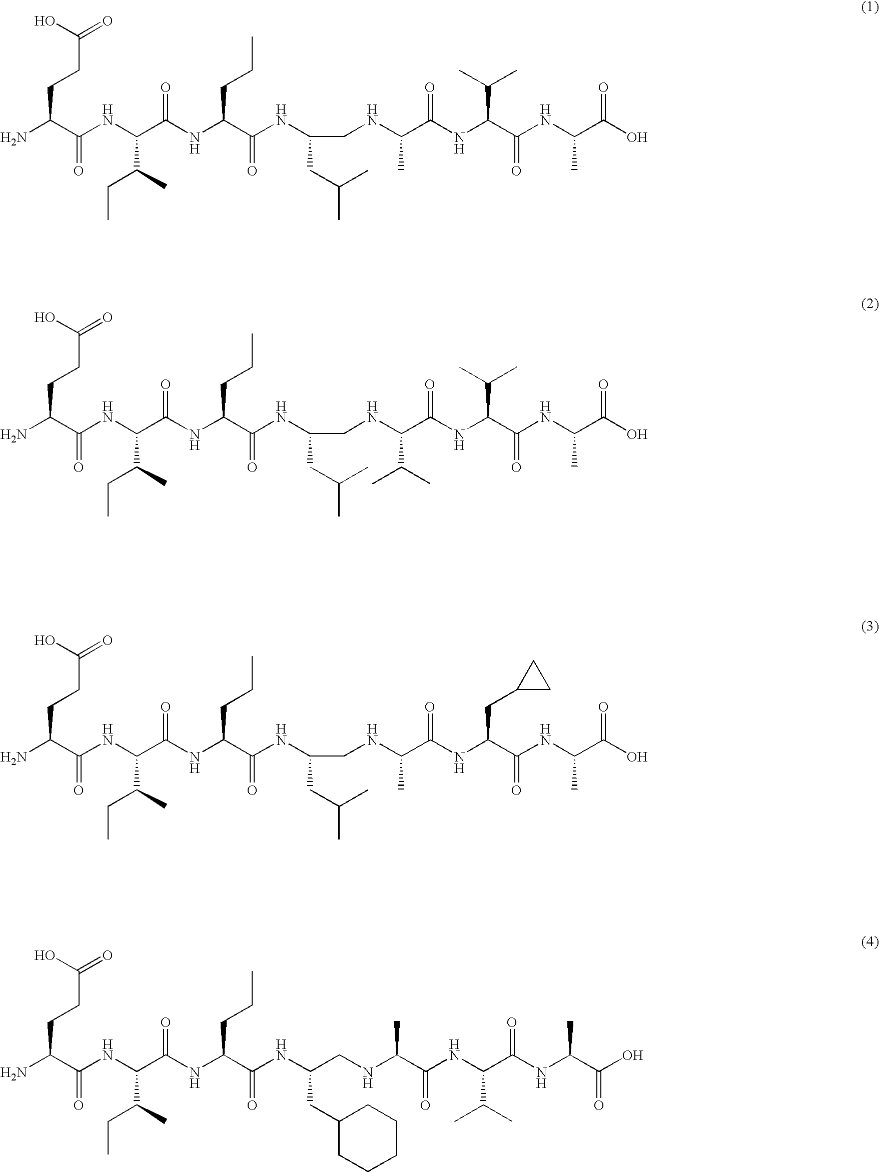

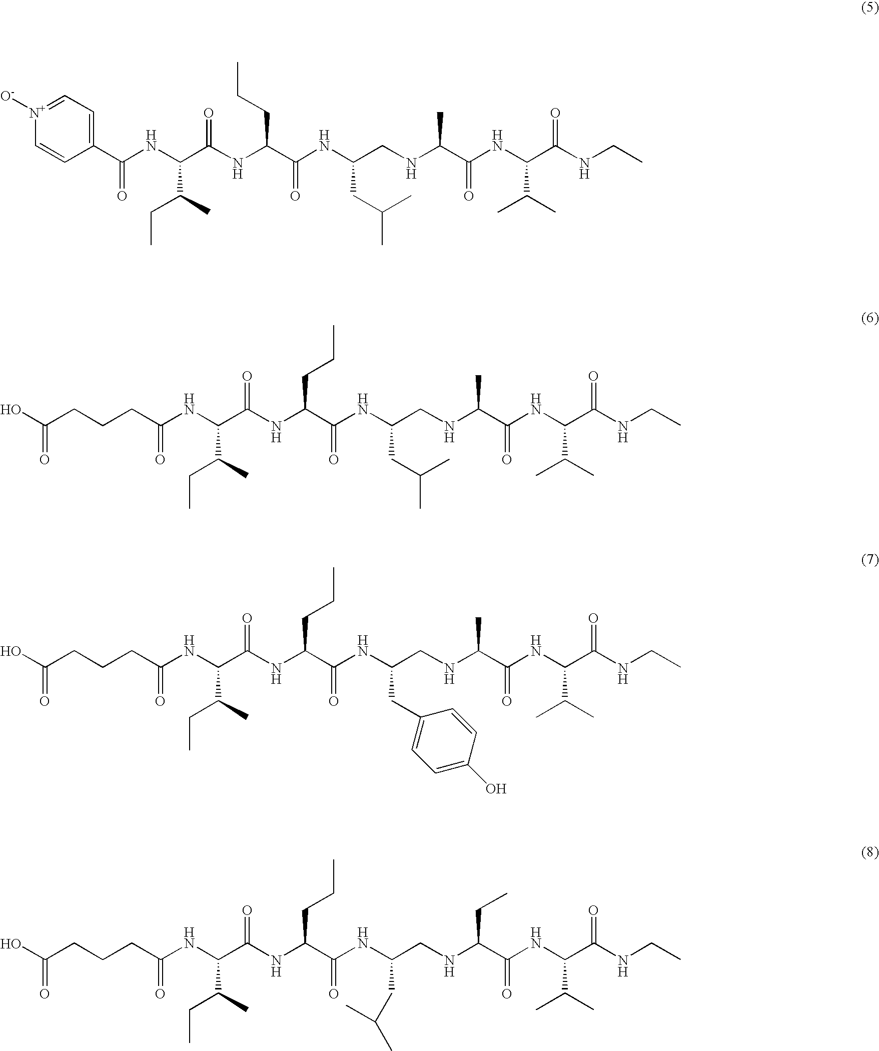

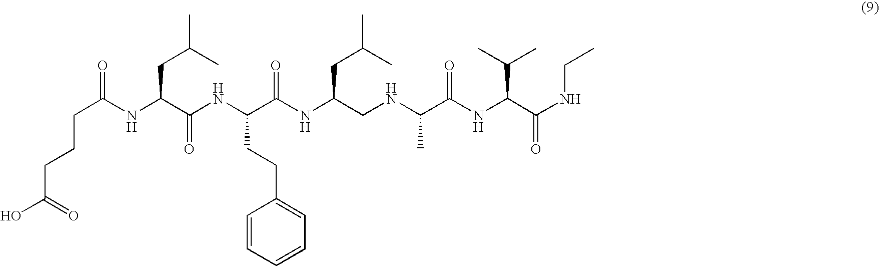

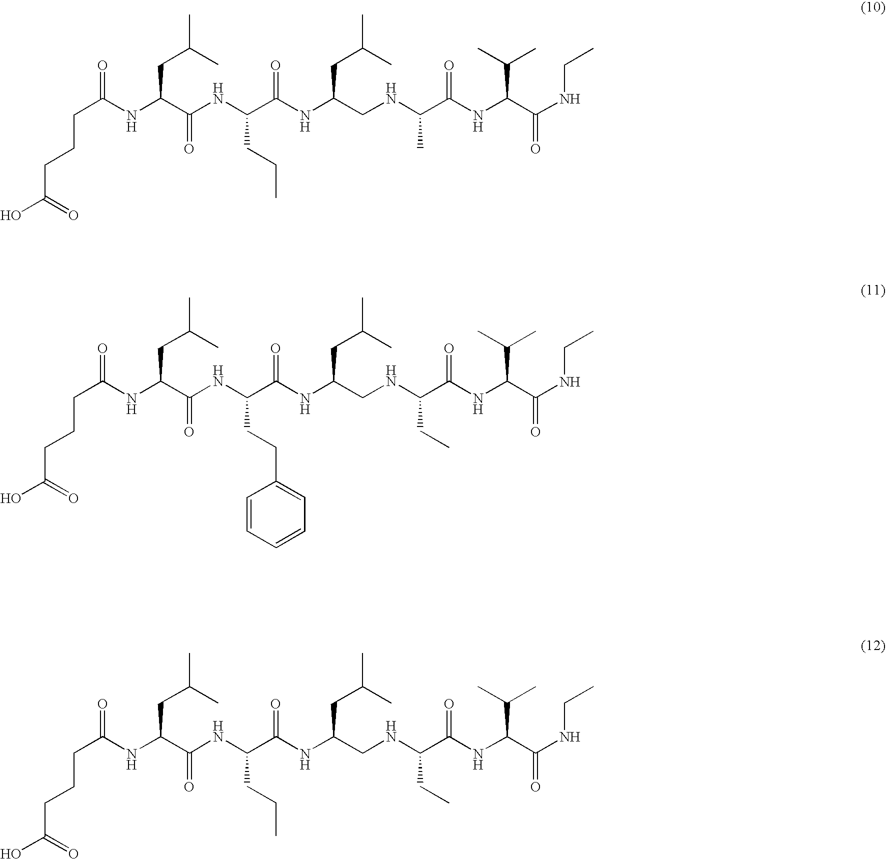

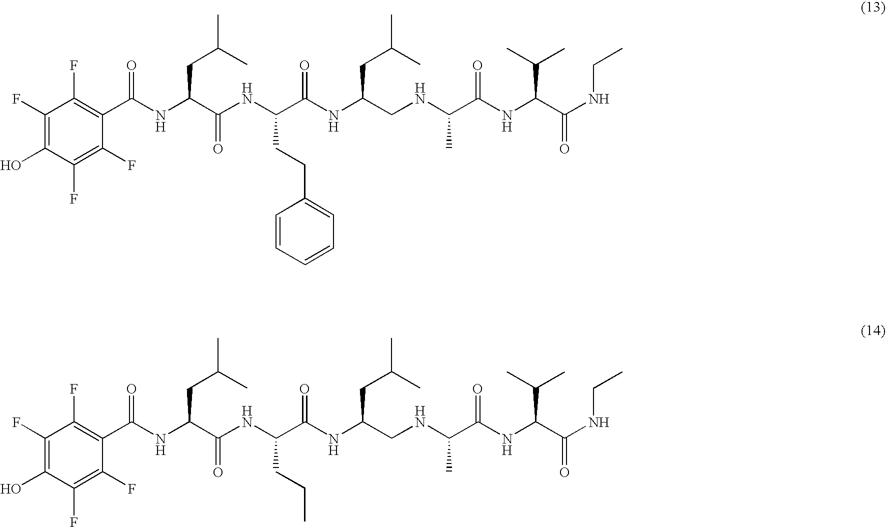

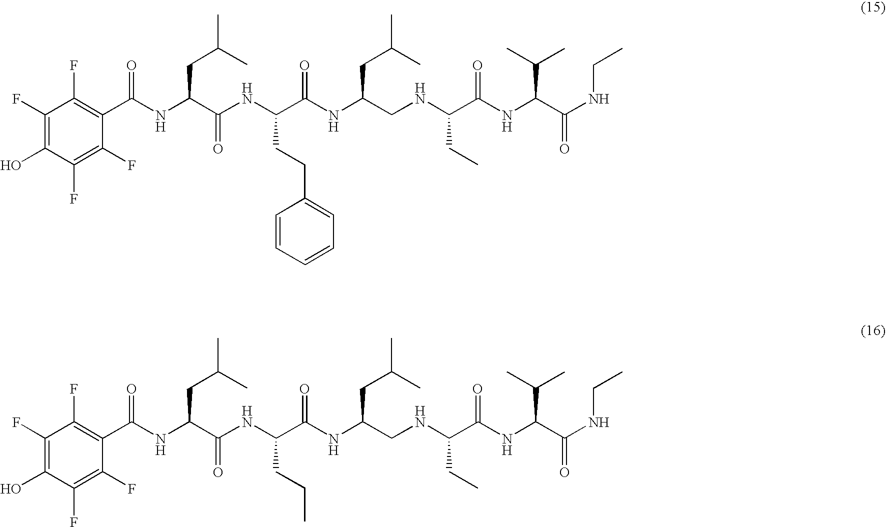

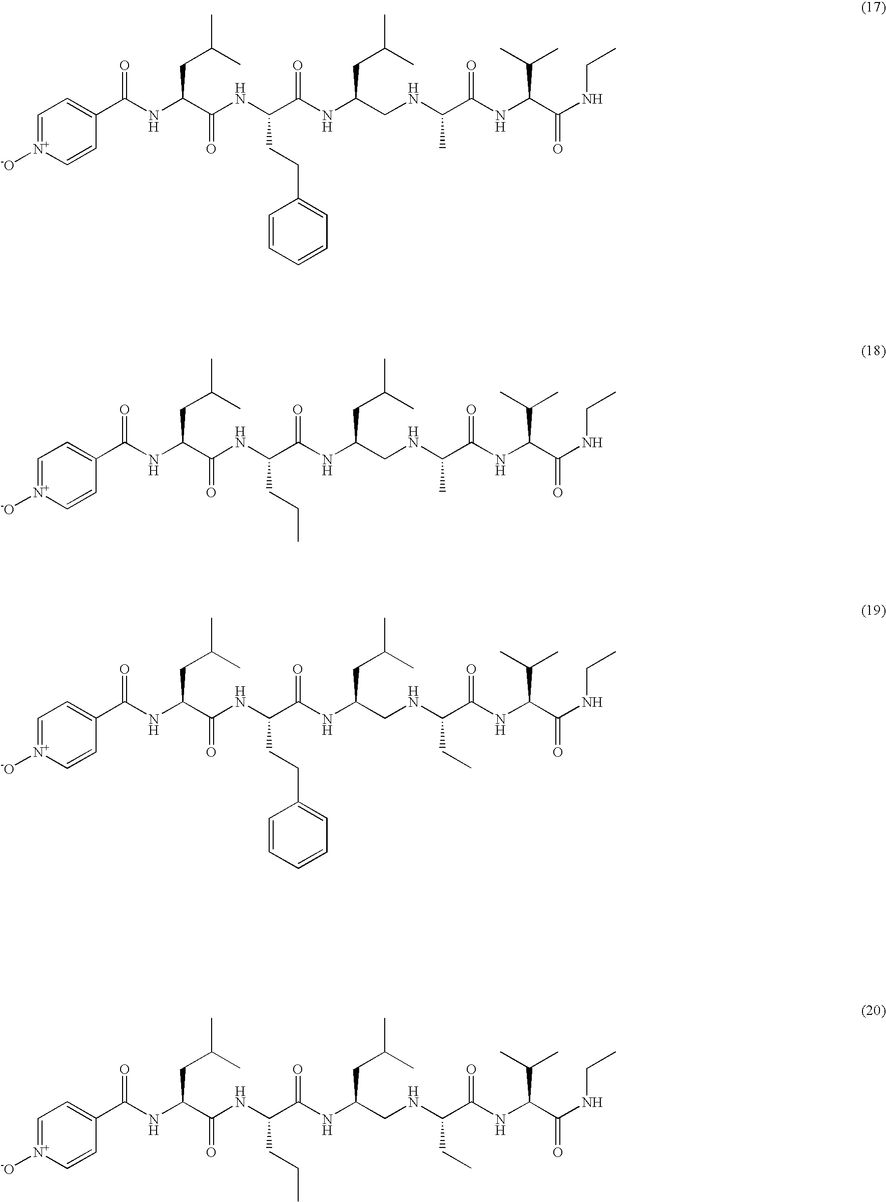

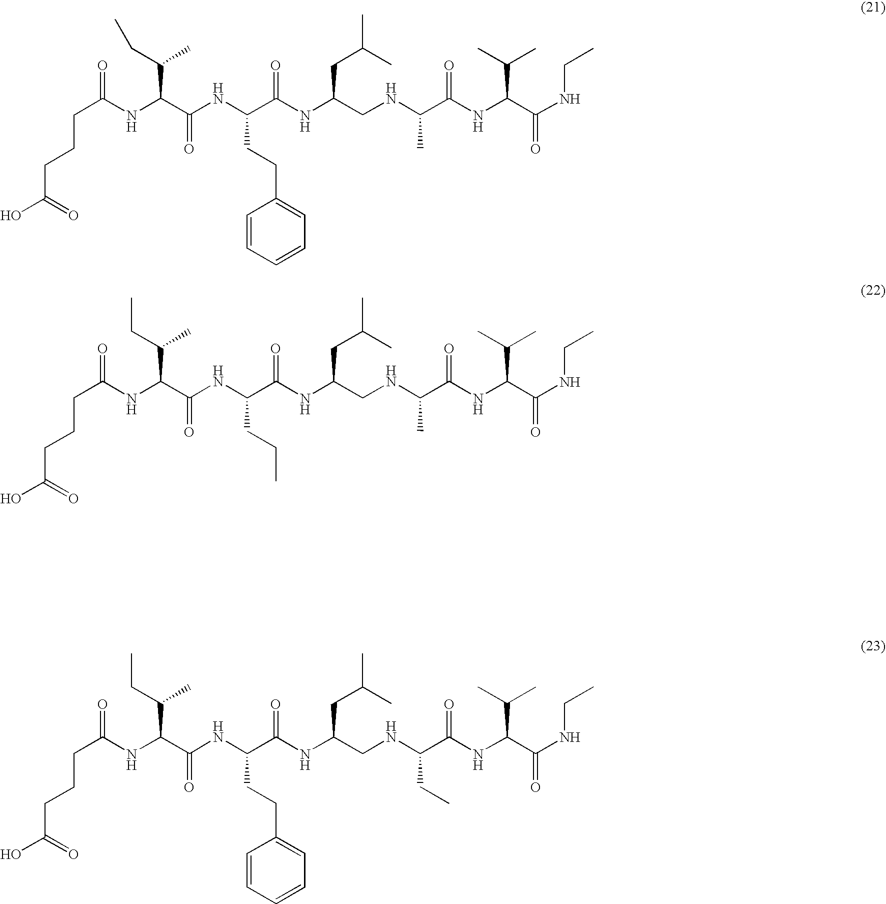

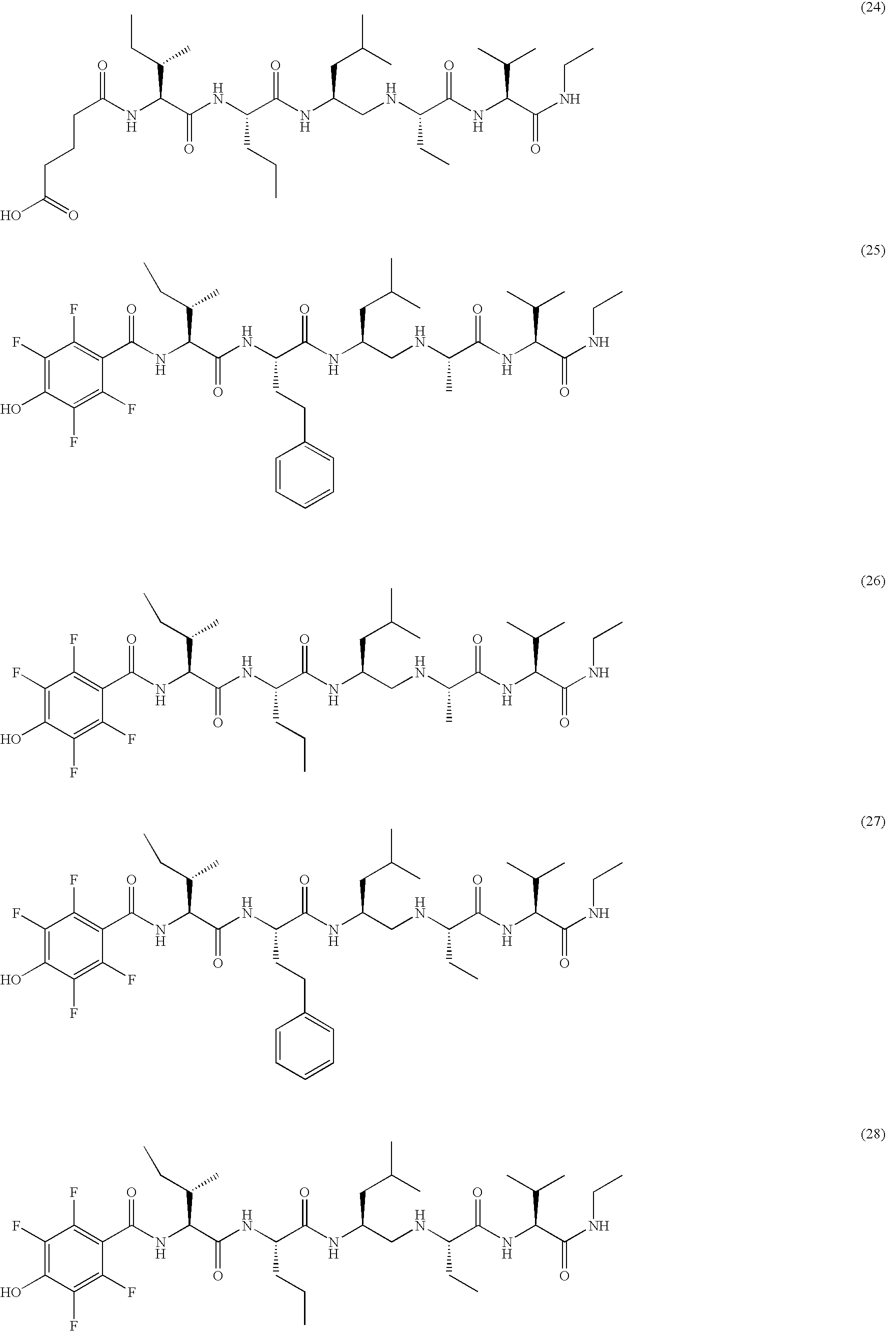

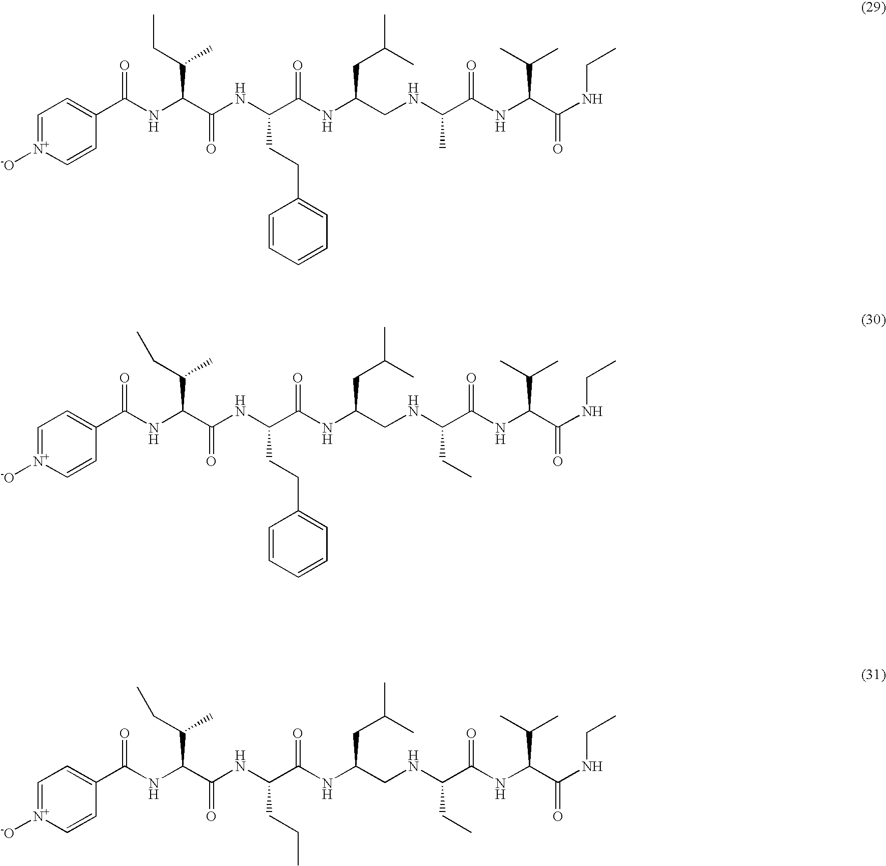

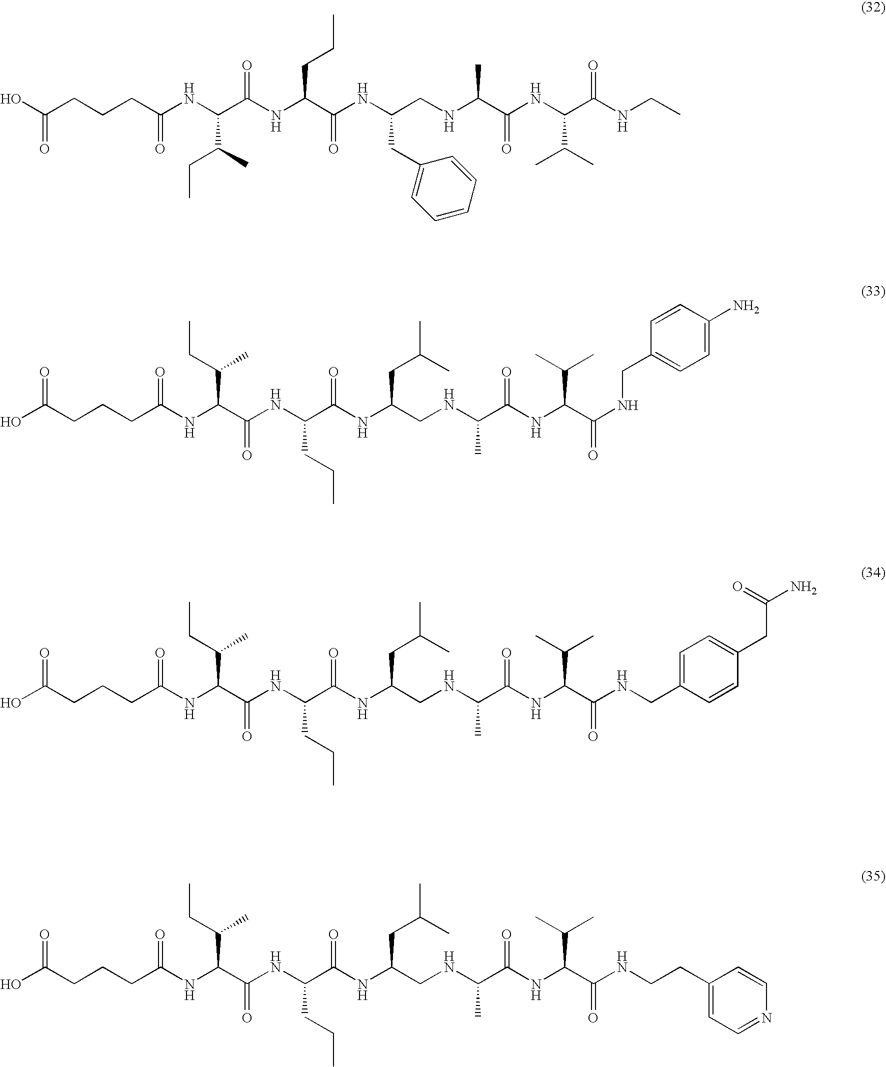

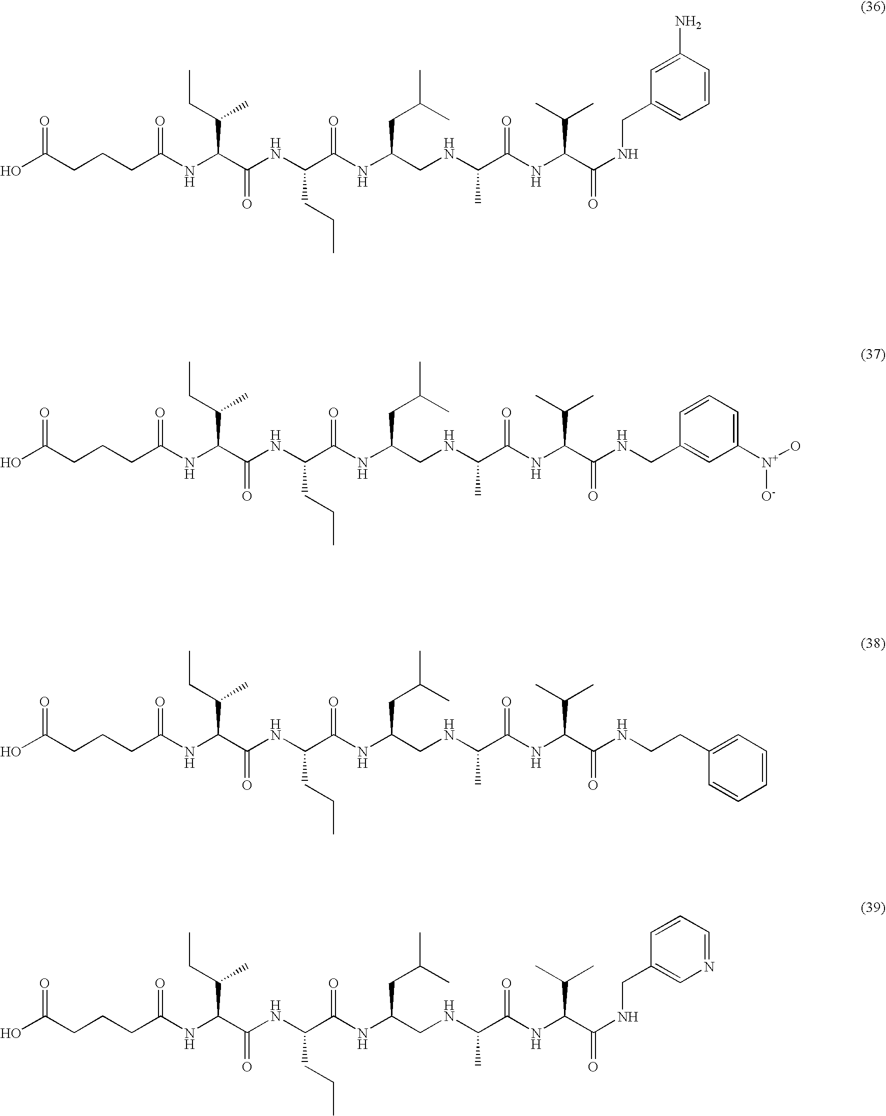

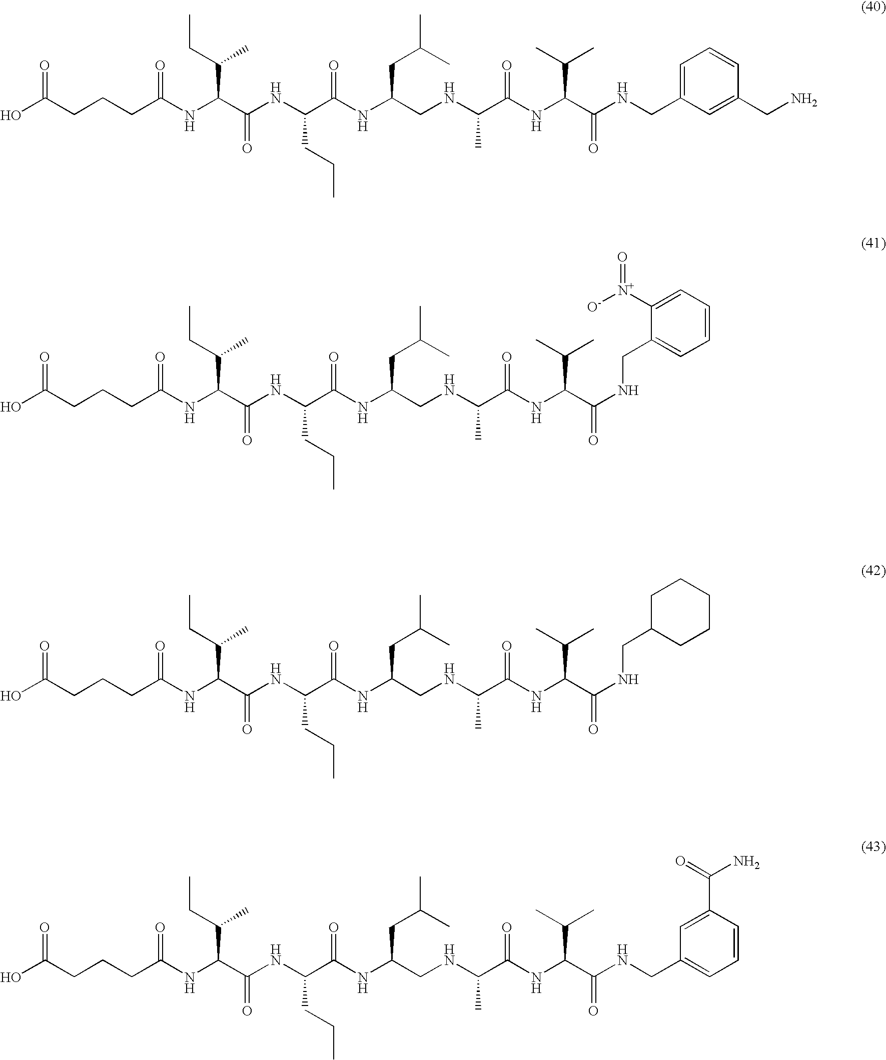

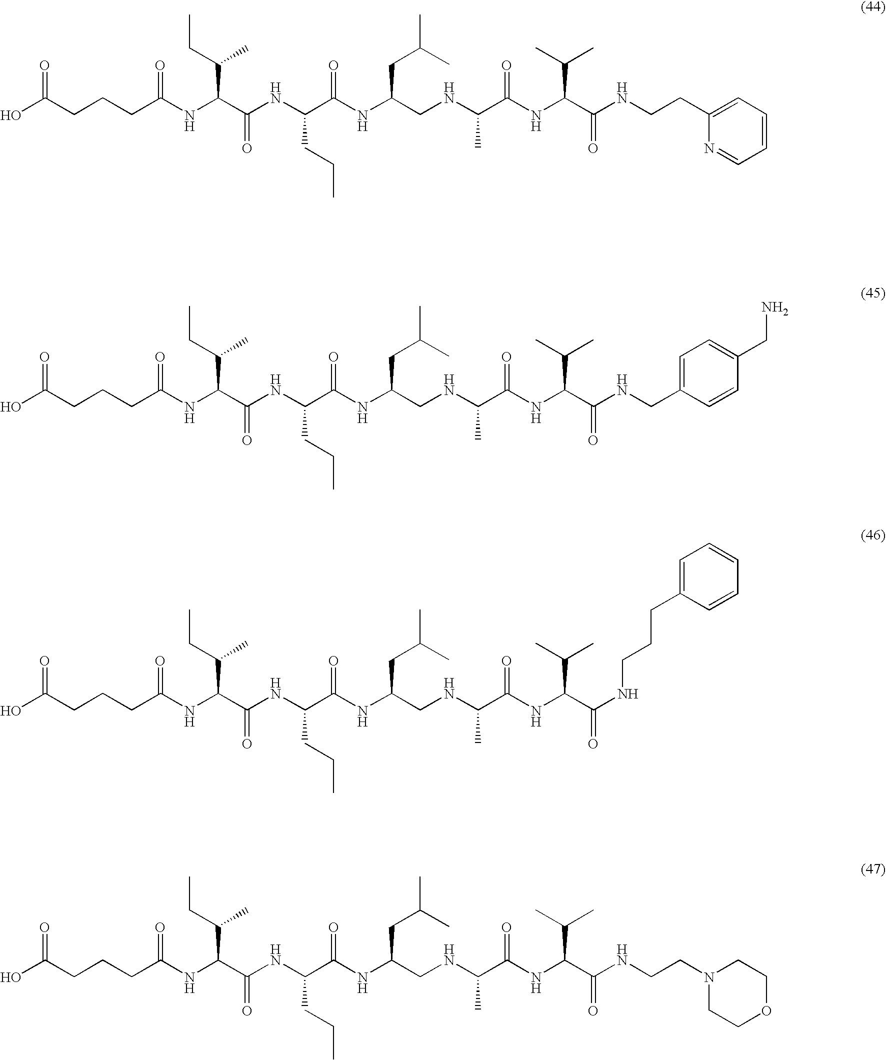

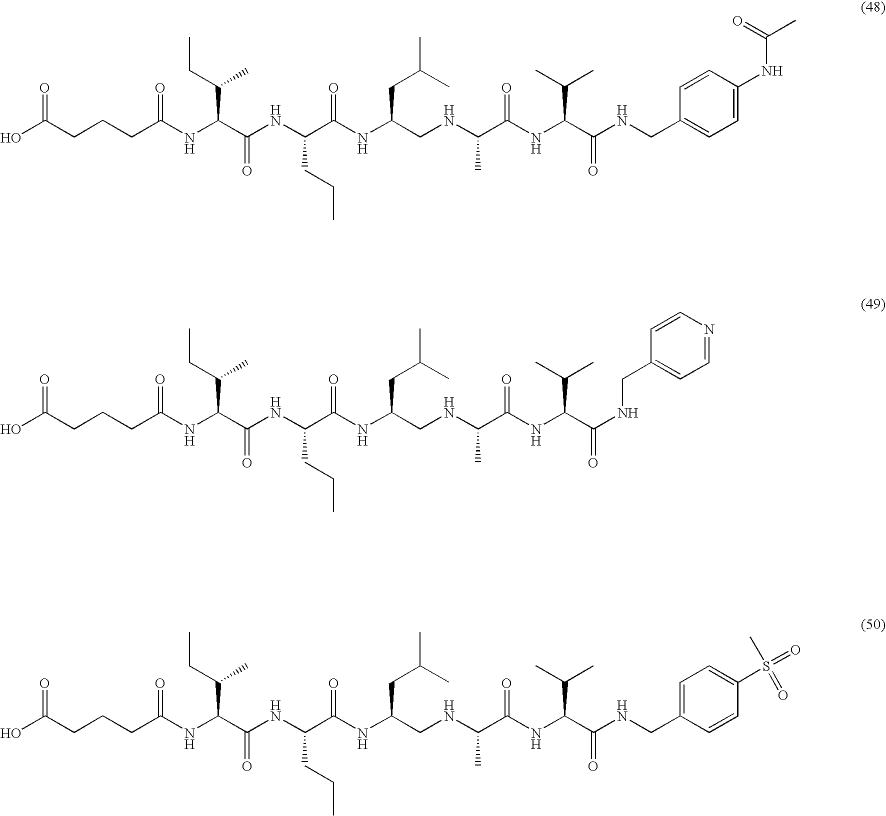

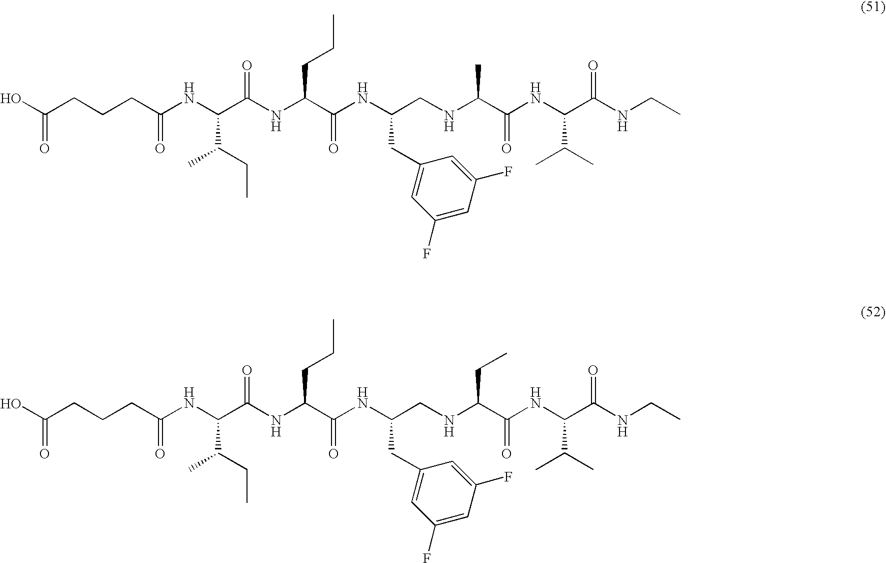

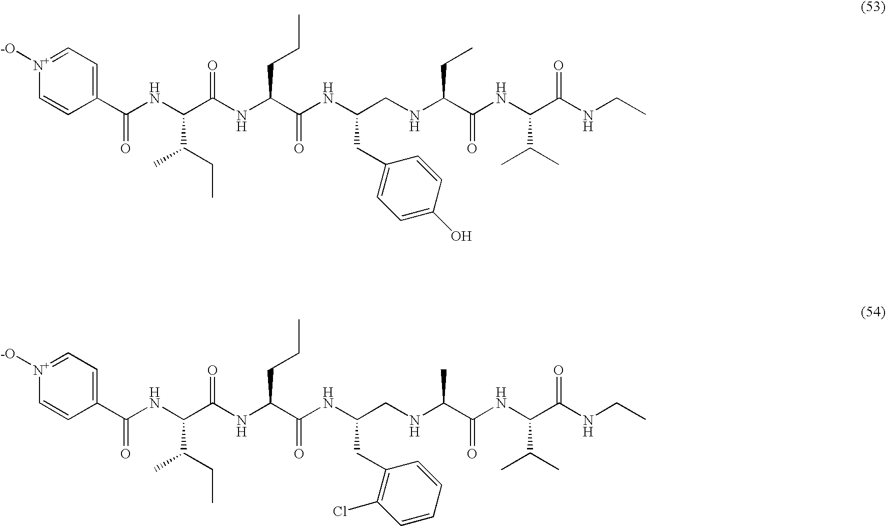

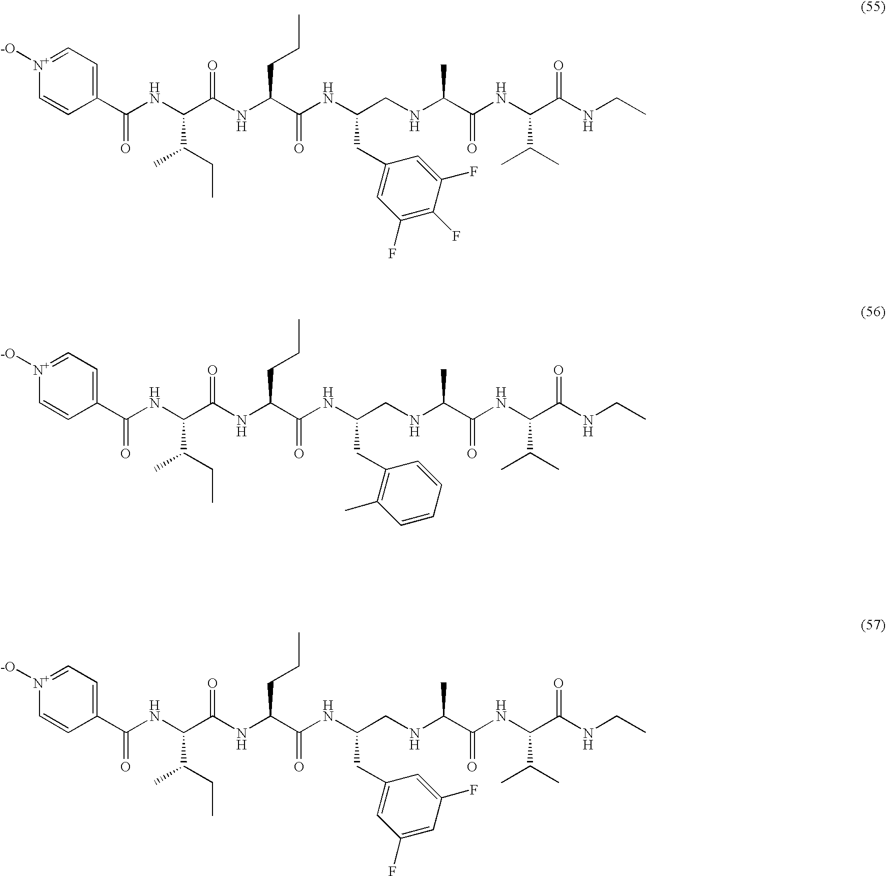

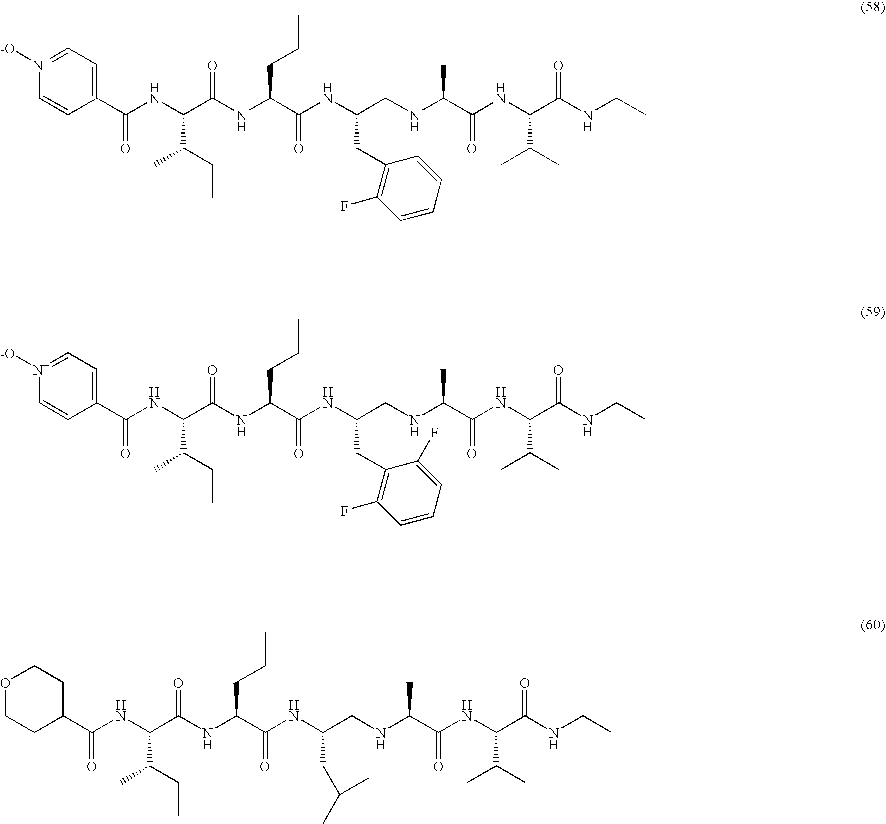

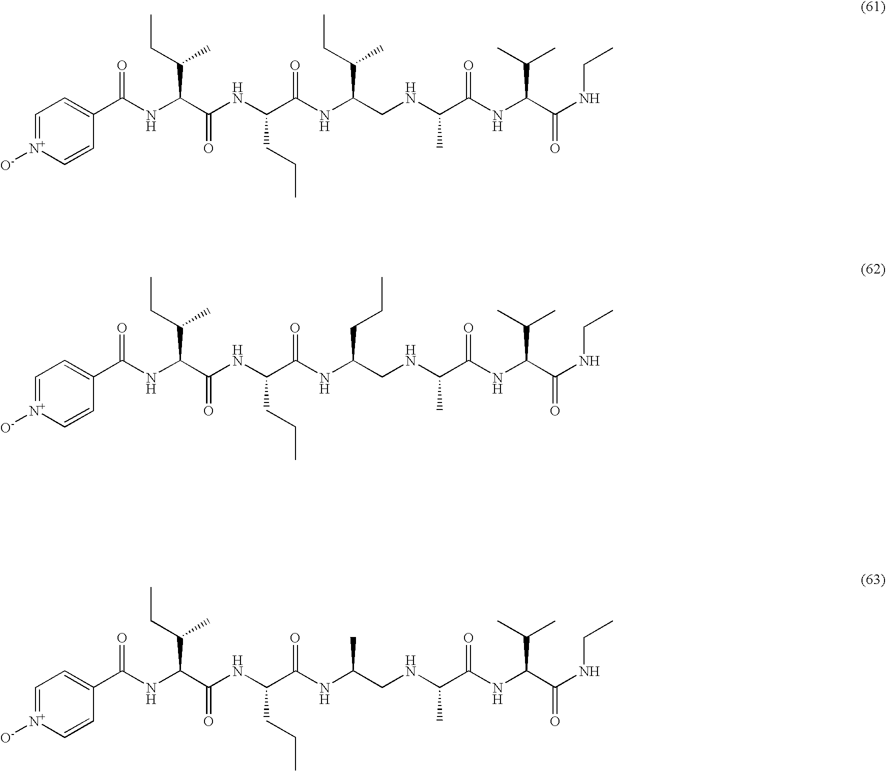

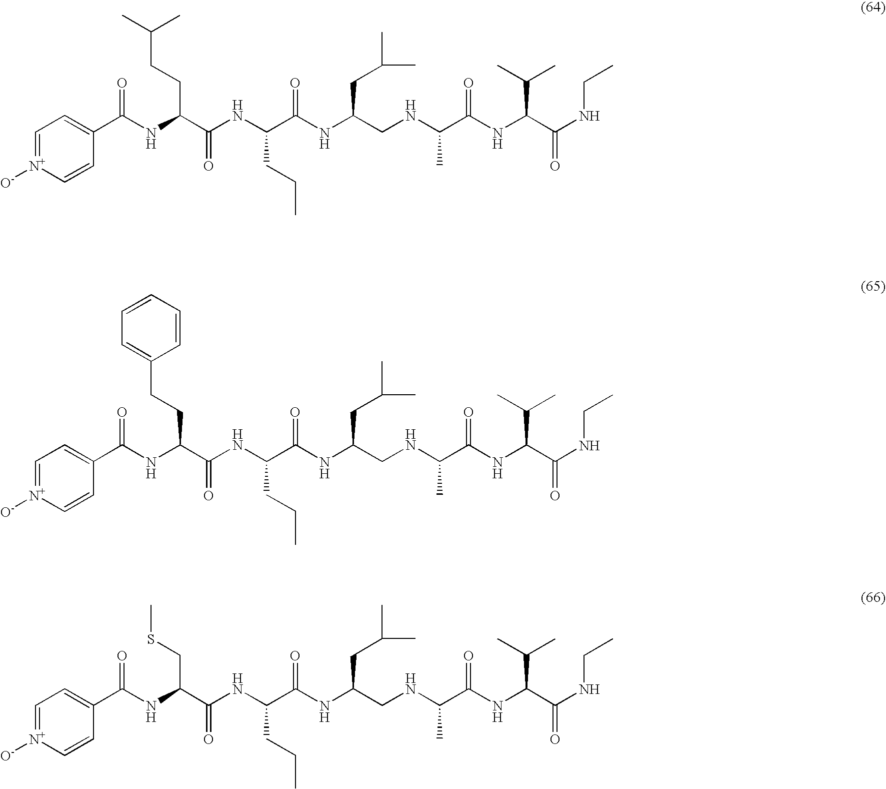

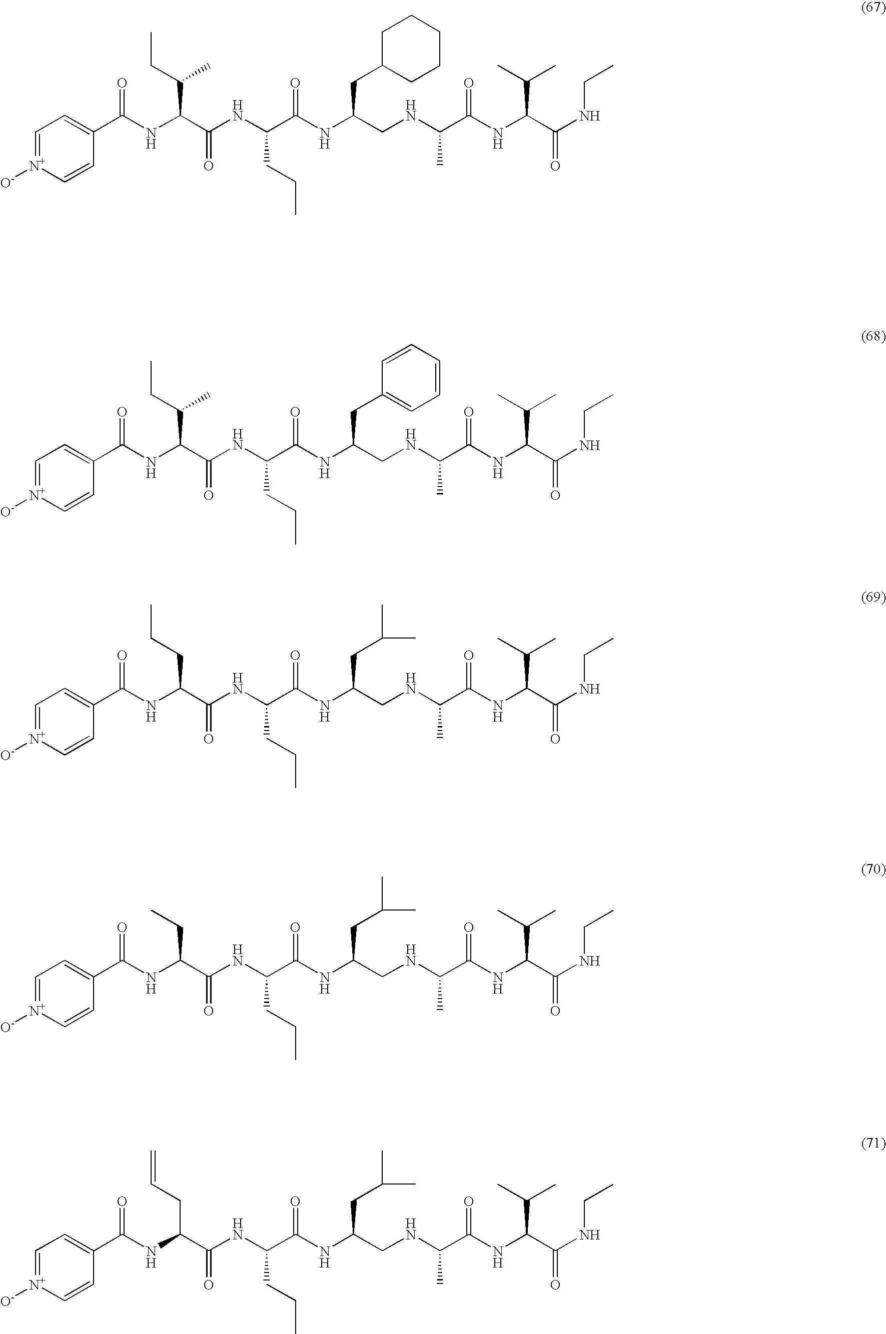

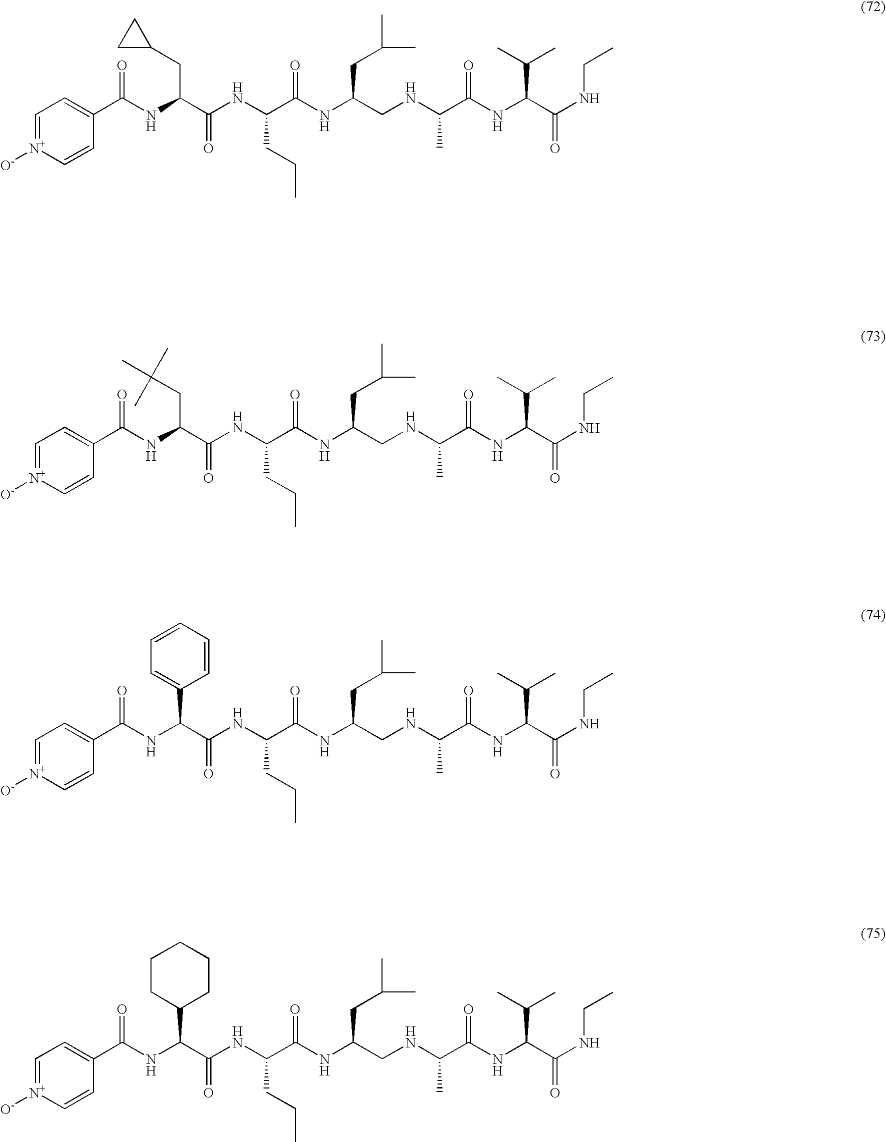

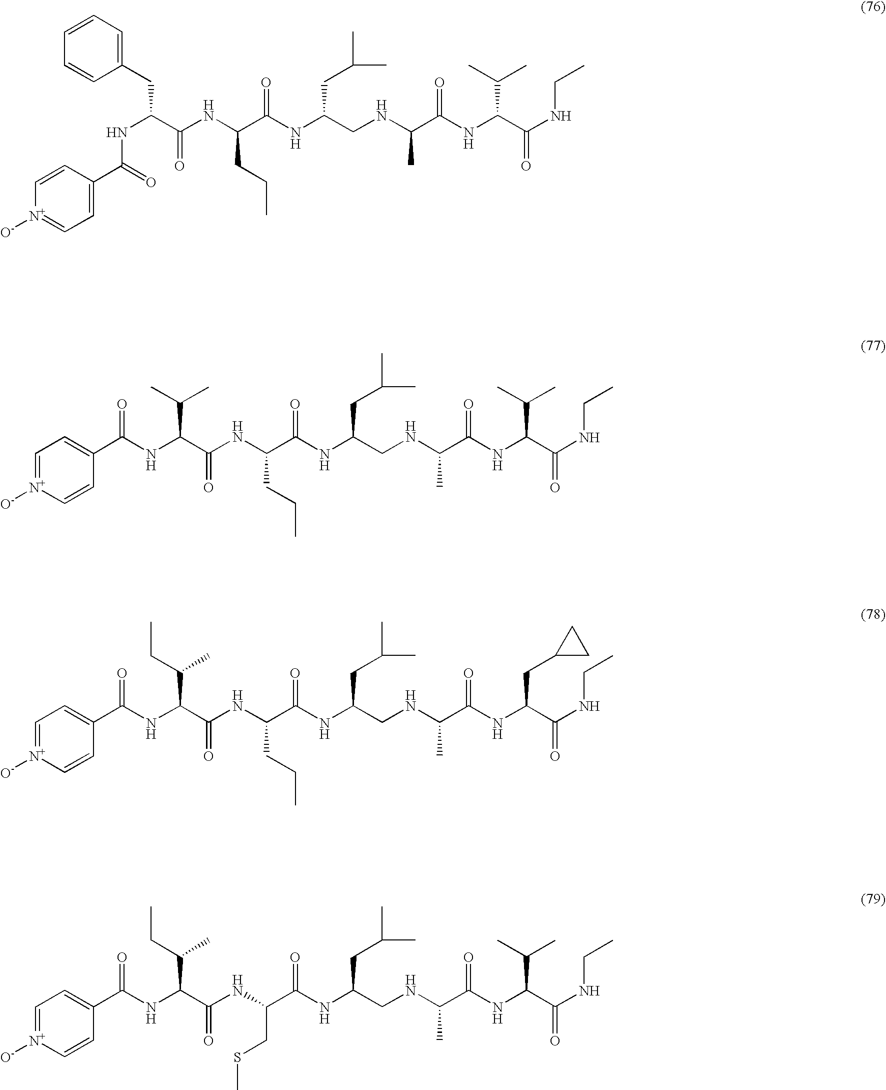

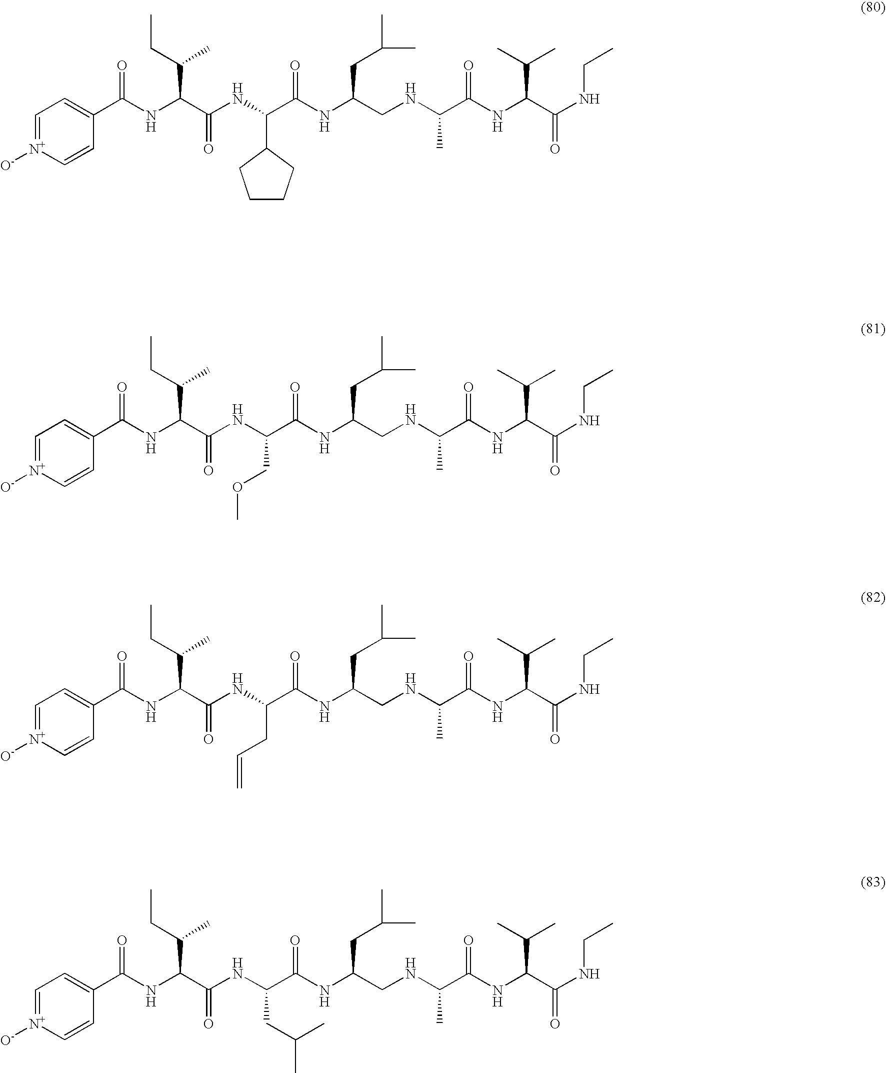

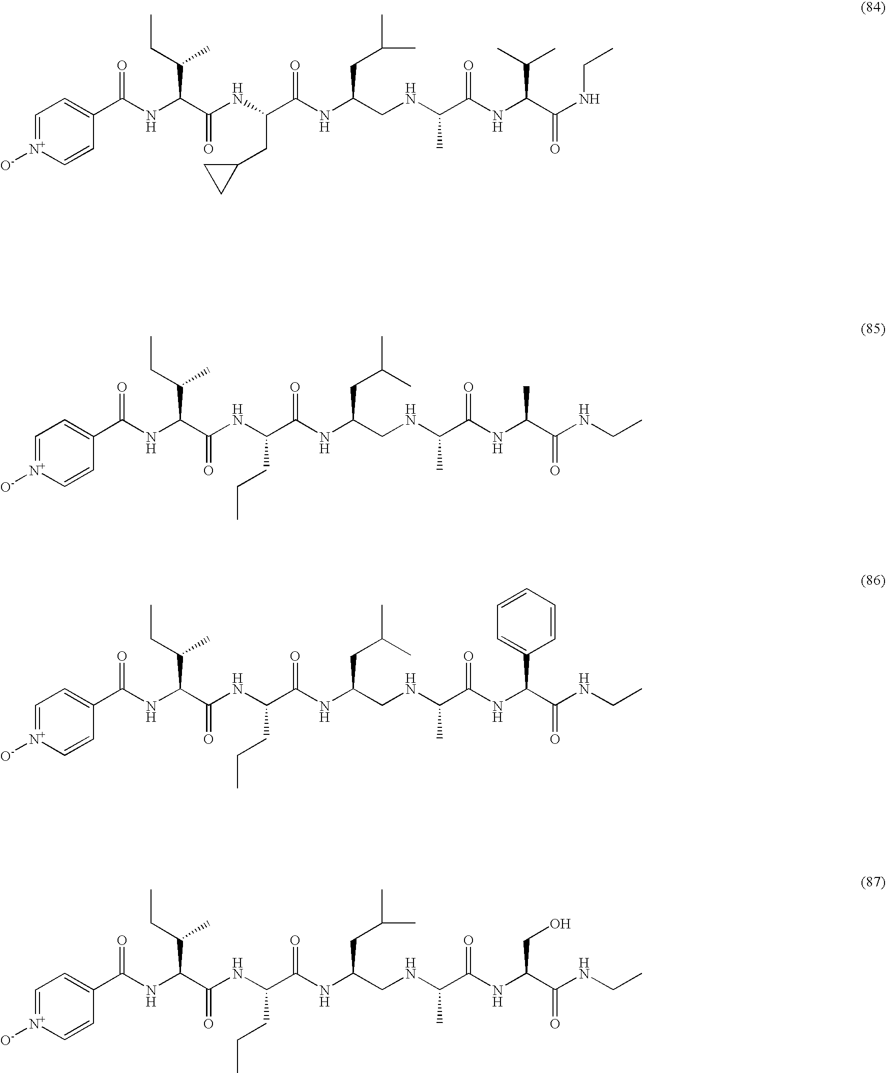

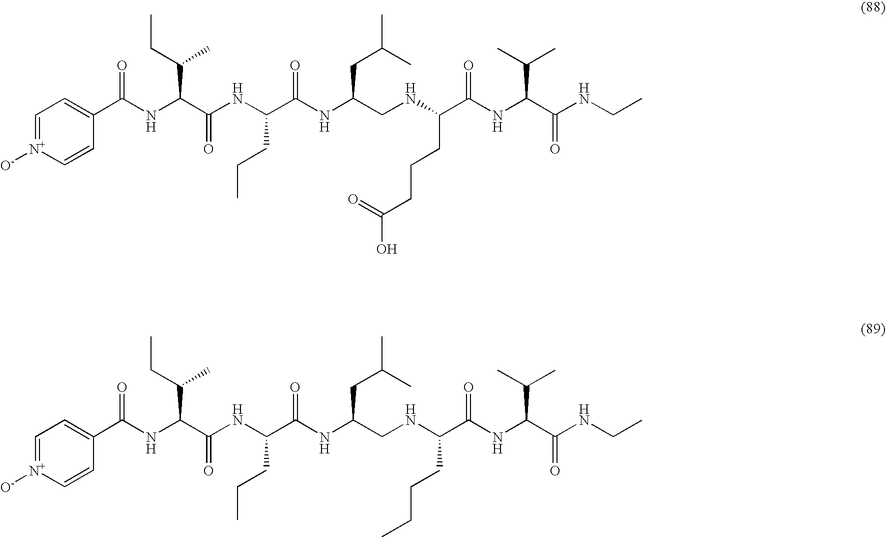

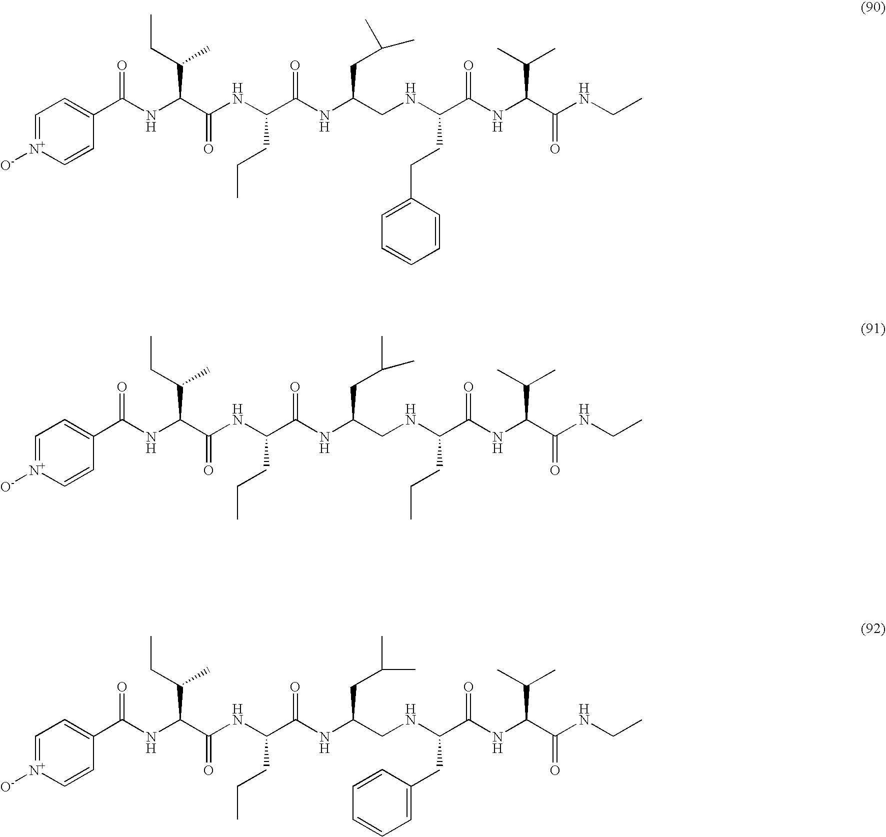

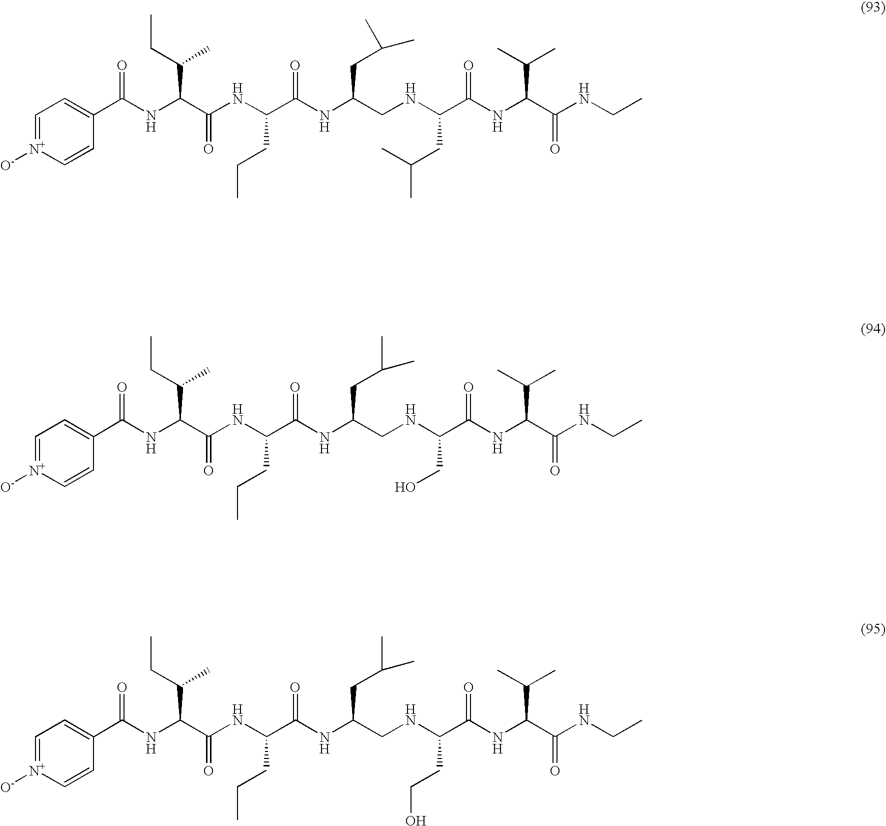

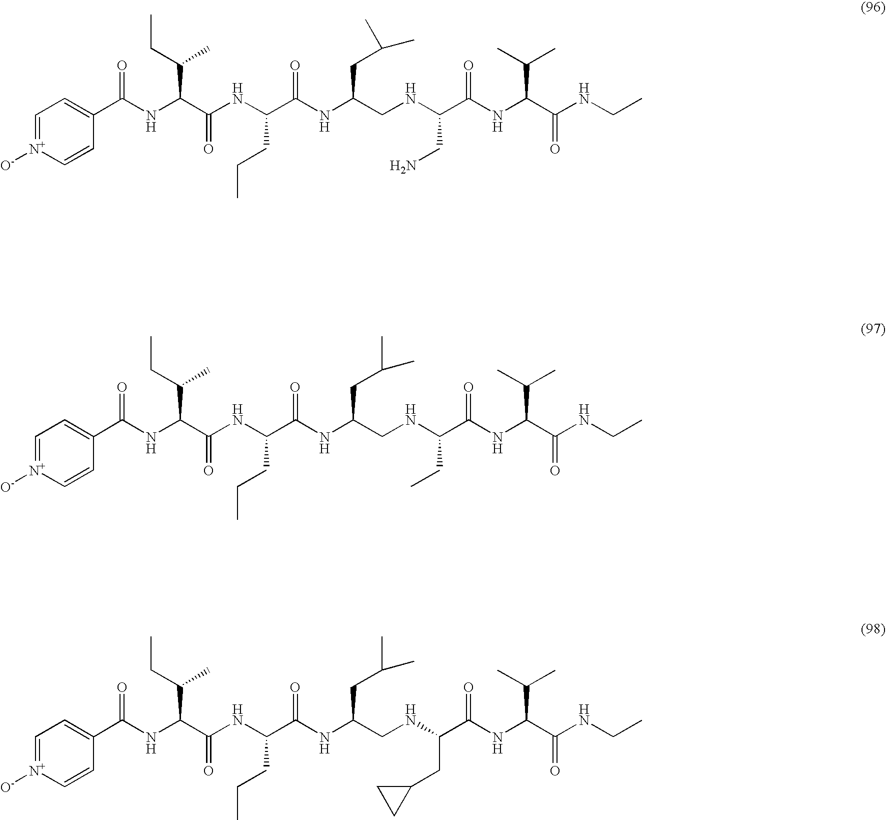

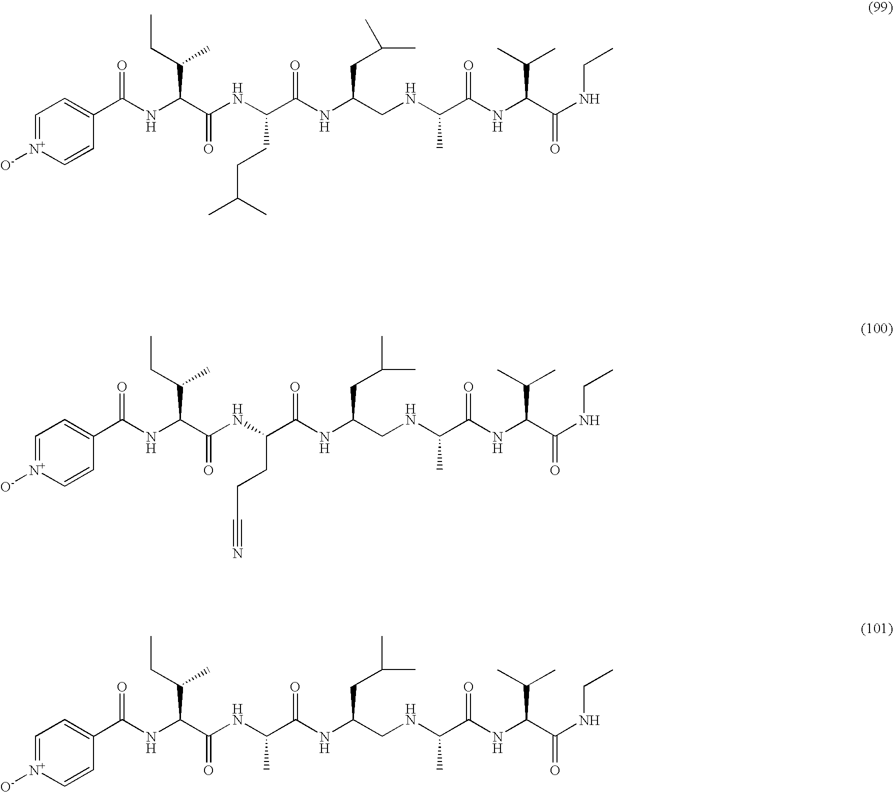

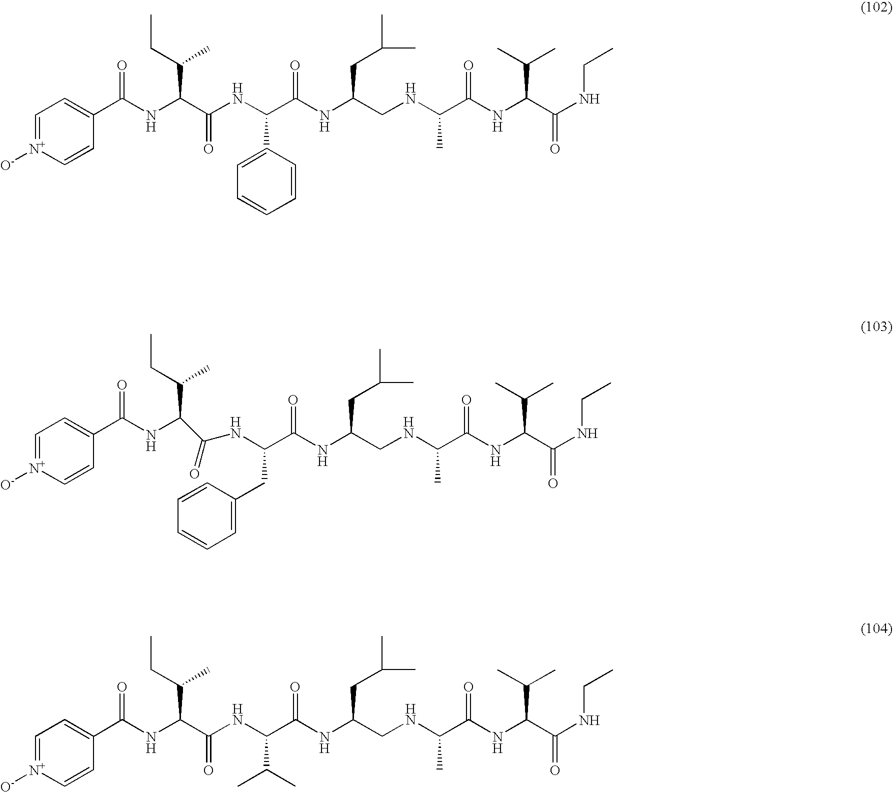

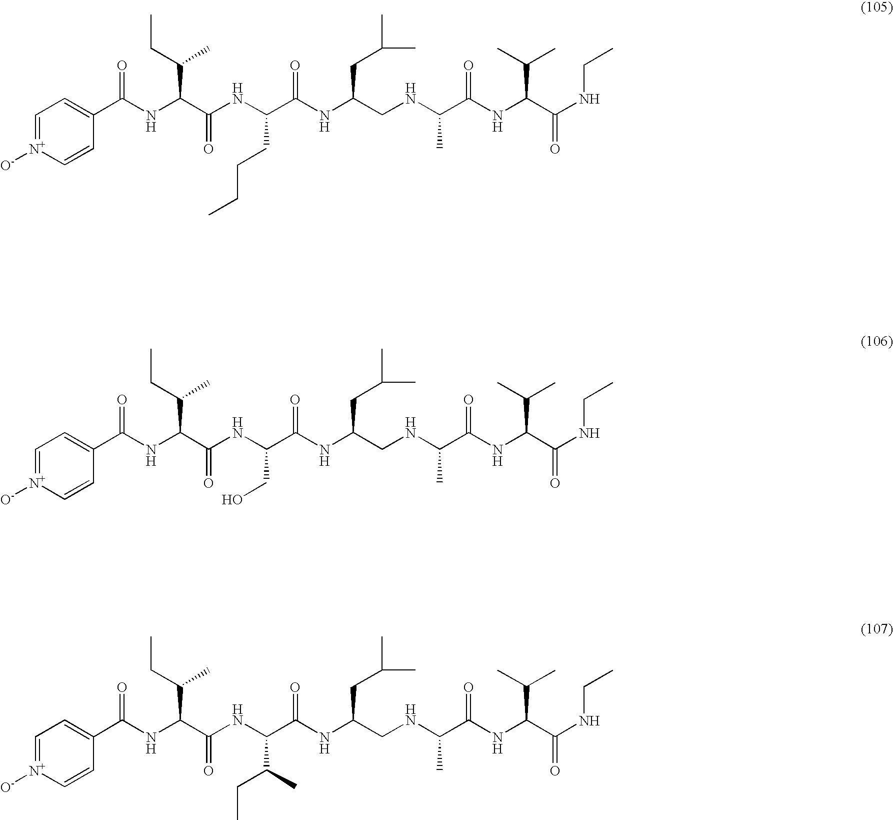

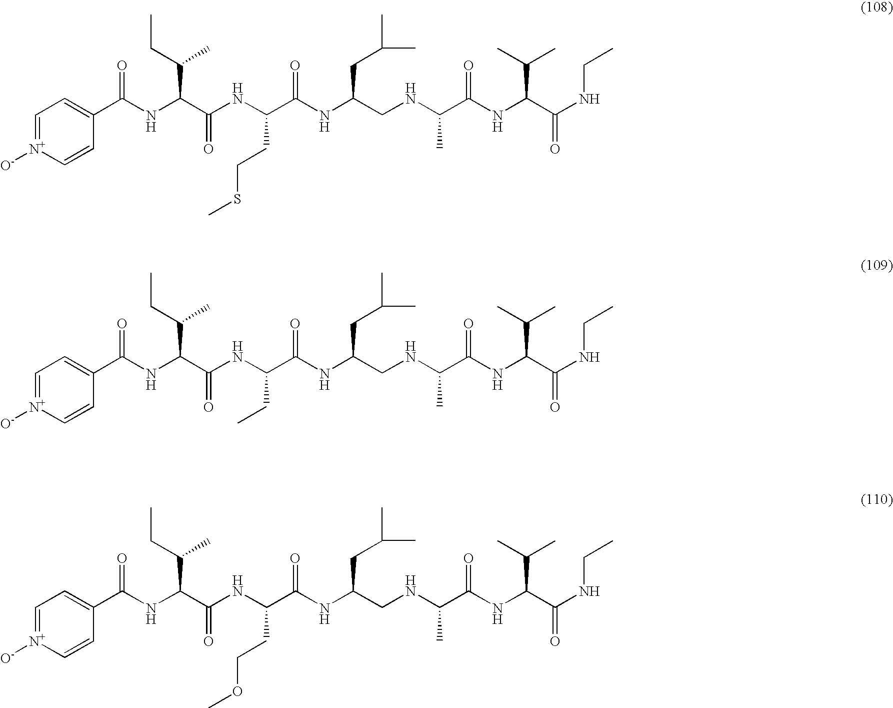

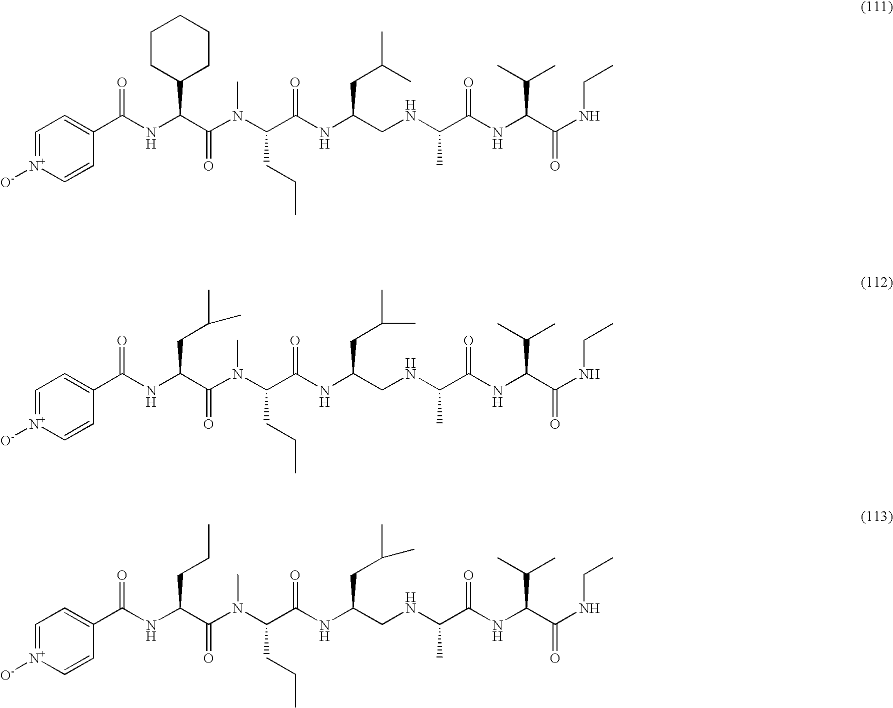

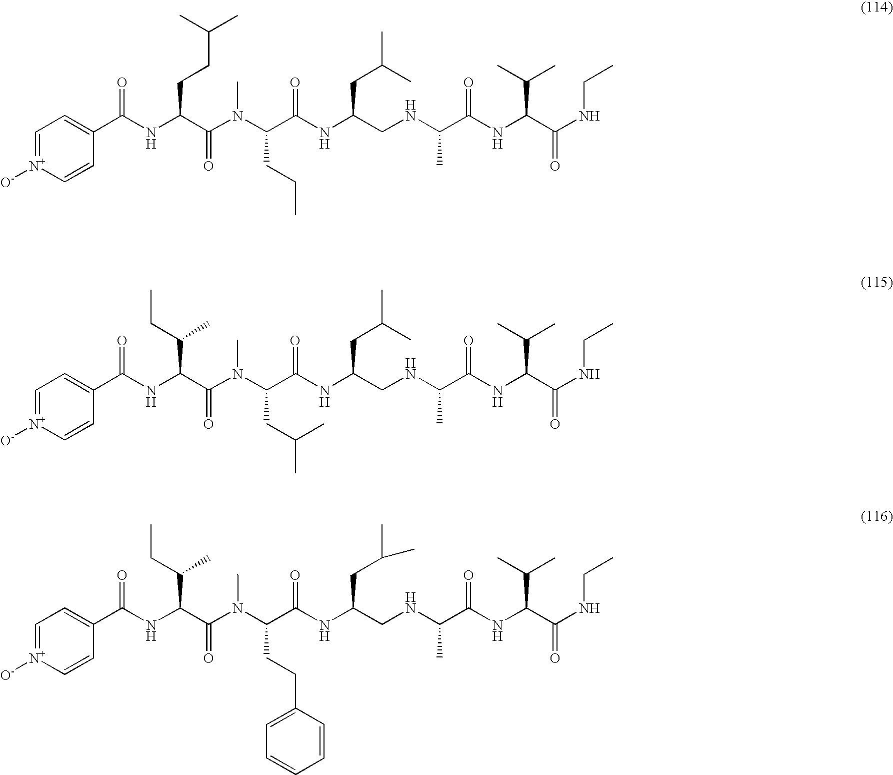

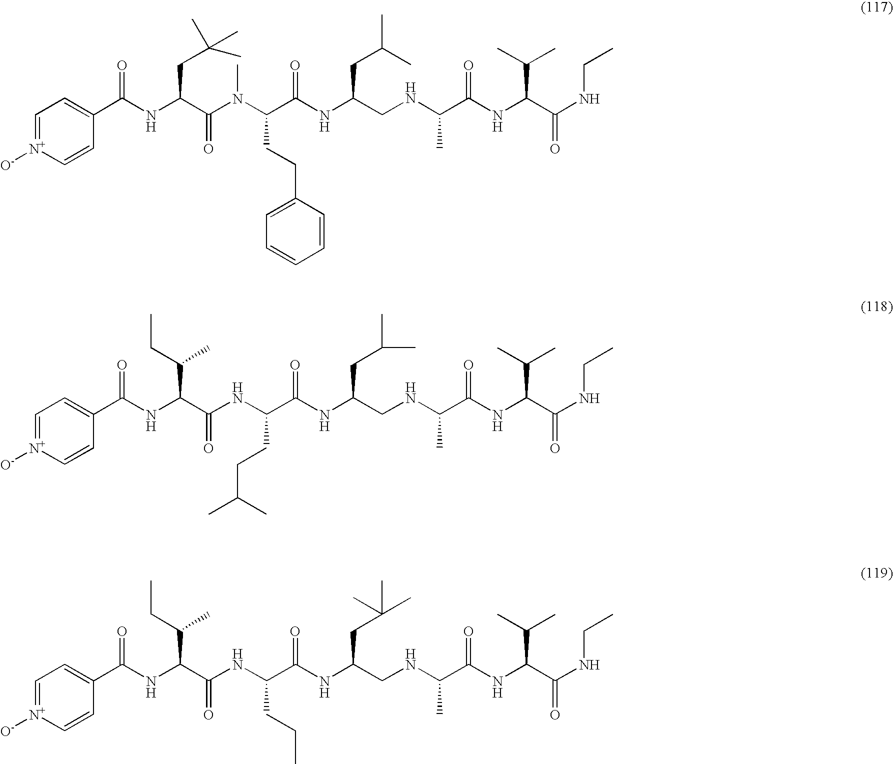

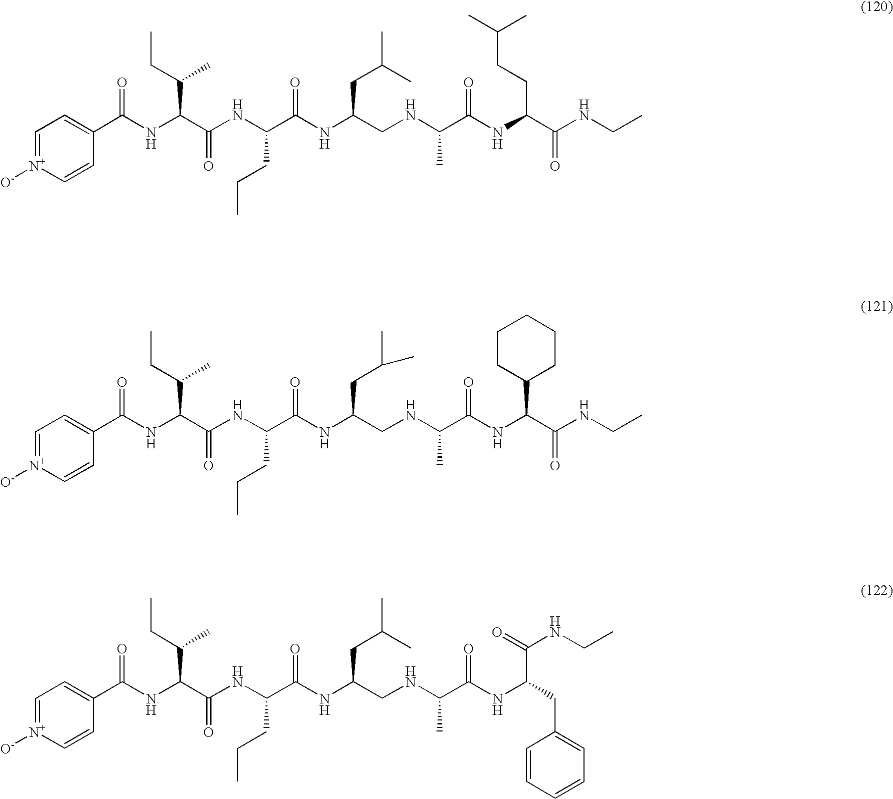

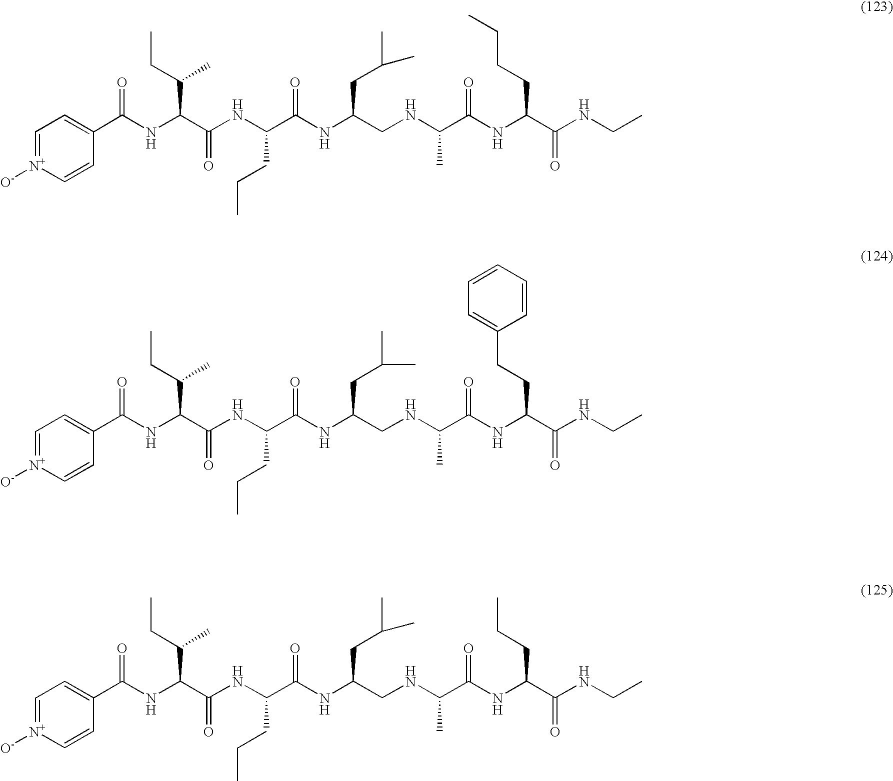

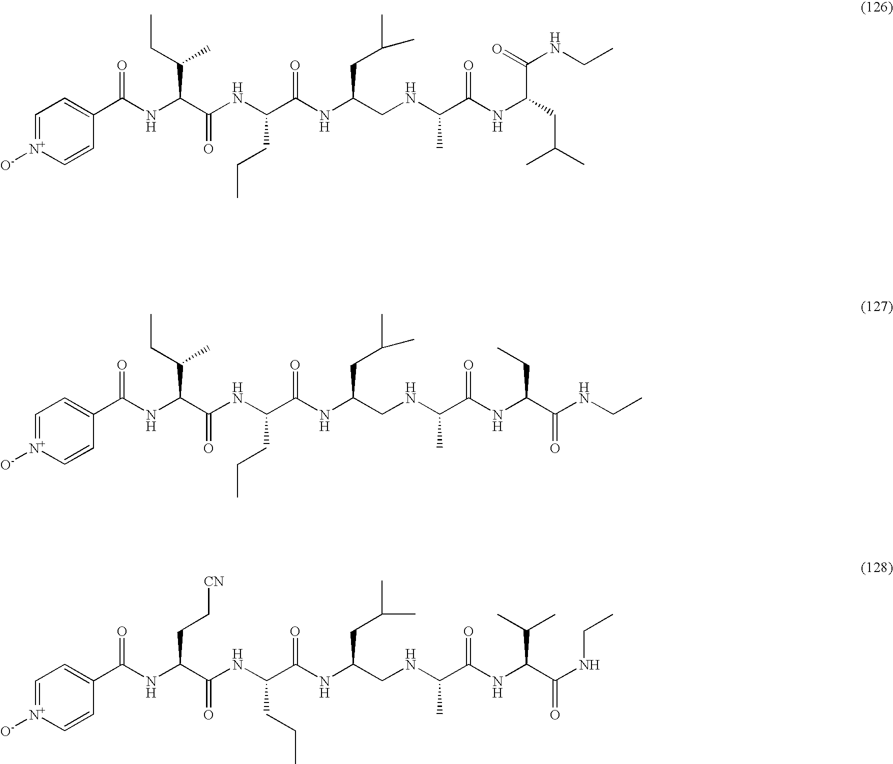

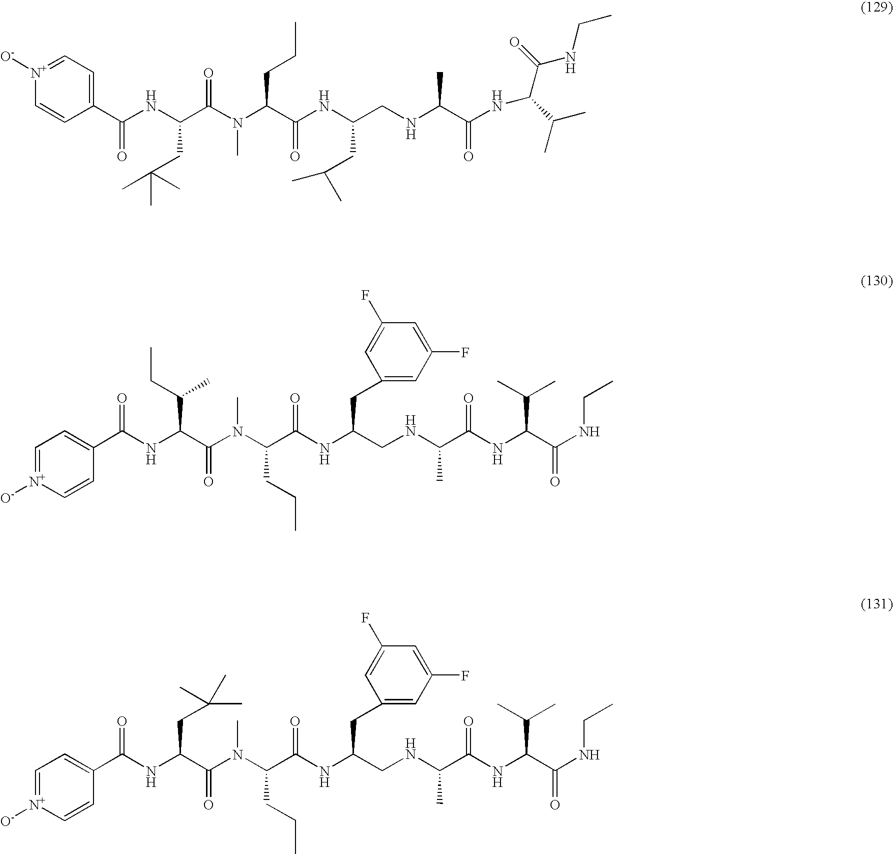

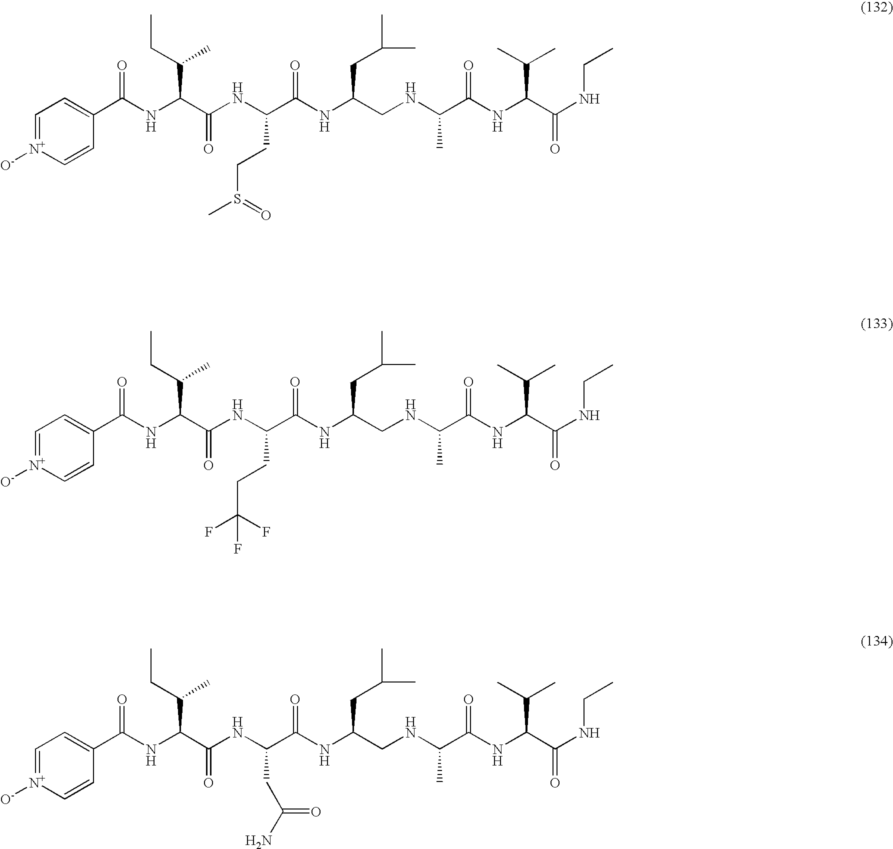

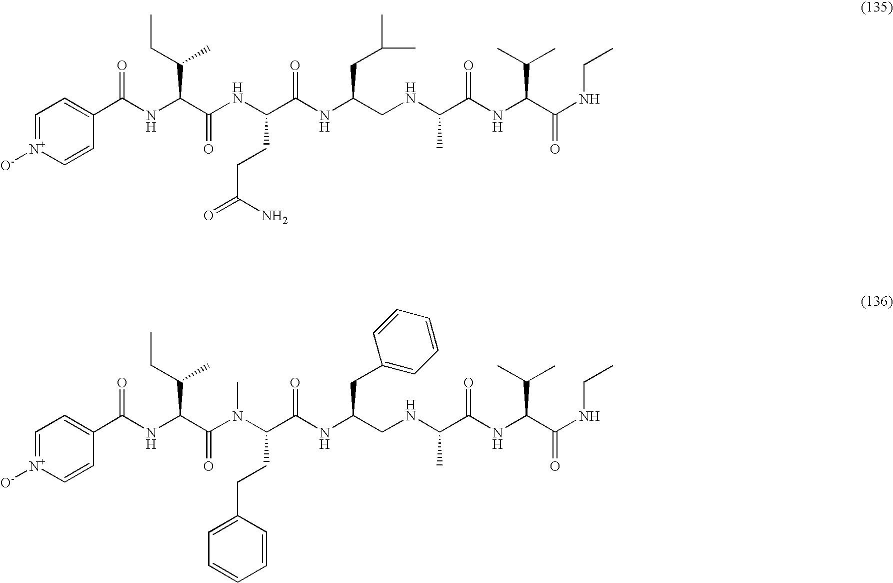

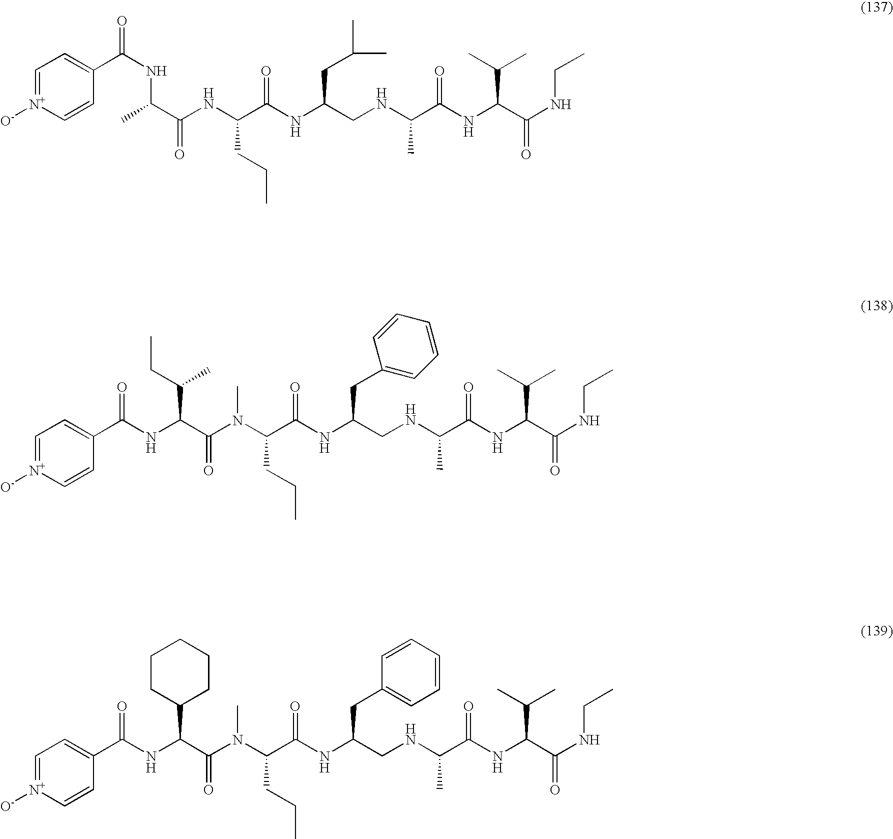

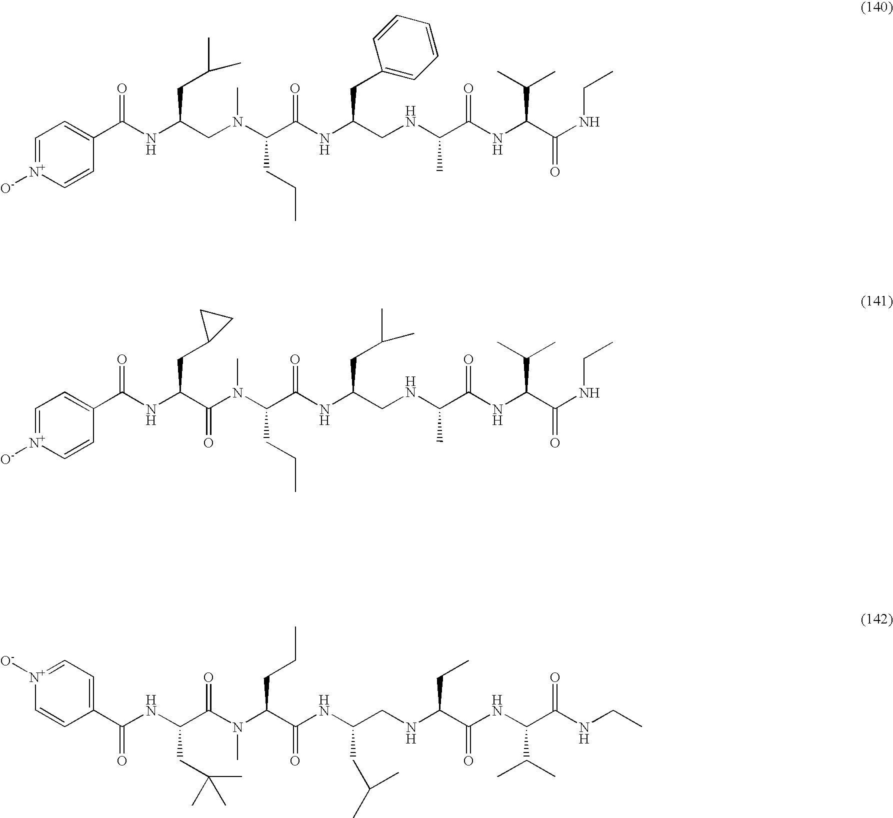

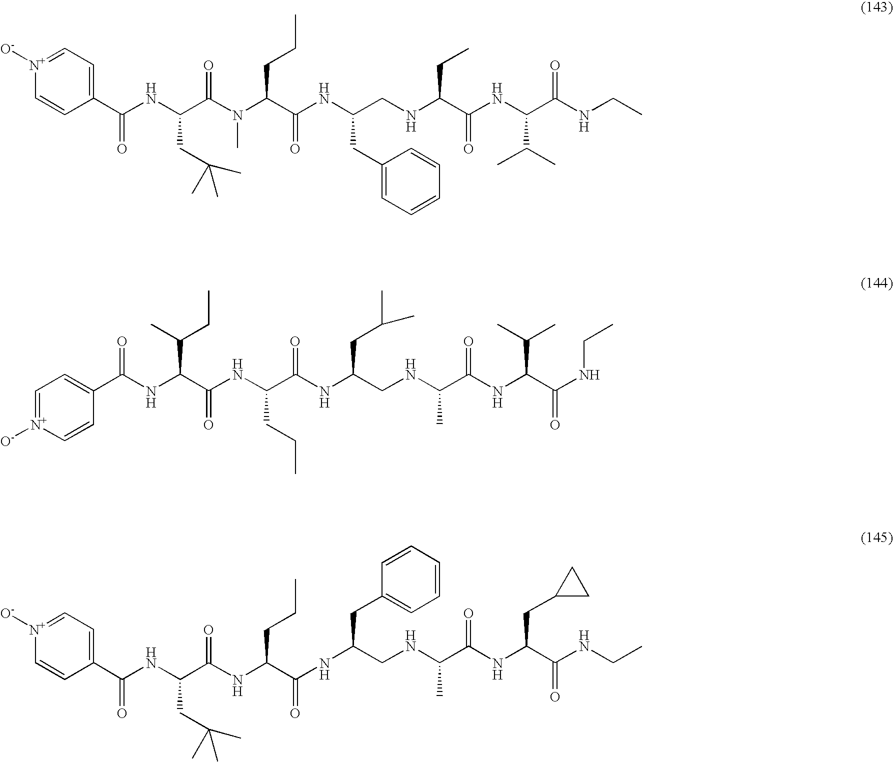

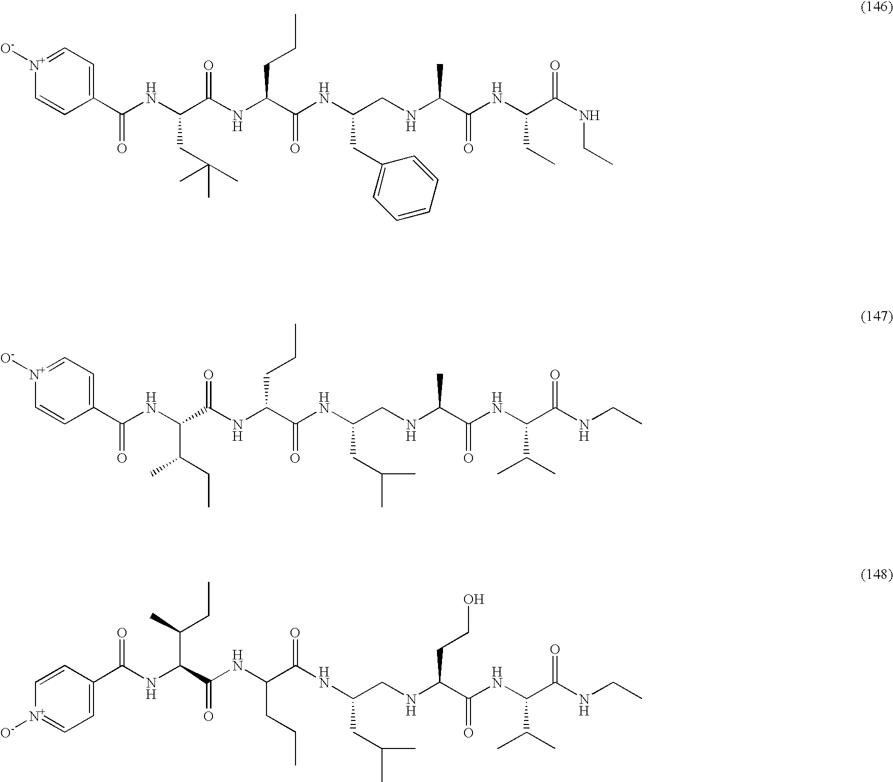

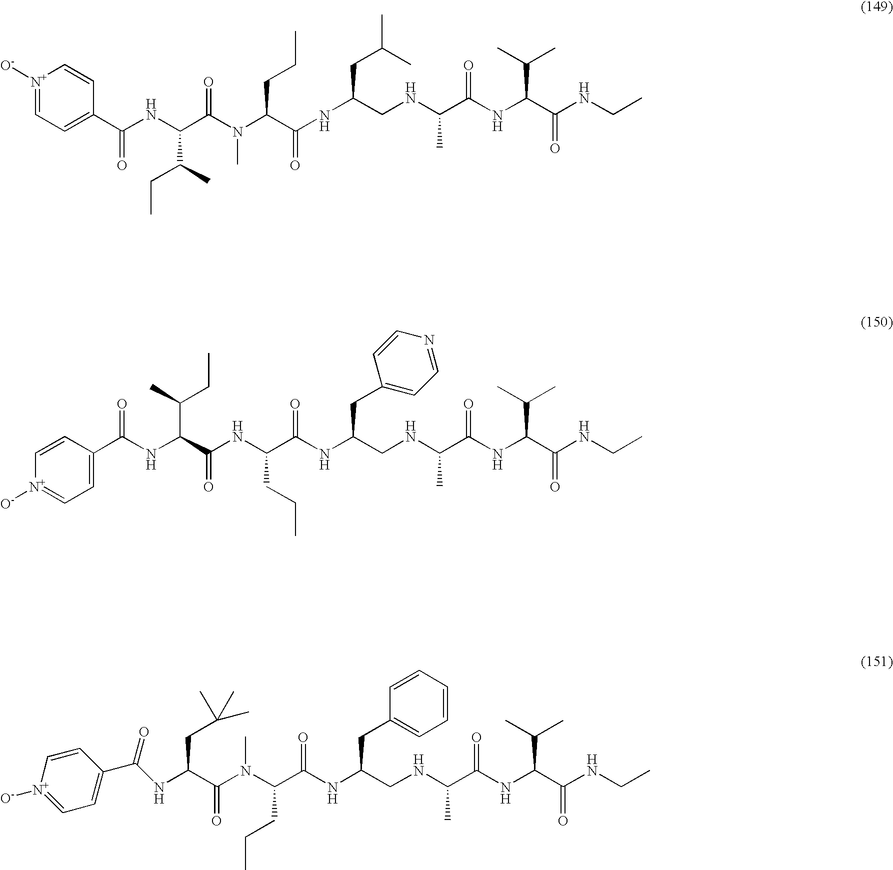

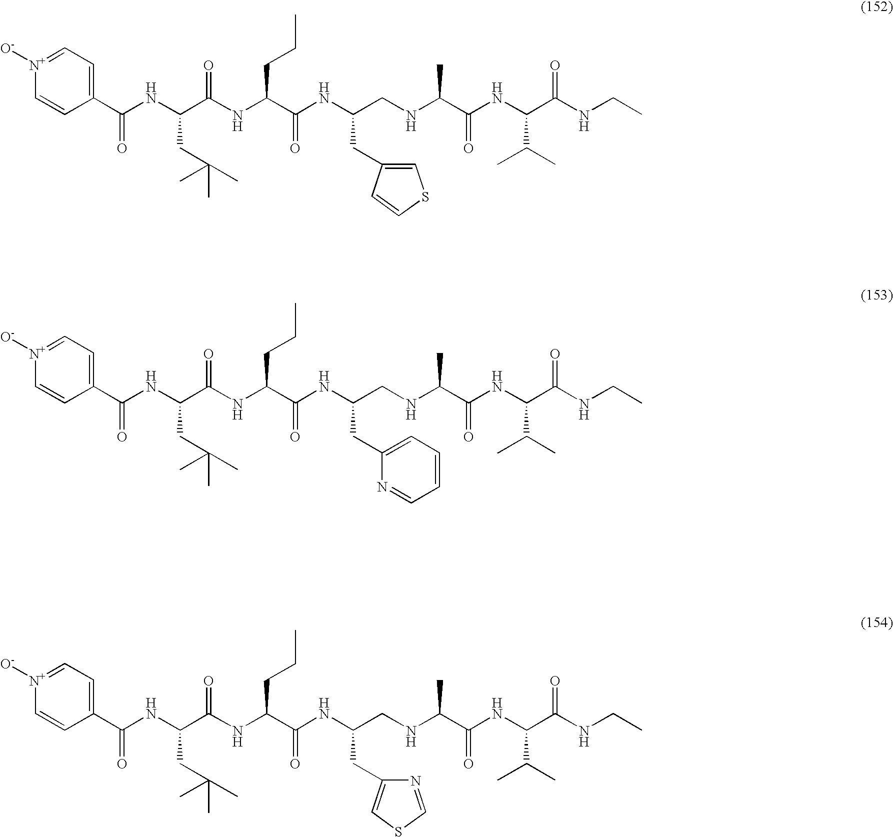

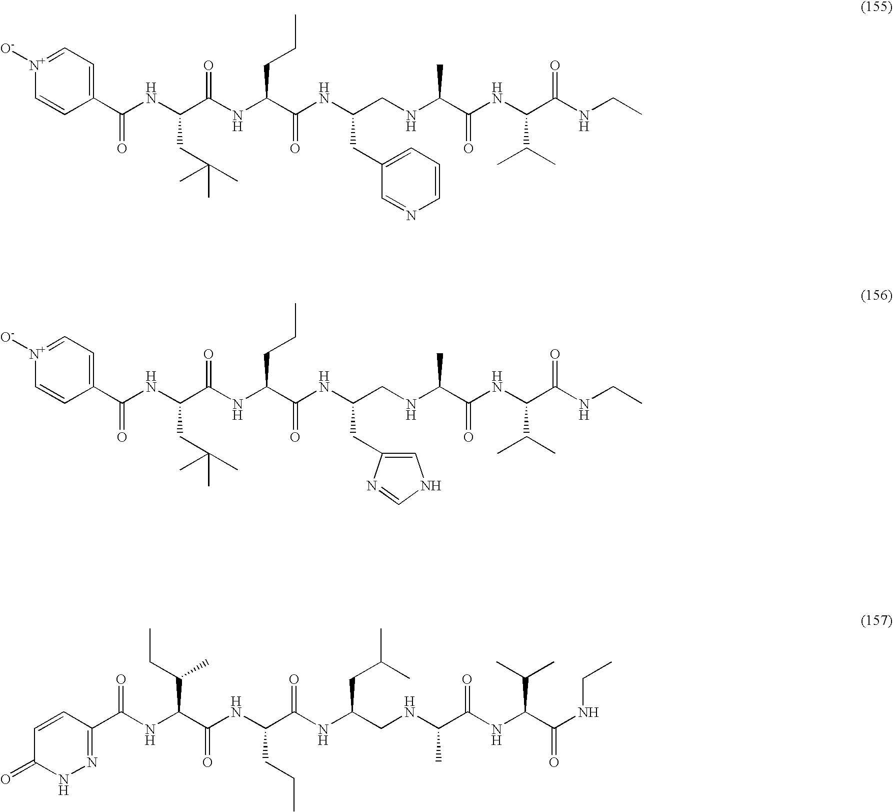

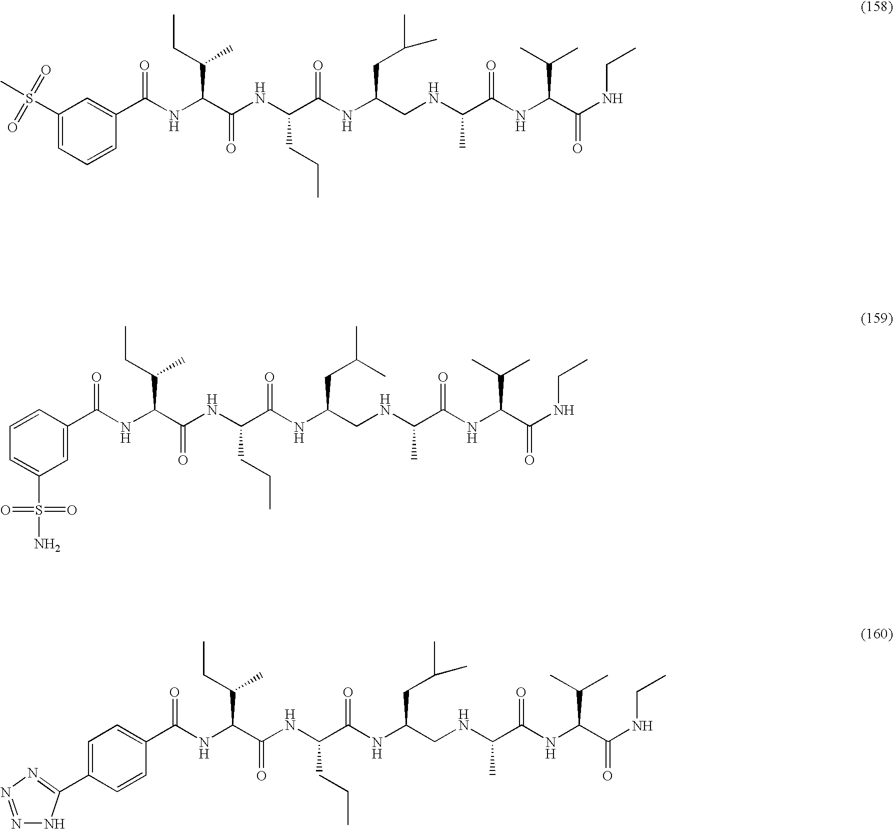

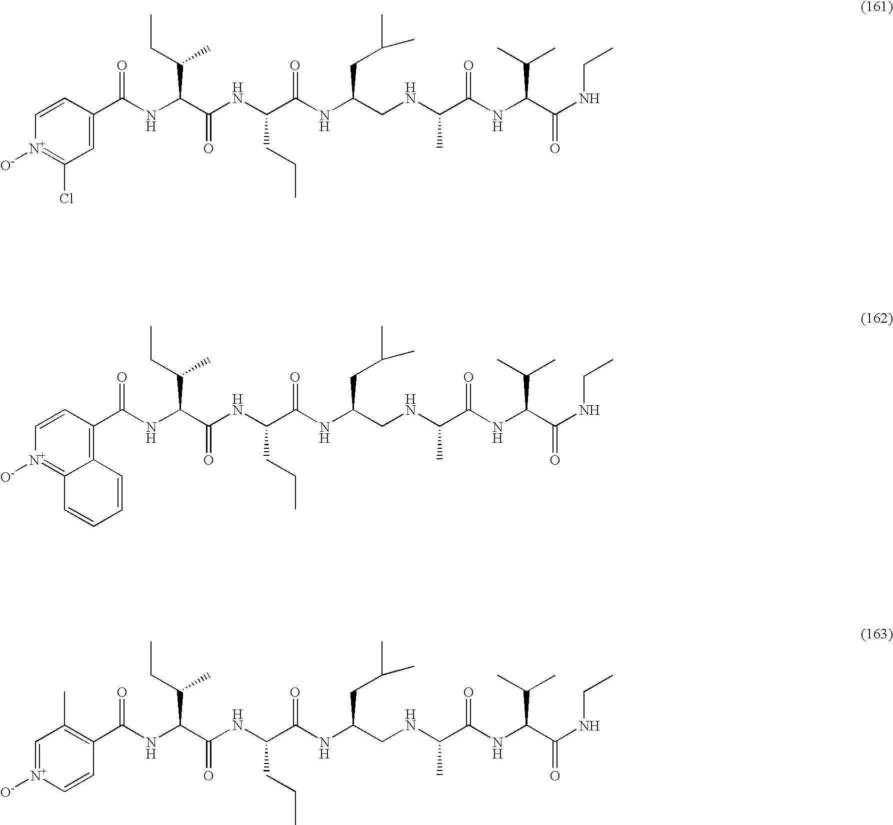

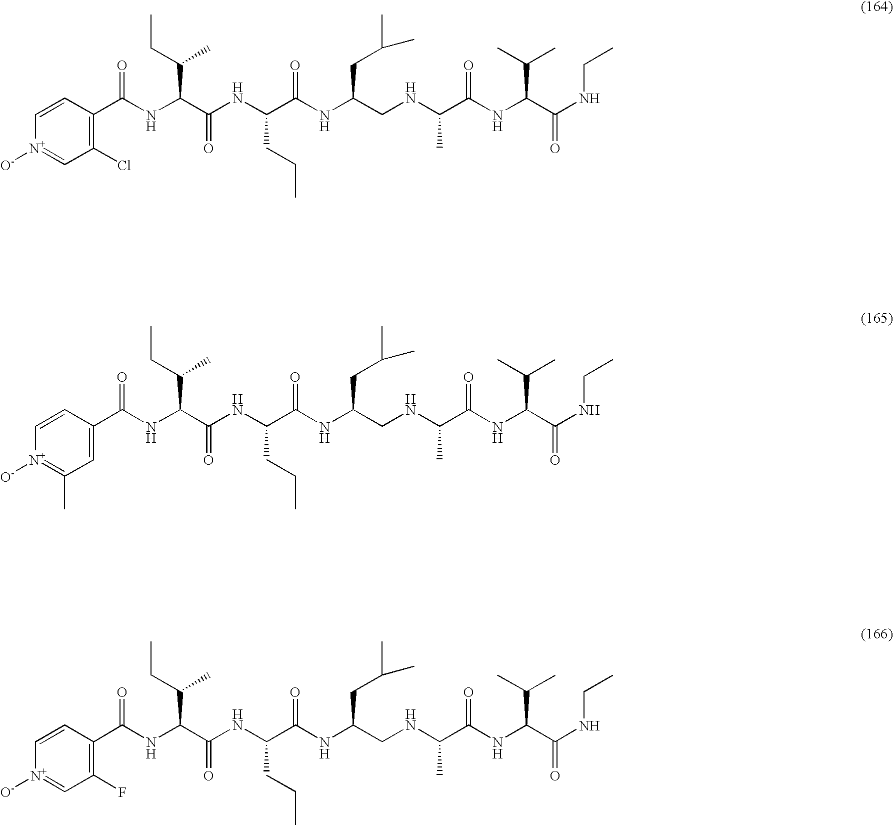

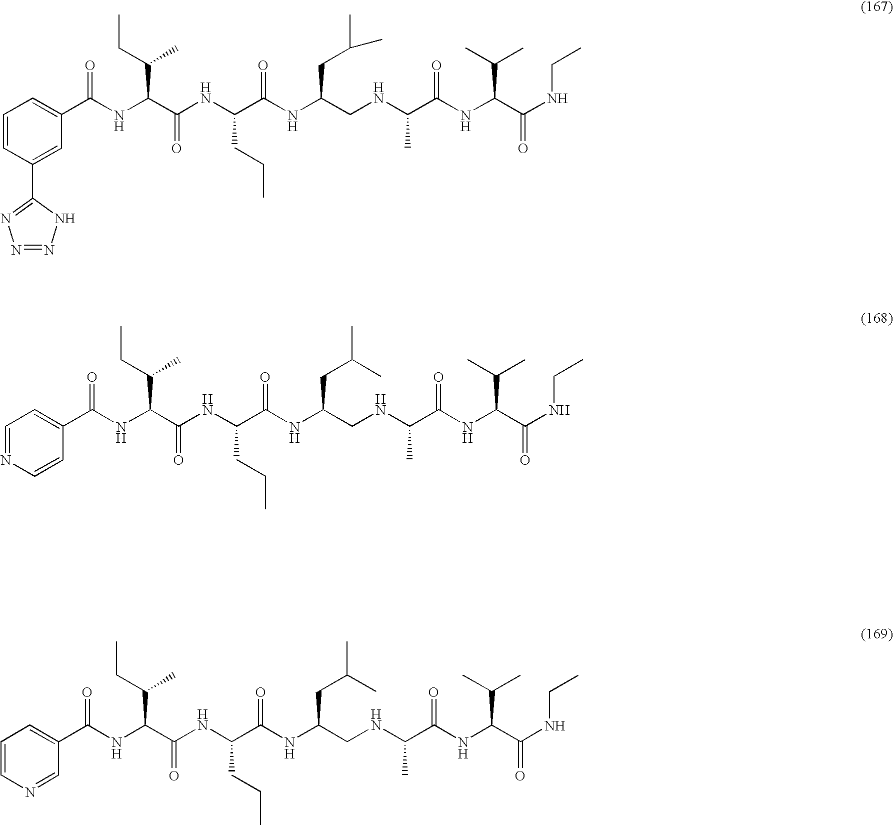

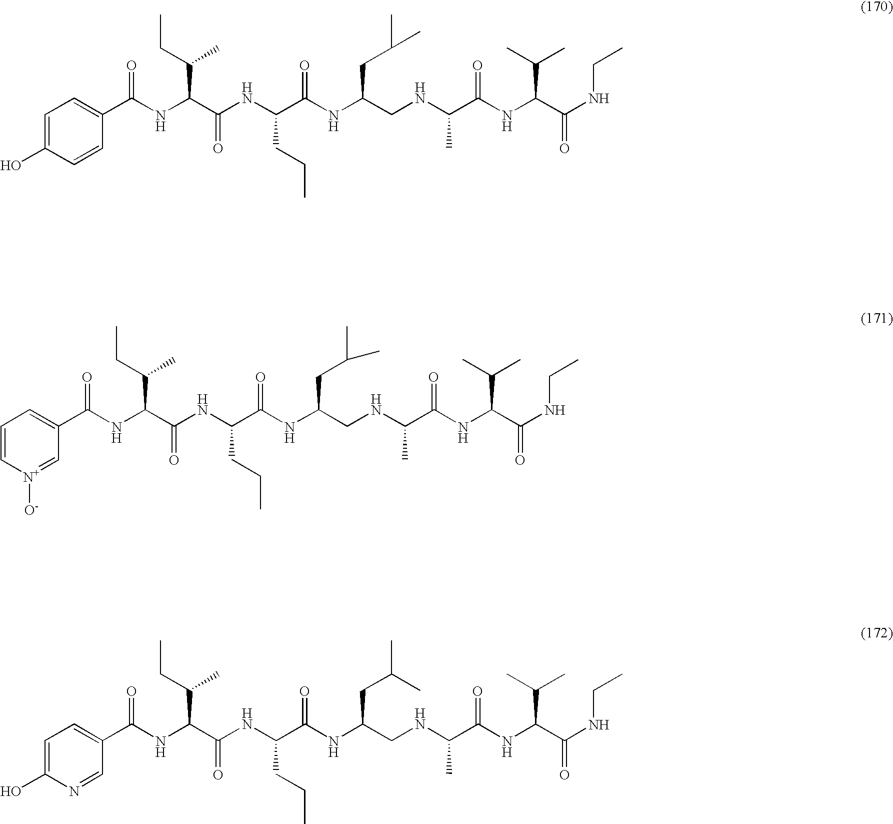

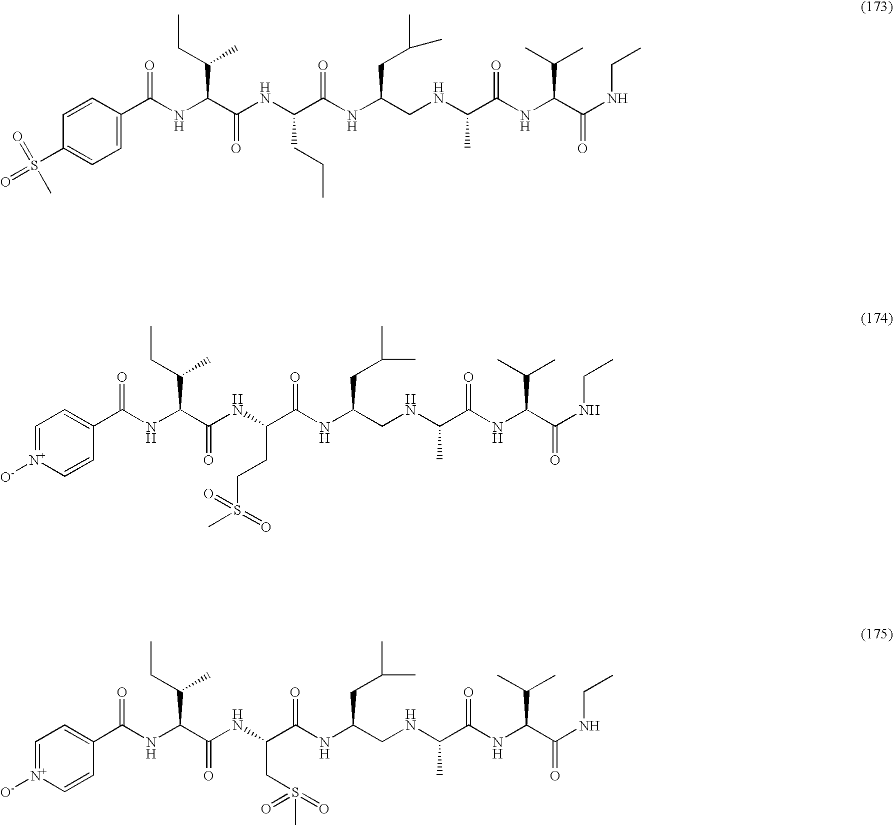

29. A pharmaceutical composition comprising the compound according to claim 1 or a pharmaceutically acceptable salt or solvate thereof.

30. The pharmaceutical composition according to claim 29 further comprising one or more additional active ingredient selected from the group consisting of atorvastatin, besipirdine, cevimeline, donepezil, eptastigmine, galantamine, glatiramer acetate, icopezil, ipidacrine, lazabemide, linopirdine, lubeluzole, memantine, metrifonate, milameline, nefiracetam, nimodipine, octreotide, rasagiline, rivastigmine, sabcomeline, sabeluzole, tacrine, valproate sodium, velnacrine, YM 796, Phenserine, and zanapezil.

31. The pharmaceutical composition according to claim 29 further comprising one or more additional antiinflammtory agents selected from the group consisting of rofecoxib, celecoxib, valdecoxib, nitroflurbiprofen, IQ-201, NCX-2216, CPI-1189, Colostrinin, ibuprofen, indomethacin, meloxicam, sulindac sulphide.

32. The pharmaceutical composition according to claim 29 further comprising one or more additional nerve growth factor and/or nerve growth modulator selected from the group consisting of: ABS-205, Inosine, KP-447, leteprinim, MCC-257, NS-521, xaliproden

33. A method of treating or preventing Alzheimer's disease, Down's syndrome, MCI (“Mild Cognitive Impairment”), Heriditary Cerebral Hemorrhage with Amyloidosis of the Dutch-Type, Cerebral Amyloid Angiopathy, Traumatic Braininjury, Stroke, Dementia, Parkinson's Disease and Parkinson's Syndrome, or central or peripheral amyloid diseases comprising administering to a patient an effective amount of the compound according to claim 1.

34. A method for inhibiting β-secretase activity, comprising exposing said β-secretase to an effective inhibitory amount of a compound according to claim 1.
